SEQUENCE LISTING (2 sequences)

SEQ ID NO: 1 - protein, 605 aa, synthetic construct
  REGION 1..605: note = Synthetic polypeptide
VISYDNYVTILDEETLKAWIAKLEKAPVFAFDTETDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENCTRLDVPLLVEVGSGENWDQAH

SEQ ID NO: 2 - protein, 606 aa, synthetic construct
  REGION 1..606: note = Synthetic polypeptide
MVISYDNYVTILDEETLKAWIAKLEKAPVFAFATATDSLDNISANLVGLSFAIEPGVAAYIPVAHDYLDAPDQISRERALELLKPLLEDEKALKVGQNLKYDRGILANYGIELRGIAFDTMLESYILNSVAGRHDMDSLAERWLKHKTITFEEIAGKGKNQLTFNQIALEEAGRYAAEDADVTLQLHLKMWPDLQKHKGPLNVFENIEMPLVPVLSRIERNGVKIDPKVLHNHSEELTLRLAELEKKAHEIAGEEFNLSSTKQLQTILFEKQGIKPLKKTPGGAPSTSEEVLEELALDYPLPKVILEYRGLAKLKSTYTDKLPLMINPKTGRVHTSYHQAVTATGRLSSTDPNLQNIPVRNEEGRRIRQAFIAPEDYVIVSADYSQIELRIMAHLSRDKGLLTAFAEGKDIHRATAAEVFGLPLETVTSEQRRSAKAINFGLIYGMSAFGLARQLNIPRKEAQKYMDLYFERYPGVLEYMERTRAQAKEQGYVETLDGRRLYLPDIKSSNGARRAAAERAAINAPMQGTAADIIKRAMIAVDAWLQAEQPRVRMIMQVHDELVFEVHKDDVDAVAKQIHQLMENSTRLDVPLLVEVGSGENWDQAH

(19) United States
(12) Patent Application Publication
(10) Pub. No.: US 2023/0193361 A1
(43) Pub. Date: Jun. 22, 2023

(54) METHODS AND COMPOSITIONS USEFUL FOR NUCLEIC ACID SEQUENCING

(71) Applicant: Singular Genomics Systems, Inc., San Diego, CA (US)

(72) Inventors: Eli N. Glezer, Del Mar, CA (US); Ronald Graham, Carlsbad, CA (US); Michael Krause, San Diego, CA (US)

(21) Appl. No.: 18/050,688

(22) Filed: Oct. 28, 2022

Related U.S. Application Data
(63) Continuation of application No. PCT/US2022/034761, filed on Jun. 23, 2022.
(60) Provisional application No. 63/214,614, filed on Jun. 24, 2021.

(51) Int. Cl. C12Q 1/6818; C12Q 1/6825

(52) U.S. Cl. CPC C12Q 1/6818; C12Q 1/6825; G01N 2458/00

(57) ABSTRACT

Disclosed herein, inter alia, are modified nucleotides and methods of using the same in nucleic acid sequencing reactions.

### CROSS-REFERENCES TO RELATED APPLICATIONS

This application claims the benefit of U.S. Provisional Application No. 63/214,614, filed Jun. 24, 2021, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 051385-550001WO_Sequence_Listing_ST25.txt, created Jun. 20, 2022, 10,871 bytes, machine format IBM-PC, MS Windows operating system, is hereby incorporated by reference.

### BACKGROUND

Typical sequencing-by-synthesis (SBS) methodologies employ serial incorporation and detection of labeled nucleotide analogues. For example, high-throughput SBS technology uses cleavable fluorescent nucleotide reversible terminator (NRT) sequencing chemistry. These cleavable fluorescent NRTs were designed based on the following rationale: each of the four nucleotide types (dA, dC, dG, dT, and/or dU) is modified by attaching a unique cleavable fluorophore to the specific location of the nucleobase and capping the 3′-OH group of the nucleotide sugar with a small reversible moiety (also referred to herein as a reversible terminator) so that they are still recognized by DNA polymerase as substrates. The reversible terminator temporarily halts the polymerase reaction after nucleotide incorporation while the fluorophore signal is detected. After incorporation and signal detection, the fluorophore and the reversible terminator are cleaved to resume the polymerase reaction in the next cycle. Typically, many polynucleotides are confined to an area of a discrete region (referred to as a cluster) on a solid support and are synchronized in their nucleotide incorporation and detection. Some strands may extend faster or slower than their surrounding counterparts, resulting in the clusters of monoclonal amplicons being out-of-phase (i.e., dephasing). During SBS, dephasing leads to signal loss and lowered base call accuracy, ultimately restricting the maximum read length produced by a sequencing device. To increase sequencing efficiency, accuracy, and permit longer sequencing read lengths, there is a need for new strategies to correct dephasing. Described herein, inter alia, are solutions to these and other problems in the art.

### BRIEF SUMMARY

In an aspect is provided a method of sequencing a template polynucleotide, the method including: a) contacting a first primer hybridized to a first template polynucleotide with a first sequencing nucleotide including a first reversible terminator moiety and a first detectable label moiety covalently bound to the first sequencing nucleotide via a first cleavable linker, incorporating the first sequencing nucleotide into the first primer with a polymerase, thereby forming a first extended primer polynucleotide, and detecting the first sequencing nucleotide; b) contacting a second primer hybridized to a second template polynucleotide with a first chase nucleotide including a first retarding moiety covalently bound to the first chase nucleotide via a first chase cleavable linker; and incorporating the first chase nucleotide into the second primer with a polymerase, thereby forming a second extended primer polynucleotide; c) removing the first reversible terminator moiety, the first detectable label moiety, and the first retarding moiety; and d) contacting the first extended primer polynucleotide with a second sequencing nucleotide including a second reversible terminator moiety and a second detectable label moiety covalently bound to the second nucleotide via a second cleavable linker, incorporating the second sequencing nucleotide into the first extended primer polynucleotide with a polymerase, thereby extending the first extended primer polynucleotide, and detecting the second sequencing nucleotide.

In an aspect is provided a method of detecting an incorporated sequencing nucleotide, the method including: i) contacting a solid support including a plurality of template polynucleotides with a plurality of chase nucleotides, wherein each chase nucleotide includes a retarding moiety covalently bound to the chase nucleotide via a cleavable linker, and wherein a first fraction of the plurality of template polynucleotides is hybridized to an unblocked primer; and a second fraction of the plurality of template polynucleotides is hybridized to a blocked primer, wherein the blocked primer includes the incorporated sequencing nucleotide at a 3′ end of the blocked primer; ii) incorporating one of the chase nucleotides into the unblocked primer with a polymerase; and iii) detecting the incorporated sequencing nucleotide.

In an aspect is provided a kit including a sequencing solution and a chase solution, wherein (a) the sequencing solution includes a plurality of sequencing nucleotides, wherein each sequencing nucleotide of the plurality of sequencing nucleotides includes a detectable label moiety and a reversible terminator; (b) the chase solution includes a plurality of chase nucleotides, wherein each chase nucleotide of the plurality of chase nucleotides includes a retardant moiety and a reversible terminator.

In an aspect is provided a sequencing solution. In embodiments, the sequencing solution includes a plurality of sequencing nucleotides, wherein each nucleotide of the plurality of sequencing nucleotides includes a detectable label moiety and a reversible terminator moiety.

In another aspect is provided a chase solution. In embodiments, the chase solution includes a plurality of chase nucleotides, wherein each nucleotide of the plurality of chase nucleotides includes a retardant moiety and a reversible terminator moiety.

In an aspect is provided a method of extending a primer, the method including contacting a primer hybridized to a template polynucleotide with a sequencing solution, followed by contacting the primer with a chase solution; and in the presence of a polymerase, incorporating a nucleotide from the sequencing solution or incorporating a nucleotide from the chase solution to extend the primer. In embodiments, the (a) the sequencing solution includes a plurality of sequencing nucleotides, (b) each nucleotide of the plurality of sequencing nucleotides includes a detectable label moiety and a first reversible terminator moiety; (c) the chase solution includes a plurality of chase nucleotides, (d) each nucleotide of the plurality of chase nucleotides including a retardant moiety and a second reversible terminator moiety, and (e) the retardant moieties differ in structure from the detectable label moieties.

In an aspect is provided a method of sequencing a plurality of template polynucleotides, the method including: (a) contacting a plurality of primers hybridized to template polynucleotides with a chase solution in the presence of a polymerase; wherein a fraction of the plurality of primers include a 3′ terminal nucleotide including a first detectable label moiety and a first reversible terminator moiety; wherein the chase solution includes a plurality of chase nucleotides, each nucleotide in the plurality of chase nucleotides including a retardant moiety and a second reversible terminator moiety; (b) detecting the first detectable label moiety of the 3′ terminal nucleotide; (c) removing the first detectable label moiety, the retardant moiety, and the first and second reversible terminator moieties from nucleotides of the plurality of primers; (d) contacting the plurality of primers hybridized to template polynucleotides with a sequencing solution, wherein the sequencing solution includes a plurality of sequencing nucleotides, each nucleotide of the plurality of sequencing nucleotides including a second detectable label moiety and a third reversible terminator moiety; and wherein a fraction of the plurality of primers incorporate a nucleotide of the plurality of sequencing nucleotides; and (e) repeating steps (a)-(d) thereby sequencing the template polynucleotides.

In yet another aspect is provided a method of sequencing a plurality of template polynucleotides, the method including: i) contacting a substrate including a plurality of immobilized template polynucleotides with a sequencing solution including a plurality of sequencing nucleotides, each nucleotide of the plurality of sequencing nucleotides including a detectable label moiety and a first reversible terminator moiety, wherein each immobilized template polynucleotide includes one or more primers hybridized thereto; and in the presence of a polymerase, extending the one or more primers with a nucleotide to generate extended primers; ii) contacting the substrate with a chase solution including a plurality of chase nucleotides, each nucleotide of the plurality of chase nucleotides including a retardant moiety and a second reversible terminator moiety; iii) detecting the detectable label moiety so as to identify one or more nucleotides incorporated into the extended primers; iv) removing the first and second reversible terminator moieties, the detectable label moiety, and the retardant moiety; and v) repeating steps i) to iv) to sequence the plurality of immobilized template polynucleotides. In embodiments, the method further includes detecting the retardant moiety prior to step iv).

In an aspect is provided a method of detecting templates in a cluster, the method including: (a) contacting a cluster including a plurality of templates with a plurality of chase nucleotides in the presence of a polymerase, each nucleotide of the plurality of chase nucleotides including a retardant moiety and a reversible terminator moiety; wherein a fraction of the plurality of templates in the cluster include reversible-terminated, labeled nucleotides incorporated at the 3′ ends of primers hybridized to the fraction of the plurality of templates; and (b) detecting one or more of the retardant moieties incorporated by primer extension, thereby detecting templates. In embodiments, the method further includes detecting the labeled nucleotides. In embodiments, the method includes removing the reversible terminator moiety, a label of the labeled nucleotides, and the retardant moiety.

In an aspect is provided a kit including a sequencing solution and a chase solution, wherein (a) the sequencing solution includes a plurality of sequencing nucleotides, (b) each nucleotide of the plurality of sequencing nucleotides include a detectable label moiety and a first reversible terminator moiety; (c) the chase solution includes a plurality of chase nucleotides, (d) each nucleotide of the plurality of chase nucleotides includes a retardant moiety and a second reversible terminator moiety, and (e) the retardant moieties differ in structure from the detectable label moieties.

### BRIEF DESCRIPTION OF THE DRAWINGS

FIG. 1. Kinetics for subsequent base incorporation following addition of three different chase nucleotides bearing 3′-reversible terminators with either no retardant moiety (RT-only), a retardant moiety (RT+retardant), and a detectable moiety (RT+dye). Each bar is the average of two measurements performed at 65° C.

FIG. 2. Cleavage halftime for different nucleotides bearing reversible terminators with either no retardant moiety (RT-only), a first retardant moiety type (RT+retardant1), a second retardant moiety type (RT+retardant2), and a detectable moiety (RT+dye). Each bar is the average of two cleavage halftimes with THPP at 55° C.

FIGS. 3A-3C. Embodiments of nucleotides containing non-fluorescent retardant moieties. FIG. 3A depicts a set of PEG retardant nucleotides; FIG. 3B depicts a set of lauric acid retardant nucleotides; FIG. 3C depicts a nucleotide comprising polymerized aromatic monomers.

FIGS. 4A-4C. Nucleotides containing a fluorescent retardant moiety. FIG. 4A: An embodiment of a synthesized nucleotide containing a retardant moiety (IR800) which has an absorption max at 774 nm (in water) and an emission max at 789 nm (in water). FIG. 4B: An embodiment of a synthesized nucleotide containing a retardant moiety (AF405) which has an absorption max at 405 nm (in water) and an emission max at 421 nm (in water). FIG. 4C: An embodiment of a synthesized nucleotide containing a retardant moiety (IR700DX) which has an absorption max at 680 nm (in water) and an emission max at 687 nm (in water).

FIGS. 5A-5C. Nucleotides containing a non-fluorescent retardant moiety FIG. 5A: An embodiment of a synthesized nucleotide containing a retardant moiety (QSY7) which has an absorption max at 560 nm (in water) and serves as a quencher from about 500 nm to about 600 nm. FIG. 5B: An embodiment of a synthesized nucleotide containing a retardant moiety (QSY9) which has an absorption max at 562 nm (in water) and serves as a quencher from about 500 nm to about 600 nm. FIG. 5C: An embodiment of a synthesized nucleotide containing a retardant moiety (BHQ1) which has an absorption max at 534 nm (in water) and serves as a quencher from about 519 to about 556 nm.

### DETAILED DESCRIPTION

The aspects and embodiments described herein relate to modified nucleotides and methods of using the same in nucleic acid sequencing reactions for improving sequencing protocols and obtaining longer sequencing reads. Additionally, the nucleotides described herein provide improved storage stability relative to a control.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure. The abbreviations used herein have their conventional meaning within the chemical and biological arts. The chemical structures and formulae set forth herein are constructed according to the standard rules of chemical valency known in the chemical arts.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together or multiple spirocyclic rings wherein at least one of the fused or spirocyclic rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together or multiple spirocyclic rings wherein at least one of the fused or spirocyclic rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together or multiple spirocyclic rings wherein at least one of the fused or spirocyclic rings is a heterocycloalkyl ring and wherein the multiple rings are attached to the parent molecular moiety through any atom contained within a heterocycloalkyl ring of the multiple rings.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a monocyclic cycloalkyl, a monocyclic cycloalkenyl, or a monocyclic heterocyclyl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a 5 or 6 membered monocyclic cycloalkyl, a 5 or 6 membered monocyclic cycloalkenyl, or a 5 or 6 membered monocyclic heterocyclyl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic cycloalkyl, a bicyclic cycloalkenyl, and a bicyclic heterocyclyl; or (ii) two other ring systems independently selected from the group consisting of a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic cycloalkyl, a bicyclic cycloalkenyl, and a bicyclic heterocyclyl; or (ii) two other ring systems independently selected from the group consisting of a monocyclic cycloalkyl, a monocyclic cycloalkenyl, and a monocyclic heterocyclyl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a monocyclic cycloalkyl, a monocyclic cycloalkenyl, or a monocyclic heterocyclyl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic cycloalkyl, a bicyclic cycloalkenyl, and a bicyclic heterocyclyl; or (ii) two ring systems independently selected from the group consisting of a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic cycloalkyl, a bicyclic cycloalkenyl, and a bicyclic heterocyclyl; or (ii) two ring systems independently selected from the group consisting of a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains one heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a monocyclic cycloalkyl, a monocyclic cycloalkenyl, or a monocyclic heterocycle. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a 5 or 6 membered monocyclic cycloalkyl, a 5 or 6 membered monocyclic cycloalkenyl, or a 5 or 6 membered monocyclic heterocyclyl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic cycloalkyl, a bicyclic cycloalkenyl, and a bicyclic heterocyclyl; or (ii) two other ring systems independently selected from the group consisting of a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic cycloalkyl, a bicyclic cycloalkenyl, and a bicyclic heterocyclyl; or (ii) two other ring systems independently selected from the group consisting of a monocyclic cycloalkyl, a monocyclic cycloalkenyl, and a monocyclic heterocyclyl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

The symbol “ [symbol] ” denotes the point of attachment of a chemical moiety to the remainder of a molecule or chemical formula. The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound (e.g., nucleotide analogue) is a chemical species set forth in the Examples section, claims, embodiments, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

As used herein, the term “isomers” refers to compounds having the same number and kind of atoms, and hence the same molecular weight, but differing in respect to the structural arrangement or configuration of the atoms.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure. The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc ⁹⁹Mo, ¹⁰⁵Pd, ¹⁰⁵Rh, ¹¹¹Ag, ¹¹¹In, ¹²³I, ¹²⁴I ¹²⁵I, ¹³¹I, ¹⁴²Pr, ¹⁴³Pr, ¹⁴⁹Pm, ¹⁵³Sm, ¹⁵⁴⁻¹⁵⁸¹Gd ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In embodiments, a detectable moiety is a moiety (e.g., monovalent form) of a detectable agent. In embodiments, a detectable label moiety is a moiety (e.g., monovalent form) of a detectable label.

The term “retardant moiety” or “retarding moiety” refers to a substance, agent (e.g., a detectable agent), or monovalent compound that, when linked to a nucleotide, is capable of slowing incorporation of the next nucleotide, in the absence of a reversible terminator. In embodiments, presence of a 3′ terminal nucleotide including a retardant moiety increases the halftime of a further nucleotide extension to a level that is about or at least about 2-fold higher, 5-fold higher, 10-fold higher, 15-fold higher, 20-fold higher, 25-fold higher, 30-fold higher, or more, as compared to the 3′ terminal nucleotide lacking a retardant moiety under conditions of a sequencing reaction. In embodiments, the retardant moiety raises the halftime of a further incorporation to at least 5-fold higher. In embodiments, the retardant moiety raises the halftime of a further incorporation to at least 10-fold higher. In embodiments, the halftime for polymerase extension of a primer including a 3′-terminal nucleotide with a retardant moiety is about or at least about 2, 3, 4, 5, 6, 7, 8, 9, 10, 12, 15, or more minutes under conditions of a sequencing reaction. In embodiments, the halftime for polymerase extension of a 3′ terminal nucleotide with a retardant moiety is at least about 5 minutes. In embodiments, the halftime for polymerase extension of a 3′ terminal nucleotide with a retardant moiety is at least about 10 minutes. In embodiments, the retardant moiety slows the incorporation of the next nucleotide by a factor of about 2 to a factor of about 20. In embodiments, the retardant moiety is detectable and does not interfere with sequencing detection (e.g., distinguishable from the detectable labels used to identify the nucleotides used in a sequencing reaction; e.g., less than 530 nm). In embodiments, the maximum emission of the retardant moiety does not significantly overlap with the maximum emission of the detectable labels used to identify the nucleotides used in a sequencing reaction. In embodiments, the emission spectrum of the retardant moiety minimally overlaps with the emission spectrum of the detectable labels used to identify the nucleotides used in a sequencing reaction. In embodiments, the degree of overlap between the retardant moiety spectrum and the detectable labels used in sequencing reactions may be quantified using means known in the art, such as the Szymkiewicz-Simpson coefficient or Jaccard index. Non-limiting examples of retardant moieties include Bodipy® 493/503, aminomethylcoumarin (AMCA), ANT, MANT, AmNS, 7-diethylaminocoumarin-3-carboxylic acid (DEAC), ATTO 390, Alexa Fluor® 350, Marina Blue, Cascade Blue, and Pacific Blue. In embodiments, the retardant moiety does not absorb and/or emit light in the same wavelengths absorbed and/or emitted as the detectable moiety. In embodiments, the retardant moiety has an emission maximum outside the range of detection for the sequencing nucleotides, which is typically about 530 nm to about 750 nm for four color sequencing or about 520 nm to about 660 nm for two color sequencing

The terms “fluorophore” or “fluorescent agent” or “fluorescent dye” are used interchangeably and refer to a substance, compound, agent (e.g., a detectable agent), or composition (e.g., compound) that can absorb light at one or more wavelengths and re-emit light at one or more longer wavelengths, relative to the one or more wavelengths of absorbed light. Examples of fluorophores that may be included in the compounds and compositions described herein include fluorescent proteins, xanthene derivatives (e.g., fluorescein, rhodamine, Oregon green, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5, DRAQ7, or CyTRAK Orange), pyrene derivatives (e.g., cascade blue and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF Dye™, DRAQ™, CyTRAK™, BODIPY™, Alexa Fluor™, DyLight Fluor™, Atto™, Tracy™, FluoProbes™, Abberior Dyes™, DY™ dyes, MegaStokes Dyes™, Sulfo Cy™, Seta™ dyes, SeTau™ dyes, Square Dyes™, Quasar™ dyes, Cal Fluor™ dyes, SureLight Dyes™, PerCP™, Phycobilisomes™, APC™, APCXL™, RPE™, and/or BPE™. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners). In embodiments, the fluorophore is an aromatic (e.g., polyaromatic) moiety having a conjugated 2-electron system. In embodiments, the fluorophore is a fluorescent dye moiety, that is, a monovalent fluorophore.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc ⁹⁹Mo, ¹⁰⁵Pd, ¹⁰⁵R, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd ¹⁶¹Tb, ¹⁶⁶Dy ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Examples of detectable agents include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent moiety or fluorescent dye moiety. In embodiments, the detectable label is a fluorescent dye. In embodiments, the detectable label is a fluorescent dye capable of exchanging energy with another fluorescent dye (e.g., fluorescence resonance energy transfer (FRET) chromophores).

The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

Descriptions of compounds (e.g., nucleotide analogues) of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “salt” refers to acid or base salts of the compounds described herein. Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. In embodiments, compounds may be presented with a positive charge, and it is understood an appropriate counter-ion (e.g., chloride ion, fluoride ion, or acetate ion) may also be present, though not explicitly shown. Likewise, for compounds having a negative charge

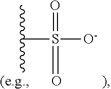

it is understood an appropriate counter-ion (e.g., a proton, sodium ion, potassium ion, or ammonium ion) may also be present, though not explicitly shown. The protonation state of the compound (e.g., a compound described herein) depends on the local environment (i.e., the pH of the environment), therefore, in embodiments, the compound may be described as having a moiety in a protonated state

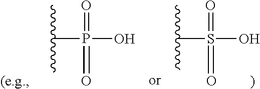

or an ionic state

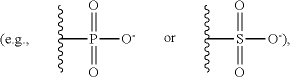

and it is understood these are interchangeable. In embodiments, the counter-ion is represented by the symbol M (e.g., M⁺ or M⁻).

The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds described herein can exist in unsolvated forms as well as solvated forms, including hydrated forms. In general, the solvated forms are equivalent to unsolvated forms and are encompassed within the scope of the present invention. Certain compounds described herein may exist in multiple crystalline or amorphous forms. In general, all physical forms are equivalent for the uses contemplated herein and are intended to be within the scope of the present invention.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Hybridize” shall mean the annealing of one single-stranded nucleic acid (such as a primer) to another nucleic acid based on the well-understood principle of sequence complementarity. In an embodiment the other nucleic acid is a single-stranded nucleic acid. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. In some embodiments, nucleic acids, or portions thereof, that are configured to hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which comprises a double-stranded portion of nucleic acid.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated, however, that the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In certain embodiments the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see, Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. A residue of a nucleic acid, as referred to herein, is a monomer of the nucleic acid (e.g., a nucleotide). The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. A “nucleic acid moiety” as used herein is a monovalent form of a nucleic acid. In embodiments, the nucleic acid moiety is attached to the 3′ or 5′ position of a nucleotide or nucleoside.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

“Nucleotide,” as used herein, refers to a nucleoside-5′-phosphate (e.g., polyphosphate) compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may comprise bases such as adenine (A), cytosine (C), guanine (G), thymine (T), uracil (U), or analogues thereof, and may comprise 1, 2, 3, 4, 5, 6, 7, 8, or more phosphates in the phosphate group. Nucleotides may be modified at one or more of the base, sugar, or phosphate group. A nucleotide may have a label or tag attached (a “labeled nucleotide” or “tagged nucleotide”). In an embodiment, the nucleotide is a deoxyribonucleotide. In another embodiment, the nucleotide is a ribonucleotide. In embodiments, nucleotides comprise 3 phosphate groups (e.g., a triphosphate group).

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In embodiments, “nucleotide analogue,” “nucleotide analog,” or “nucleotide derivative” shall mean an analogue of adenine (A), cytosine (C), guanine (G), thymine (T), or uracil (U) (that is, an analogue or derivative of a nucleotide comprising the base A, G, C, T or U), comprising a phosphate group, which may be recognized by DNA or RNA polymerase (whichever is applicable) and may be incorporated into a strand of DNA or RNA (whichever is appropriate). Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

A “nucleoside” is structurally similar to a nucleotide, but is missing the phosphate moieties that are present in a nucleotide. An example of a nucleoside analogue would be one in which the label is linked to the base and there is no phosphate group attached to the sugar molecule. “Nucleoside,” as used herein, refers to a glycosyl compound consisting of a nucleobase and a 5-membered ring sugar (e.g., either ribose or deoxyribose). Nucleosides may comprise bases such as adenine (A), cytosine (C), guanine (G), thymine (T), uracil (U), or analogues thereof. Nucleosides may be modified at the base and/or and the sugar. In an embodiment, the nucleoside is a deoxyribonucleoside. In another embodiment, the nucleoside is a ribonucleoside.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate groups include —NH₂, —COOH, —COOCH₃, —N-hydroxysuccinimide, -maleimide,

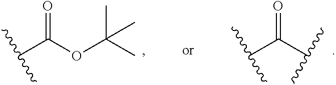

In embodiments, the bioconjugate reactive group may be protected (e.g., with a protecting group). Additional examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | Resulting |
| bioconjugate | bioconjugate | Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotri azines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or streptavidin to form a avidin-biotin complex or streptavidin-biotin complex.

The term “nucleobase” or “base” as used herein refers to a purine or pyrimidine compound, or a derivative thereof, that may be a constituent of nucleic acid (i.e., DNA or RNA, or a derivative thereof). In embodiments, the nucleobase is a divalent purine or pyrimidine, or derivative thereof. In embodiments, the nucleobase is a monovalent purine or pyrimidine, or derivative thereof. In embodiments, the base is a derivative of a naturally occurring DNA or RNA base (e.g., a base analogue). In embodiments the base is a hybridizing base. In embodiments the base hybridizes to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is adenine, guanine, uracil, cytosine, thymine, hypoxanthine, xanthine, theobromine, caffeine, uric acid, or isoguanine, which may be optionally substituted or modified. In embodiments, the base is adenine, guanine, hypoxanthine, xanthine, theobromine, caffeine, uric acid, or isoguanine, which may be optionally substituted or modified.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. In embodiments, a cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). In embodiments, a cleavable linker is a self-immolative linker, a trivalent linker, or a linker capable of dendritic amplification of signal, or a self-immolative dendrimer containing linker (e.g., all as described in US 2007/0009980, US 2006/0003383, and US 2009/0047699, which are incorporated by reference in their entirety for any purpose). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (N₂H₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or formamidopyrimidine DNA glycosylase Fpg. In embodiments, the scissile linkage site includes a sequence-specific nicking site having a nucleotide sequence that is recognized and nicked by a nicking endonuclease enzyme or a uracil DNA glycosylase. The term “self-immolative” referring to a linker is used in accordance with its well understood meaning in Chemistry and Biology as used in US 2007/0009980, US 2006/0003383, and US 2009/0047699, which are incorporated by reference in their entirety for any purpose. In embodiments, “self-immolative” referring to a linker refers to a linker that is capable of additional cleavage following initial cleavage by an external stimulus. The term dendrimer is used in accordance with its well understood meaning in Chemistry. In embodiments, the term “self-immolative dendrimer” is used as described in US 2007/0009980, US 2006/0003383, and US 2009/0047699, which are incorporated by reference in their entirety for any purpose and in embodiments refers to a dendrimer that is capable of releasing all of its tail units through a self-immolative fragmentation following initial cleavage by an external stimulus.

A “photocleavable linker” (e.g., including or consisting of an o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of a reducing agent (e.g., tris(3-hydroxypropyl)phosphine). In embodiments, the cleavable linker is a dialkylketal linker (Binaulda S., et al., Chem. Commun., 2013, 49, 2082-2102; Shenoi R. A., et al., J. Am. Chem. Soc., 2012, 134, 14945-14957), an azo linker (Rathod, K. M., et al., Chem. Sci. Tran., 2013, 2, 25-28; Leriche G., et al., Eur. J Org. Chem., 2010, 23, 4360-64), an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent and the agent that cleaves each cleavable linker is different. In embodiments, an orthogonally cleavable linker is a cleavable linker that, following cleavage, the two separated entities (e.g., fluorescent dye, bioconjugate reactive group) do not further react and form a new orthogonally cleavable linker.

The term “orthogonal detectable label” or “orthogonal detectable moiety” as used herein refer to a detectable label (e.g., fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox-labeled tetrazine, Alexa488-labeled SHA, Cy5-labeled streptavidin, and R6G-labeled dibenzocyclooctyne.

As used herein, the term “modified nucleotide” refers to a nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is described herein. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. As used herein, the terms “blocking moiety,” “reversible blocking group,” “reversible terminator” and “reversible terminator moiety” are used in accordance with their plain and ordinary meanings and refer to a cleavable moiety which does not interfere with incorporation of a nucleotide comprising it by a polymerase (e.g., DNA polymerase, modified DNA polymerase), but prevents further strand extension until removed (“unblocked”). For example, a reversible terminator may refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. The nucleotides may be modified with reversible terminators useful in methods provided herein and may be 3-O-blocked reversible or 3-unblocked reversible terminators. In nucleotides with 3-O-blocked reversible terminators, the blocking group may be represented as —OR [reversible terminating (capping) group], wherein 0 is the oxygen atom of the 3-OH of the pentose and R is the blocking group, while the label is linked to the base, which acts as a reporter and can be cleaved. The 3-O-blocked reversible terminators are known in the art, and may be, for instance, a 3′-ONH₂ reversible terminator, a 3-O-allyl reversible terminator, or a 3-O-azidomethyl reversible terminator. In embodiments, the reversible terminator moiety is

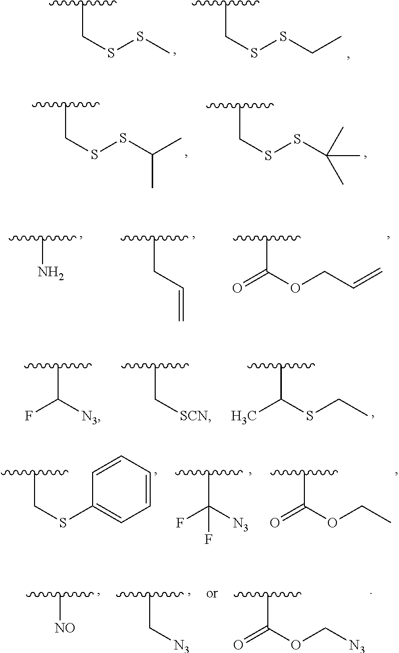

The term “thio-trigger moiety” refers to a substituent having the formula

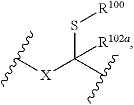

wherein X is —O—, —NH—, or —S—; R¹⁰⁰ is —SO₃H, —SR¹⁰² or —CN; and R¹⁰² and R^{102a} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the thio-trigger moiety has the formula:

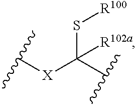

wherein X is —O—, and R¹⁰⁰ and R^{102a} are as described herein. In embodiments, the thio-trigger moiety has the formula:

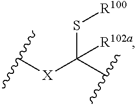

wherein X is —NH—, and R¹⁰⁰ and R^{102a} are as described herein. Additional examples of linkers containing thio-trigger moieties may be found in U.S. Pat. No. 10,822,653.

A “thio-trigger containing linker” refers to a covalent linker that includes a thio-trigger moiety. When a reducing agent (e.g., dithiothreitol, THPP, or TCEP) contacts a thio-trigger containing linker, the heteroatom represented by the symbol X (e.g., oxygen) of the thio-trigger moiety is reduced and breaks the linker apart into two separate moieties.

The term “polymerase-compatible cleavable moiety” or “reversible terminator” as used herein refers to a cleavable moiety which does not interfere with a function of a polymerase (e.g., DNA polymerase, modified DNA polymerase, in incorporating the nucleotide, to which the polymerase-compatible cleavable moiety is attached, to the 3′ end of the newly formed nucleotide strand). Methods for determining the function of a polymerase contemplated herein are described in B. Rosenblum et al. (Nucleic Acids Res. 1997 Nov. 15; 25(22): 4500-4504); and Z. Zhu et al. (Nucleic Acids Res. 1994 Aug. 25; 22(16): 3418-3422), which are incorporated by reference herein in their entirety for all purposes. In embodiments the polymerase-compatible cleavable moiety does not decrease the function of a polymerase relative to the absence of the polymerase-compatible cleavable moiety. In embodiments, the polymerase-compatible cleavable moiety does not negatively affect DNA polymerase recognition. In embodiments, the polymerase-compatible cleavable moiety does not negatively affect (e.g., limit) the read length of the DNA polymerase. Additional examples of a polymerase-compatible cleavable moiety may be found in U.S. Pat. No. 6,664,079, Ju J. et al. (2006) Proc Natl Acad Sci USA 103(52):19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. Additional examples of a polymerase-compatible cleavable moiety may be found in U.S. Pat. Nos. 6,214,987 and 5,872,244, which are incorporated herein by reference in their entirety for all purposes. In embodiments, a polymerase-compatible cleavable moiety includes an azido moiety or a dithiol linking moiety. In embodiments, the polymerase-compatible cleavable moiety is —NH₂, —CN, —CH₃, C₂-C₆ allyl (e.g., —CH₂—CH═CH₂), methoxyalkyl (e.g., —CH₂—O—CH₃), or —CH₂N₃. In embodiments, the polymerase-compatible cleavable moiety comprises a disulfide moiety. In embodiments, the polymerase-compatible cleavable moiety includes a hydrocarbyl. In embodiments, the polymerase-compatible cleavable moiety includes an ester (O—C(O)R^{Z}′ wherein R^{Z}′ is any alkyl or aryl group which can include a formate, benzoyl formate, acetate, substituted acetate, propionate, and other esters as described in Green, T. W. (Protective Groups in Organic Chemistry, Wiley & Sons, New York, 1981)). In embodiments, the polymerase-compatible cleavable moiety includes an ether (O—R^{ZZ} wherein R^{ZZ} can be substituted or unsubstituted alkyl such as methyl, substituted methyl, ethyl, substituted ethyl, allyl, substituted benzyl, silyl, or any other ether used to transiently protect hydroxyls and similar groups). In embodiments, the polymerase-compatible cleavable moiety includes —O—CH₂(OC₂H₅)_{M}CH₃ wherein M is an integer from 1 to 10. In embodiments, the polymerase-compatible cleavable moiety includes a phosphate, phosphoramidate, phosphoramide, toluic acid ester, benzoic ester, acetic acid ester, or ethoxyethyl ether. In embodiments, the polymerase-compatible cleavable moiety includes a disulfide moiety. In embodiments, a polymerase-compatible cleavable moiety is a cleavable moiety on a nucleotide, nucleobase, nucleoside, or nucleic acid that does not interfere with a function of a polymerase (e.g., DNA polymerase, modified DNA polymerase). In embodiments, the reversible terminator moiety is

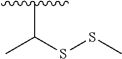 [SMILES: *C(C)SSC]

as described in U.S. Pat. No. 10,738,072, which is incorporated herein by reference for all purposes. For example, a nucleotide including a reversible terminator moiety may be represented by the formula:

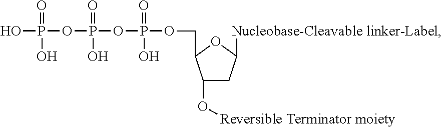

where the nucleobase is adenine or adenine analogue, thymine or thymine analogue, guanine or guanine analogue, or cytosine or cytosine analogue.

The term “polymerase,” as used herein, refers to any natural or non-naturally occurring enzyme or other catalyst that is capable of catalyzing a polymerization reaction, such as the polymerization of nucleotide monomers to form a nucleic acid polymer. Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase, 9° N polymerase (exo-)A485L/Y409V, Phi29 DNA Polymerase ((p29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9°N polymerase (exo−), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). As used herein, the term “thermophilic nucleic acid polymerase” refers to a family of DNA polymerases (e.g., 9°N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or γ-phosphate labeled nucleotides (e.g., Therminator γ: D141A/E143A/W355A/L408W/R460A/Q461S/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′->5′ direction, releasing deoxyribonucleoside 5′-monophosphates one after another. Methods for quantifying exonuclease activity are known in the art, see for example Southworth et al, PNAS Vol 93, 8281-8285 (1996).

As used herein, the terms “polynucleotide primer” and “primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis. In embodiments, an oligonucleotide is a primer configured for extension by a polymerase when the primer is annealed completely or partially to a complementary nucleic acid template. A primer is often a single stranded nucleic acid. In embodiments, a primer, or portion thereof, is substantially complementary to a portion of an adapter. In embodiments, a primer has a length of 200 nucleotides or less. In embodiments, a primer has a length of 10 to 150 nucleotides, 15 to 150 nucleotides, 5 to 100 nucleotides, 5 to 50 nucleotides or 10 to 50 nucleotides. In embodiments, an oligonucleotide may be immobilized to a solid support

The phrase “stringent hybridization conditions” refers to conditions under which a primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

As used herein, the term “depletion polynucleotide” refers to a polynucleotide capable of being extended by a depletion polymerase, wherein the depletion polymerase incorporates one or more 3′-OH nucleotide(s). In embodiments, the depletion polynucleotide includes a homopolymer sequence (e.g., a polyT sequence). In embodiments, the depletion polynucleotide is a single polynucleotide comprising a hairpin structure and a 5′ overhang. In embodiments, the depletion polynucleotides include a depletion primer annealed to a depletion template, wherein the depletion primer has a free 3′-OH. A depletion polynucleotide may alternatively be referred to herein as a depletion oligonucleotide or depletion oligonucleotide template. In embodiments, the depletion polynucleotide is immobilized to a solid support. In embodiments, the depletion polynucleotide is free in solution. In embodiments, the depletion polynucleotide includes 10, 20, 30, 40, 50, 60, 70, 80, 90, 100, 150, 200, 250, or more nucleotide bases. The depletion polynucleotide can be of any suitable length. In embodiments, the depletion polynucleotide is about 10, 15, 20, 25, 30, or more nucleotides in length. In embodiments, the depletion polynucleotide is 10-50, 15-30, or 20-25 nucleotides in length. In embodiments, the depletion primer and the depletion template are portions of a single polynucleotide. In embodiments, the depletion primer and the depletion template are portions of a single polynucleotide including a loop structure. As used herein, the term “loop region” or “loop” refers to a region of a single polynucleotide that is between sequences of the depletion primer and the depletion template, and remains single-stranded when depletion primer and depletion template are hybridized to one another. In embodiments, the loop includes about 10 to about 20 random nucleotides.

As used herein, the term “depletion polymerase” refers to a polymerase capable of incorporating 3′-OH nucleotides, and incapable of incorporating optionally labeled, 3′-O-blocked reversible terminator nucleotides. In embodiments, the depletion polymerase is a polymerase described herein. In embodiments, the depletion polymerase includes a Klenow fragment, or mutant thereof. In embodiments, the depletion polymerase includes a Klenow fragment. In embodiments, the depletion polymerase is a Klenow fragment, or a mutant thereof. In embodiments, the depletion polymerase is a bacterial DNA polymerase, eukaryotic DNA polymerase, archaeal DNA polymerase, viral DNA polymerase, or phage DNA polymerases. In embodiments, the depletion polymerase is active at a temperature of about 2° C.-65° C., about 2° C.-10° C., or about 4° C.-37° C. In embodiments, the depletion polymerase is active at about 4° C. In embodiments, the depletion polymerase is active at about 37° C. In embodiments, the depletion polymerase is active at about 42° C. In embodiments, the depletion polymerase is not thermostable above 65° C. In embodiments, the depletion polymerase is not thermostable above 55° C. In embodiments, the depletion polymerase is not thermostable above 50° C. In embodiments, the depletion polymerase is not thermostable above 45° C.

As used herein, the term “nucleotide cyclase” refers to an enzyme capable of cyclizing a 3′-OH nucleotide, and incapable of cyclizing an optionally labeled, 3′-O-blocked reversible terminator nucleotide.

As used herein, the terms “solid support” and “substrate” and “solid surface” refers to discrete solid or semi-solid surfaces to which a plurality of primers may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. Solid supports in the form of discrete particles may be referred to herein as “beads,” which alone does not imply or require any particular shape. A bead can be non-spherical in shape. A solid support may further comprise a polymer or hydrogel on the surface to which the primers are attached (e.g., the splint primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008).

Where a range of values is provided herein, it is understood that each intervening value, to the tenth of the unit (if appropriate) of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

While various embodiments of the invention are shown and described herein, it will be understood by those skilled in the art that such embodiments are provided by way of example only. Numerous variations, changes, and substitutes may occur to those skilled in the art without departing from the invention. It should be understood that various alternatives to the embodiments of the invention described herein may be employed.

As used herein, and unless stated otherwise, each of the following terms shall be used in accordance with their plain and ordinary meaning, for example: A indicates the presence of Adenine; C indicates the presence of Cytosine; DNA is Deoxyribonucleic acid; G indicates the presence of Guanine; RNA is Ribonucleic acid; T indicates the presence of Thymine; and U indicates the presence of Uracil. In embodiments, each of the following terms shall have the definition set forth below A—Adenine; C—Cytosine; DNA—Deoxyribonucleic acid; G—Guanine; RNA—Ribonucleic acid; T—Thymine; and U—Uracil.

The term “reaction vessel” is used in accordance with its ordinary meaning in chemistry or chemical engineering, and refers to a container having an inner volume in which a reaction takes place. In embodiments, the reaction vessel may be designed to provide suitable reaction conditions such as reaction volume, reaction temperature or pressure, and stirring or agitation, which may be adjusted to ensure that the reaction proceeds with a desired, sufficient or highest efficiency for producing a product from the chemical reaction. In embodiments, the reaction vessel is a container for liquid, gas or solid. In embodiments, the reaction vessel may include an inlet, an outlet, a reservoir and the like. In embodiments, the reaction vessel is connected to a pump (e.g., vacuum pump), a controller (e.g., CPU), or a monitoring device (e.g., UV detector or spectrophotometer). In embodiments, the reaction vessel is a flow cell. In embodiments, the reaction vessel is within a sequencing device.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “kit” is used in accordance with its plain ordinary meaning and refers to any delivery system for delivering materials or reagents for carrying out a method of the invention. Such delivery systems include systems that allow for the storage, transport, or delivery of reaction reagents (e.g., nucleotides, enzymes, nucleic acid templates, etc. in the appropriate containers) and/or supporting materials (e.g., buffers, written instructions for performing the reaction, etc.) from one location to another location. For example, kits include one or more enclosures (e.g., boxes) containing the relevant reaction reagents and/or supporting materials. Such contents may be delivered to the intended recipient together or separately. For example, a first container may contain an enzyme, while a second container contains nucleotides. In embodiments, the kit includes vessels containing one or more enzymes, primers, adaptors, or other reagents as described herein. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer.

As used herein, the terms “sequencing”, “sequence determination”, “determining a nucleotide sequence”, and the like include determination of a partial or complete sequence information, including the identification, ordering, or locations of the nucleotides that comprise the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “sequencing reaction mixture” is used in accordance with its plain and ordinary meaning and refers to an aqueous mixture that contains the reagents necessary to allow a dNTP or dNTP analogue to add a nucleotide to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride). As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used herein, the term “extension”, “extending,” or “elongation” is used in accordance with its plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) polynucleotide strand.

As used herein, the term “sequencing read” is used in accordance with its plain and ordinary meaning and refers to an inferred sequence of base pairs (or base pair probabilities) corresponding to all or part of a single DNA fragment. Sequencing technologies vary in the length of reads produced. A sequencing read may include 10, 20, 30, 40, 50, 60, 70, 80, 90, 100, 150, 200, 250, or more nucleotide bases. Reads of length 20-40 base pairs (bp) are referred to as ultra-short. Typical sequencers produce read lengths in the range of about 100-500 bp. Read length is a factor which can affect the results of biological studies. For example, longer read lengths improve the resolution of de novo genome assembly and detection of structural variants. In some embodiments, a sequencing read may include 500, 600, 700, 800, 900, 1000, 1100, 1200, 1300, 1400, 1500, or more nucleotide bases. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

The methods and kits of the present disclosure may be applied, mutatis mutandis, to the sequencing of RNA, or to determining the identity of a ribonucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics (e.g., a G4™ sequencing platform), Illumina™, Inc. (e.g. HiSeq™, MiSeq™, NextSeq™, or NovaSeq™ systems), Life Technologies™ (e.g. ABI PRISM™, or SOLiD™ systems), Pacific Biosciences (e.g. systems using SMRT™ Technology such as the Sequel™ or RS II™ systems), or Qiagen (e.g. Genereader™ system).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a kit including a sequencing solution and a chase solution. In embodiments, the sequencing solution includes a plurality of sequencing nucleotides, wherein each sequencing nucleotide of the plurality of sequencing nucleotides includes a detectable label moiety and a reversible terminator. In embodiments, the chase solution includes a plurality of chase nucleotides, wherein each chase nucleotide of the plurality of chase nucleotides includes a retardant moiety and a reversible terminator. In embodiments, the sequencing solution includes components necessary to incorporate a detectable nucleotide into a polynucleotide strand (e.g., a primer) hybridized to a template. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, particles, labeled nucleotides, and/or modified nucleotides), buffers, salts, and/or labels (e.g., fluorophores). In embodiments, each solution is provided in a separate container. In embodiments, the kit included one or more components as described in US 2022/0136048, which is incorporated herein by reference in its entirety. The kit includes one or more of the compositions as described herein. In embodiments, the includes one or more DNA polymerases. In embodiments, the kit includes additional components, such as one or more primers, modified and/or unmodified deoxynucleotide triphosphates (dNTPs), buffers, quantification reagents, e.g., intercalating reagents, or reagents binding to the minor groove, (e.g., PicoGreen (Molecular Probes), SybrGreen (Molecular Probes), ethidium bromide, Gelstar (Cambrex) and Vista Green (Amersham)).

In embodiments, the individual components of the kit can be alternatively contained either together in one storage container or separately in two or more storage containers (e.g., separate bottles or vials). In embodiments, the solution (e.g., the chase solution and/or the sequencing solution) may include a depletion polymerase. In embodiments, the depletion polymerase includes a Klenow fragment (e.g., Klenow (3′→5′ exo−)) polymerase. In embodiments, the depletion polymerase is a Klenow fragment polymerase. In embodiments, the depletion polymerase is a Klenow polymerase. In embodiments, the depletion polymerase is a Klentaq polymerase. “Klenow fragment” as used herein means any C-terminal fragment of a family A DNA polymerase which has polymerase activity but no 5′→3′ exonuclease activity. In embodiments, additional mutations may be introduced to remove 5′-3′ exonuclease activity. In embodiments, the depletion polymerase is a Klenow fragment or mutant thereof, soluble guanylyl cyclase or mutant thereof, or a terminal deoxynucleotidyl transferase (TdT).

In embodiments, the depletion polymerase is a polymerase including an amino acid sequence that is at least 80% identical to a continuous 500 amino acid sequence within SEQ ID NO: 1, at least one mutation at amino acid position 32 or an amino acid position functionally equivalent to amino acid position 32; a mutation at amino acid position 34 or an amino acid position functionally equivalent to amino acid position 34; or a mutation at amino acid position 584 or an amino acid position functionally equivalent to amino acid position 584.

In embodiments, the nucleotide cyclase is a soluble guanylyl cyclase (also known as guanyl cyclase, guanylyl cyclase, or GC). In embodiments, the cyclase is soluble guanylyl cyclase (e.g., soluble guanylyl cyclase α1β1, as described in Beste et al Biochemistry. 2012; 51(1):194-204), which has both purinyl and pyrimidinyl cyclase activity and can serve to cyclize all potential nucleotides present in a nucleotide solution (e.g., A, C, G, T/U).

In an aspect is provided a composition including a plurality of primers bound to nucleic acid templates, a fraction of the plurality of primers include a free 3′-OH, another fraction of the plurality of primers include an incorporated labeled nucleotide including a reversible terminator, wherein each reversible terminator is bound to the 3-oxygen of the deoxyribose, wherein a label is bound via a chemically cleavable linker; and another fraction of the plurality of primers include an incorporated nucleotide including a reversible terminator and a retarding moiety, wherein each reversible terminator is bound to the 3-oxygen of the deoxyribose, and wherein the retarding moiety is bound via a chemically cleavable linker. In embodiments, the primers or the nucleic acid templates are immobilized to a solid support. In embodiments, the nucleic acid templates are immobilized to a solid support.

In embodiments, the sequencing solution of the kit includes i) a plurality of adenine nucleotides, or analogs thereof, ii) a plurality of thymine nucleotides, or analogs thereof or a plurality of uracil nucleotides, or analogs thereof, iii) a plurality of cytosine nucleotides, or analogs thereof; and iv) a plurality of guanine nucleotides, or analogs thereof. In embodiments, the plurality of adenine nucleotides may include analogs such as 7-deaza-adenine. In embodiments, the plurality of adenine nucleotides includes a label attached through a cleavable linker, as described herein, to the 7-position of deaza-adenine. In embodiments, the plurality of adenine nucleotides includes a reversible terminator moiety, as described herein, to cap the —OH group at the 3′-position of the deoxyribose. In embodiments, the plurality of thymine nucleotides includes a label attached through a cleavable linker, as described herein, to the 5-position of thymine. In embodiments, the plurality of thymine nucleotides includes a reversible terminator moiety, as described herein, to cap the —OH group at the 3′-position of the deoxyribose. In embodiments, the plurality of uracil nucleotides includes a label attached through a cleavable linker, as described herein, to the 5-position of uracil. In embodiments, the plurality of thymine nucleotides includes a reversible terminator moiety, as described herein, to cap the —OH group at the 3′-position of the deoxyribose. In embodiments, the plurality of cytosine nucleotides includes a label attached through a cleavable linker, as described herein, to the 5-position of cytosine. In embodiments, the plurality of cytosine nucleotides includes a reversible terminator moiety, as described herein, to cap the —OH group at the 3′-position of the deoxyribose. In embodiments, the plurality of cytosine nucleotides includes a reversible terminator moiety, as described herein, to cap the —OH group at the 3′-position of the deoxyribose. In embodiments, the plurality of guanine nucleotides may include analogs such as 7-deaza-guanine. In embodiments, the plurality of guanine nucleotides includes a label attached through a cleavable linker, as described herein, to the 7-position of deaza-guanine. In embodiments, the plurality of guanine nucleotides includes a reversible terminator moiety, as described herein, to cap the —OH group at the 3′-position of the deoxyribose. In embodiments, the nucleotides within a plurality of nucleotides are differently labeled. For example, the composition may include a plurality of nucleotide analogues covalently linked (e.g., covalently linked with a cleavable linker) to a first dye; a plurality of nucleotide analogues covalently linked (e.g., covalently linked with a cleavable linker) to a second dye; a plurality of nucleotide analogues covalently linked (e.g., covalently linked with a cleavable linker) to a third dye; a plurality of nucleotide analogues covalently linked (e.g., covalently linked with a cleavable linker) to a fourth dye; wherein each dye is spectrally distinct from each other. In embodiments, the composition includes a plurality of adenine or adenine analogues covalently linked (e.g., covalently linked with a cleavable linker) to a first dye; a plurality of thymine or thymine analogues covalently linked (e.g., covalently linked with a cleavable linker) to a second dye; a plurality of guanine or guanine analogues covalently linked (e.g., covalently linked with a cleavable linker) to a third dye; a plurality of cytosine or cytosine analogues covalently linked (e.g., covalently linked with a cleavable linker) to a fourth dye; wherein each dye is spectrally distinct from each other.

In embodiments, the label on the i) plurality of adenine nucleotides, or analogs thereof, ii) a plurality of thymine nucleotides, or analogs thereof or a plurality of uracil nucleotides, or analogs thereof, iii) a plurality of cytosine nucleotides, or analogs thereof; and iv) a plurality of guanine nucleotides is detectable. In embodiments, the plurality of adenine nucleotides, or analogs thereof has a first detectable label. In embodiments, the plurality of thymine nucleotides, or analogs thereof or a plurality of uracil nucleotides, or analogs thereof has a second detectable label. In embodiments, the plurality of cytosine nucleotides, or analogs thereof has a third detectable label. In embodiments, the plurality of guanine nucleotides has a fourth detectable label. In embodiments, the first, second, third and fourth detectable labels are all different from each other. In embodiments, the first, second, third and fourth detectable labels are the same. In embodiments, first, second, third and fourth detectable labels are each a fluorescent dye moiety. In embodiments, embodiments, first, second, third and fourth detectable labels are each independently a detectable moiety as described in Table 1. In embodiments, the detectable label is associated with the nucleobase (e.g., detecting the label identifies the nucleobase to which it is linked).

In embodiments, the chase solution of the kit includes a plurality of chase nucleotides, wherein each chase nucleotide of the plurality of chase nucleotides includes a retardant moiety and a reversible terminator. In embodiments, the chase solution of the kit includes i) a plurality of adenine nucleotides, or analogs thereof; ii) a plurality of thymine nucleotides, or analogs thereof or a plurality of uracil nucleotides, or analogs thereof; iii) a plurality of cytosine nucleotides, or analogs thereof; and iv) a plurality of guanine nucleotides, or analogs thereof. In embodiments, the plurality of adenine nucleotides may include analogs such as 7-deaza-adenine. In embodiments, the plurality of adenine nucleotides includes a retardant moiety attached through a cleavable linker, as described herein, to the 7-position of deaza-adenine. In embodiments, the plurality of adenine nucleotides includes a reversible terminator moiety, as described herein, to cap the —OH group at the 3′-position of the deoxyribose. In embodiments, the plurality of thymine nucleotides includes a retardant moiety attached through a cleavable linker, as described herein, to the 5-position of thymine. In embodiments, the plurality of thymine nucleotides includes a reversible terminator moiety, as described herein, to cap the —OH group at the 3′-position of the deoxyribose. In embodiments, the plurality of uracil nucleotides includes a retardant moiety attached through a cleavable linker, as described herein, to the 5-position of uracil. In embodiments, the plurality of thymine nucleotides includes a reversible terminator moiety, as described herein, to cap the —OH group at the 3′-position of the deoxyribose. In embodiments, the plurality of cytosine nucleotides includes a retardant moiety attached through a cleavable linker, as described herein, to the 5-position of cytosine. In embodiments, the plurality of cytosine nucleotides includes a reversible terminator moiety, as described herein, to cap the —OH group at the 3′-position of the deoxyribose. In embodiments, the plurality of cytosine nucleotides includes a retardant moiety, as described herein, to cap the —OH group at the 3′-position of the deoxyribose. In embodiments, the plurality of guanine nucleotides may include analogs such as 7-deaza-guanine. In embodiments, the plurality of guanine nucleotides includes a retardant moiety attached through a cleavable linker, as described herein, to the 7-position of deaza-guanine. In embodiments, the plurality of guanine nucleotides includes a reversible terminator moiety, as described herein, to cap the —OH group at the 3′-position of the deoxyribose. In embodiments, each of chase nucleotides comprise the same retardant moiety (e.g., each nucleotide type, dATP, dTTP, dCTP, and dGTP, all include the same chemical moiety, albeit individually linked to the retarding moiety). In embodiments, the retardant moiety is:

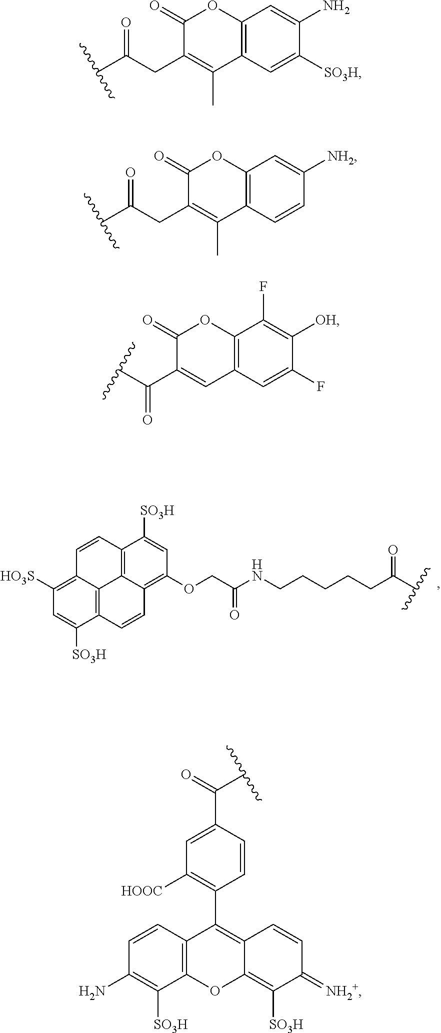

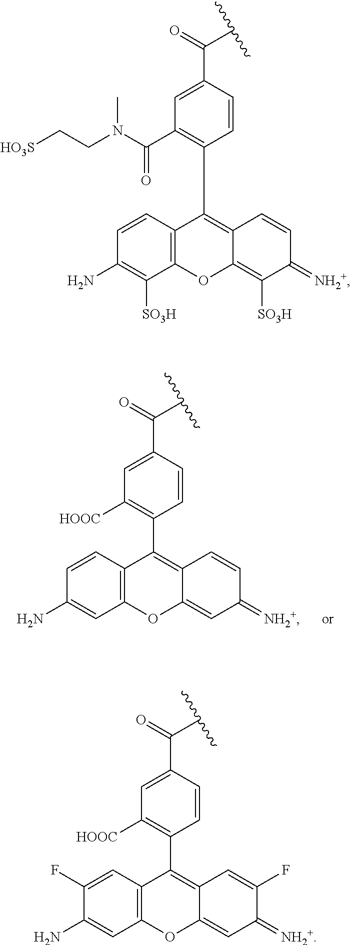

In embodiments, the retardant moiety is

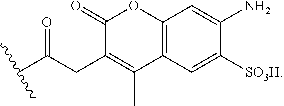 [SMILES: *C(=O)Cc1c(C)c2cc(S(=O)(=O)O)c(N)cc2oc1=O]

In embodiments, the retardant moiety is

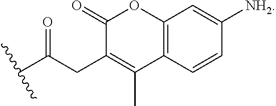 [SMILES: *C(=O)Cc1c(C)c2ccc(N)cc2oc1=O]

In embodiments, the retardant moiety is

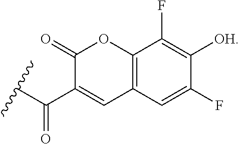 [SMILES: *C(=O)c1cc2cc(F)c(O)c(F)c2oc1=O]

In embodiments, the retardant moiety is

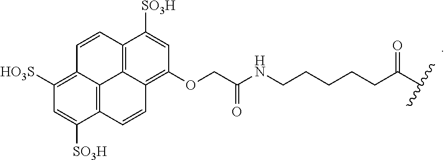 [SMILES: *C(=O)CCCCCNC(=O)COc1cc(S(=O)(=O)O)c2ccc3c(S(=O)(=O)O)cc(S(=O)(=O)O)c4ccc1c2c43]

In embodiments, the retardant moiety is

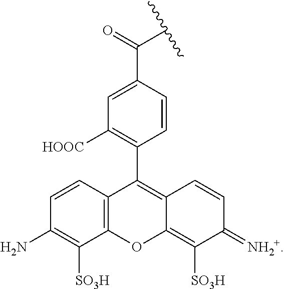

In embodiments, the retardant moiety is

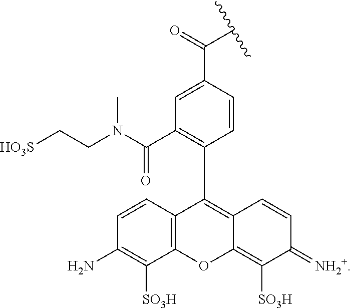

In embodiments, the retardant moiety is

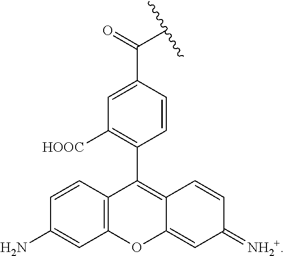

In embodiments, the retardant moiety is

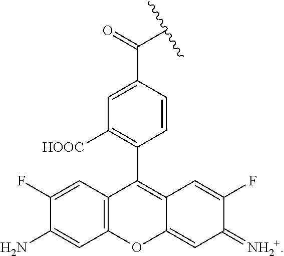

In an aspect is provided a sequencing solution. In embodiments, the sequencing solution includes components necessary to incorporate a detectable nucleotide into a polynucleotide strand (e.g., a primer) hybridized to a template. In embodiments, the sequencing solution includes a plurality of sequencing nucleotides, wherein each nucleotide of the plurality of sequencing nucleotides includes a detectable label moiety and a reversible terminator moiety. In embodiments, each nucleotide of the plurality of sequencing nucleotides has the formula:

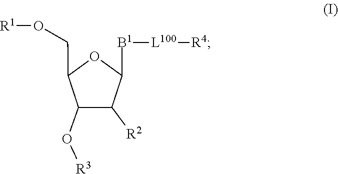

wherein, B¹ is a nucleobase; R¹ is hydrogen, a monophosphate moiety, polyphosphate moiety (e.g., a triphosphate), nucleic acid moiety, or a thiotriphosphate; R² is hydrogen or —OH; R³ is independently a reversible terminator; R⁴ is independently a detectable label moiety; and L¹⁰⁰ is a cleavable linker. In embodiments, the sequencing solution does not include chase nucleotides.

In embodiments, B¹ is a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

In embodiments, B¹ is

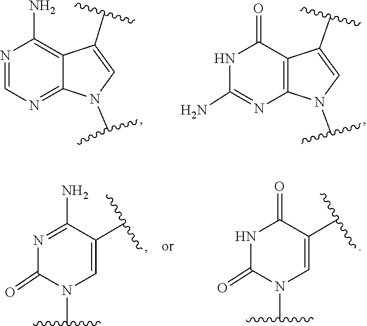 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B¹ is

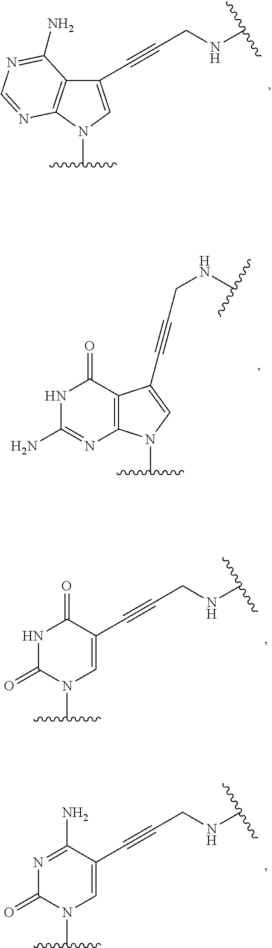 [SMILES: *NCC#Cc1cn(*)c2ncnc(N)c12 ; *NCC#Cc1cn(*)c2nc(N)[nH]c(=O)c12 ; *NCC#Cc1cn(*)c(=O)[nH]c1=O ; *NCC#Cc1cn(*)c(=O)nc1N]

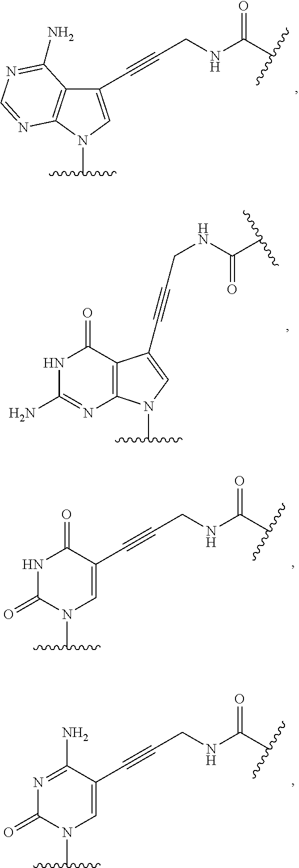 [SMILES: *C(=O)NCC#Cc1cn(*)c2ncnc(N)c12 ; *C(=O)NCC#Cc1cn(*)c2nc(N)[nH]c(=O)c12 ; *C(=O)NCC#Cc1cn(*)c(=O)[nH]c1=O ; *C(=O)NCC#Cc1cn(*)c(=O)nc1N]

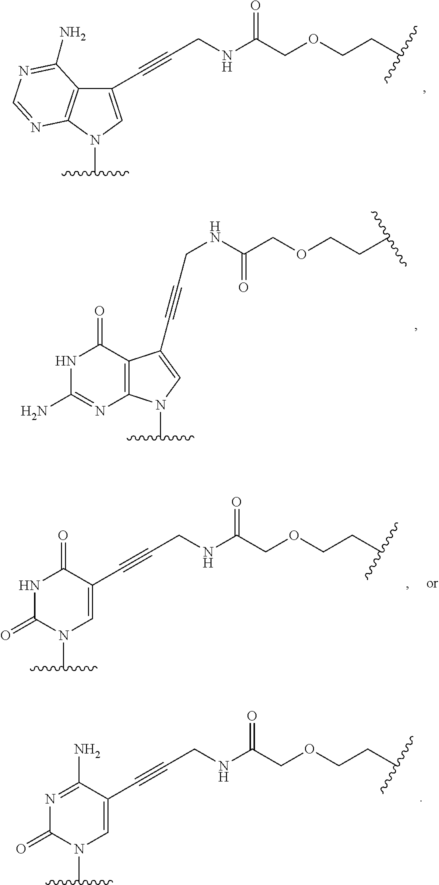 [SMILES: *CCOCC(=O)NCC#Cc1cn(*)c2ncnc(N)c12 ; *CCOCC(=O)NCC#Cc1cn(*)c2nc(N)[nH]c(=O)c12 ; *CCOCC(=O)NCC#Cc1cn(*)c(=O)[nH]c1=O ; *CCOCC(=O)NCC#Cc1cn(*)c(=O)nc1N]

In embodiments, B¹ is

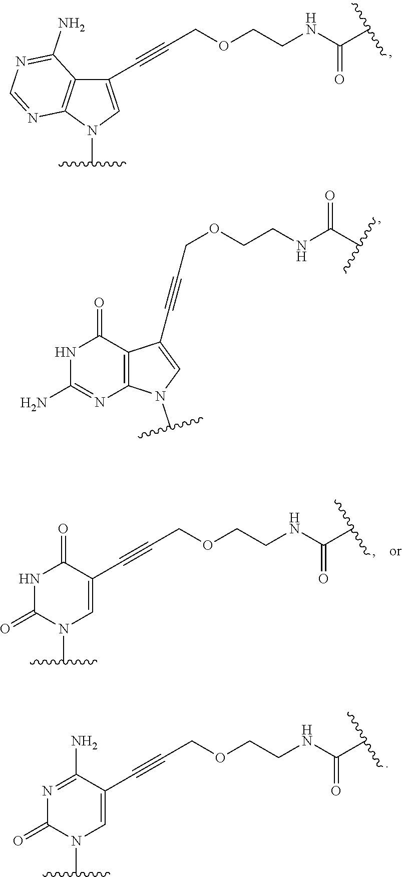 [SMILES: *C(=O)NCCOCC#Cc1cn(*)c2ncnc(N)c12 ; *C(=O)NCCOCC#Cc1cn(*)c2nc(N)[nH]c(=O)c12 ; *C(=O)NCCOCC#Cc1cn(*)c(=O)[nH]c1=O ; *C(=O)NCCOCC#Cc1cn(*)c(=O)nc1N]

In embodiments, B¹ is

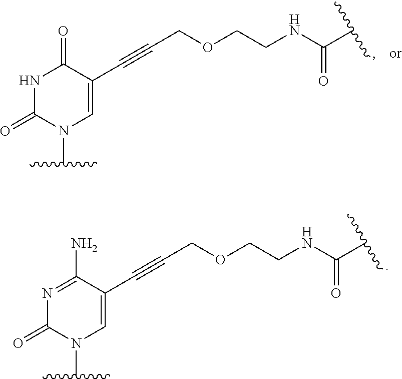 [SMILES: *C(=O)NCCOCC#Cc1cn(*)c(=O)[nH]c1=O ; *C(=O)NCCOCC#Cc1cn(*)c(=O)nc1N]

In embodiments, B¹ is

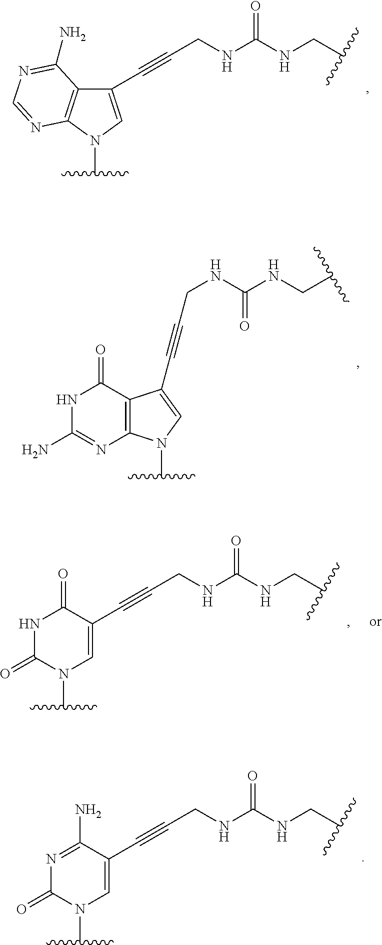 [SMILES: *CNC(=O)NCC#Cc1cn(*)c2ncnc(N)c12 ; *CNC(=O)NCC#Cc1cn(*)c2nc(N)[nH]c(=O)c12 ; *CNC(=O)NCC#Cc1cn(*)c(=O)[nH]c1=O ; *CNC(=O)NCC#Cc1cn(*)c(=O)nc1N]

In embodiments, B¹ is

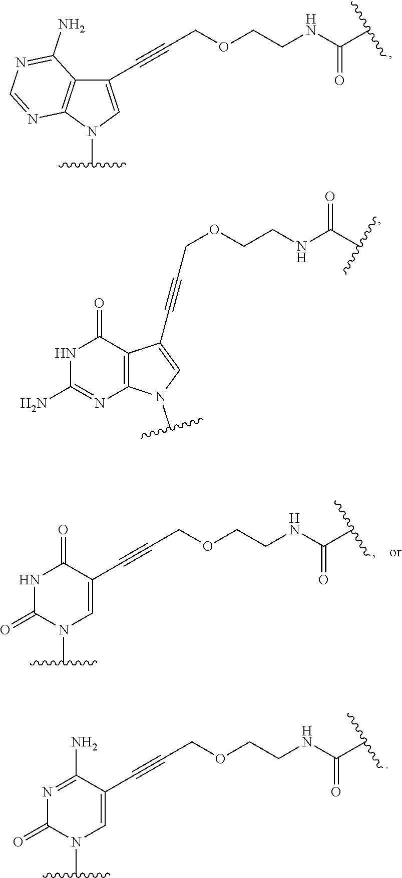 [SMILES: *C(=O)NCCOCC#Cc1cn(*)c2ncnc(N)c12 ; *C(=O)NCCOCC#Cc1cn(*)c2nc(N)[nH]c(=O)c12 ; *C(=O)NCCOCC#Cc1cn(*)c(=O)[nH]c1=O ; *C(=O)NCCOCC#Cc1cn(*)c(=O)nc1N]

In embodiments, R¹ is independently a monophosphate moiety or a derivative thereof (e.g., including a phosphoramidate moiety, phosphorothioate moiety, phosphorodithioate moiety, or methylphosphoroamidite moiety), polyphosphate moiety or derivative thereof (e.g., including a phosphoramidate, phosphorothioate, phosphorodithioate, or methylphosphoroamidite), or nucleic acid moiety or derivative thereof (e.g., including a phosphoramidate, phosphorothioate, phosphorodithioate, or methylphosphoroamidite). In embodiments, R¹ is a nucleic acid moiety. In embodiments, R¹ is a monophosphate moiety, polyphosphate moiety, or nucleic acid moiety. In embodiments, R¹ is a monophosphate moiety. In embodiments, R¹ is a polyphosphate moiety. In embodiments, R¹ is a nucleic acid moiety. In embodiments, R¹ is hydrogen. In embodiments, R¹ is a triphosphate, having the formula:

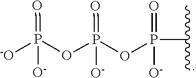

In embodiments, R¹ is a triphosphate, having the formula:

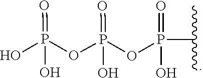 [SMILES: *P(=O)(O)OP(=O)(O)OP(=O)(O)O]

In embodiments, R¹ is a thiotriphosphate, having the formula:

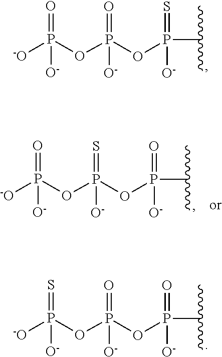

In embodiments, R¹ is a thiotriphosphate, having the formula:

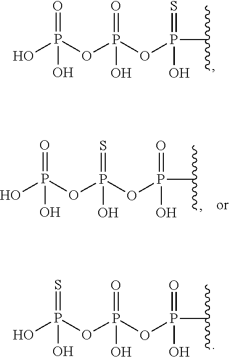 [SMILES: *P(O)(=S)OP(=O)(O)OP(=O)(O)O ; *P(=O)(O)OP(O)(=S)OP(=O)(O)O ; *P(=O)(O)OP(=O)(O)OP(O)(O)=S]

In embodiments, R² is hydrogen. In embodiments, R² is —OH.

In embodiments, R³ is a reversible terminator. For example, the reversible terminator may include a known reversible terminator moiety, such as azidomethyl moiety, disulfide moiety, nitrobenzyl moiety, allyl moiety, or an allyloxycarbonyl (See, for example, Metzker et al., “Termination of DNA synthesis by novel 3′-modified deoxyribonucleoside triphosphates,” Nucleic Acids Res., 22:4259-4267, 1994; and U.S. Pat. Nos. 5,872,244; 6,232,465; 6,214,987; 5,808,045; 5,763,594, and 5,302,509. Typically, reversible terminators require contact with a cleaving agent (e.g., a reducing agent or an acid) or suitable radiation (e.g., UV) to remove the reversible terminator and expose a 3′-OH on the nucleotide. In embodiments, the reversible terminator moiety is

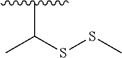 [SMILES: *C(C)SSC]

as described in U.S. Pat. No. 10,738,072, which is incorporated herein by reference for all purposes. In embodiments, the reversible terminator moiety is cyanoethenyl, allenyl, formaldehyde oximyl, acrylaldehyde oximyl, propionaldehyde oximyl, cyanoethenaldehyde oximyl, cis-cyanoethenyl, trans-cyanoethenyl, cis-cyanofluoroethenyl, trans-cyanofluoroethenyl, biscyanoethenyl, bisfluoroethenyl, cis-propenyl, trans-propenyl, nitroethenyl, acetoethenyl, methylcarbonoethenyl, amidoethenyl, methylsulfonoethenyl, methylsulfonoethyl, formimidate, formhydroxymate, vinyloethenyl, ethylenoethenyl, cyanoethylenyl, nitroethylenyl, amidoethylenyl, for example the reversible terminator moieties as described in U.S. Publication 2019/0144482, which is incorporated herein by reference for all purposes. In embodiments, the reversible terminator moiety includes an alkyne moiety (e.g., a propargyl moiety), for example the reversible terminator moieties as described in U.S. Publication 2015/0050697, which is incorporated herein by reference for all purposes. In embodiments, the reversible terminator moiety includes a phosphate diester group as described in U.S. Publication 2014/0242579, which is incorporated herein by reference for all purposes.

In embodiments, R³ is

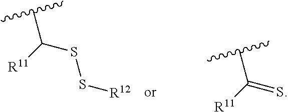

R¹¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, —NR¹³R¹⁴, substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20 membered, 8 to 20 membered, 2 to 10 membered, 3 to 10 membered, 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered). R¹² is unsubstituted alkyl (e.g., C₁-C₂₀, C₁₀-C₂₀, C₁-C₈, C₁-C₆, or C₁-C₄). R¹³ and R¹⁴ are each independently hydrogen, substituted or unsubstituted alkyl (e.g., C₁-C₂₀, C₁₀-C₂₀, C₁-C₈, C₁-C₆, or C₁-C₄), or substituted or unsubstituted heteroalkyl (e.g., 2 to 20 membered, 8 to 20 membered, 2 to 10 membered, 3 to 10 membered, 2 to 8 membered, 2 to 6 membered, or 2 to 4 membered).

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when Ru is substituted, it is substituted with at least one lower substituent group.

In embodiments, R¹¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, or —NR¹³R¹⁴. In embodiments, R¹¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹¹ is hydrogen. In embodiments, R¹¹ is R^{11A}-substituted or unsubstituted alkyl, R^{11A}-substituted or unsubstituted heteroalkyl, R^{11A}-substituted or unsubstituted cycloalkyl, R^{11A}-substituted or unsubstituted heterocycloalkyl, R^{11A}-substituted or unsubstituted aryl, or R^{11A}-substituted or unsubstituted heteroaryl. In embodiments, R¹¹ is —NH₂, —NH(CH₃), or —N(CH₃)₂.

In embodiments, R¹¹ is unsubstituted C₁-C₆ or C₁-C₄ alkyl. In embodiments, R¹¹ is unsubstituted C₁-C₄ alkyl. In embodiments, R¹¹ is unsubstituted methyl. In embodiments, R¹¹ is unsubstituted C₂ alkyl. In embodiments, R¹¹ is unsubstituted C₃ alkyl. In embodiments, R¹¹ is unsubstituted C₄ alkyl. In embodiments, R¹¹ is unsubstituted C₅ alkyl. In embodiments, R¹¹ is unsubstituted C₆ alkyl. In embodiments, R¹¹ is unsubstituted C₁-C₆ or C₁-C₄ saturated alkyl. In embodiments, R¹¹ is unsubstituted C₁-C₄ saturated alkyl. In embodiments, R¹¹ is unsubstituted C₁-C₆ saturated alkyl. In embodiments, R¹¹ is unsubstituted methyl. In embodiments, R¹¹ is unsubstituted C₂ saturated alkyl. In embodiments, R¹¹ is unsubstituted C₃ saturated alkyl. In embodiments, R¹¹ is unsubstituted C₄ saturated alkyl. In embodiments, R¹¹ is unsubstituted C₅ saturated alkyl. In embodiments, R¹¹ is unsubstituted C₆ saturated alkyl. In embodiments, R¹¹ is R^{11A}-substituted C₁-C₆ or C₁-C₄ alkyl. In embodiments, R¹¹ is R^{11A}-substituted C₁-C₄ alkyl. In embodiments, R¹¹ is R^{11A}-substituted methyl. In embodiments, R¹¹ is R^{11A}-substituted C₂ alkyl. In embodiments, R¹¹ is R^{11A}-substituted C₃ alkyl. In embodiments, R¹¹ is R^{11A}-substituted C₄ alkyl. In embodiments, R¹¹ is R^{11A}-substituted C₅ alkyl. In embodiments, R¹¹ is R^{11A}-substituted C₆ alkyl. In embodiments, R¹¹ is R^{11A}-substituted or unsubstituted aryl (e.g., C₆-C₁₀, C₁₀, or phenyl). In embodiments, R¹¹ is R^{11A}-substituted aryl (e.g., C₆-C₁₀, C₁₀, or phenyl). In embodiments, R¹¹ is unsubstituted aryl (e.g., C₆-C₁₀, C₁₀, or phenyl). In embodiments, R¹ is unsubstituted phenyl. In embodiments, R¹¹ is R^{11A}-substituted or unsubstituted heteroaryl (e.g., 5 to 10, 5 to 9, or 5 to 6 membered). In embodiments, R¹¹ is R^{11A}-substituted heteroaryl (e.g., 5 to 10, 5 to 9, or 5 to 6 membered). In embodiments, R¹¹ is unsubstituted heteroaryl (e.g., 5 to 10, 5 to 9, or 5 to 6 membered). In embodiments, R¹¹ is a R^{11A}-substituted or unsubstituted 5 membered heteroaryl. In embodiments, R¹¹ is a R^{11A}-substituted or unsubstituted 6 membered heteroaryl. In embodiments, R¹¹ is a R^{11A}-substituted or unsubstituted 7 membered heteroaryl. In embodiments, R¹¹ is an unsubstituted 5 membered heteroaryl. In embodiments, R¹¹ is an unsubstituted 6 membered heteroaryl. In embodiments, R¹¹ is an unsubstituted 7 membered heteroaryl.

In embodiments, R¹¹ is

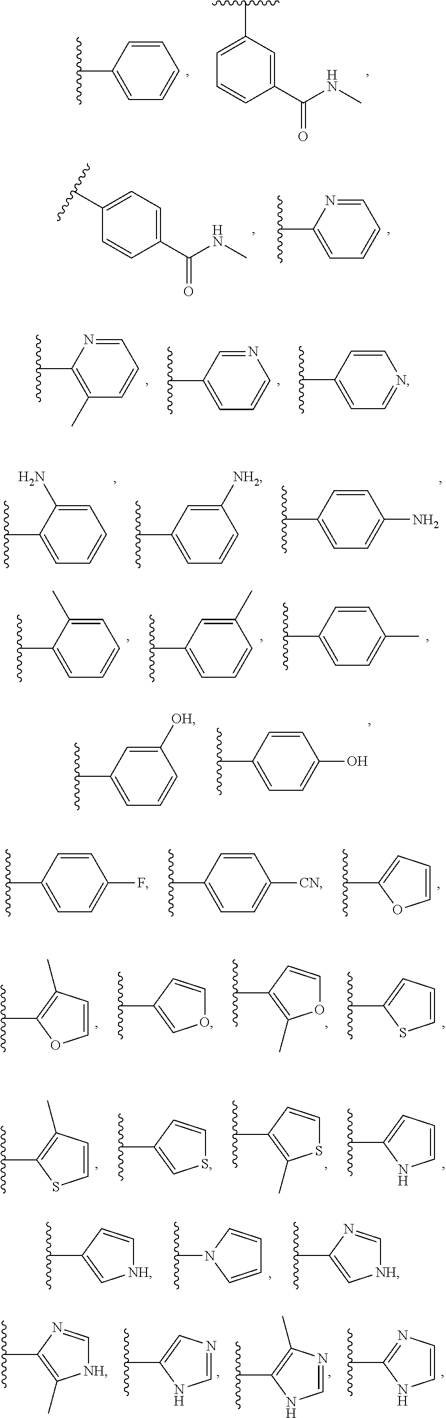

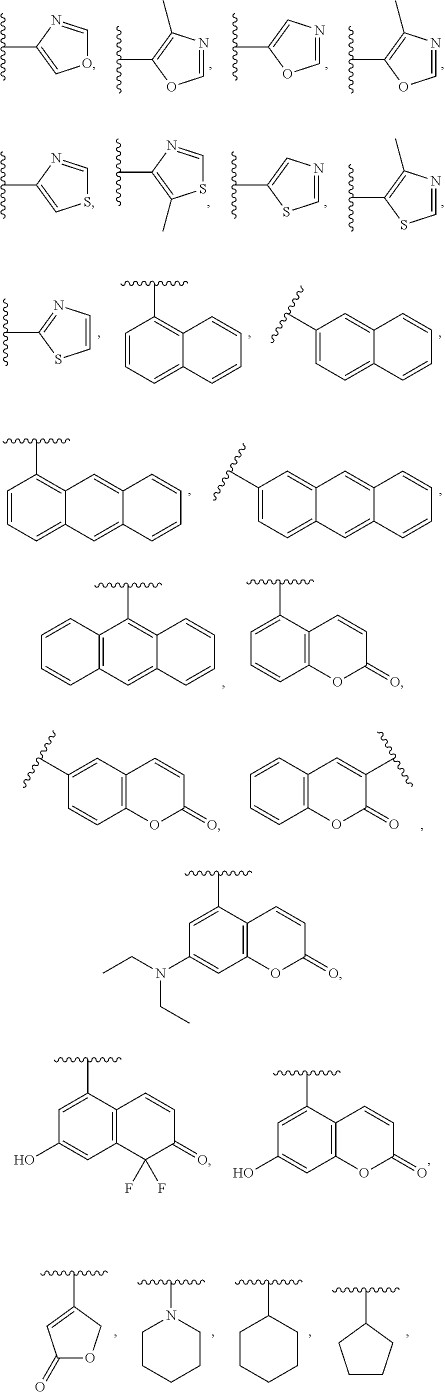

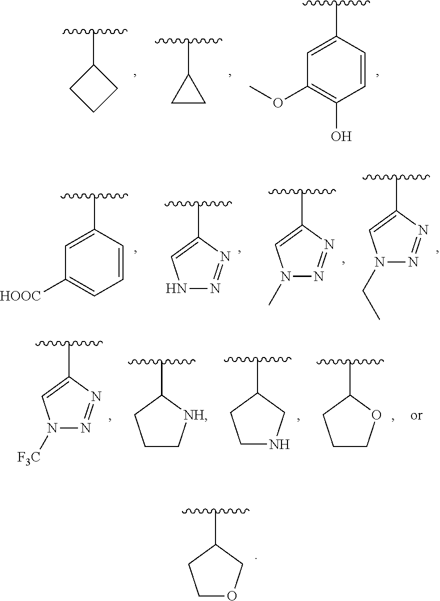

In embodiments, R¹¹ is

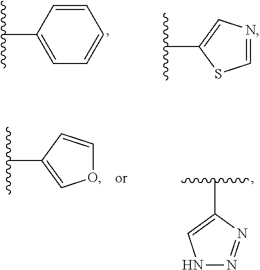 [SMILES: *c1ccccc1 ; *c1cncs1 ; *c1ccoc1 ; *c1c[nH]nn1]

In embodiments, R¹² is unsubstituted C₁-C₆ or C₁-C₄ alkyl. In embodiments, R¹² is unsubstituted C₁-C₄ alkyl. In embodiments, R¹² is unsubstituted C₁-C₆ alkyl. In embodiments, R¹² is unsubstituted methyl. In embodiments, R¹² is unsubstituted C₂ alkyl. In embodiments, R¹² is unsubstituted C₃ alkyl. In embodiments, R¹² is unsubstituted C₄ alkyl. In embodiments, R¹² is unsubstituted C₅ alkyl. In embodiments, R¹² is unsubstituted C₆ alkyl.

In embodiments, a substituted R¹³ (e.g., substituted alkyl and/or substituted heteroalkyl) is substituted with at least one substituent group, size-limited substituent group, or lower substituent group; wherein if the substituted R¹³ is substituted with a plurality of groups selected from substituent groups, size-limited substituent groups, and lower substituent groups; each substituent group, size-limited substituent group, and/or lower substituent group may optionally be different. In embodiments, when R¹³ is substituted, it is substituted with at least one substituent group. In embodiments, when R¹³ is substituted, it is substituted with at least one size-limited substituent group. In embodiments, when R¹³ is substituted, it is substituted with at least one lower substituent group.

In embodiments, R¹³ is hydrogen, substituted or unsubstituted alkyl, or substituted or unsubstituted heteroalkyl. In embodiments, R¹³ is hydrogen. In embodiments, R¹³ is R^{13A}-substituted or unsubstituted alkyl, or R^{13A}-substituted or unsubstituted heteroalkyl. In embodiments, R¹³ is unsubstituted C₁-C₆ or C₁-C₄ alkyl. In embodiments, R¹³ is unsubstituted C₁-C₄ alkyl. In embodiments, R¹³ is unsubstituted methyl. In embodiments, R¹³ is unsubstituted C₂ alkyl. In embodiments, R¹³ is unsubstituted C₃ alkyl. In embodiments, R¹³ is unsubstituted C₄ alkyl. In embodiments, R¹³ is unsubstituted C₅ alkyl. In embodiments, R¹³ is unsubstituted C₆ alkyl. In embodiments, R¹³ is unsubstituted C₁-C₆ or C₁-C₄ saturated alkyl. In embodiments, R¹³ is unsubstituted C₁-C₄ saturated alkyl. In embodiments, R¹³ is unsubstituted C₁-C₆ saturated alkyl. In embodiments, R¹³ is unsubstituted methyl. In embodiments, R¹³ is unsubstituted C₂ saturated alkyl. In embodiments, R¹³ is unsubstituted C₃ saturated alkyl. In embodiments, R¹³ is unsubstituted C₄ saturated alkyl. In embodiments, R¹³ is unsubstituted C₅ saturated alkyl. In embodiments, R¹³ is unsubstituted C₆ saturated alkyl. In embodiments, R¹³ is R^{13A}-substituted C₁-C₆ or C₁-C₄ alkyl. In embodiments, R¹³ is R^{13A}-substituted C₁-C₄ alkyl. In embodiments, R¹³ is R^{13A}-substituted methyl. In embodiments, R¹³ is R^{13A}-substituted C₂ alkyl. In embodiments, R¹³ is R^{13A}-substituted C₃ alkyl. In embodiments, R¹³ is R^{13A}-substituted C₄ alkyl. In embodiments, R¹³ is R^{13A}-substituted C₅ alkyl. In embodiments, R¹³ is R^{13A}-substituted C₆ alkyl. In embodiments, R¹³ is R^{13A}-substituted or unsubstituted 2 to 8 membered heteroalkyl. In embodiments, R¹³ is R^{13A}-substituted or unsubstituted 2 to 6 membered heteroalkyl. In embodiments, R¹³ is R^{13A}-substituted or unsubstituted 2 to 4 membered heteroalkyl. In embodiments, R¹³ is unsubstituted 2 to 8 membered heteroalkyl. In embodiments, R¹³ is unsubstituted 2 to 6 membered heteroalkyl. In embodiments, R¹³ is unsubstituted 2 to 4 membered heteroalkyl.

In embodiments, a substituted R¹⁴ (e.g., substituted alkyl and/or substituted heteroalkyl) is substituted with at least one substituent group, size-limited substituent group, or lower substituent group; wherein if the substituted R¹⁴ is substituted with a plurality of groups selected from substituent groups, size-limited substituent groups, and lower substituent groups; each substituent group, size-limited substituent group, and/or lower substituent group may optionally be different. In embodiments, when R¹⁴ is substituted, it is substituted with at least one substituent group. In embodiments, when R¹⁴ is substituted, it is substituted with at least one size-limited substituent group. In embodiments, when R¹⁴ is substituted, it is substituted with at least one lower substituent group.

In embodiments, R¹⁴ is hydrogen, substituted or unsubstituted alkyl, or substituted or unsubstituted heteroalkyl. In embodiments, R¹⁴ is hydrogen. In embodiments, R¹⁴ is R^{14A}-substituted or unsubstituted alkyl, or R^{14A}-substituted or unsubstituted heteroalkyl. In embodiments, R¹⁴ is unsubstituted C₁-C₆ or C₁-C₄ alkyl. In embodiments, R¹⁴ is unsubstituted C₁-C₄ alkyl. In embodiments, R¹⁴ is unsubstituted methyl. In embodiments, R¹⁴ is unsubstituted C₂ alkyl. In embodiments, R¹⁴ is unsubstituted C₃ alkyl. In embodiments, R¹⁴ is unsubstituted C₄ alkyl. In embodiments, R¹⁴ is unsubstituted C₅ alkyl. In embodiments, R¹⁴ is unsubstituted C₆ alkyl. In embodiments, R¹⁴ is unsubstituted C₁-C₆ or C₁-C₄ saturated alkyl. In embodiments, R¹⁴ is unsubstituted C₁-C₄ saturated alkyl. In embodiments, R¹⁴ is unsubstituted C₁-C₆ saturated alkyl. In embodiments, R¹⁴ is unsubstituted methyl. In embodiments, R¹⁴ is unsubstituted C₂ saturated alkyl. In embodiments, R¹⁴ is unsubstituted C₃ saturated alkyl. In embodiments, R¹⁴ is unsubstituted C₄ saturated alkyl. In embodiments, R¹⁴ is unsubstituted C₅ saturated alkyl. In embodiments, R¹⁴ is unsubstituted C₆ saturated alkyl. In embodiments, R¹⁴ is R^{14A}-substituted C₁-C₆ or C₁-C₄ alkyl. In embodiments, R¹⁴ is R^{14A}-substituted C₁-C₄ alkyl. In embodiments, R¹⁴ is R^{14A}-substituted methyl. In embodiments, R¹⁴ is R^{14A}-substituted C₂ alkyl. In embodiments, R¹⁴ is R^{14A}-substituted C₃ alkyl. In embodiments, R¹⁴ is R^{14A}-substituted C₄ alkyl. In embodiments, R¹⁴ is R^{14A}-substituted C₅ alkyl. In embodiments, R¹⁴ is R^{14A}-substituted C₆ alkyl. In embodiments, R¹⁴ is R^{14A}-substituted or unsubstituted 2 to 8 membered heteroalkyl. In embodiments, R¹⁴ is R^{14A}-substituted or unsubstituted 2 to 6 membered heteroalkyl. In embodiments, R¹⁴ is R^{14A}-substituted or unsubstituted 2 to 4 membered heteroalkyl. In embodiments, R¹⁴ is unsubstituted 2 to 8 membered heteroalkyl. In embodiments, R¹⁴ is unsubstituted 2 to 6 membered heteroalkyl. In embodiments, R¹⁴ is unsubstituted 2 to 4 membered heteroalkyl.

R^{11A}, R^{13A}, and R^{14A} are each independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R³ is —NH₂, —CN, —CH₃, C₂-C₆ allyl (e.g., —CH₂—CH═CH₂), methoxyalkyl (e.g., —CH₂—O—CH₃ or —CH₂—O—CH₂—CH═CH), or —CH₂N₃. In embodiments, R³ is —CH₂N₃. In embodiments, R³ is

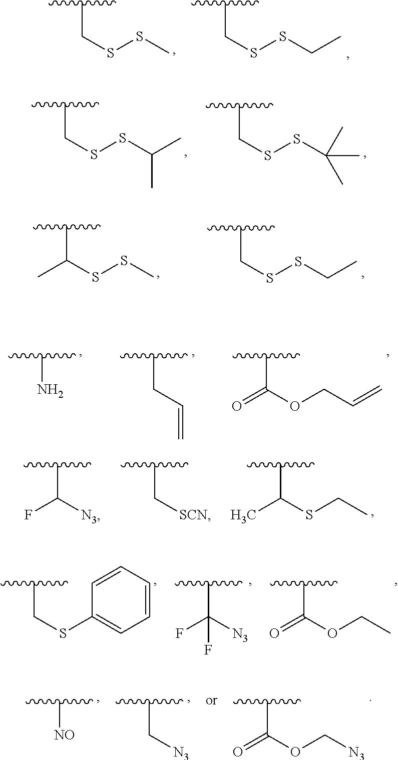

In embodiments, R³ is

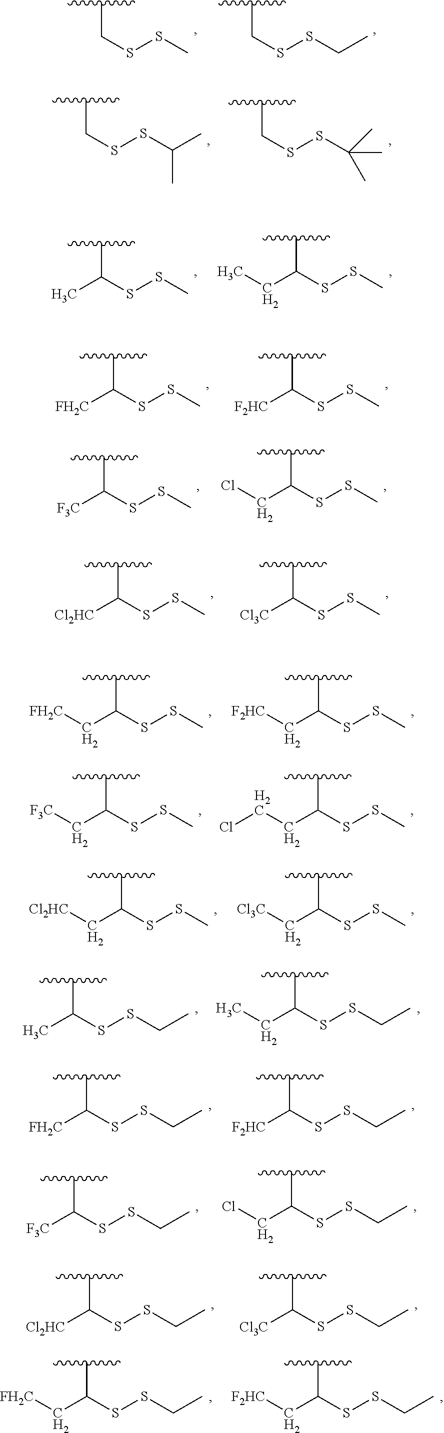

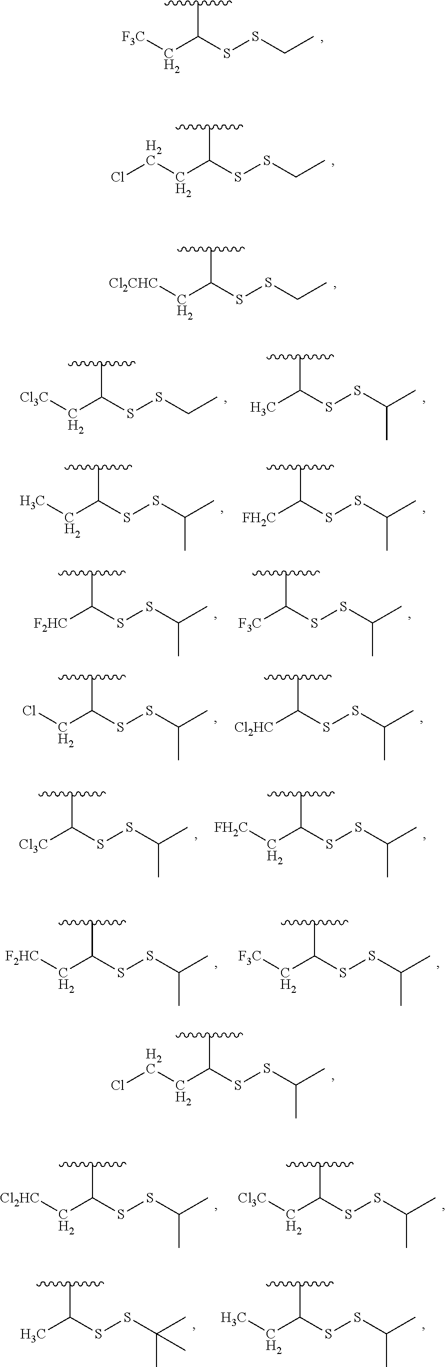

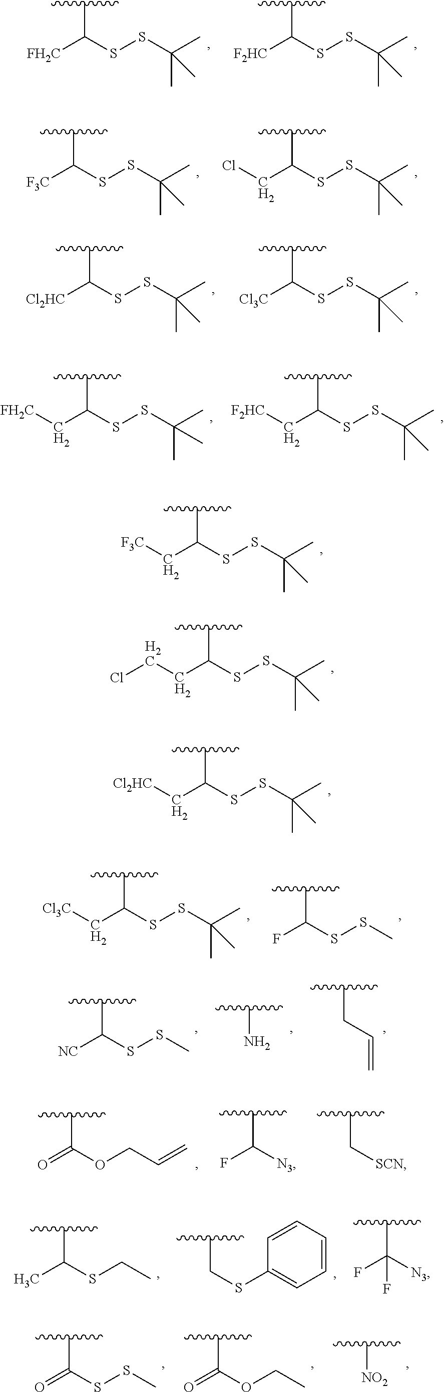

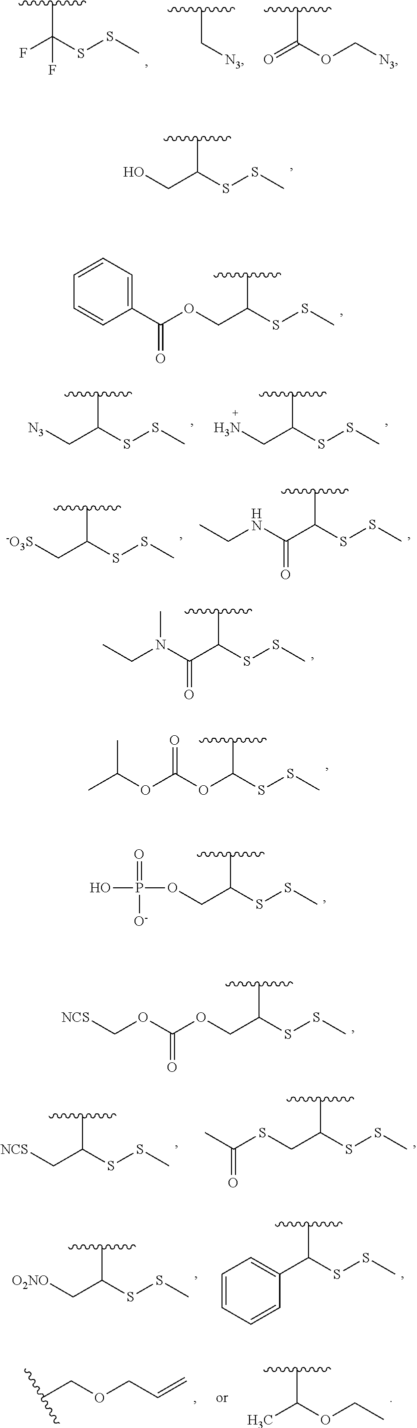

In embodiments, R³ is

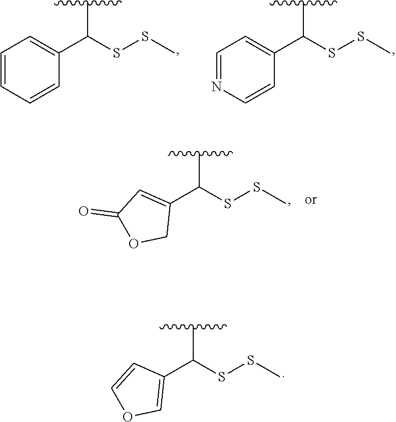

In embodiments, R³ is

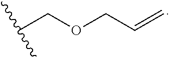 [SMILES: *COCC=C]

In embodiments, R³ is

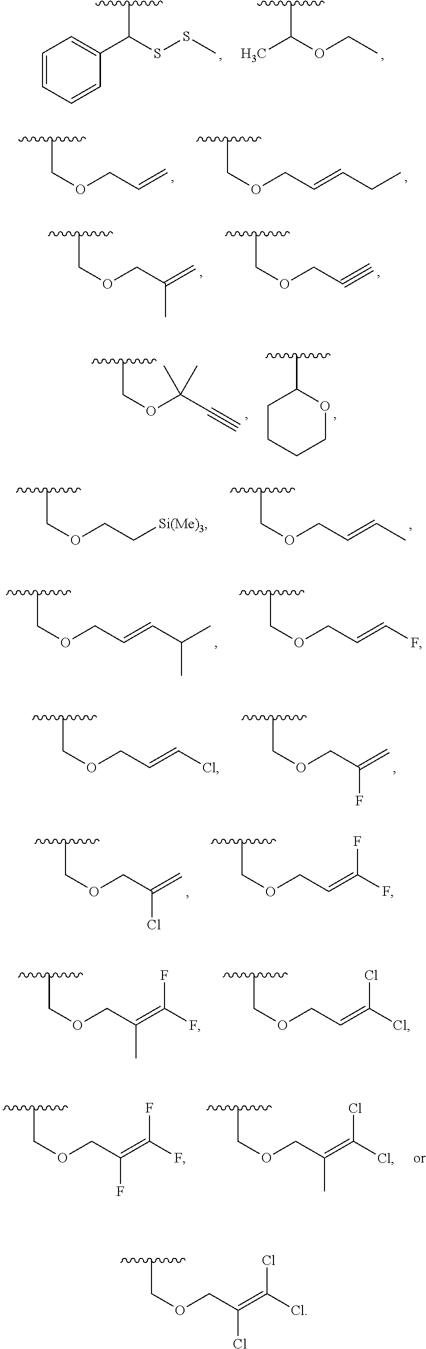

In embodiments, L¹⁰⁰ is a cleavable linker including an azido (i.e., —N₃) moiety or a dithio (i.e., —S—S—) moiety. In embodiments, L¹⁰⁰ is a cleavable linker including:

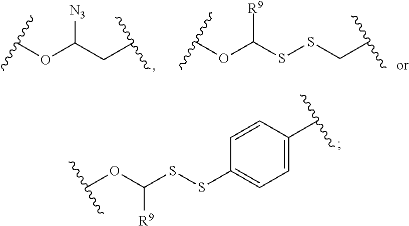

wherein, R⁹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹ is substituted or unsubstituted alkyl. In embodiments, R⁹ is substituted or unsubstituted aryl, or substituted or unsubstituted heteroaryl. In embodiments, L¹⁰⁰ includes

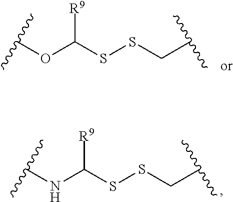

wherein R⁹ is as described herein. In embodiments, L¹⁰⁰ includes

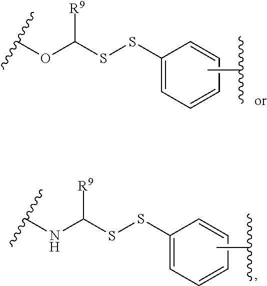

wherein R⁹ is as described herein. In embodiments, L¹⁰⁰ includes

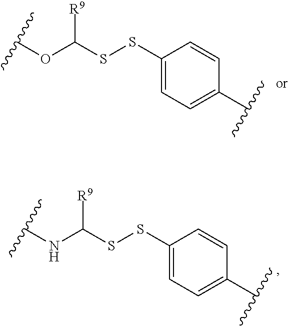

wherein R⁹ is as described herein.

In embodiments, L¹⁰⁰ is a cleavable linker comprising an azido moiety, a disulfide moiety, or an alkoxyalkyl moiety. In embodiments, L¹⁰⁰ is

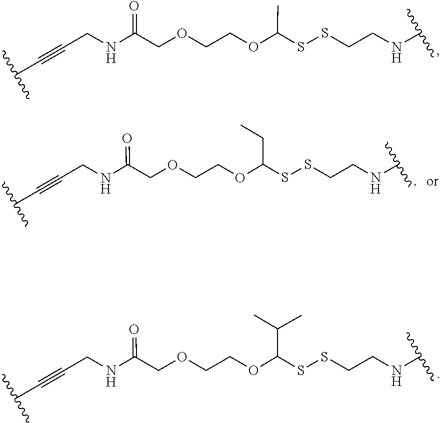 [SMILES: *C#CCNC(=O)COCCOC(C)SSCCN* ; *C#CCNC(=O)COCCOC(CC)SSCCN* ; *C#CCNC(=O)COCCOC(SSCCN*)C(C)C]

In embodiments, L¹⁰⁰ is

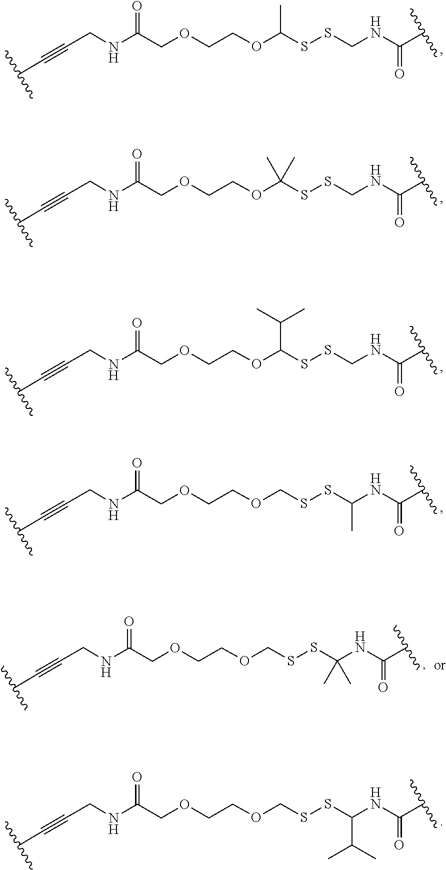 [SMILES: *C#CCNC(=O)COCCOC(C)SSCNC(*)=O ; *C#CCNC(=O)COCCOC(C)(C)SSCNC(*)=O ; *C#CCNC(=O)COCCOC(SSCNC(*)=O)C(C)C ; *C#CCNC(=O)COCCOCSSC(C)NC(*)=O ; *C#CCNC(=O)COCCOCSSC(C)(C)NC(*)=O ; *C#CCNC(=O)COCCOCSSC(NC(*)=O)C(C)C]

In embodiments, L¹⁰⁰ is

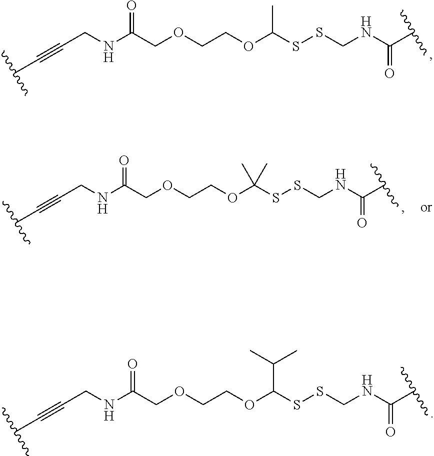 [SMILES: *C#CCNC(=O)COCCOC(C)SSCNC(*)=O ; *C#CCNC(=O)COCCOC(C)(C)SSCNC(*)=O ; *C#CCNC(=O)COCCOC(SSCNC(*)=O)C(C)C]

In embodiments, L¹⁰⁰ is

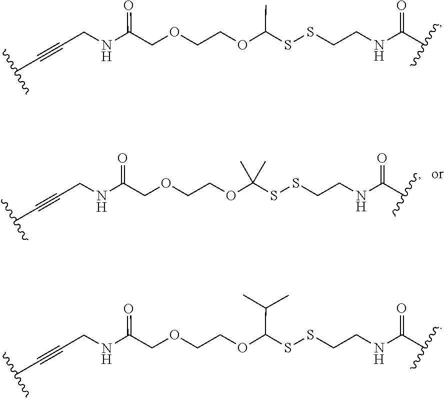 [SMILES: *C#CCNC(=O)COCCOC(C)SSCCNC(*)=O ; *C#CCNC(=O)COCCOC(C)(C)SSCCNC(*)=O ; *C#CCNC(=O)COCCOC(SSCCNC(*)=O)C(C)C]

In embodiments, L¹⁰⁰ is

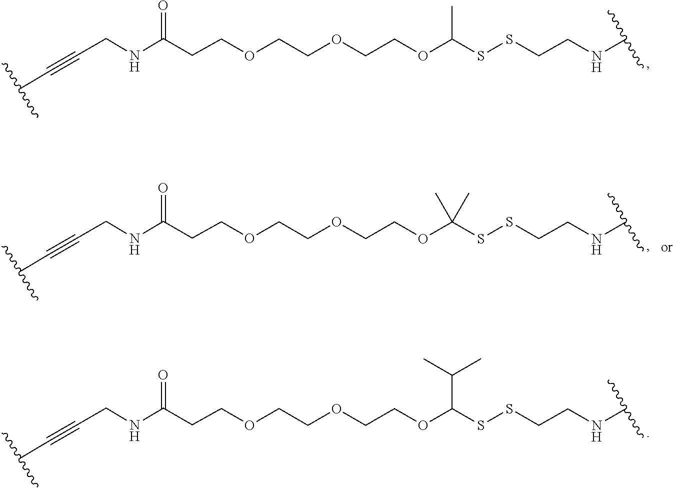 [SMILES: *C#CCNC(=O)CCOCCOCCOC(C)SSCCN* ; *C#CCNC(=O)CCOCCOCCOC(C)(C)SSCCN* ; *C#CCNC(=O)CCOCCOCCOC(SSCCN*)C(C)C]

In embodiments, L¹⁰⁰ is

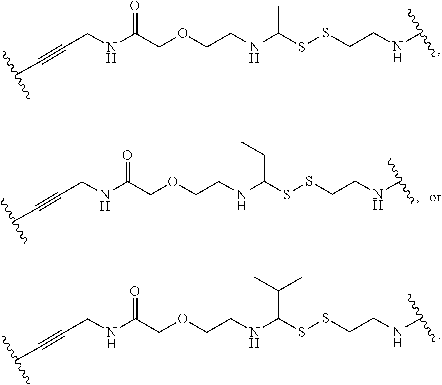 [SMILES: *C#CCNC(=O)COCCNC(C)SSCCN* ; *C#CCNC(=O)COCCNC(CC)SSCCN* ; *C#CCNC(=O)COCCNC(SSCCN*)C(C)C]

In embodiments, L¹⁰⁰ is

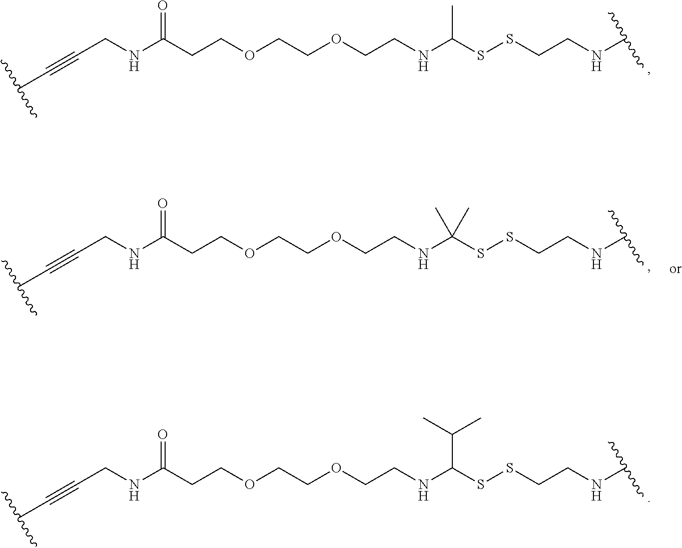 [SMILES: *C#CCNC(=O)CCOCCOCCNC(C)SSCCN* ; *C#CCNC(=O)CCOCCOCCNC(C)(C)SSCCN* ; *C#CCNC(=O)CCOCCOCCNC(SSCCN*)C(C)C]

In embodiments, L¹⁰⁰ is

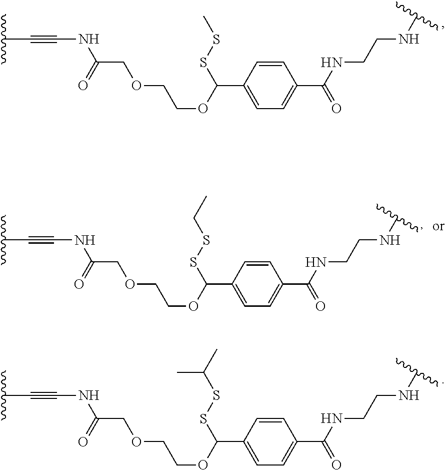 [SMILES: *C#CNC(=O)COCCOC(SSC)c1ccc(C(=O)NCCN*)cc1 ; *C#CNC(=O)COCCOC(SSCC)c1ccc(C(=O)NCCN*)cc1 ; *C#CNC(=O)COCCOC(SSC(C)C)c1ccc(C(=O)NCCN*)cc1]

In embodiments, L¹⁰⁰ is

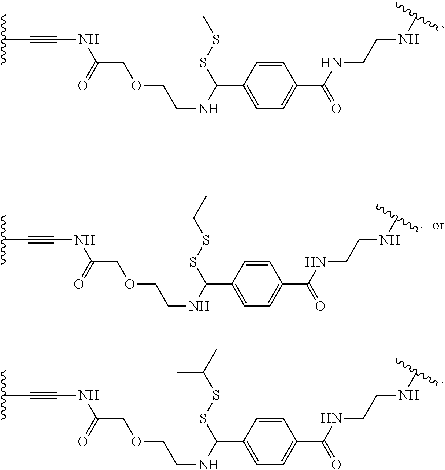 [SMILES: *C#CNC(=O)COCCNC(SSC)c1ccc(C(=O)NCCN*)cc1 ; *C#CNC(=O)COCCNC(SSCC)c1ccc(C(=O)NCCN*)cc1 ; *C#CNC(=O)COCCNC(SSC(C)C)c1ccc(C(=O)NCCN*)cc1]

In embodiments, L¹⁰⁰ is

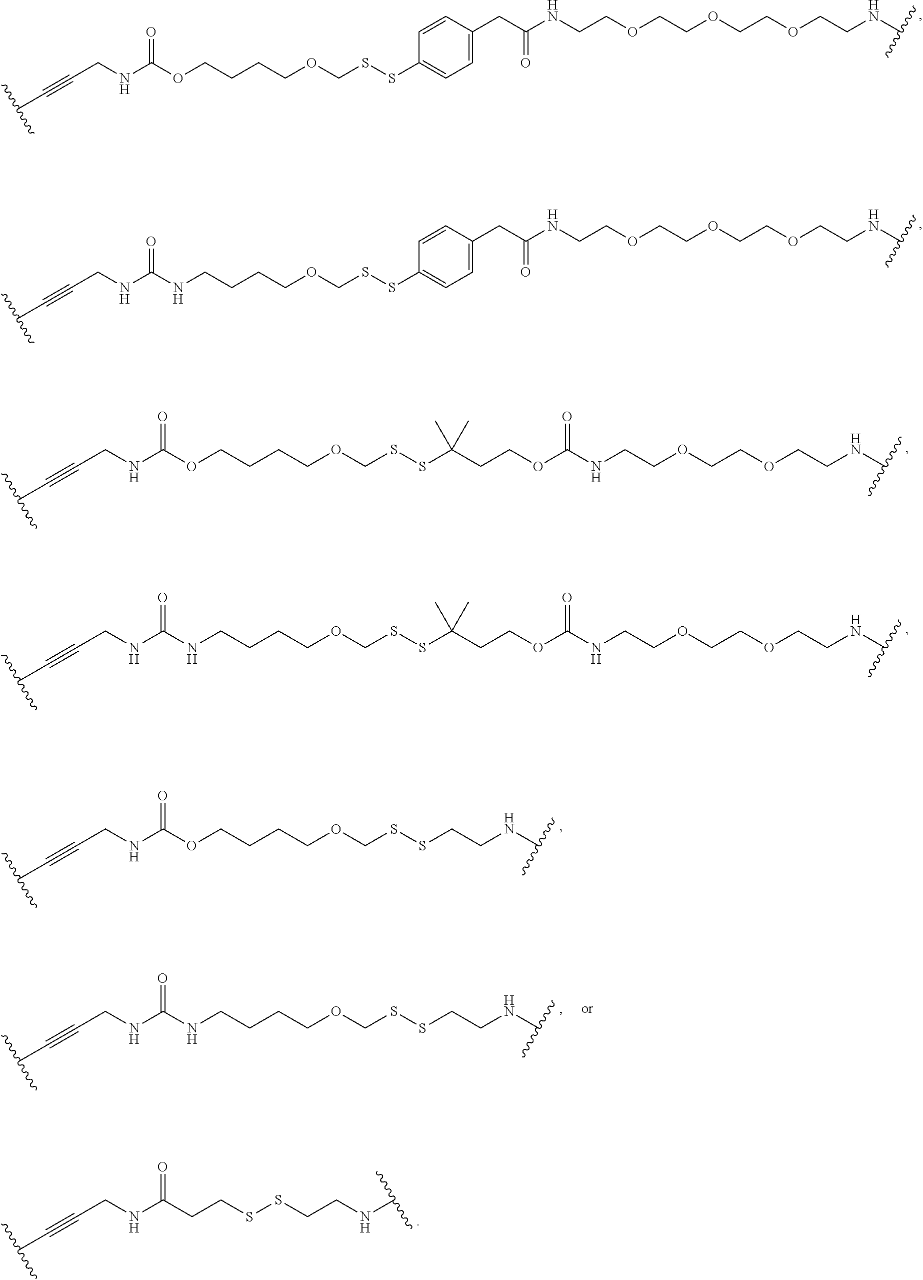

In embodiments, L¹⁰⁰ is

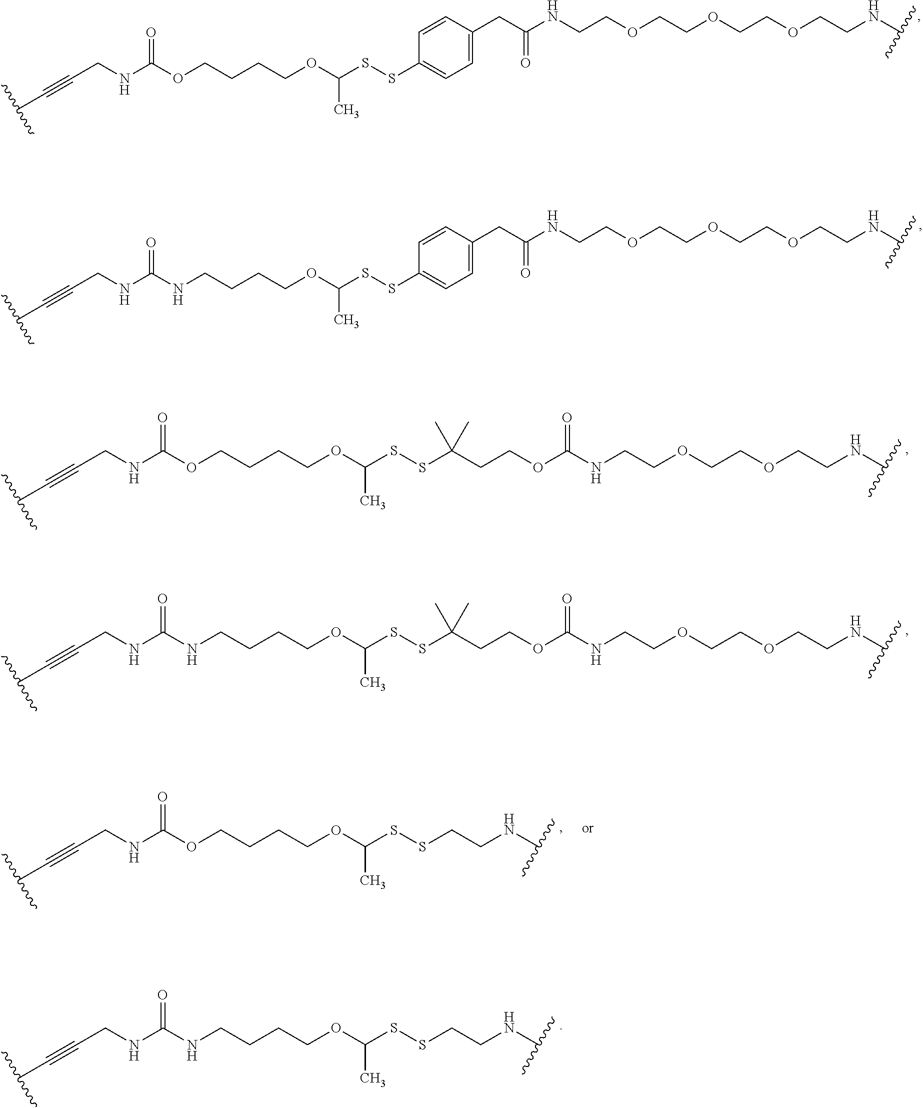 [SMILES: *C#CCNC(=O)OCCCCOC(C)SSc1ccc(CC(=O)NCCOCCOCCOCCN*)cc1 ; *C#CCNC(=O)NCCCCOC(C)SSc1ccc(CC(=O)NCCOCCOCCOCCN*)cc1 ; *C#CCNC(=O)OCCCCOC(C)SSC(C)(C)CCOC(=O)NCCOCCOCCN* ; *C#CCNC(=O)NCCCCOC(C)SSC(C)(C)CCOC(=O)NCCOCCOCCN* ; *C#CCNC(=O)OCCCCOC(C)SSCCN* ; *C#CCNC(=O)NCCCCOC(C)SSCCN*]

In embodiments, L¹⁰⁰ is

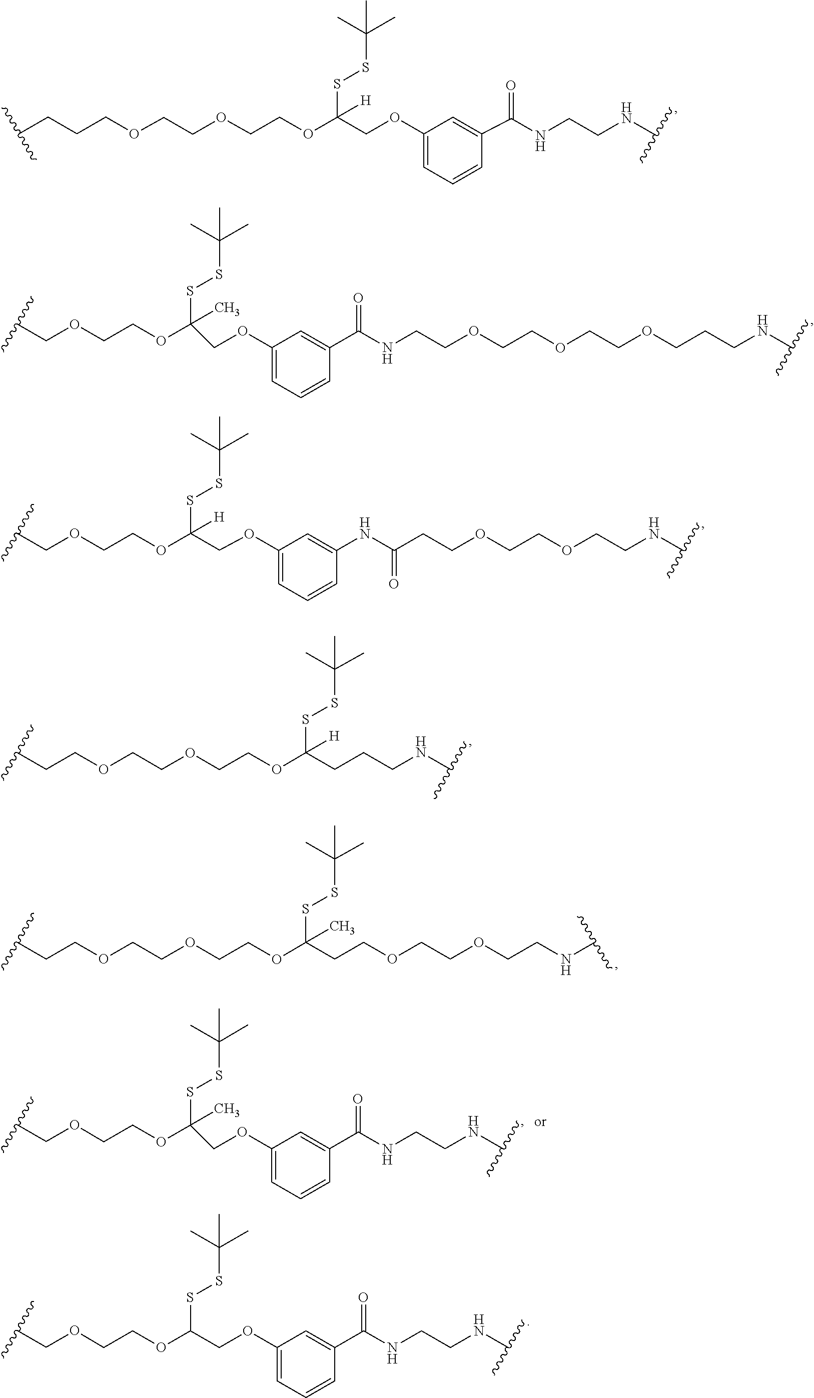

In embodiments, L¹⁰⁰ is -L¹⁰¹-L¹⁰²-L¹⁰³-L¹⁰⁴-L¹⁰⁵-. L¹⁰¹, L¹⁰², L¹⁰³, L¹⁰⁴, and L¹⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, thio-trigger moiety, substituted or unsubstituted alkylene (e.g., —CH(OH)— or —C(CH₂)—), substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹⁰¹, L¹⁰², L¹⁰³, L¹⁰⁴, and L¹⁰⁵ independently includes PEG. In embodiments, L¹⁰¹, L¹⁰², L¹⁰³, L¹⁰⁴, and L¹⁰⁵ independently includes

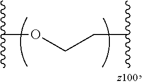

wherein z100 is independently an integer from 1 to 8. In embodiments, z100 is 1. In embodiments, z100 is 2. In embodiments, z100 is 3. In embodiments, z100 is 4. In embodiments, z100 is 5. In embodiments, z100 is 6. In embodiments, z100 is 7. In embodiments, z100 is 8. In embodiments, z100 is an integer from 2 to 8. In embodiments, z100 is an integer from 4 to 6.

In embodiments, at least one of L¹⁰¹, L¹⁰², L¹⁰³, L¹⁰⁴, and L¹⁰⁵ independently includes

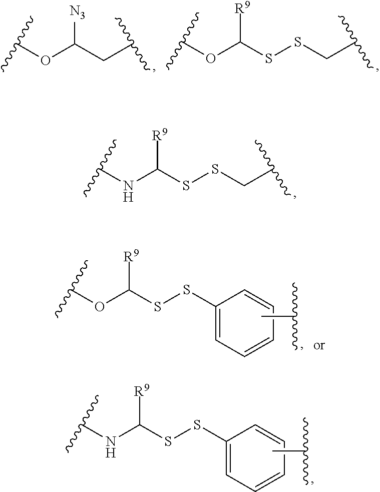

wherein R⁹ is as described herein.

In embodiments, L¹⁰⁰ is -L¹⁰¹-L¹⁰²-L¹⁰³-L¹⁰⁴-L¹⁰⁵-. In embodiments, L¹⁰¹, L¹⁰², L¹⁰³, L¹⁰⁴, and L¹⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰⁰ is -L¹⁰¹-O—CH(N₃)-L¹⁰³-L¹⁰⁴-L¹⁰⁵-; and L¹⁰¹, L¹⁰³, L¹⁰⁴, and L¹⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹⁰¹ is independently a substituted or unsubstituted C₁-C₄ alkylene or substituted or unsubstituted 8 to 20 membered heteroalkylene; L¹⁰³ is independently a bond or substituted or unsubstituted 2 to 10 membered heteroalkylene; L¹⁰⁴ is independently a bond, substituted or unsubstituted 4 to 18 membered heteroalkylene, or substituted or unsubstituted phenylene; L¹⁰⁵ is independently bond or substituted or unsubstituted 4 to 18 membered heteroalkylene. In embodiments, L¹⁰¹, L¹⁰², L¹⁰³, L¹⁰⁴, and/or L¹⁰⁵ are independently a bond, —NH—, —S—, —O—, —C(O)—, —C(O)O—, —OC(O)—, —CH(OH)—, —NHC(O)—, —C(O)NH—, —NHC(O)NH—, —NHC(NH)NH—, —C(S)—, or —C(CH₂)—. In embodiments, L¹⁰¹ is independently a substituted or unsubstituted C₁-C₄ alkylene or substituted or unsubstituted 8 to 20 membered heteroalkylene; L¹⁰³ is independently a bond or substituted or unsubstituted 2 to 10 membered heteroalkylene; L¹⁰⁴ is independently a bond, substituted or unsubstituted 4 to 18 membered heteroalkylene, or substituted or unsubstituted phenylene; and L¹⁰⁵ is independently bond or substituted or unsubstituted 4 to 18 membered heteroalkylene. In embodiments, L¹⁰¹ is independently a substituted or unsubstituted C₁-C₄ alkylene or substituted or unsubstituted 8 to 20 membered heteroalkylene. In embodiments, L¹⁰¹ is independently an oxo-substituted C₁-C₄ alkylene or an oxo-substituted 8 to 20 membered heteroalkylene. In embodiments, L¹⁰³ is independently a bond or substituted or unsubstituted 2 to 10 membered heteroalkylene. In embodiments, L¹⁰³ is independently a bond or an unsubstituted 2 to 10 membered heteroalkylene. In embodiments, L¹⁰⁴ is independently a bond, substituted or unsubstituted 4 to 18 membered heteroalkylene, or substituted or unsubstituted phenylene. In embodiments, L¹⁰⁵ is independently a bond or substituted or unsubstituted 4 to 18 membered heteroalkylene. In embodiments, L¹⁰⁵ is independently a bond or an oxo-substituted 4 to 18 membered heteroalkylene. In embodiments, L¹⁰⁵ is independently a bond or an unsubstituted 4 to 18 membered heteroalkylene.

In embodiments, L¹⁰⁰ is -L¹⁰¹-SS-L¹⁰³-L¹⁰⁴-L¹⁰⁵-. In embodiments, L¹⁰¹, L¹⁰⁴, and L¹⁰⁵ are independently a bond, —NH—, —O—, —C(O)—, —C(O)NH—, —NHC(O)—, —NHC(O)NH—, —C(O)O—, —OC(O)—, substituted or unsubstituted alkylene (e.g., —CH(OH)— or —C(CH₂)—), substituted or unsubstituted heteroalkylene, substituted or unsubstituted cycloalkylene, substituted or unsubstituted heterocycloalkylene, substituted or unsubstituted arylene, or substituted or unsubstituted heteroarylene; and L¹⁰³ is a bond or unsubstituted phenylene.

In embodiments, L¹⁰¹ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹⁰¹ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20 membered, 8 to 20 membered, 2 to 10 membered, 3 to 10 membered, 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L¹⁰ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond, —NH—, —NR¹⁰¹—, —S—, —O—, —C(O)—, —C(O)O—, —OC(O)—, —NHC(O)—, —C(O)NH—, —NHC(O)NH—, —NHC(NH)NH—, —C(S)—, R¹⁰¹-substituted or unsubstituted alkylene (e.g., C₁-C₂₀, C₁₀-C₂₀, C₁-C₈, C₁-C₆, or C₁-C₄), R¹⁰¹-substituted or unsubstituted heteroalkylene (e.g., 2 to 20 membered, 8 to 20 membered, 2 to 10 membered, 3 to 10 membered, 2 to 8 membered, 2 to 6 membered, or 2 to 4 membered), R¹⁰¹-substituted or unsubstituted cycloalkylene (e.g., C₃-C₈, C₃-C₆, or C₅-C₆), R¹⁰¹-substituted or unsubstituted heterocycloalkylene (e.g., 3 to 8, 3 to 6, or 5 to 6 membered), R¹⁰¹-substituted or unsubstituted arylene (e.g., C₆-C₁₀, C₁₀, or phenylene), or R¹⁰¹-substituted or unsubstituted heteroarylene (e.g., 5 to 10, 5 to 9, or 5 to 6 membered). In embodiments, L¹⁰ is a bond. In embodiments, L¹⁰ is —NH—. In embodiments, L¹⁰ is —NR¹⁰¹—. In embodiments, L¹⁰ is —S—. In embodiments, L¹⁰ is —O—. In embodiments, L¹⁰ is —C(O)—. In embodiments, L¹⁰ is —C(O)O—. In embodiments, L¹⁰ is —OC(O)—. In embodiments, L¹⁰¹ is —NHC(O)—. In embodiments, L¹⁰¹ is —C(O)NH—. In embodiments, L¹⁰ is —NHC(O)NH—. In embodiments, L¹⁰ is —NHC(NH)NH—. In embodiments, L¹⁰ is —C(S)—. In embodiments, L¹⁰¹ is R¹⁰¹-substituted or unsubstituted C₁-C₂₀ alkylene. In embodiments, L¹⁰¹ is R¹⁰¹-substituted or unsubstituted 2 to 20 membered heteroalkylene. In embodiments, L¹⁰¹ is R¹⁰¹-substituted or unsubstituted 3 to 10 membered heteroalkylene. In embodiments, L¹⁰ is R¹⁰¹-substituted or unsubstituted C₃-C₈ cycloalkylene. In embodiments, L¹⁰¹ is R¹⁰¹-substituted or unsubstituted 3 to 8 membered heterocycloalkylene. In embodiments, L¹⁰¹ is R¹⁰¹-substituted or unsubstituted C₆-C₁₀ arylene. In embodiments, L¹⁰ is R¹⁰¹-substituted or unsubstituted 5 to 10 membered heteroarylene. In embodiments, L¹⁰¹ is a bond, —NH—, —NR¹⁰¹—, —S—, —O—, —C(O)—, —C(O)O—, —OC(O)—, —NHC(O)—, —C(O)NH—, —NHC(O)NH—, —NHC(NH)NH—, —C(S)—, —CH(OH)—, or —C(CH₂)—. In embodiments, L¹⁰¹ is a bond. In embodiments, L¹⁰¹ is —NH—. In embodiments, L¹⁰¹ is —NR⁰¹—. In embodiments, L¹⁰¹ is —S—. In embodiments, L¹⁰¹ is —O—. In embodiments, L¹⁰¹ is —C(O)—. In embodiments, L¹⁰¹ is —C(O)O—. In embodiments, L¹⁰¹ is —OC(O)—. In embodiments, L¹⁰¹ is —NHC(O)—. In embodiments, L¹⁰¹ is —C(O)NH—. In embodiments, L¹⁰¹ is —NHC(O)NH—. In embodiments, L¹⁰¹ is —NHC(NH)NH—. In embodiments, L¹⁰¹ is —C(S)—. In embodiments, L¹⁰¹ is —CH(OH)—. In embodiments, L¹⁰¹ is —C(CH₂)—. In embodiments, L¹⁰¹ is —(CH₂CH₂O)_{b}—. In embodiments, L¹⁰¹ is —CCCH₂(OCH₂CH₂)ₐ—NHC(O)—(CH₂)_{c}(OCH₂CH₂)_{b}—. In embodiments, L¹⁰¹ is —CHCHCH₂—NHC(O)—(CH₂)_{c}(OCH₂CH₂)_{b}—. In embodiments, L¹⁰¹ is —CCCH₂—NHC(O)—(CH₂)_{c}(OCH₂CH₂)_{b}—. In embodiments, L¹⁰¹ is —CCCH₂—. The symbol a is an integer from 0 to 8. In embodiments, a is 1. In embodiments, a is 0. The symbol b is an integer from 0 to 8. In embodiments, b is 0. In embodiments, b is 1 or 2. In embodiments, b is an integer from 2 to 8. In embodiments, b is 1. The symbol c is an integer from 0 to 8. In embodiments, c is 0. In embodiments, c is 1. In embodiments, c is 2. In embodiments, c is 3.

R¹⁰¹ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{101A}-substituted or unsubstituted alkyl (e.g., C₁-C₂₀, C₁₀-C₂₀, C₁-C₈, C₁-C₆, or C₁-C₄), R^{101A}-substituted or unsubstituted heteroalkyl (e.g., 2 to 20, 8 to 20, 2 to 10, 2 to 8, 2 to 6, or 2 to 4 membered), R^{101A}-substituted or unsubstituted cycloalkyl (e.g., C₃-C₈, C₃-C₆, or C₅-C₆), R^{101A}-substituted or unsubstituted heterocycloalkyl (e.g., 3 to 8, 3 to 6, or 5 to 6 membered), R^{101A}-substituted or unsubstituted aryl (e.g., C₆-C₁₀, C₁₀, or phenyl), or R^{101A}-substituted or unsubstituted heteroaryl (e.g., 5 to 10, 5 to 9, or 5 to 6 membered).

In embodiments, R¹⁰¹ is independently —NH₂. In embodiments, R¹⁰¹ is independently —OH. In embodiments, R¹⁰¹ is independently halogen. In embodiments, R¹⁰¹ is independently —CN. In embodiments, R¹⁰¹ is independently oxo. In embodiments, R¹⁰¹ is independently —CF₃. In embodiments, R¹⁰¹ is independently —COOH. In embodiments, R¹⁰¹ is independently —CONH₂. In embodiments, R¹⁰¹ is independently —F. In embodiments, R¹⁰¹ is independently —Cl. In embodiments, R¹⁰¹ is independently —Br. In embodiments, R¹⁰¹ is independently —I.

In embodiments, L¹⁰² is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰² is a bond, —NH—, —S—, —O—, —C(O)—, —C(O)O—, —OC(O)—, —NHC(O)—, —C(O)NH—, —NHC(O)NH—, —NHC(NH)NH—, —C(S)—, —SS—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L¹⁰² is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L¹⁰² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond, —NH—, —OCH(R¹⁰²)—, —OCH(CH₂R¹⁰²)—, —OCH(CH₂CN)—, —S—, —O—, —C(O)—, —C(O)O—, —OC(O)—, —NHC(O)—, —C(O)NH—, —NHC(O)NH—, —NHC(NH)NH—, —C(S)—, —SS—, R¹⁰²-substituted or unsubstituted alkylene (e.g., C₁-C₂₀, C₁₀-C₂₀, C₁-C₈, C₁-C₆, or C₁-C₄), R¹⁰²-substituted or unsubstituted heteroalkylene (e.g., 2 to 20, 8 to 20, 2 to 10, 2 to 8, 2 to 6, or 2 to 4 membered), R¹⁰²-substituted or unsubstituted cycloalkylene (e.g., C₃-C₈, C₃-C₆, or C₅-C₆), R¹⁰²-substituted or unsubstituted heterocycloalkylene (e.g., 3 to 8, 3 to 6, or 5 to 6 membered), R¹⁰²-substituted or unsubstituted arylene (e.g., C₆-C₁₀, C₁₀, or phenylene), or R¹⁰²-substituted or unsubstituted heteroarylene (e.g., 5 to 10, 5 to 9, or 5 to 6 membered). In embodiments, L¹⁰² is a bond. In embodiments, L¹⁰² is —NH—. In embodiments, L¹⁰² is —OC(—SSR¹⁰²)(CH₃)—. In embodiments, L¹⁰² is —OC(—SCN)(CH₃)—. In embodiments, L¹⁰² is —OC(N₃)(CH₃)—. In embodiments, L¹⁰² is —OCH(—SSR¹⁰²)—. In embodiments, L¹⁰² is —OCH(—SCN)—. In embodiments, L¹⁰² is —OCH(N₃)—. In embodiments, L¹⁰² is —OCH(R¹⁰²)—. In embodiments, L¹⁰² is —OCH(CH₂R¹⁰²)—. In embodiments, L¹⁰² is —OCH(CH₂CN)—. In embodiments, L¹⁰² is —S—. In embodiments, L¹⁰² is —O—. In embodiments, L¹⁰² is —C(O)—. In embodiments, L¹⁰² is —C(O)O—. In embodiments, L¹⁰² is —OC(O)—. In embodiments, L¹⁰² is —NHC(O)—. In embodiments, L¹⁰² is —C(O)NH—. In embodiments, L¹⁰² is —NHC(O)NH—. In embodiments, L¹⁰² is —NHC(NH)NH—. In embodiments, L¹⁰² is —C(S)—. In embodiments, L¹⁰² is —SS—. In embodiments, L¹⁰² is R¹⁰²-substituted or unsubstituted C₁-C₂₀ alkylene. In embodiments, L¹⁰² is R¹⁰²-substituted or unsubstituted 2 to 20 membered heteroalkylene. In embodiments, L¹⁰² is R¹⁰²-substituted or unsubstituted C₃-C₈ cycloalkylene. In embodiments, L¹⁰² is R¹⁰²-substituted or unsubstituted 3 to 8 membered heterocycloalkylene. In embodiments, L¹⁰² is R¹⁰²-substituted or unsubstituted C₆-C₁₀ arylene. In embodiments, L¹⁰² is R¹⁰²-substituted or unsubstituted phenylene. In embodiments, L¹⁰² is R¹⁰²-substituted or unsubstituted 5 to 10 membered heteroarylene.

R¹⁰² is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered).

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{102A}-substituted or unsubstituted alkyl (e.g., C₁-C₂₀, C₁₀-C₂₀, C₁-C₈, C₁-C₆, or C₁-C₄), R^{102A}-substituted or unsubstituted heteroalkyl (e.g., 2 to 20, 8 to 20, 2 to 10, 2 to 8, 2 to 6, or 2 to 4 membered), R^{102A}-substituted or unsubstituted cycloalkyl (e.g., C₃-C₈, C₃-C₆, or C₅-C₆), R^{102A}-substituted or unsubstituted heterocycloalkyl (e.g., 3 to 8, 3 to 6, or 5 to 6 membered), R^{102A}-substituted or unsubstituted aryl (e.g., C₆-C₁₀, C₁₀, or phenyl), or R^{102A}-substituted or unsubstituted heteroaryl (e.g., 5 to 10, 5 to 9, or 5 to 6 membered).

In embodiments, R¹⁰² is independently —NH₂. In embodiments, R¹⁰² is independently —OH. In embodiments, R¹⁰² is independently halogen. In embodiments, R¹⁰² is independently —CN. In embodiments, R¹⁰² is independently oxo. In embodiments, R¹⁰² is independently —CF₃. In embodiments, R¹⁰² is independently —COOH. In embodiments, R¹⁰² is independently —CONH₂. In embodiments, R¹⁰² is independently —F. In embodiments, R¹⁰² is independently —Cl. In embodiments, R¹⁰² is independently —Br. In embodiments, R¹⁰² is independently —I.

In embodiments, R¹⁰² is independently unsubstituted alkyl (e.g., C₁-C₂₀, C₁₀-C₂₀, C₁-C₈, C₁-C₆, or C₁-C₄). In embodiments, R¹⁰² is independently unsubstituted C₁-C₆ alkyl. In embodiments, R¹⁰² is independently unsubstituted C₁-C₄ alkyl. In embodiments, R¹⁰² is independently unsubstituted methyl. In embodiments, R¹⁰² is independently unsubstituted tert-butyl. In embodiments, R¹⁰² is independently hydrogen.

In embodiments, L¹⁰³ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰³ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L¹⁰³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond, —NH—, —NR¹⁰³—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, R¹⁰³-substituted or unsubstituted alkylene (e.g., C₁-C₂₀, C₁₀-C₂₀, C₁-C₈, C₁-C₆, or C₁-C₄), R¹⁰³-substituted or unsubstituted heteroalkylene (e.g., 2 to 20 membered, 8 to 20 membered, 5 to 16 membered, 2 to 10, 2 to 8, 2 to 6, or 2 to 4 membered), R¹⁰³-substituted or unsubstituted cycloalkylene (e.g., C₃-C₈, C₃-C₆, or C₅-C₆), R¹⁰³-substituted or unsubstituted heterocycloalkylene (e.g., 3 to 8, 3 to 6, or 5 to 6 membered), R¹⁰³-substituted or unsubstituted arylene (e.g., C₆-C₁₀, C₁₀, or phenylene), or R¹⁰³-substituted or unsubstituted heteroarylene (e.g., 5 to 10, 5 to 9, or 5 to 6 membered). In embodiments, L¹⁰³ is a bond. In embodiments, L¹⁰³ is —NH—. In embodiments, L¹⁰³ is —NR¹⁰³—. In embodiments, L¹⁰³ is —S—. In embodiments, L¹⁰³ is —O—. In embodiments, L¹⁰³ is —C(O)—. In embodiments, L¹⁰³ is —C(O)O—. In embodiments, L¹⁰³ is —OC(O)—. In embodiments, L¹⁰³ is —NHC(O)—. In embodiments, L¹⁰³ is —C(O)NH—. In embodiments, L¹⁰³ is —NHC(O)NH—. In embodiments, L¹⁰³ is —NHC(NH)NH—. In embodiments, L¹⁰³ is —C(S)—. In embodiments, L¹⁰³ is —N═N—. In embodiments, L¹⁰³ is —SS—. In embodiments, L¹⁰³ is R¹⁰³-substituted or unsubstituted C₁-C₂₀ alkylene. In embodiments, L¹⁰³ is R¹⁰³-substituted or unsubstituted 2 to 20 membered heteroalkylene. In embodiments, L¹⁰³ is R¹⁰³-substituted or unsubstituted 5 to 16 membered heteroalkylene. In embodiments, L¹⁰³ is R¹⁰³-substituted or unsubstituted 2 to 10 membered heteroalkylene. In embodiments, L¹⁰³ is R¹⁰³-substituted or unsubstituted C₃-C₈ cycloalkylene. In embodiments, L¹⁰³ is R¹⁰³-substituted or unsubstituted 3 to 8 membered heterocycloalkylene. In embodiments, L¹⁰³ is R¹⁰³-substituted or unsubstituted C₆-C₁₀ arylene. In embodiments, L¹⁰³ is R¹⁰³-substituted or unsubstituted 5 to 10 membered heteroarylene.

In embodiments, L¹⁰³ is a bond, —NH—, —NR¹⁰³—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, —CH(OH)—, or —C(CH₂)—. In embodiments, L¹⁰³ is a bond. In embodiments, L¹⁰³ is —NH—. In embodiments, L¹⁰³ is —NR¹⁰³—. In embodiments, L¹⁰³ is —S—. In embodiments, L¹⁰³ is —O—. In embodiments, L¹⁰³ is —C(O)—. In embodiments, L¹⁰³ is —C(O)O—. In embodiments, L¹⁰³ is —OC(O)—. In embodiments, L¹⁰³ is —NHC(O)—. In embodiments, L¹⁰³ is —C(O)NH—. In embodiments, L¹⁰³ is —NHC(O)NH—. In embodiments, L¹⁰³ is —NHC(NH)NH—. In embodiments, L¹⁰³ is —C(S)—. In embodiments, L¹⁰³ is —N═N—. In embodiments, L¹⁰³ is —SS—. In embodiments, L¹⁰³ is —CH(OH)—. In embodiments, L¹⁰³ is —C(CH₂)—. In embodiments, L¹⁰³ is —(CH₂CH₂O)_{d}—. In embodiments, L¹⁰³ is —(CH₂O)_{d}—. In embodiments, L¹⁰³ is —(CH₂)_{d}—. In embodiments, L¹⁰³ is —(CH₂)_{d}—NH—. In embodiments, L¹⁰³ is -(unsubstituted phenylene)-. In embodiments, L¹⁰³ is

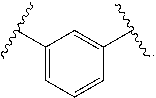 [SMILES: *c1cccc(*)c1]

In embodiments, L¹⁰³ is -(unsubstituted phenylene)-C(O)NH—. In embodiments, L¹⁰³ is

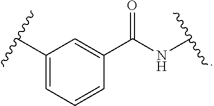 [SMILES: *NC(=O)c1cccc(*)c1]

In embodiments, L¹⁰³ is -(unsubstituted phenylene)-NHC(O)—. In embodiments, L¹⁰³ is

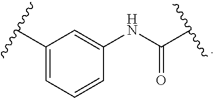 [SMILES: *C(=O)Nc1cccc(*)c1]

The symbol d is an integer from 0 to 8. In embodiments, d is 3. In embodiments, d is 1. In embodiments, d is 2. In embodiments, d is 0.

R¹⁰³ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{103A}-substituted or unsubstituted alkyl (e.g., C₁-C₂₀, C₁₀-C₂₀, C₁-C₈, C₁-C₆, or C₁-C₄), R^{103A}-substituted or unsubstituted heteroalkyl (e.g., 2 to 20, 8 to 20, 2 to 10, 2 to 8, 2 to 6, or 2 to 4 membered), R^{103A}-substituted or unsubstituted cycloalkyl (e.g., C₃-C₈, C₃-C₆, or C₅-C₆), R^{103A}-substituted or unsubstituted heterocycloalkyl (e.g., 3 to 8, 3 to 6, or 5 to 6 membered), R^{103A}-substituted or unsubstituted aryl (e.g., C₆-C₁₀, C₁₀, or phenyl), or R^{103A}-substituted or unsubstituted heteroaryl (e.g., 5 to 10, 5 to 9, or 5 to 6 membered).

In embodiments, R¹⁰³ is independently —NH₂. In embodiments, R¹⁰³ is independently —OH. In embodiments, R¹⁰³ is independently halogen. In embodiments, R¹⁰³ is independently —CN. In embodiments, R¹⁰³ is independently oxo. In embodiments, R¹⁰³ is independently —CF₃. In embodiments, R¹⁰³ is independently —COOH. In embodiments, R¹⁰³ is independently —CONH₂. In embodiments, R¹⁰³ is independently —F. In embodiments, R¹⁰³ is independently —Cl. In embodiments, R¹⁰³ is independently —Br. In embodiments, R¹⁰³ is independently —I.

In embodiments, L¹⁰⁴ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰⁴ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20 membered, 8 to 20 membered, 5 to 16 membered, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L¹⁰⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is a bond, —NH—, —NR¹⁰⁴—, —S—, —O—, —C(O)—, —C(O)O—, —OC(O)—, —NHC(O)—, —C(O)NH—, —NHC(O)NH—, —NHC(NH)NH—, —C(S)—, R¹⁰⁴-substituted or unsubstituted alkylene (e.g., C₁-C₂₀, C₁₀-C₂₀, C₁-C₈, C₁-C₆, or C₁-C₄), R¹⁰⁴-substituted or unsubstituted heteroalkylene (e.g., 2 to 20 membered, 8 to 20 membered, 5 to 16 membered, 2 to 10, 2 to 8, 2 to 6, or 2 to 4 membered), R¹⁰⁴-substituted or unsubstituted cycloalkylene (e.g., C₃-C₈, C₃-C₆, or C₅-C₆), R¹⁰⁴-substituted or unsubstituted heterocycloalkylene (e.g., 3 to 8, 3 to 6, or 5 to 6 membered), R¹⁰⁴-substituted or unsubstituted arylene (e.g., C₆-C₁₀, C₁₀, or phenylene), or R¹⁰⁴-substituted or unsubstituted heteroarylene (e.g., 5 to 10, 5 to 9, or 5 to 6 membered). In embodiments, L¹⁰⁴ is a bond. In embodiments, L¹⁰⁴ is —NH—. In embodiments, L¹⁰⁴ is —NR¹⁰⁴—. In embodiments, L¹⁰⁴ is —S—. In embodiments, L¹⁰⁴ is —O—. In embodiments, L¹⁰⁴ is —C(O)—. In embodiments, L¹⁰⁴ is —C(O)O—. In embodiments, L¹⁰⁴ is —OC(O)—. In embodiments, L¹⁰⁴ is —NHC(O)—. In embodiments, L¹⁰⁴ is —C(O)NH—. In embodiments, L¹⁰⁴ is —NHC(O)NH—. In embodiments, L¹⁰⁴ is —NHC(NH)NH—. In embodiments, L¹⁰⁴ is —C(S)—. In embodiments, L¹⁰⁴ is R¹⁰⁴-substituted or unsubstituted C₁-C₂₀ alkylene. In embodiments, L¹⁰⁴ is R¹⁰⁴-substituted or unsubstituted 2 to 20 membered heteroalkylene. In embodiments, L¹⁰⁴ is R¹⁰⁴-substituted or unsubstituted 5 to 16 membered heteroalkylene. In embodiments, L¹⁰⁴ is R¹⁰⁴-substituted or unsubstituted 2 to 10 membered heteroalkylene. In embodiments, L¹⁰⁴ is R¹⁰⁴-substituted or unsubstituted C₃-C₈ cycloalkylene. In embodiments, L¹⁰⁴ is R¹⁰⁴-substituted or unsubstituted 3 to 8 membered heterocycloalkylene. In embodiments, L¹⁰⁴ is R¹⁰⁴-substituted or unsubstituted C₆-C₁₀ arylene. In embodiments, L¹⁰⁴ is R¹⁰⁴-substituted or unsubstituted 5 to 10 membered heteroarylene. In embodiments, L¹⁰⁴ is R¹⁰⁴-substituted or unsubstituted phenylene.

In embodiments, L¹⁰⁴ is a bond, —NH—, —NR¹⁰⁴—, —S—, —O—, —C(O)—, —C(O)O—, —OC(O)—, —NHC(O)—, —C(O)NH—, —NHC(O)NH—, —NHC(NH)NH—, —C(S)—, —CH(OH)—, or —C(CH₂)—. In embodiments, L¹⁰⁴ is a bond. In embodiments, L¹⁰⁴ is —NH—. In embodiments, L¹⁰⁴ is —NR¹⁰⁴—. In embodiments, L¹⁰⁴ is —S—. In embodiments, L¹⁰⁴ is —O—. In embodiments, L¹⁰⁴ is —C(O)—. In embodiments, L¹⁰⁴ is —C(O)O—. In embodiments, L¹⁰⁴ is —OC(O)—. In embodiments, L¹⁰⁴ is —NHC(O)—. In embodiments, L¹⁰⁴ is —C(O)NH—. In embodiments, L¹⁰⁴ is —NHC(O)NH—. In embodiments, L¹⁰⁴ is —NHC(NH)NH—. In embodiments, L¹⁰⁴ is —C(S)—. In embodiments, L¹⁰⁴ is —CH(OH)—. In embodiments, L¹⁰⁴ is —C(CH₂)—.

In embodiments, L¹⁰⁴ is —(CH₂CH₂O)ₑ—. In embodiments, L¹⁰⁴ is —(CH₂O)ₑ—. In embodiments, L¹⁰⁴ is —(CH₂)ₑ—. In embodiments, L¹⁰⁴ is —(CH₂)ₑ—NH—. In embodiments, L¹⁰⁴ is -(unsubstituted phenylene)-. In embodiments, L¹⁰⁴ is

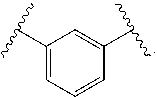 [SMILES: *c1cccc(*)c1]

In embodiments, L¹⁰⁴ is -(unsubstituted phenylene)-C(O)NH—. In embodiments, L¹⁰⁴ is

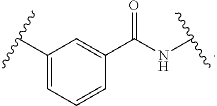 [SMILES: *NC(=O)c1cccc(*)c1]

In embodiments, L¹⁰⁴ is -(unsubstituted phenylene)-NHC(O)—. In embodiments, L¹⁰⁴ is

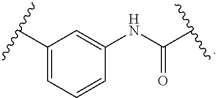 [SMILES: *C(=O)Nc1cccc(*)c1]

The symbol e is an integer from 0 to 8. In embodiments, e is 3. In embodiments, e is 1. In embodiments, e is 2.

R¹⁰⁴ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{104A}-substituted or unsubstituted alkyl (e.g., C₁-C₂₀, C₁₀-C₂₀, C₁-C₈, C₁-C₆, or C₁-C₄), R^{104A}-substituted or unsubstituted heteroalkyl (e.g., 2 to 20, 8 to 20, 2 to 10, 2 to 8, 2 to 6, or 2 to 4 membered), R^{104A}-substituted or unsubstituted cycloalkyl (e.g., C₃-C₈, C₃-C₆, or C₅-C₆), R^{104A}-substituted or unsubstituted heterocycloalkyl (e.g., 3 to 8, 3 to 6, or 5 to 6 membered), R^{104A}-substituted or unsubstituted aryl (e.g., C₆-C₁₀, C₁₀, or phenyl), or R^{104A}-substituted or unsubstituted heteroaryl (e.g., 5 to 10, 5 to 9, or 5 to 6 membered).

In embodiments, R¹⁰⁴ is independently —NH₂. In embodiments, R¹⁰⁴ is independently —OH. In embodiments, R¹⁰⁴ is independently halogen. In embodiments, R¹⁰⁴ is independently —CN. In embodiments, R¹⁰⁴ is independently oxo. In embodiments, R¹⁰⁴ is independently —CF₃. In embodiments, R¹⁰⁴ is independently —COOH. In embodiments, R¹⁰⁴ is independently —CONH₂. In embodiments, R¹⁰⁴ is independently —F. In embodiments, R¹⁰⁴ is independently —Cl. In embodiments, R¹⁰⁴ is independently —Br. In embodiments, R¹⁰⁴ is independently —I.

In embodiments, L¹⁰⁵ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰⁵ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20 membered, 8 to 20 membered, 5 to 16 membered, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L¹⁰⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is a bond, —NH—, —NR¹⁰⁵—, —S—, —O—, —C(O)—, —C(O)O—, —OC(O)—, —NHC(O)—, —C(O)NH—, —NHC(O)NH—, —NHC(NH)NH—, —C(S)—, R¹⁰⁵-substituted or unsubstituted alkylene (e.g., C₁-C₂₀, C₁₀-C₂₀, C₁-C₈, C₁-C₆, or C₁-C₄), R¹⁰⁵-substituted or unsubstituted heteroalkylene (e.g., 2 to 20 membered, 8 to 20 membered, 5 to 16 membered, 2 to 10, 2 to 8, 2 to 6, or 2 to 4 membered), R¹⁰⁵-substituted or unsubstituted cycloalkylene (e.g., C₃-C₈, C₃-C₆, or C₅-C₆), R¹⁰⁵-substituted or unsubstituted heterocycloalkylene (e.g., 3 to 8, 3 to 6, or 5 to 6 membered), R¹⁰⁵-substituted or unsubstituted arylene (e.g., C₆-C₁₀, C₁₀, or phenylene), or R¹⁰⁵-substituted or unsubstituted heteroarylene (e.g., 5 to 10, 5 to 9, or 5 to 6 membered). In embodiments, L¹⁰⁵ is a bond. In embodiments, L¹⁰⁵ is —NH—. In embodiments, L¹⁰⁵ is —NR¹⁰⁵—. In embodiments, L¹⁰⁵ is —S—. In embodiments, L¹⁰⁵ is —O—. In embodiments, L¹⁰⁵ is —C(O)—. In embodiments, L¹⁰⁵ is —C(O)O—. In embodiments, L¹⁰⁵ is —OC(O)—. In embodiments, L¹⁰⁵ is —NHC(O)—. In embodiments, L¹⁰⁵ is —C(O)NH—. In embodiments, L¹⁰⁵ is —NHC(O)NH—. In embodiments, L¹⁰⁵ is —NHC(NH)NH—. In embodiments, L¹⁰⁵ is —C(S)—. In embodiments, L¹⁰⁵ is R¹⁰⁵-substituted or unsubstituted C₁-C₂₀ alkylene. In embodiments, L¹⁰⁵ is R¹⁰⁵-substituted or unsubstituted 2 to 20 membered heteroalkylene. In embodiments, L¹⁰⁵ is R¹⁰⁵-substituted or unsubstituted 5 to 16 membered heteroalkylene. In embodiments, L¹⁰⁵ is R¹⁰⁵-substituted or unsubstituted 2 to 10 membered heteroalkylene. In embodiments, L¹⁰⁵ is R¹⁰⁵-substituted or unsubstituted C₃-C₈ cycloalkylene. In embodiments, L¹⁰⁵ is R¹⁰⁵-substituted or unsubstituted 3 to 8 membered heterocycloalkylene. In embodiments, L¹⁰⁵ is R¹⁰⁵-substituted or unsubstituted C₆-C₁₀ arylene. In embodiments, L¹⁰⁵ is R¹⁰⁵-substituted or unsubstituted 5 to 10 membered heteroarylene.

In embodiments, L¹⁰⁵ is a bond, —NH—, —NR¹⁰⁵—, —S—, —O—, —C(O)—, —C(O)O—, —OC(O)—, —NHC(O)—, —C(O)NH—, —NHC(O)NH—, —NHC(NH)NH—, —C(S)—, —CH(OH)—, or —C(CH₂)—. In embodiments, L¹⁰⁵ is a bond. In embodiments, L¹⁰⁵ is —NH—. In embodiments, L¹⁰⁵ is —NR¹⁰⁵—. In embodiments, L¹⁰⁵ is —S—. In embodiments, L¹⁰⁵ is —O—. In embodiments, L¹⁰⁵ is —C(O)—. In embodiments, L¹⁰⁵ is —C(O)O—. In embodiments, L¹⁰⁵ is —OC(O)—. In embodiments, L¹⁰⁵ is —NHC(O)—. In embodiments, L¹⁰⁵ is —C(O)NH—. In embodiments, L¹⁰⁵ is —NHC(O)NH—. In embodiments, L¹⁰⁵ is —NHC(NH)NH—. In embodiments, L¹⁰⁵ is —C(S)—. In embodiments, L¹⁰⁵ is —CH(OH)—. In embodiments, L¹⁰⁵ is —C(CH₂)—.

In embodiments, L¹⁰⁵ is —(CH₂CH₂O)_{f}—. In embodiments, L¹⁰⁵ is —(CH₂O)_{f}—. In embodiments, L¹⁰⁵ is —(CH₂)_{f}—. In embodiments, L¹⁰⁵ is —(CH₂)_{f}—NH—. In embodiments, L¹⁰⁵ is —C(O)NH(CH₂)_{f}—NH—. In embodiments, L¹⁰⁵ is —(CH₂CH₂O)_{f}—(CH₂)_{g}—NH—. In embodiments, L¹⁰⁵ is —(CH₂)_{g}—. In embodiments, L¹⁰⁵ is —(CH₂)_{g}—NH—. In embodiments, L¹⁰⁵ is —NHC(O)—(CH₂)_{f}—NH—. In embodiments, L¹⁰⁵ is —NHC(O)—(CH₂)_{f}—NH—. In embodiments, L¹⁰⁵ is —NHC(O)—(CH₂CH₂O)_{f}—(CH₂)_{g}—NH—. In embodiments, L¹⁰⁵ is —NHC(O)—(CH₂)_{g}—. In embodiments, L¹⁰⁵ is —NHC(O)—(CH₂)_{g}—NH—. In embodiments, L¹⁰⁵ is —C(O)NH(CH₂)_{f}—NH—. In embodiments, L¹⁰⁵ is —C(O)NH—(CH₂CH₂O)_{f}—(CH₂)_{g}—NH—. In embodiments, L¹⁰⁵ is —C(O)NH—(CH₂)_{g}—. In embodiments, L¹⁰⁵ is —C(O)NH—(CH₂)_{g}—NH—. The symbol f is an integer from 0 to 8. In embodiments, f is 3. In embodiments, f is 1. In embodiments, f is 2. In embodiments, f is 0. The symbol g is an integer from 0 to 8. In embodiments, g is 3. In embodiments, g is 1. In embodiments, g is 2. In embodiments, g is 0.

R¹⁰⁵ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{105A}-substituted or unsubstituted alkyl (e.g., C₁-C₂₀, C₁₀-C₂₀, C₁-C₈, C₁-C₆, or C₁-C₄), R^{105A}-substituted or unsubstituted heteroalkyl (e.g., 2 to 20, 8 to 20, 2 to 10, 2 to 8, 2 to 6, or 2 to 4 membered), R^{105A}-substituted or unsubstituted cycloalkyl (e.g., C₃-C₈, C₃-C₆, or C₅-C₆), R^{105A}-substituted or unsubstituted heterocycloalkyl (e.g., 3 to 8, 3 to 6, or 5 to 6 membered), R^{105A}-substituted or unsubstituted aryl (e.g., C₆-C₁₀, C₁₀, or phenyl), or R^{105A}-substituted or unsubstituted heteroaryl (e.g., 5 to 10, 5 to 9, or 5 to 6 membered).

In embodiments, R¹⁰⁵ is independently —NH₂. In embodiments, R¹⁰⁵ is independently —OH. In embodiments, R¹⁰⁵ is independently halogen. In embodiments, R¹⁰⁵ is independently —CN. In embodiments, R¹⁰⁵ is independently oxo. In embodiments, R¹⁰⁵ is independently —CF₃. In embodiments, R¹⁰⁵ is independently —COOH. In embodiments, R¹⁰⁵ is independently —CONH₂. In embodiments, R¹⁰⁵ is independently —F. In embodiments, R¹⁰⁵ is independently —Cl. In embodiments, R¹⁰⁵ is independently —Br. In embodiments, R¹⁰⁵ is independently —I. R^{101A}, R^{102A}, R^{103A}, R^{104A}, and R^{105A} are each independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰⁰ is

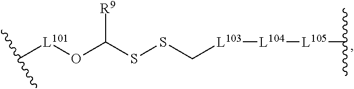

wherein L¹⁰¹, L¹⁰³, L¹⁰⁴, L¹⁰⁵, and R⁹ are as described herein. In embodiments, L¹⁰⁰ is

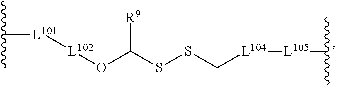

wherein L¹⁰¹, L¹⁰², L¹⁰⁴, L¹⁰⁵, and R⁹ are as described herein. In embodiments, L¹⁰⁰ is

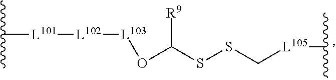

wherein L¹⁰¹, L¹⁰², L¹⁰³, L¹⁰⁵, and R⁹ are as described herein. In embodiments, L¹⁰⁰ is

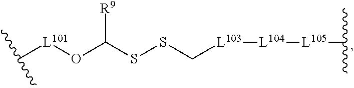

wherein L¹⁰¹, L¹⁰³, L¹⁰⁴, L¹⁰⁵, and R⁹ are as described herein. In embodiments, L¹⁰⁰ is

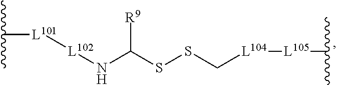

wherein L¹⁰¹, L¹⁰², L¹⁰⁴, L¹⁰⁵, and R⁹ are as described herein. In embodiments, L¹⁰⁰ is

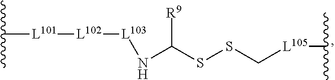

wherein L¹⁰¹, L¹⁰², L¹⁰³, L¹⁰⁵, and R⁹ are as described herein.

In embodiments, L¹⁰⁰ is -L¹⁰¹-O—CH(N₃)-L¹⁰³-L¹⁰⁴-L¹⁰⁵-; and L¹⁰¹, L¹⁰³, L¹⁰⁴, and L¹⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹⁰⁰ is -L¹⁰¹-O—CH(N₃)-L¹⁰³-L¹⁰⁴-L¹⁰⁵-; wherein L¹⁰¹ is independently a substituted or unsubstituted C₁-C₄ alkylene or substituted or unsubstituted 8 to 20 membered heteroalkylene; L¹⁰³ is independently a bond or substituted or unsubstituted 2 to 10 membered heteroalkylene; L¹⁰⁴ is independently a bond, substituted or unsubstituted 4 to 18 membered heteroalkylene, or substituted or unsubstituted phenylene; and L¹⁰⁵ is independently bond or substituted or unsubstituted 4 to 18 membered heteroalkylene. In embodiments, L¹⁰⁰ is -L¹⁰¹-O—CH(N₃)—CH₂—O-L¹⁰⁴-L¹⁰⁵-; wherein L¹⁰¹ and L¹⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and L¹⁰⁴ is unsubstituted phenylene.

In embodiments, L¹⁰⁰ is

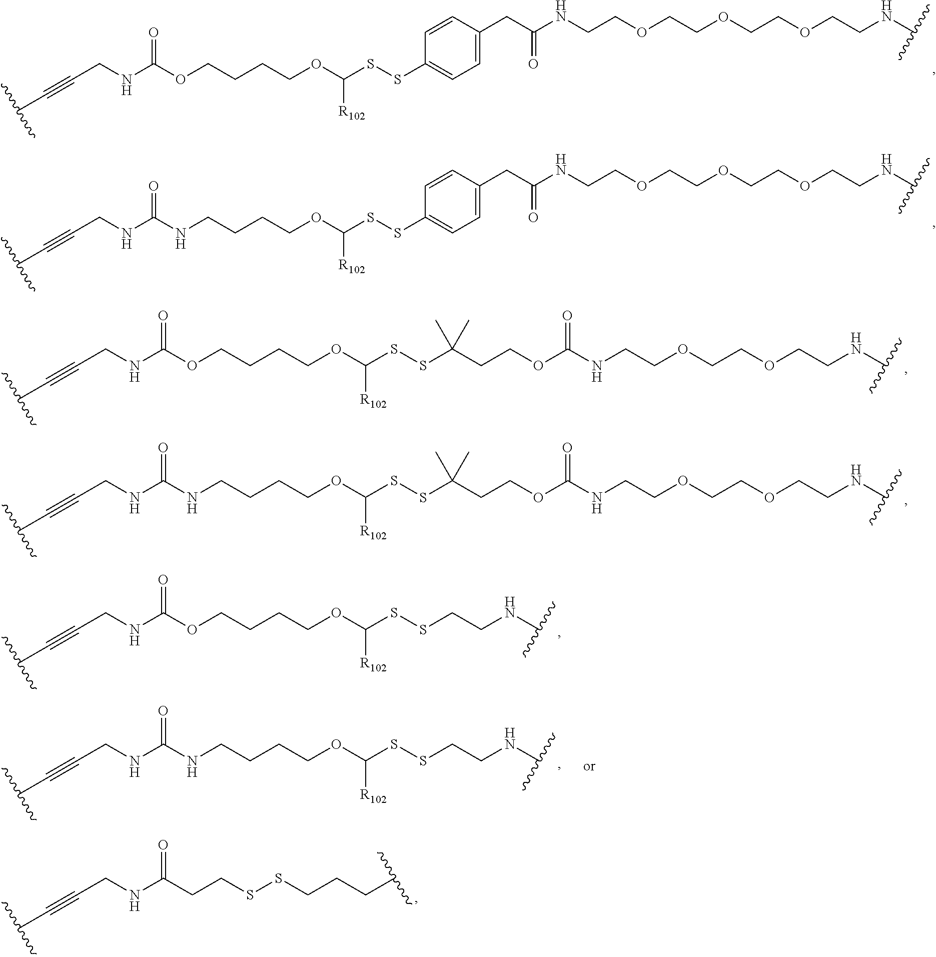

wherein R¹⁰² is as described herein.

In embodiments, L¹⁰⁰ is

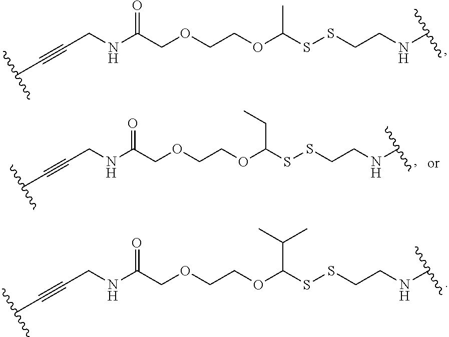 [SMILES: *C#CCNC(=O)COCCOC(C)SSCCN* ; *C#CCNC(=O)COCCOC(CC)SSCCN* ; *C#CCNC(=O)COCCOC(SSCCN*)C(C)C]

In embodiments, L¹⁰⁰ is

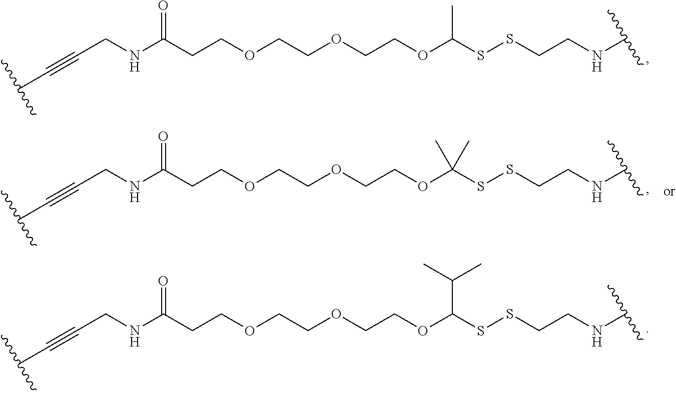 [SMILES: *C#CCNC(=O)CCOCCOCCOC(C)SSCCN* ; *C#CCNC(=O)CCOCCOCCOC(C)(C)SSCCN* ; *C#CCNC(=O)CCOCCOCCOC(SSCCN*)C(C)C]

In embodiments, L¹⁰⁰ is

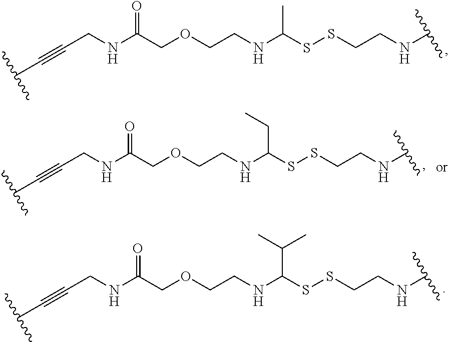 [SMILES: *C#CCNC(=O)COCCNC(C)SSCCN* ; *C#CCNC(=O)COCCNC(CC)SSCCN* ; *C#CCNC(=O)COCCNC(SSCCN*)C(C)C]

In embodiments, L¹⁰⁰ is

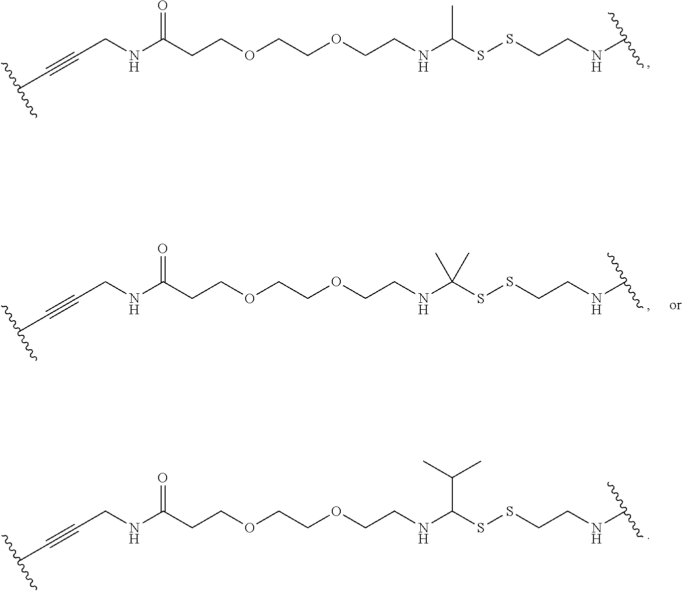 [SMILES: *C#CCNC(=O)CCOCCOCCNC(C)SSCCN* ; *C#CCNC(=O)CCOCCOCCNC(C)(C)SSCCN* ; *C#CCNC(=O)CCOCCOCCNC(SSCCN*)C(C)C]

In embodiments, R⁹ is substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered). In embodiments, R⁹ is hydrogen.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is R¹⁰-substituted or unsubstituted alkyl (e.g., C₁-C₂₀, C₁₀-C₂₀, C₁-C₈, C₁-C₆, or C₁-C₄), R¹⁰-substituted or unsubstituted heteroalkyl (e.g., 2 to 20, 8 to 20, 2 to 10, 2 to 8, 2 to 6, or 2 to 4 membered), R¹⁰-substituted or unsubstituted cycloalkyl (e.g., C₃-C₈, C₃-C₆, or C₅-C₆), R¹⁰-substituted or unsubstituted heterocycloalkyl (e.g., 3 to 8, 3 to 6, or 5 to 6 membered), R¹⁰-substituted or unsubstituted aryl (e.g., C₆-C₁₀, C₁₀, or phenyl), or R¹⁰-substituted or unsubstituted heteroaryl (e.g., 5 to 10, 5 to 9, or 5 to 6 membered). In embodiments, R⁹ is R¹⁰-substituted or unsubstituted alkyl (e.g., C₁-C₂₀, C₁₀-C₂₀, C₁-C₈, C₁-C₆, or C₁-C₄), R¹⁰-substituted or unsubstituted heteroalkyl (e.g., 2 to 20, 8 to 20, 2 to 10, 2 to 8, 2 to 6, or 2 to 4 membered), R¹⁰-substituted or unsubstituted cycloalkyl (e.g., C₃-C₈, C₃-C₆, or C₅-C₆), R¹⁰-substituted or unsubstituted heterocycloalkyl (e.g., 3 to 8, 3 to 6, or 5 to 6 membered), R¹⁰-substituted or unsubstituted aryl (e.g., C₆-C₁₀, C₁₀, or phenyl), or R¹⁰-substituted or unsubstituted heteroaryl (e.g., 5 to 10, 5 to 9, or 5 to 6 membered). In embodiments, R⁹ is unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

R¹⁰ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R⁹ is independently unsubstituted alkyl (e.g., C₁-C₂₀, C₁₀-C₂₀, C₁-C₈, C₁-C₆, or C₁-C₄). In embodiments, R⁹ is independently unsubstituted C₁-C₆ alkyl. In embodiments, R⁹ is independently unsubstituted C₁-C₄ alkyl. In embodiments, R⁹ is independently unsubstituted methyl. In embodiments, R⁹ is independently unsubstituted ethyl. In embodiments, R⁹ is independently unsubstituted propyl. In embodiments, R⁹ is independently unsubstituted tert-butyl.

In embodiments, R⁹ is independently unsubstituted C₃-C₈ cycloalkyl. In embodiments, R⁹ is independently unsubstituted C₃-C₆ cycloalkyl. In embodiments, R⁹ is independently unsubstituted C₅-C₆ cycloalkyl. In embodiments, R⁹ is independently unsubstituted 3 to 8 membered heterocycloalkyl. In embodiments, R⁹ is independently unsubstituted 3 to 6 membered heterocycloalkyl. In embodiments, R⁹ is independently unsubstituted 5 to 6 membered heterocycloalkyl. In embodiments, R⁹ is independently unsubstituted phenyl. In embodiments, R⁹ is independently unsubstituted 5 to 6 membered heteroaryl. In embodiments, R⁹ is independently unsubstituted 5 membered heteroaryl. In embodiments, R⁹ is independently unsubstituted 6 membered heteroaryl.

In embodiments, R⁹ is

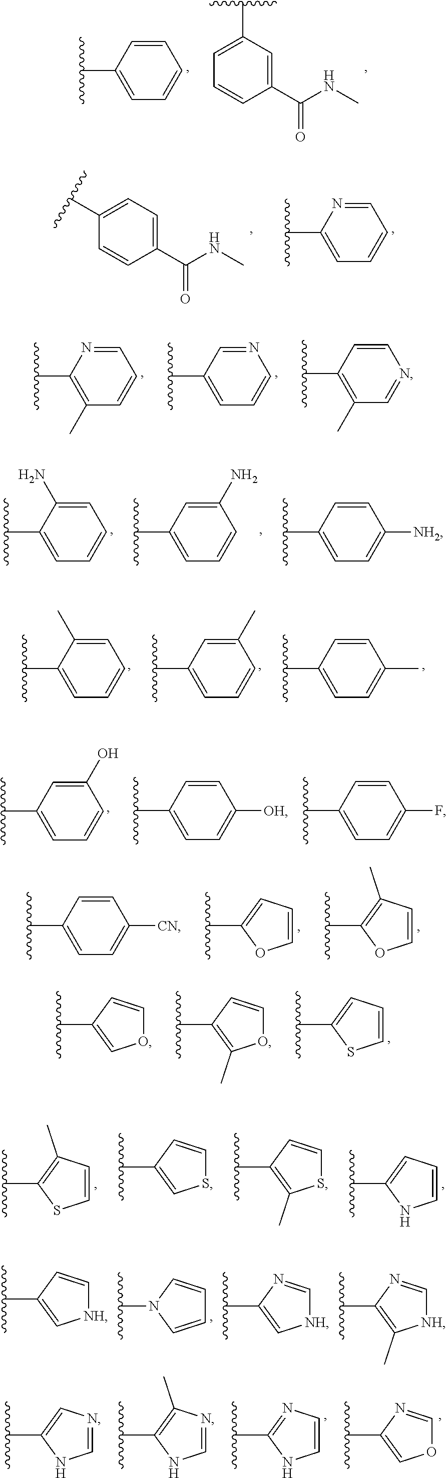

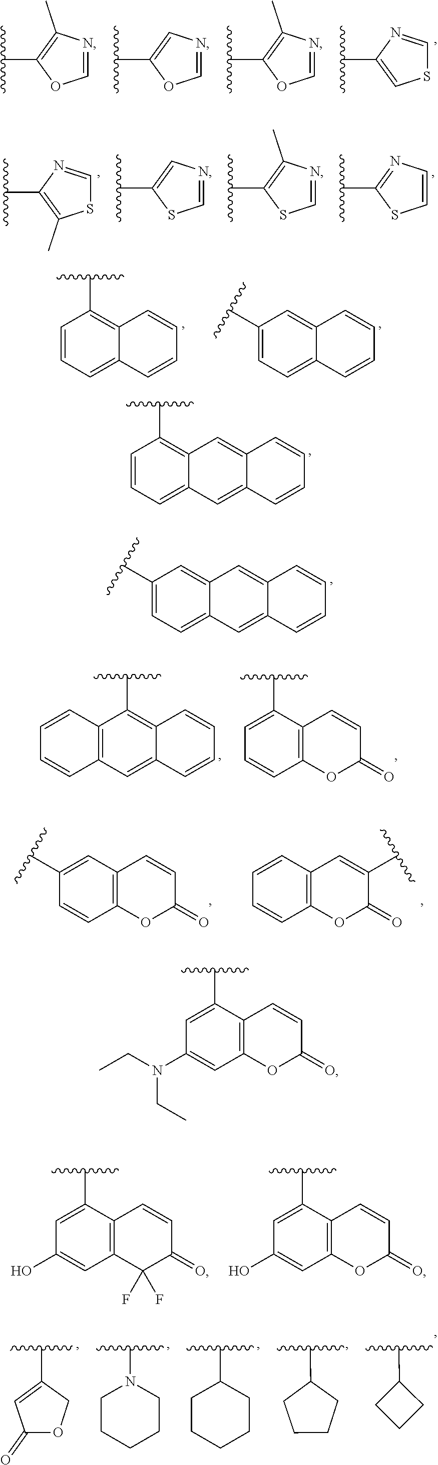

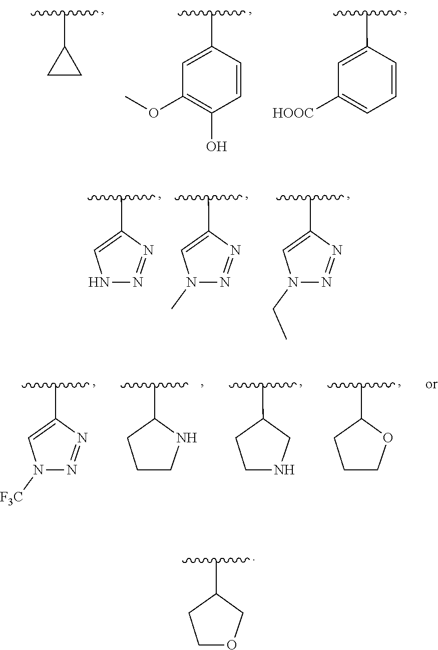

In embodiments, L¹⁰⁰ includes

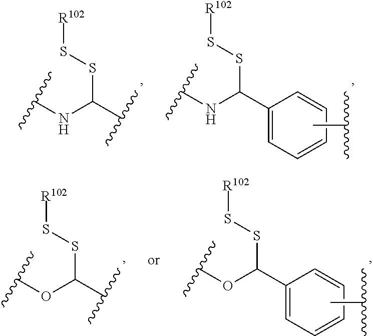

wherein R¹⁰² is unsubstituted C₁-C₄ alkyl. In embodiments, L¹⁰⁰ is a cleavable linker including:

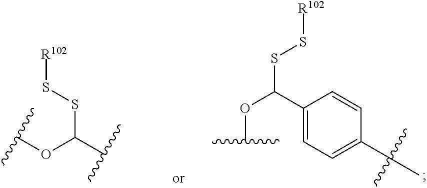

wherein R¹⁰² is as described herein. In embodiments, L¹⁰⁰ includes

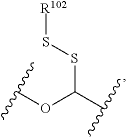

wherein R¹⁰² is as described herein. In embodiments, L¹⁰⁰ includes

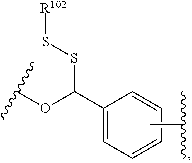

wherein R¹⁰² is as described herein. In embodiments, at least one of L¹⁰¹, L¹⁰², L¹⁰³, L¹⁰⁴, and L¹⁰⁵ independently includes

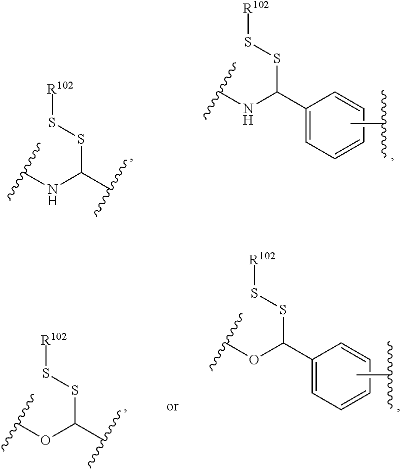

wherein R¹⁰² is as described herein. In embodiments, R¹⁰² is unsubstituted C₁-C₄ alkyl. In embodiments, R¹⁰² is unsubstituted C₁ alkyl. In embodiments, R¹⁰² is unsubstituted C₂ alkyl. In embodiments, R¹⁰² is unsubstituted C₃ alkyl. In embodiments, R¹⁰² is unsubstituted C₄ alkyl.

In embodiments, L¹⁰⁰ is

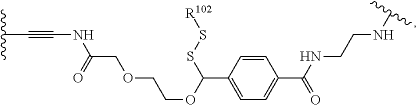

wherein R¹⁰² is as described herein. In embodiments, L¹⁰⁰ is

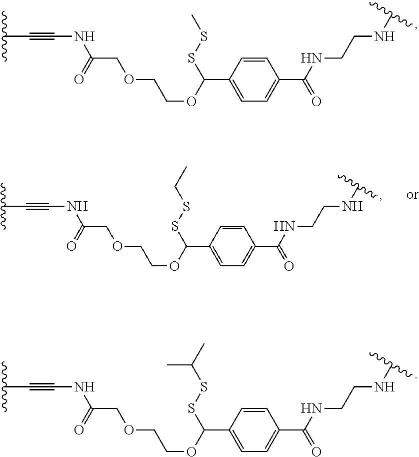 [SMILES: *C#CNC(=O)COCCOC(SSC)c1ccc(C(=O)NCCN*)cc1 ; *C#CNC(=O)COCCOC(SSCC)c1ccc(C(=O)NCCN*)cc1 ; *C#CNC(=O)COCCOC(SSC(C)C)c1ccc(C(=O)NCCN*)cc1]

In embodiments, L¹⁰⁰ is

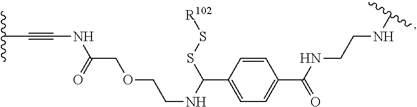

wherein R¹⁰² is as described herein. In embodiments, L¹⁰⁰ is

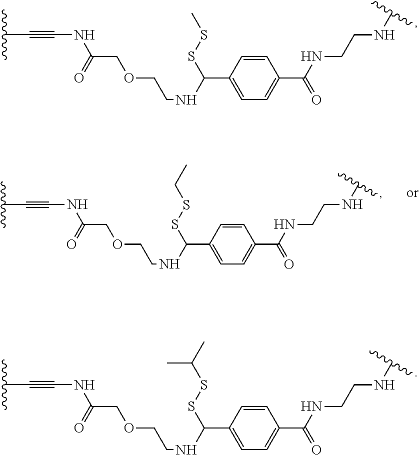 [SMILES: *C#CNC(=O)COCCNC(SSC)c1ccc(C(=O)NCCN*)cc1 ; *C#CNC(=O)COCCNC(SSCC)c1ccc(C(=O)NCCN*)cc1 ; *C#CNC(=O)COCCNC(SSC(C)C)c1ccc(C(=O)NCCN*)cc1]

In embodiments, L¹⁰⁰ is

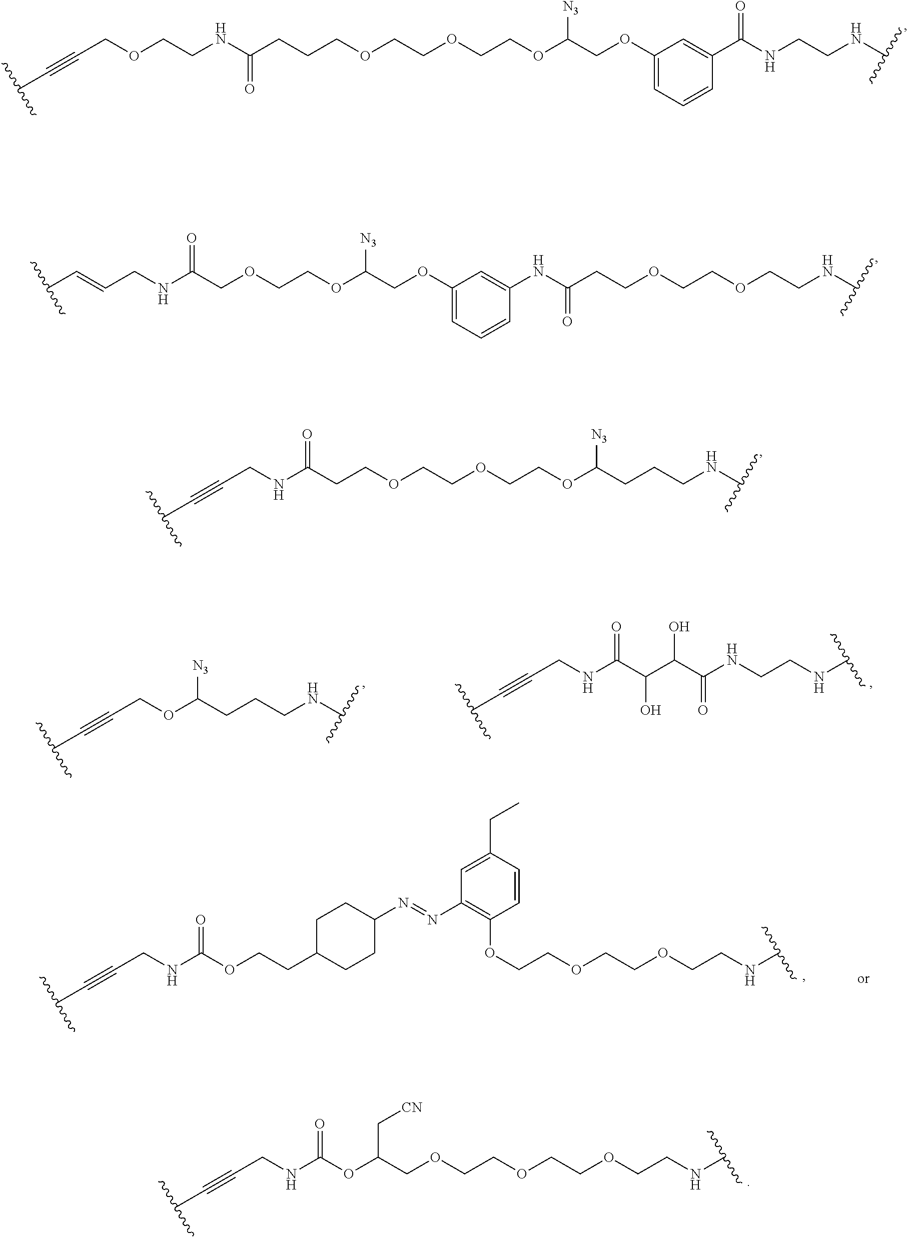

In embodiments, R⁴ is independently a detectable label moiety. In embodiments, R⁴ is a fluorescent dye moiety. In embodiments, R⁴ is a detectable moiety described herein. In embodiments, R⁴ is a detectable moiety described in Table 1. In embodiments, R⁴ is a fluorescent dye moiety wherein the maximum emission of the fluorescent dye moiety is greater than about 530, 540, or 550 nm. In embodiments, R⁴ is a fluorescent dye moiety wherein the maximum emission of the fluorescent dye moiety is greater than 530 nm. In embodiments, R⁴ is a fluorescent dye moiety wherein the maximum emission of the fluorescent dye moiety is less than about 700, 690, or 680 nm. In embodiments, R⁴ is a fluorescent dye moiety wherein the maximum emission of the fluorescent dye moiety is less than 680 nm. In embodiments, R⁴ is a fluorescent dye moiety wherein the maximum emission of the fluorescent dye moiety is greater than about 530 and less than about 680 nm. In embodiments, R⁴ is a fluorescent dye moiety wherein the maximum emission of the fluorescent dye moiety is greater than 530 and less than 680 nm. For example, R⁴ may be any fluorescent moiety described in US Publication 2020/0216682, which is incorporated herein by reference.

TABLE 1
Detectable label moieties to be used in selected embodiments.

| Nucleoside/nucleotide | | |
|---|---|---|
| abbreviation | Dye name | λmax (nm) |
| dG | Atto 532 | 532 |
| dG | Atto Rho 6G | 535 |
| dG | R6G | 534 |
| dG | Tet | 521 |
| dA | Atto Rho 11 | 572 |
| dA | Atto 565 | 564 |
| dA | Alexa Fluor 568 | 578 |
| dA | dTamra | 578 |
| dC | Alexa Fluor 647 | 650 |
| dC | Atto 647N | 644 |
| dC | Janelia Fluor 646 | 646 |
| dT | Alexa Fluor 680 | 682 |
| dT | Alexa Fluor 700 | 696 |
| dT | CF680R | 680 |

In embodiments, R⁴ is

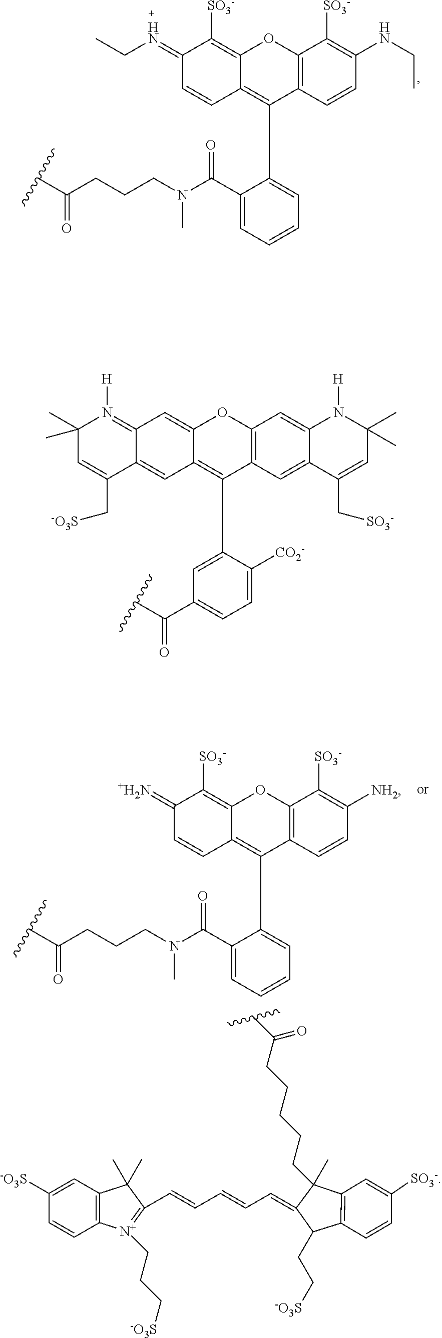

In another aspect is provided a chase solution. In embodiments, the chase solution includes components necessary to incorporate a modified nucleotide into a polynucleotide strand (e.g., a primer) hybridized to a template. In embodiments, the chase solution includes a plurality of chase nucleotides, wherein each nucleotide of the plurality of chase nucleotides includes a retardant moiety and a reversible terminator moiety. In embodiments, each nucleotide of the plurality of chase nucleotides has the formula:

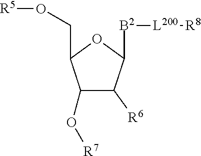

(II); wherein, B² is a nucleobase; R⁵ is a triphosphate or thiotriphosphate; R⁶ is hydrogen or —OH; R⁷ is independently a reversible terminator or hydrogen; R⁸ is independently a retardant moiety; and L²⁰⁰ is a cleavable linker. In embodiments, the chase solution does not include sequencing nucleotides.

In embodiments, B² is a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

In embodiments, B² is a universal nucleobase. A “universal nucleobase,” as used herein, refers to a nucleobase analog that is capable of forming a base pair to any of the four natural nucleotide bases (e.g., cytosine (C), guanine (G), adenine (A), or thymine (T)). Thus, any other base may be paired with a universal base analog in a double-stranded polynucleotide. Universal base analogs may be divided into hydrogen bonding bases and pi-stacking bases. Hydrogen bonding bases form hydrogen bonds with any of the natural nucleobases. The hydrogen bonds formed by hydrogen bonding bases are weaker than the hydrogen bonds between natural nucleobases. Pi-stacking bases are non-hydrogen bonding, hydrophobic, aromatic bases that stabilize duplex polynucleotides by stacking interactions. Examples of hydrogen bonding bases include, but are not limited to, hypoxanthine (inosine), 7-deazahypoxanthine, 2-azahypoxanthine, 2-hydroxypurine, purine, and 4-Amino-TH-pyrazolo [3,4-d]pyrimidine. In embodiments, universal base analogs included in the bases in a universal region of a universal template strand are hydrogen bonding bases. In embodiments, all universal base analogs included in the bases in the universal region are inosine or derivatives thereof. Examples of pi-stacking bases include, but are not limited to, nitroimidazole, indole, benzimidazole, 5-fluoroindole, 5-nitroindole, N-indol-5-yl-formamide, isoquinoline, and methylisoquinoline. Examples of universal bases are discussed in Berger et al., Universal Bases for Hybridization, Replication and Chain Termination, Nucleic Acids Research 2000, August 1, 28(15) pp. 2911-2914; David Loakes, The Applications of Universal DNA Base Analogs, 29(12) Nucleic Acids Research 2437 (2001); and Feng Liang et al., Universal base analogs and their applications in DNA sequencing technology, 3 RSC Advances 14910-14928 (2013).

In embodiments, B² is

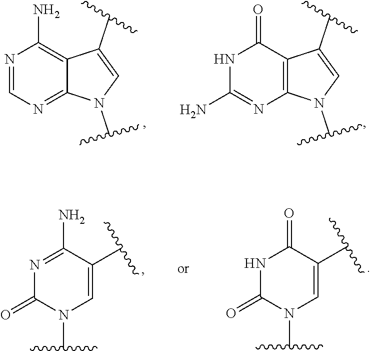 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B² is

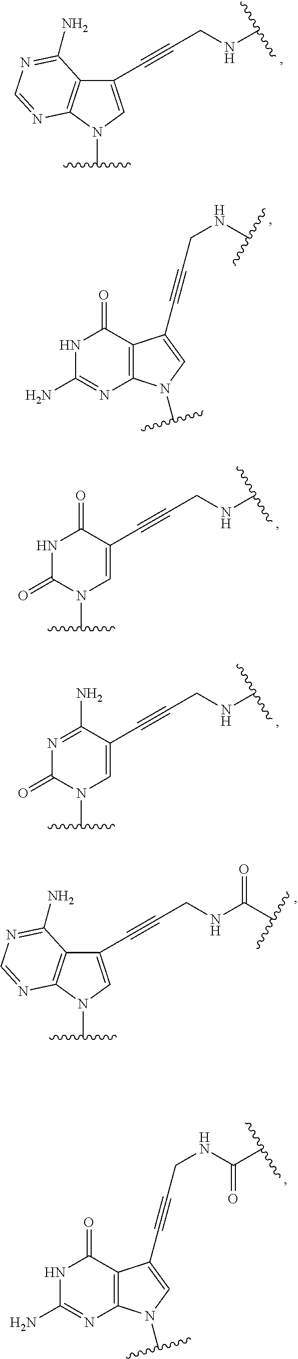 [SMILES: *NCC#Cc1cn(*)c2ncnc(N)c12 ; *NCC#Cc1cn(*)c2nc(N)[nH]c(=O)c12 ; *NCC#Cc1cn(*)c(=O)[nH]c1=O ; *NCC#Cc1cn(*)c(=O)nc1N ; *C(=O)NCC#Cc1cn(*)c2ncnc(N)c12 ; *C(=O)NCC#Cc1cn(*)c2nc(N)[nH]c(=O)c12]

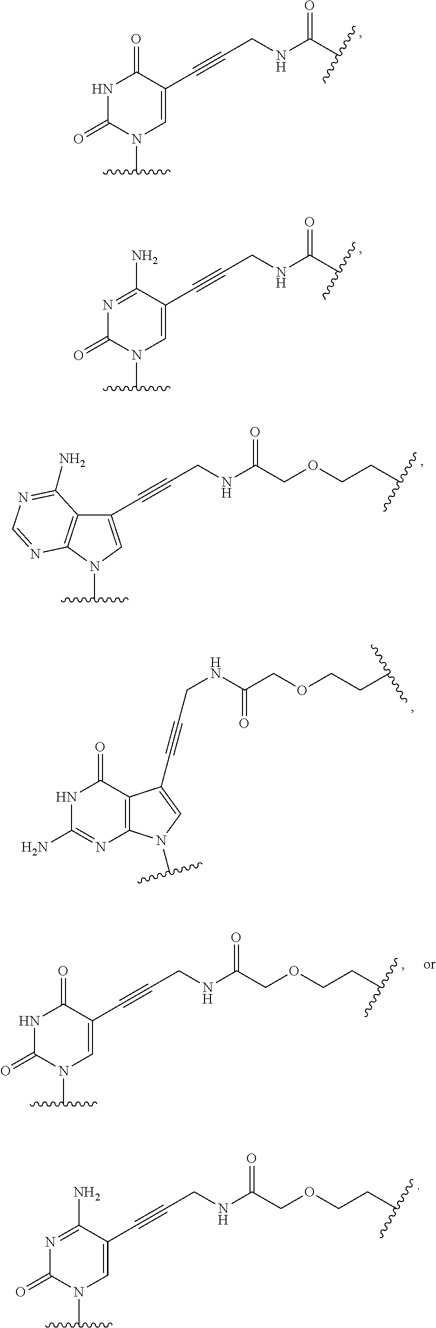 [SMILES: *C(=O)NCC#Cc1cn(*)c(=O)[nH]c1=O ; *C(=O)NCC#Cc1cn(*)c(=O)nc1N ; *CCOCC(=O)NCC#Cc1cn(*)c2ncnc(N)c12 ; *CCOCC(=O)NCC#Cc1cn(*)c2nc(N)[nH]c(=O)c12 ; *CCOCC(=O)NCC#Cc1cn(*)c(=O)[nH]c1=O ; *CCOCC(=O)NCC#Cc1cn(*)c(=O)nc1N]

In embodiments, B² is

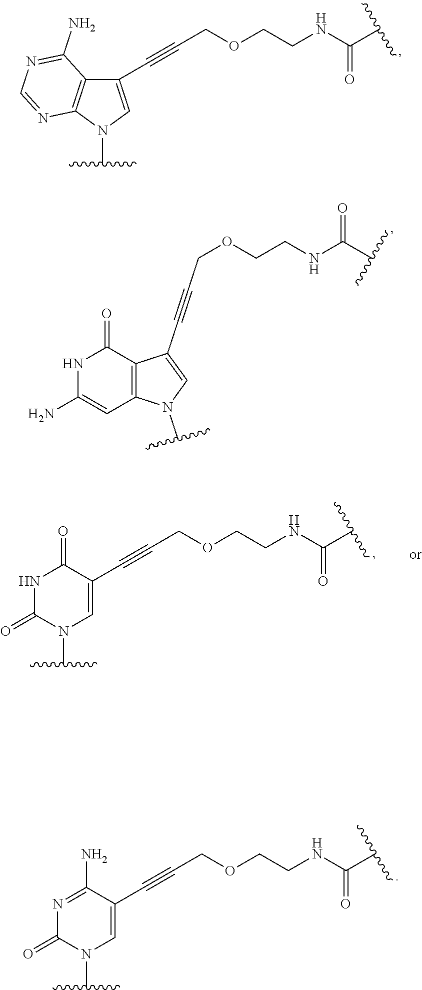 [SMILES: *C(=O)NCCOCC#Cc1cn(*)c2ncnc(N)c12 ; *C(=O)NCCOCC#Cc1cn(*)c2cc(N)[nH]c(=O)c12 ; *C(=O)NCCOCC#Cc1cn(*)c(=O)[nH]c1=O ; *C(=O)NCCOCC#Cc1cn(*)c(=O)nc1N]

In embodiments, B² is

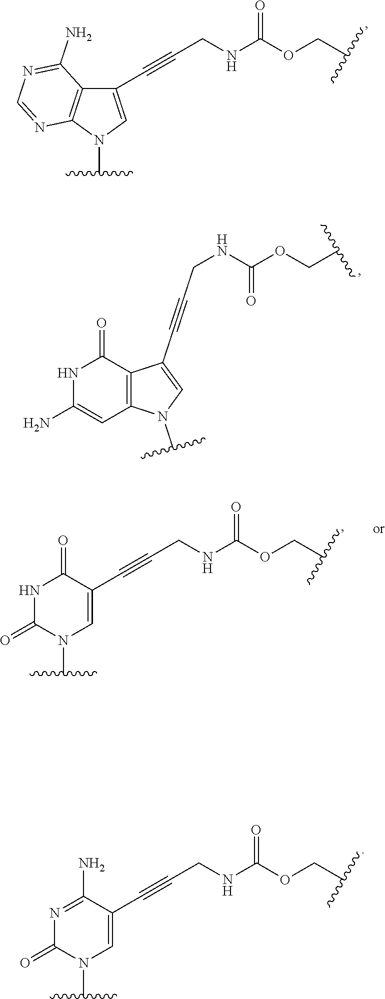 [SMILES: *COC(=O)NCC#Cc1cn(*)c2ncnc(N)c12 ; *COC(=O)NCC#Cc1cn(*)c2cc(N)[nH]c(=O)c12 ; *COC(=O)NCC#Cc1cn(*)c(=O)[nH]c1=O ; *COC(=O)NCC#Cc1cn(*)c(=O)nc1N]

In embodiments, B² is

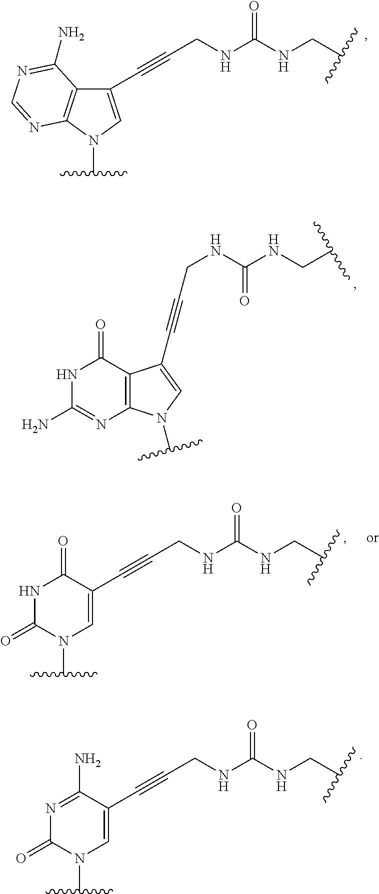 [SMILES: *CNC(=O)NCC#Cc1cn(*)c2ncnc(N)c12 ; *CNC(=O)NCC#Cc1cn(*)c2nc(N)[nH]c(=O)c12 ; *CNC(=O)NCC#Cc1cn(*)c(=O)[nH]c1=O ; *CNC(=O)NCC#Cc1cn(*)c(=O)nc1N]

In embodiments, B² is

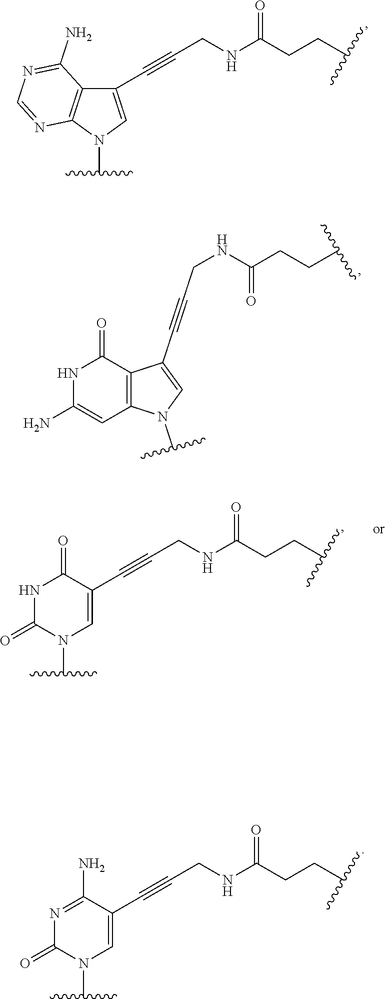 [SMILES: *CCC(=O)NCC#Cc1cn(*)c2ncnc(N)c12 ; *CCC(=O)NCC#Cc1cn(*)c2cc(N)[nH]c(=O)c12 ; *CCC(=O)NCC#Cc1cn(*)c(=O)[nH]c1=O ; *CCC(=O)NCC#Cc1cn(*)c(=O)nc1N]

In embodiments, R⁵ is independently a monophosphate moiety or a derivative thereof (e.g., including a phosphoramidate moiety, phosphorothioate moiety, phosphorodithioate moiety, or methylphosphoroamidite moiety), polyphosphate moiety or derivative thereof (e.g., including a phosphoramidate, phosphorothioate, phosphorodithioate, or methylphosphoroamidite), or nucleic acid moiety or derivative thereof (e.g., including a phosphoramidate, phosphorothioate, phosphorodithioate, or methylphosphoroamidite). In embodiments, R⁵ is a nucleic acid moiety. In embodiments, R⁵ is a monophosphate moiety, polyphosphate moiety, or nucleic acid moiety. In embodiments, R⁵ is a monophosphate moiety. In embodiments, R⁵ is a polyphosphate moiety. In embodiments, R⁵ is a nucleic acid moiety. In embodiments, R⁵ is hydrogen. In embodiments, R⁵ is a triphosphate, having the formula:

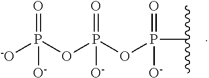

In embodiments, R⁵ is a triphosphate, having the formula:

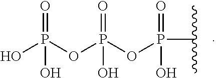 [SMILES: *P(=O)(O)OP(=O)(O)OP(=O)(O)O]

In embodiments, R⁵ is a thiotriphosphate, having the formula:

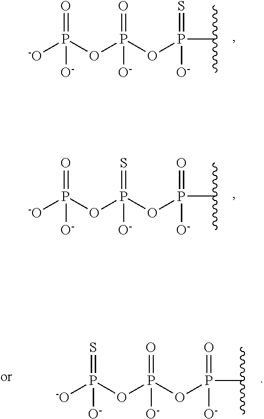

In embodiments, R⁵ is a thiotriphosphate, having the formula:

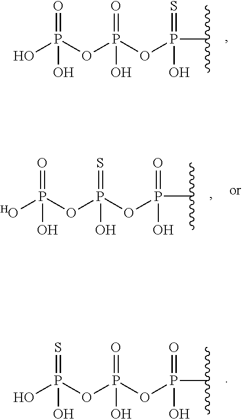 [SMILES: *P(O)(=S)OP(=O)(O)OP(=O)(O)O ; *P(=O)(O)OP(O)(=S)OP(=O)(O)O ; *P(=O)(O)OP(=O)(O)OP(O)(O)=S]

In embodiments, R⁶ is hydrogen. In embodiments, R⁶ is —OH.

In embodiments, R⁷ is hydrogen. In embodiments, R⁷ is a reversible terminator. For example, the reversible terminator may include a known reversible terminator moiety, such as azidomethyl moiety, disulfide moiety, nitrobenzyl moiety, allyl moiety, or an allyloxycarbonyl (See, for example, Metzker et al., “Termination of DNA synthesis by novel 3′-modified deoxyribonucleoside triphosphates,” Nucleic Acids Res., 22:4259-4267, 1994; and U.S. Pat. Nos. 5,872,244; 6,232,465; 6,214,987; 5,808,045; 5,763,594, and 5,302,509. Typically, reversible terminators require contact with a cleaving agent (e.g., a reducing agent or an acid) or suitable radiation (e.g., UV) to remove the reversible terminator and expose a 3′-OH on the nucleotide. In embodiments, the reversible terminator moiety is

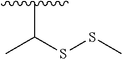 [SMILES: *C(C)SSC]

as described in U.S. Pat. No. 10,738,072, which is incorporated herein by reference for all purposes. In embodiments, the reversible terminator moiety is cyanoethenyl, allenyl, formaldehyde oximyl, acrylaldehyde oximyl, propionaldehyde oximyl, cyanoethenaldehyde oximyl, cis-cyanoethenyl, trans-cyanoethenyl, cis-cyanofluoroethenyl, trans-cyanofluoroethenyl, biscyanoethenyl, bisfluoroethenyl, cis-propenyl, trans-propenyl, nitroethenyl, acetoethenyl, methylcarbonoethenyl, amidoethenyl, methylsulfonoethenyl, methylsulfonoethyl, formimidate, formhydroxymate, vinyloethenyl, ethylenoethenyl, cyanoethylenyl, nitroethylenyl, amidoethylenyl, for example the reversible terminator moieties as described in U.S. Publication 2019/0144482, which is incorporated herein by reference for all purposes. In embodiments, the reversible terminator moiety includes an alkyne moiety (e.g., a propargyl moiety), for example the reversible terminator moieties as described in U.S. Publication 2015/0050697, which is incorporated herein by reference for all purposes. In embodiments, the reversible terminator moiety includes a phosphate diester group as described in U.S. Publication 2014/0242579, which is incorporated herein by reference for all purposes.

In embodiments, R⁷ is

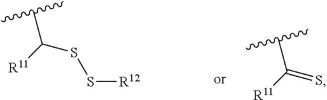

wherein R¹¹ and R¹² are as described herein, including embodiments. In embodiments, R⁷ is —NH₂, —CN, —CH₃, C₂-C₆ allyl (e.g., —CH₂—CH═CH₂), methoxyalkyl (e.g., —CH₂—O—CH₃ or —CH₂—O—CH₂—CH═CH), or —CH₂N₃. In embodiments, R⁷ is —CH₂N₃. In embodiments, R⁷ is

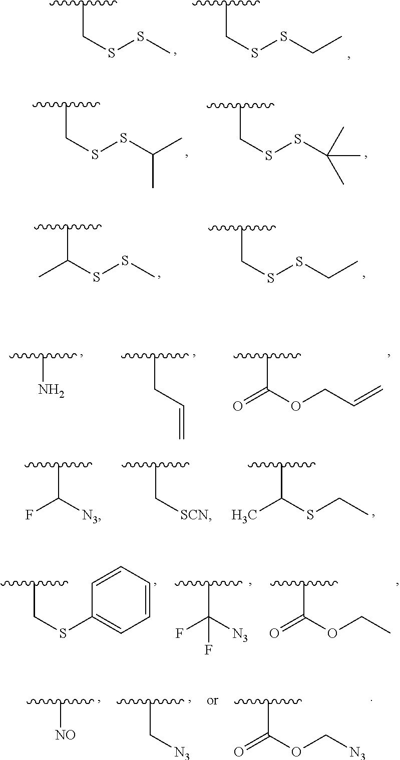

In embodiments R⁷ is

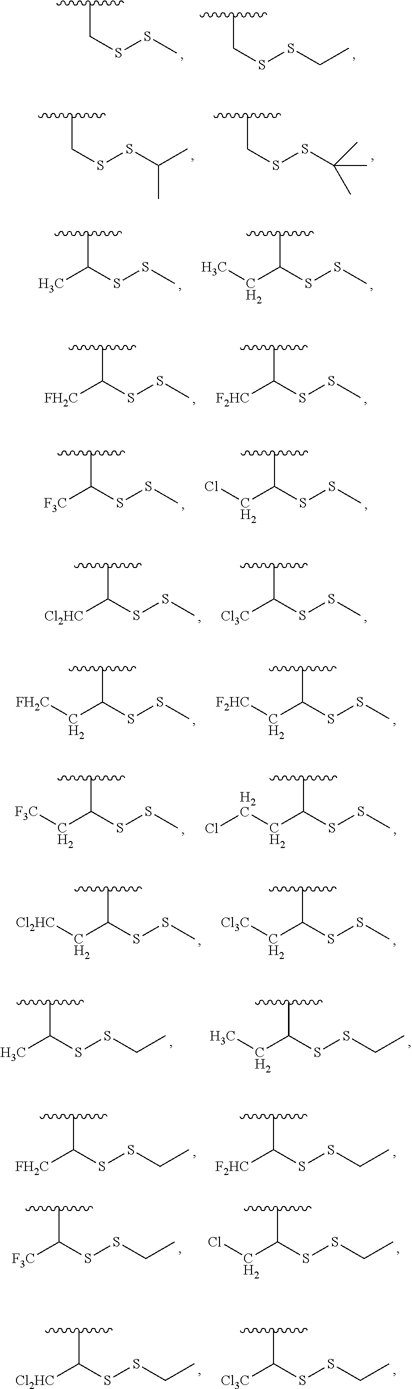

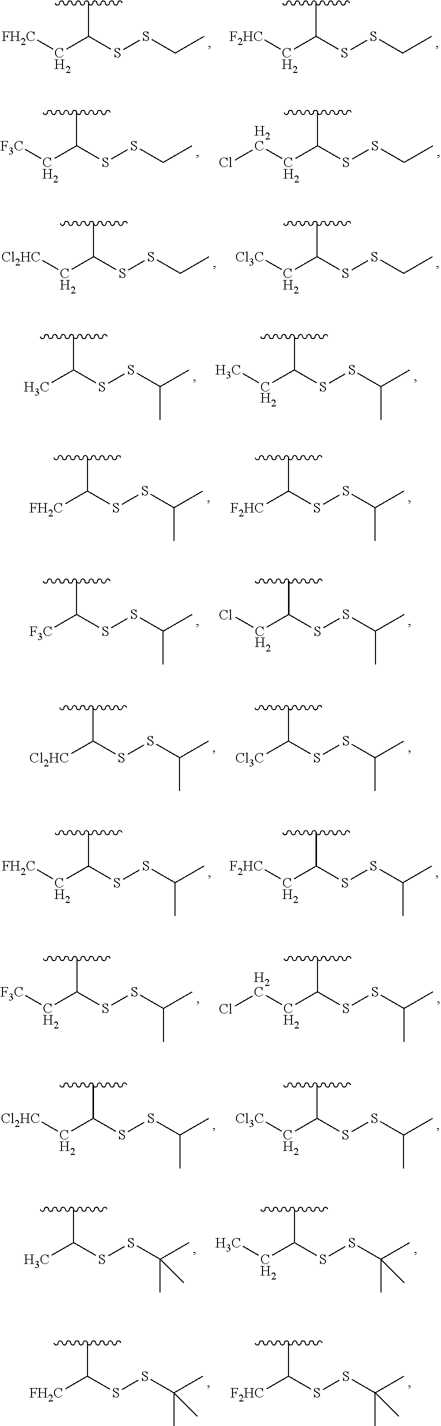

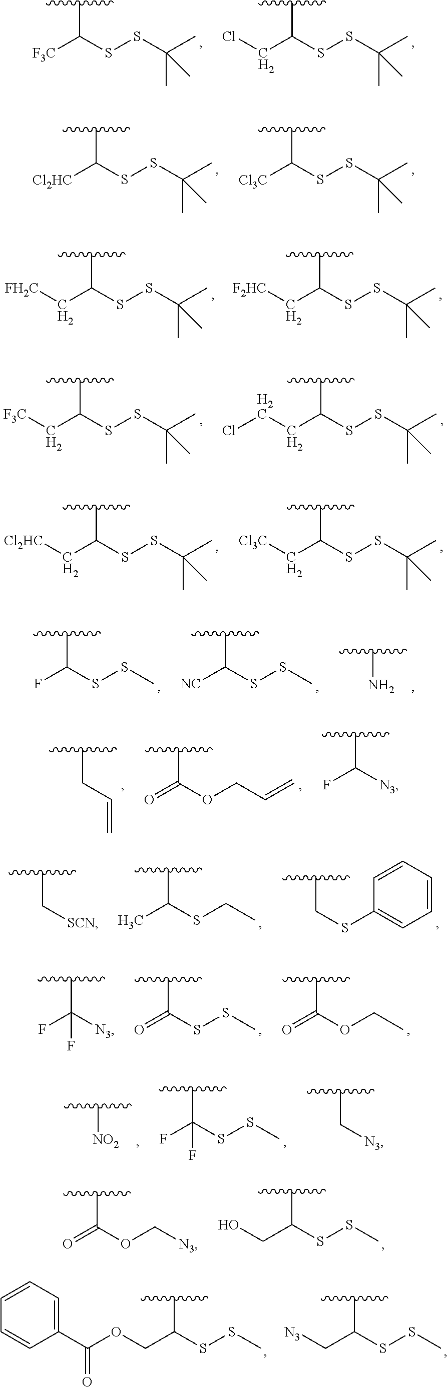

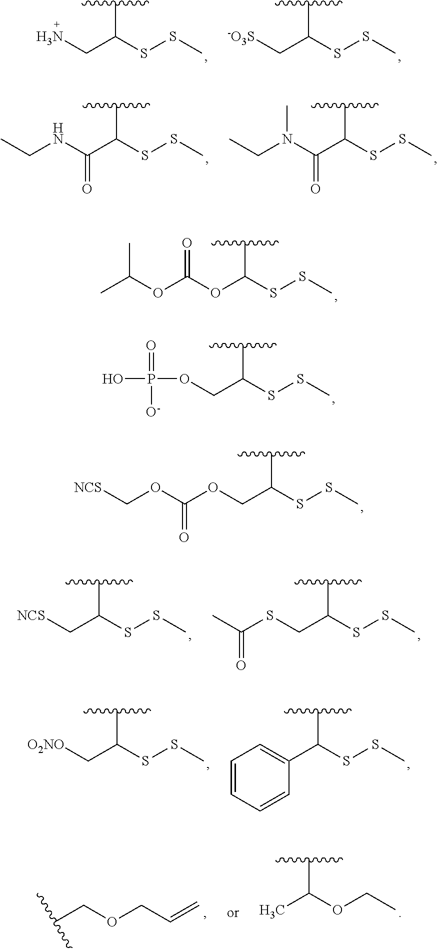

In embodiments, R⁷ is

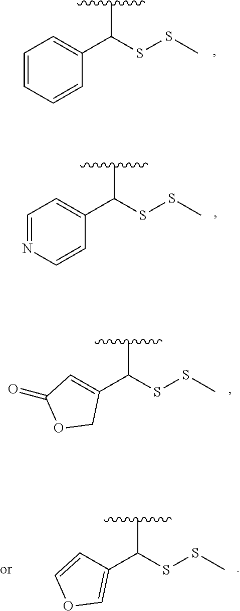

In embodiments, R⁷ is

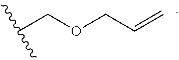 [SMILES: *COCC=C]

In embodiments, L²⁰⁰ is a cleavable linker including an azido (i.e., —N₃) moiety or a dithio (i.e., —S—S—) moiety. In embodiments, L²⁰⁰ is a cleavable linker including:

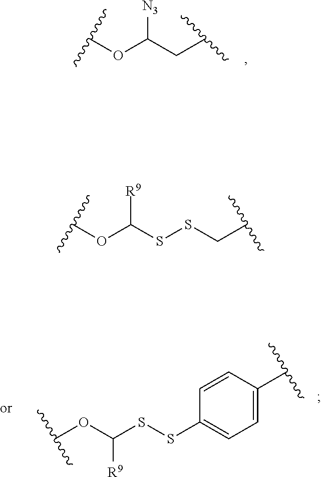

wherein, R⁹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹ is substituted or unsubstituted alkyl. In embodiments, R⁹ is substituted or unsubstituted aryl, or substituted or unsubstituted heteroaryl. In embodiments, L²⁰⁰ includes

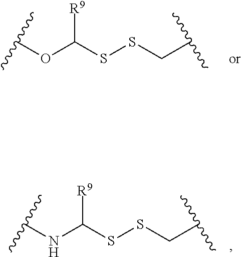

wherein R⁹ is as described herein. In embodiments, L²⁰⁰ includes

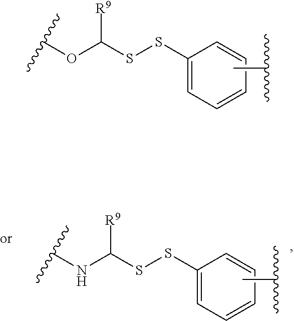

wherein R⁹ is as described herein. In embodiments, L²⁰⁰ includes

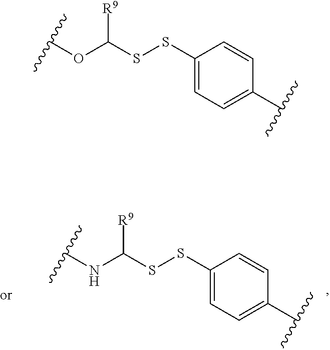

wherein R⁹ is as described herein.

In embodiments, L²⁰⁰ is -L²⁰¹-L²⁰²-L²⁰³-L²⁰⁴-L²⁰⁵-. L²⁰¹, L²⁰², L²⁰³, L²⁰⁴, and L²⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, thio-trigger moiety, substituted or unsubstituted alkylene (e.g., —CH(OH)— or —C(CH₂)—), substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L²⁰¹, L²⁰², L²⁰³, L²⁰⁴, and L²⁰⁵ independently includes PEG. In embodiments, L²⁰¹, L²⁰², L²⁰³, L²⁰⁴, and L²⁰⁵ independently includes

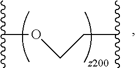

wherein z200 is independently an integer from 1 to 8. In embodiments, z200 is 1. In embodiments, z200 is 2. In embodiments, z200 is 3. In embodiments, z200 is 4. In embodiments, z200 is 5. In embodiments, z200 is 6. In embodiments, z200 is 7. In embodiments, z200 is 8. In embodiments, z200 is an integer from 2 to 8. In embodiments, z200 is an integer from 4 to 6.

In embodiments, at least one of L²⁰¹, L²⁰², L²⁰³, L²⁰⁴, and L²⁰⁵ independently includes

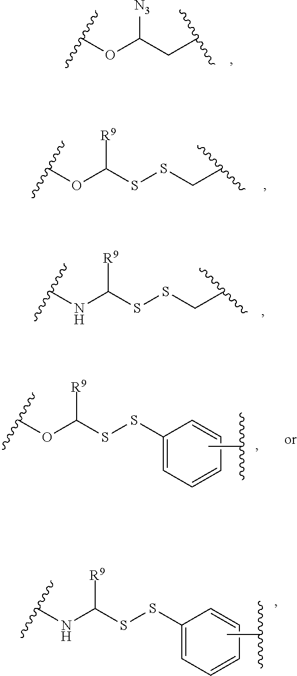

wherein R⁹ is as described herein.

In embodiments, L²⁰⁰ is -L²⁰¹-L²⁰²-L²⁰³-L²⁰⁴-L²⁰⁵-. In embodiments, L²⁰¹, L²⁰², L²⁰³, L²⁰⁴, and L²⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L²⁰¹ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L²⁰¹ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20 membered, 8 to 20 membered, 2 to 10 membered, 3 to 10 membered, 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L²⁰¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰¹ is substituted with a plurality of groups selected from substituent groups, size-limited substituent groups, and lower substituent groups; each substituent group, size-limited substituent group, and/or lower substituent group may optionally be different. In embodiments, when L²⁰¹ is substituted, it is substituted with at least one substituent group. In embodiments, when L²⁰¹ is substituted, it is substituted with at least one size-limited substituent group. In embodiments, when L²⁰¹ is substituted, it is substituted with at least one lower substituent group.

In embodiments, L²⁰¹ is a bond, —NH—, —NR²⁰¹—, —S—, —O—, —C(O)—, —C(O)O—, —OC(O)—, —NHC(O)—, —C(O)NH—, —NHC(O)NH—, —NHC(NH)NH—, —C(S)—, R²⁰¹-substituted or unsubstituted alkylene (e.g., C₁-C₂₀, C₁₀-C₂₀, C₁-C₈, C₁-C₆, or C₁-C₄), R²⁰¹-substituted or unsubstituted heteroalkylene (e.g., 2 to 20 membered, 8 to 20 membered, 2 to 10 membered, 3 to 10 membered, 2 to 8 membered, 2 to 6 membered, or 2 to 4 membered), R²⁰¹-substituted or unsubstituted cycloalkylene (e.g., C₃-C₈, C₃-C₆, or C₅-C₆), R²⁰¹-substituted or unsubstituted heterocycloalkylene (e.g., 3 to 8, 3 to 6, or 5 to 6 membered), R²⁰¹-substituted or unsubstituted arylene (e.g., C₆-C₁₀, C₁₀, or phenylene), or R²⁰¹-substituted or unsubstituted heteroarylene (e.g., 5 to 10, 5 to 9, or 5 to 6 membered). In embodiments, L²⁰¹ is a bond. In embodiments, L²⁰¹ is —NH—. In embodiments, L²⁰¹ is —NR²⁰¹—. In embodiments, L²⁰¹ is —S—. In embodiments, L²⁰¹ is —O—. In embodiments, L²⁰¹ is —C(O)—. In embodiments, L²⁰¹ is —C(O)O—. In embodiments, L²⁰¹ is —OC(O)—. In embodiments, L²⁰¹ is —NHC(O)—. In embodiments, L²⁰¹ is —C(O)NH—. In embodiments, L²⁰¹ is —NHC(O)NH—. In embodiments, L²⁰¹ is —NHC(NH)NH—. In embodiments, L²⁰¹ is —C(S)—. In embodiments, L²⁰¹ is R²⁰¹-substituted or unsubstituted C₁-C₂₀ alkylene. In embodiments, L²⁰¹ is R²⁰¹-substituted or unsubstituted 2 to 20 membered heteroalkylene. In embodiments, L²⁰¹ is R²⁰¹-substituted or unsubstituted 3 to 10 membered heteroalkylene. In embodiments, L²⁰¹ is R²⁰¹-substituted or unsubstituted C₃-C₈ cycloalkylene. In embodiments, L²⁰¹ is R²⁰¹-substituted or unsubstituted 3 to 8 membered heterocycloalkylene. In embodiments, L²⁰¹ is R²⁰¹-substituted or unsubstituted C₆-C₁₀ arylene. In embodiments, L²⁰¹ is R²⁰¹-substituted or unsubstituted 5 to 10 membered heteroarylene. In embodiments, L²⁰¹ is a bond, —NH—, —NR²⁰¹—, —S—, —O—, —C(O)—, —C(O)O—, —OC(O)—, —NHC(O)—, —C(O)NH—, —NHC(O)NH—, —NHC(NH)NH—, —C(S)—, —CH(OH)—, or —C(CH₂)—. In embodiments, L²⁰¹ is a bond. In embodiments, L²⁰¹ is —NH—. In embodiments, L²⁰¹ is —NR²⁰¹—. In embodiments, L²⁰¹ is —S—. In embodiments, L²⁰¹ is —O—. In embodiments, L²⁰¹ is —C(O)—. In embodiments, L²⁰¹ is —C(O)O—. In embodiments, L²⁰¹ is —OC(O)—. In embodiments, L²⁰¹ is —NHC(O)—. In embodiments, L²⁰¹ is —C(O)NH—. In embodiments, L²⁰¹ is —NHC(O)NH—. In embodiments, L²⁰¹ is —NHC(NH)NH—. In embodiments, L²⁰¹ is —C(S)—. In embodiments, L²⁰¹ is —CH(OH)—. In embodiments, L²⁰¹ is —C(CH₂)—. In embodiments, L²⁰¹ is —(CH₂CH₂O)_{b}—. In embodiments, L²⁰¹ is —CCCH₂(OCH₂CH₂)ₐ—NHC(O)—(CH₂)_{c}(OCH₂CH₂)_{b}—. In embodiments, L²⁰¹ is —CHCHCH₂—NHC(O)—(CH₂)_{c}(OCH₂CH₂)_{b}—. In embodiments, L²⁰¹ is —CCCH₂—NHC(O)—(CH₂)_{c}(OCH₂CH₂)_{b}—. In embodiments, L²⁰¹ is —CCCH₂—. The symbol a is an integer from 0 to 8. In embodiments, a is 1. In embodiments, a is 0. The symbol b is an integer from 0 to 8. In embodiments, b is 0. In embodiments, b is 1 or 2. In embodiments, b is an integer from 2 to 8. In embodiments, b is 1. The symbol c is an integer from 0 to 8. In embodiments, c is 0. In embodiments, c is 1. In embodiments, c is 2. In embodiments, c is 3.

R²⁰¹ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{201A}-substituted or unsubstituted alkyl (e.g., C₁-C₂₀, C₁₀-C₂₀, C₁-C₈, C₁-C₆, or C₁-C₄), R^{201A}-substituted or unsubstituted heteroalkyl (e.g., 2 to 20, 8 to 20, 2 to 10, 2 to 8, 2 to 6, or 2 to 4 membered), R^{201A}-substituted or unsubstituted cycloalkyl (e.g., C₃-C₈, C₃-C₆, or C₅-C₆), R^{201A}-substituted or unsubstituted heterocycloalkyl (e.g., 3 to 8, 3 to 6, or 5 to 6 membered), R^{201A}-substituted or unsubstituted aryl (e.g., C₆-C₁₀, C₁₀, or phenyl), or R^{201A}-substituted or unsubstituted heteroaryl (e.g., 5 to 10, 5 to 9, or 5 to 6 membered).

In embodiments, R²⁰¹ is independently —NH₂. In embodiments, R²⁰¹ is independently —OH. In embodiments, R²⁰¹ is independently halogen. In embodiments, R²⁰¹ is independently —CN. In embodiments, R²⁰¹ is independently oxo. In embodiments, R²⁰¹ is independently —CF₃. In embodiments, R²⁰¹ is independently —COOH. In embodiments, R²⁰¹ is independently —CONH₂. In embodiments, R²⁰¹ is independently —F. In embodiments, R²⁰¹ is independently —Cl. In embodiments, R²⁰¹ is independently —Br. In embodiments, R²⁰¹ is independently —I.

In embodiments, L²⁰² is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L²⁰² is a bond, —NH—, —S—, —O—, —C(O)—, —C(O)O—, —OC(O)—, —NHC(O)—, —C(O)NH—, —NHC(O)NH—, —NHC(NH)NH—, —C(S)—, —SS—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L²⁰² is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L²⁰² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰² is substituted with a plurality of groups selected from substituent groups, size-limited substituent groups, and lower substituent groups; each substituent group, size-limited substituent group, and/or lower substituent group may optionally be different. In embodiments, when L²⁰² is substituted, it is substituted with at least one substituent group. In embodiments, when L²⁰² is substituted, it is substituted with at least one size-limited substituent group. In embodiments, when L²⁰² is substituted, it is substituted with at least one lower substituent group.

In embodiments, L²⁰² is a bond, —NH—, —OCH(R²⁰²)—, —OCH(CH₂R²⁰²)—, —OCH(CH₂CN)—, —S—, —O—, —C(O)—, —C(O)O—, —OC(O)—, —NHC(O)—, —C(O)NH—, —NHC(O)NH—, —NHC(NH)NH—, —C(S)—, —SS—, R²⁰²-substituted or unsubstituted alkylene (e.g., C₁-C₂₀, C₁₀-C₂₀, C₁-C₈, C₁-C₆, or C₁-C₄), R²⁰²-substituted or unsubstituted heteroalkylene (e.g., 2 to 20, 8 to 20, 2 to 10, 2 to 8, 2 to 6, or 2 to 4 membered), R²⁰²-substituted or unsubstituted cycloalkylene (e.g., C₃-C₈, C₃-C₆, or C₅-C₆), R²⁰²-substituted or unsubstituted heterocycloalkylene (e.g., 3 to 8, 3 to 6, or 5 to 6 membered), R²⁰²-substituted or unsubstituted arylene (e.g., C₆-C₁₀, C₁₀, or phenylene), or R²⁰²-substituted or unsubstituted heteroarylene (e.g., 5 to 10, 5 to 9, or 5 to 6 membered). In embodiments, L²⁰² is a bond. In embodiments, L²⁰² is —NH—. In embodiments, L²⁰² is —OC(—SSR²⁰²)(CH₃)—. In embodiments, L²⁰² is —OC(—SCN)(CH₃)—. In embodiments, L²⁰² is —OC(N₃)(CH₃)—. In embodiments, L²⁰² is —OCH(—SSR²⁰²)—. In embodiments, L²⁰² is —OCH(—SCN)—. In embodiments, L²⁰² is —OCH(N₃)—. In embodiments, L²⁰² is —OCH(R²⁰²)—. In embodiments, L²⁰² is —OCH(CH₂R²⁰²)—. In embodiments, L²⁰² is —OCH(CH₂CN)—. In embodiments, L²⁰² is —S—. In embodiments, L²⁰² is —O—. In embodiments, L²⁰² is —C(O)—. In embodiments, L²⁰² is —C(O)O—. In embodiments, L²⁰² is —OC(O)—. In embodiments, L²⁰² is —NHC(O)—. In embodiments, L²⁰² is —C(O)NH—. In embodiments, L²⁰² is —NHC(O)NH—. In embodiments, L²⁰² is —NHC(NH)NH—. In embodiments, L²⁰² is —C(S)—. In embodiments, L²⁰² is —SS—. In embodiments, L²⁰² is R²⁰²-substituted or unsubstituted C₁-C₂₀ alkylene. In embodiments, L²⁰² is R²⁰²-substituted or unsubstituted 2 to 20 membered heteroalkylene. In embodiments, L²⁰² is R²⁰²-substituted or unsubstituted C₃-C₈ cycloalkylene. In embodiments, L²⁰² is R²⁰²-substituted or unsubstituted 3 to 8 membered heterocycloalkylene. In embodiments, L²⁰² is R²⁰²-substituted or unsubstituted C₆-C₁₀ arylene. In embodiments, L²⁰² is R²⁰²-substituted or unsubstituted phenylene. In embodiments, L²⁰² is R²⁰²-substituted or unsubstituted 5 to 10 membered heteroarylene.

R²⁰² is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered).

In embodiments, a substituted R²⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰² is substituted with a plurality of groups selected from substituent groups, size-limited substituent groups, and lower substituent groups; each substituent group, size-limited substituent group, and/or lower substituent group may optionally be different. In embodiments, when R²⁰² is substituted, it is substituted with at least one substituent group. In embodiments, when R²⁰² is substituted, it is substituted with at least one size-limited substituent group. In embodiments, when R²⁰² is substituted, it is substituted with at least one lower substituent group.

In embodiments, R²⁰² is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{202A}-substituted or unsubstituted alkyl (e.g., C₁-C₂₀, C₁₀-C₂₀, C₁-C₈, C₁-C₆, or C₁-C₄), R^{202A}-substituted or unsubstituted heteroalkyl (e.g., 2 to 20, 8 to 20, 2 to 10, 2 to 8, 2 to 6, or 2 to 4 membered), R^{202A}-substituted or unsubstituted cycloalkyl (e.g., C₃-C₈, C₃-C₆, or C₅-C₆), R^{202A}-substituted or unsubstituted heterocycloalkyl (e.g., 3 to 8, 3 to 6, or 5 to 6 membered), R^{202A}-substituted or unsubstituted aryl (e.g., C₆-C₁₀, C₁₀, or phenyl), or R^{202A}-substituted or unsubstituted heteroaryl (e.g., 5 to 10, 5 to 9, or 5 to 6 membered).

In embodiments, R²⁰² is independently —NH₂. In embodiments, R²⁰² is independently —OH. In embodiments, R²⁰² is independently halogen. In embodiments, R²⁰² is independently —CN. In embodiments, R²⁰² is independently oxo. In embodiments, R²⁰² is independently —CF₃. In embodiments, R²⁰² is independently —COOH. In embodiments, R²⁰² is independently —CONH₂. In embodiments, R²⁰² is independently —F. In embodiments, R²⁰² is independently —Cl. In embodiments, R²⁰² is independently —Br. In embodiments, R²⁰² is independently —I.

In embodiments, R²⁰² is independently unsubstituted alkyl (e.g., C₁-C₂₀, C₁₀-C₂₀, C₁-C₈, C₁-C₆, or C₁-C₄). In embodiments, R²⁰² is independently unsubstituted C₁-C₆ alkyl. In embodiments, R²⁰² is independently unsubstituted C₁-C₄ alkyl. In embodiments, R²⁰² is independently unsubstituted methyl. In embodiments, R²⁰² is independently unsubstituted tert-butyl. In embodiments, R²⁰² is independently hydrogen.

In embodiments, L²⁰³ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L²⁰³ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L²⁰³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰³ is substituted with a plurality of groups selected from substituent groups, size-limited substituent groups, and lower substituent groups; each substituent group, size-limited substituent group, and/or lower substituent group may optionally be different. In embodiments, when L²⁰³ is substituted, it is substituted with at least one substituent group. In embodiments, when L²⁰³ is substituted, it is substituted with at least one size-limited substituent group. In embodiments, when L²⁰³ is substituted, it is substituted with at least one lower substituent group.

In embodiments, L²⁰³ is a bond, —NH—, —NR²⁰³—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, R²⁰³-substituted or unsubstituted alkylene (e.g., C₁-C₂₀, C₁₀-C₂₀, C₁-C₈, C₁-C₆, or C₁-C₄), R²⁰³-substituted or unsubstituted heteroalkylene (e.g., 2 to 20 membered, 8 to 20 membered, 5 to 16 membered, 2 to 10, 2 to 8, 2 to 6, or 2 to 4 membered), R²⁰³-substituted or unsubstituted cycloalkylene (e.g., C₃-C₈, C₃-C₆, or C₅-C₆), R²⁰³-substituted or unsubstituted heterocycloalkylene (e.g., 3 to 8, 3 to 6, or 5 to 6 membered), R²⁰³-substituted or unsubstituted arylene (e.g., C₆-C₁₀, C₁₀, or phenylene), or R²⁰³-substituted or unsubstituted heteroarylene (e.g., 5 to 10, 5 to 9, or 5 to 6 membered). In embodiments, L²⁰³ is a bond. In embodiments, L²⁰³ is —NH—. In embodiments, L²⁰³ is —NR²⁰³—. In embodiments, L²⁰³ is —S—. In embodiments, L²⁰³ is —O—. In embodiments, L²⁰³ is —C(O)—. In embodiments, L²⁰³ is —C(O)O—. In embodiments, L²⁰³ is —OC(O)—. In embodiments, L²⁰³ is —NHC(O)—. In embodiments, L²⁰³ is —C(O)NH—. In embodiments, L²⁰³ is —NHC(O)NH—. In embodiments, L²⁰³ is —NHC(NH)NH—. In embodiments, L²⁰³ is —C(S)—. In embodiments, L²⁰³ is —N═N—. In embodiments, L²⁰³ is —SS—. In embodiments, L²⁰³ is R²⁰³-substituted or unsubstituted C₁-C₂₀ alkylene. In embodiments, L²⁰³ is R²⁰³-substituted or unsubstituted 2 to 20 membered heteroalkylene. In embodiments, L²⁰³ is R²⁰³-substituted or unsubstituted 5 to 16 membered heteroalkylene. In embodiments, L²⁰³ is R²⁰³-substituted or unsubstituted 2 to 10 membered heteroalkylene. In embodiments, L²⁰³ is R²⁰³-substituted or unsubstituted C₃-C₈ cycloalkylene. In embodiments, L²⁰³ is R²⁰³-substituted or unsubstituted 3 to 8 membered heterocycloalkylene. In embodiments, L²⁰³ is R²⁰³-substituted or unsubstituted C₆-C₁₀ arylene. In embodiments, L²⁰³ is R²⁰³-substituted or unsubstituted 5 to 10 membered heteroarylene.

In embodiments, L²⁰³ is a bond, —NH—, —NR²⁰³—, —S—, —O—, —C(O)—, —C(O)O—, —OC(O)—, —NHC(O)—, —C(O)NH—, —NHC(O)NH—, —NHC(NH)NH—, —C(S)—, —N═N—, —SS—, —CH(OH)—, or —C(CH₂)—. In embodiments, L²⁰³ is a bond. In embodiments, L²⁰³ is —NH—. In embodiments, L²⁰³ is —NR²⁰³—. In embodiments, L²⁰³ is —S—. In embodiments, L²⁰³ is —O—. In embodiments, L²⁰³ is —C(O)—. In embodiments, L²⁰³ is —C(O)O—. In embodiments, L²⁰³ is —OC(O)—. In embodiments, L²⁰³ is —NHC(O)—. In embodiments, L²⁰³ is —C(O)NH—. In embodiments, L²⁰³ is —NHC(O)NH—. In embodiments, L²⁰³ is —NHC(NH)NH—. In embodiments, L²⁰³ is —C(S)—. In embodiments, L²⁰³ is —N═N—. In embodiments, L²⁰³ is —SS—. In embodiments, L²⁰³ is —CH(OH)—. In embodiments, L²⁰³ is —C(CH₂)—. In embodiments, L²⁰³ is —(CH₂CH₂O)_{d}—. In embodiments, L²⁰³ is —(CH₂O)_{d}—. In embodiments, L²⁰³ is —(CH₂)_{d}—. In embodiments, L²⁰³ is —(CH₂)_{d}—NH—. In embodiments, L²⁰³ is -(unsubstituted phenylene)-. In embodiments, L²⁰³ is

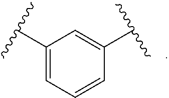 [SMILES: *c1cccc(*)c1]

In embodiments, L²⁰³ is -(unsubstituted phenylene)-C(O)NH—. In embodiments, L²⁰³ is

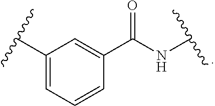 [SMILES: *NC(=O)c1cccc(*)c1]

In embodiments, L²⁰³ is -(unsubstituted phenylene)-NHC(O)—. In embodiments, L²⁰³ is

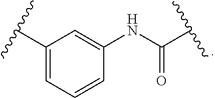 [SMILES: *C(=O)Nc1cccc(*)c1]

The symbol d is an integer from 0 to 8. In embodiments, d is 3. In embodiments, d is 1. In embodiments, d is 2. In embodiments, d is 0.

R²⁰³ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{203A}-substituted or unsubstituted alkyl (e.g., C₁-C₂₀, C₁₀-C₂₀, C₁-C₈, C₁-C₆, or C₁-C₄), R^{203A}-substituted or unsubstituted heteroalkyl (e.g., 2 to 20, 8 to 20, 2 to 10, 2 to 8, 2 to 6, or 2 to 4 membered), R^{203A}-substituted or unsubstituted cycloalkyl (e.g., C₃-C₈, C₃-C₆, or C₅-C₆), R^{203A}-substituted or unsubstituted heterocycloalkyl (e.g., 3 to 8, 3 to 6, or 5 to 6 membered), R^{203A}-substituted or unsubstituted aryl (e.g., C₆-C₁₀, C₁₀, or phenyl), or R^{203A}-substituted or unsubstituted heteroaryl (e.g., 5 to 10, 5 to 9, or 5 to 6 membered).

In embodiments, R²⁰³ is independently —NH₂. In embodiments, R²⁰³ is independently —OH. In embodiments, R²⁰³ is independently halogen. In embodiments, R²⁰³ is independently —CN. In embodiments, R²⁰³ is independently oxo. In embodiments, R²⁰³ is independently —CF₃. In embodiments, R²⁰³ is independently —COOH. In embodiments, R²⁰³ is independently —CONH₂. In embodiments, R²⁰³ is independently —F. In embodiments, R²⁰³ is independently —Cl. In embodiments, R²⁰³ is independently —Br. In embodiments, R²⁰³ is independently —I.

In embodiments, L²⁰⁴ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L²⁰⁴ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20 membered, 8 to 20 membered, 5 to 16 membered, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L²⁰⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰⁴ is substituted with a plurality of groups selected from substituent groups, size-limited substituent groups, and lower substituent groups; each substituent group, size-limited substituent group, and/or lower substituent group may optionally be different. In embodiments, when L²⁰⁴ is substituted, it is substituted with at least one substituent group. In embodiments, when L²⁰⁴ is substituted, it is substituted with at least one size-limited substituent group. In embodiments, when L²⁰⁴ is substituted, it is substituted with at least one lower substituent group.

In embodiments, L²⁰⁴ is a bond, —NH—, —NR²⁰⁴—, —S—, —O—, —C(O)—, —C(O)O—, —OC(O)—, —NHC(O)—, —C(O)NH—, —NHC(O)NH—, —NHC(NH)NH—, —C(S)—, R²⁰⁴-substituted or unsubstituted alkylene (e.g., C₁-C₂₀, C₁₀-C₂₀, C₁-C₈, C₁-C₆, or C₁-C₄), R²⁰⁴-substituted or unsubstituted heteroalkylene (e.g., 2 to 20 membered, 8 to 20 membered, 5 to 16 membered, 2 to 10, 2 to 8, 2 to 6, or 2 to 4 membered), R²⁰⁴-substituted or unsubstituted cycloalkylene (e.g., C₃-C₈, C₃-C₆, or C₅-C₆), R²⁰⁴-substituted or unsubstituted heterocycloalkylene (e.g., 3 to 8, 3 to 6, or 5 to 6 membered), R²⁰⁴-substituted or unsubstituted arylene (e.g., C₆-C₁₀, C₁₀, or phenylene), or R²⁰⁴-substituted or unsubstituted heteroarylene (e.g., 5 to 10, 5 to 9, or 5 to 6 membered). In embodiments, L²⁰⁴ is a bond. In embodiments, L²⁰⁴ is —NH—. In embodiments, L²⁰⁴ is —NR²⁰⁴—. In embodiments, L²⁰⁴ is —S—. In embodiments, L²⁰⁴ is —O—. In embodiments, L²⁰⁴ is —C(O)—. In embodiments, L²⁰⁴ is —C(O)O—. In embodiments, L²⁰⁴ is —OC(O)—. In embodiments, L²⁰⁴ is —NHC(O)—. In embodiments, L²⁰⁴ is —C(O)NH—. In embodiments, L²⁰⁴ is —NHC(O)NH—. In embodiments, L²⁰⁴ is —NHC(NH)NH—. In embodiments, L²⁰⁴ is —C(S)—. In embodiments, L²⁰⁴ is R²⁰⁴-substituted or unsubstituted C₁-C₂₀ alkylene. In embodiments, L²⁰⁴ is R²⁰⁴-substituted or unsubstituted 2 to 20 membered heteroalkylene. In embodiments, L²⁰⁴ is R²⁰⁴-substituted or unsubstituted 5 to 16 membered heteroalkylene. In embodiments, L²⁰⁴ is R²⁰⁴-substituted or unsubstituted 2 to 10 membered heteroalkylene. In embodiments, L²⁰⁴ is R²⁰⁴-substituted or unsubstituted C₃-C₈ cycloalkylene. In embodiments, L²⁰⁴ is R²⁰⁴-substituted or unsubstituted 3 to 8 membered heterocycloalkylene. In embodiments, L²⁰⁴ is R²⁰⁴-substituted or unsubstituted C₆-C₁₀ arylene. In embodiments, L²⁰⁴ is R²⁰⁴-substituted or unsubstituted 5 to 10 membered heteroarylene. In embodiments, L²⁰⁴ is R²⁰⁴-substituted or unsubstituted phenylene.

In embodiments, L²⁰⁴ is a bond, —NH—, —NR²⁰⁴—, —S—, —O—, —C(O)—, —C(O)O—, —OC(O)—, —NHC(O)—, —C(O)NH—, —NHC(O)NH—, —NHC(NH)NH—, —C(S)—, —CH(OH)—, or —C(CH₂)—. In embodiments, L²⁰⁴ is a bond. In embodiments, L²⁰⁴ is —NH—. In embodiments, L²⁰⁴ is —NR²⁰⁴—. In embodiments, L²⁰⁴ is —S—. In embodiments, L²⁰⁴ is —O—. In embodiments, L²⁰⁴ is —C(O)—. In embodiments, L²⁰⁴ is —C(O)O—. In embodiments, L²⁰⁴ is —OC(O)—. In embodiments, L²⁰⁴ is —NHC(O)—. In embodiments, L²⁰⁴ is —C(O)NH—. In embodiments, L²⁰⁴ is —NHC(O)NH—. In embodiments, L²⁰⁴ is —NHC(NH)NH—. In embodiments, L²⁰⁴ is —C(S)—. In embodiments, L²⁰⁴ is —CH(OH)—. In embodiments, L²⁰⁴ is —C(CH₂)—.

In embodiments, L²⁰⁴ is —(CH₂CH₂O)ₑ—. In embodiments, L²⁰⁴ is —(CH₂O)ₑ—. In embodiments, L²⁰⁴ is —(CH₂)ₑ—. In embodiments, L²⁰⁴ is —(CH₂)ₑ—NH—. In embodiments, L²⁰⁴ is -(unsubstituted phenylene)-. In embodiments, L²⁰⁴ is

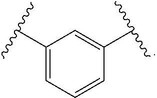 [SMILES: *c1cccc(*)c1]

In embodiments, L²⁰⁴ is -(unsubstituted phenylene)-C(O)NH—. In embodiments, L²⁰⁴ is

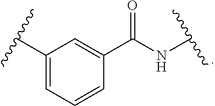 [SMILES: *NC(=O)c1cccc(*)c1]

In embodiments, L²⁰⁴ is -(unsubstituted phenylene)-NHC(O)—. In embodiments, L²⁰⁴ is

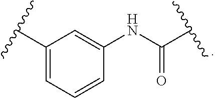 [SMILES: *C(=O)Nc1cccc(*)c1]

The symbol e is an integer from 0 to 8. In embodiments, e is 3. In embodiments, e is 1. In embodiments, e is 2.

R²⁰⁴ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{204A}-substituted or unsubstituted alkyl (e.g., C₁-C₂₀, C₁₀-C₂₀, C₁-C₈, C₁-C₆, or C₁-C₄), R^{204A}-substituted or unsubstituted heteroalkyl (e.g., 2 to 20, 8 to 20, 2 to 10, 2 to 8, 2 to 6, or 2 to 4 membered), R^{204A}-substituted or unsubstituted cycloalkyl (e.g., C₃-C₈, C₃-C₆, or C₅-C₆), R^{204A}-substituted or unsubstituted heterocycloalkyl (e.g., 3 to 8, 3 to 6, or 5 to 6 membered), R^{204A}-substituted or unsubstituted aryl (e.g., C₆-C₁₀, C₁₀, or phenyl), or R^{204A}-substituted or unsubstituted heteroaryl (e.g., 5 to 10, 5 to 9, or 5 to 6 membered).

In embodiments, R²⁰⁴ is independently —NH₂. In embodiments, R²⁰⁴ is independently —OH. In embodiments, R²⁰⁴ is independently halogen. In embodiments, R²⁰⁴ is independently —CN. In embodiments, R²⁰⁴ is independently oxo. In embodiments, R²⁰⁴ is independently —CF₃. In embodiments, R²⁰⁴ is independently —COOH. In embodiments, R²⁰⁴ is independently —CONH₂. In embodiments, R²⁰⁴ is independently —F. In embodiments, R²⁰⁴ is independently —Cl. In embodiments, R²⁰⁴ is independently —Br. In embodiments, R²⁰⁴ is independently —I.

In embodiments, L²⁰⁵ is independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L²⁰⁵ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20 membered, 8 to 20 membered, 5 to 16 membered, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered).

In embodiments, a substituted L²⁰⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰⁵ is substituted with a plurality of groups selected from substituent groups, size-limited substituent groups, and lower substituent groups; each substituent group, size-limited substituent group, and/or lower substituent group may optionally be different. In embodiments, when L²⁰⁵ is substituted, it is substituted with at least one substituent group. In embodiments, when L²⁰⁵ is substituted, it is substituted with at least one size-limited substituent group. In embodiments, when L²⁰⁵ is substituted, it is substituted with at least one lower substituent group.

In embodiments, L²⁰⁵ is a bond, —NH—, —NR²⁰⁵—, —S—, —O—, —C(O)—, —C(O)O—, —OC(O)—, —NHC(O)—, —C(O)NH—, —NHC(O)NH—, —NHC(NH)NH—, —C(S)—, R²⁰⁵-substituted or unsubstituted alkylene (e.g., C₁-C₂₀, C₁₀-C₂₀, C₁-C₈, C₁-C₆, or C₁-C₄), R²⁰⁵-substituted or unsubstituted heteroalkylene (e.g., 2 to 20 membered, 8 to 20 membered, 5 to 16 membered, 2 to 10, 2 to 8, 2 to 6, or 2 to 4 membered), R²⁰⁵-substituted or unsubstituted cycloalkylene (e.g., C₃-C₈, C₃-C₆, or C₅-C₆), R²⁰⁵-substituted or unsubstituted heterocycloalkylene (e.g., 3 to 8, 3 to 6, or 5 to 6 membered), R²⁰⁵-substituted or unsubstituted arylene (e.g., C₆-C₁₀, C₁₀, or phenylene), or R²⁰⁵-substituted or unsubstituted heteroarylene (e.g., 5 to 10, 5 to 9, or 5 to 6 membered). In embodiments, L²⁰⁵ is a bond. In embodiments, L²⁰⁵ is —NH—. In embodiments, L²⁰⁵ is —NR²⁰⁵—. In embodiments, L²⁰⁵ is —S—. In embodiments, L²⁰⁵ is —O—. In embodiments, L²⁰⁵ is —C(O)—. In embodiments, L²⁰⁵ is —C(O)O—. In embodiments, L²⁰⁵ is —OC(O)—. In embodiments, L²⁰⁵ is —NHC(O)—. In embodiments, L²⁰⁵ is —C(O)NH—. In embodiments, L²⁰⁵ is —NHC(O)NH—. In embodiments, L²⁰⁵ is —NHC(NH)NH—. In embodiments, L²⁰⁵ is —C(S)—. In embodiments, L²⁰⁵ is R²⁰⁵-substituted or unsubstituted C₁-C₂₀ alkylene. In embodiments, L²⁰⁵ is R²⁰⁵-substituted or unsubstituted 2 to 20 membered heteroalkylene. In embodiments, L²⁰⁵ is R²⁰⁵-substituted or unsubstituted 5 to 16 membered heteroalkylene. In embodiments, L²⁰⁵ is R²⁰⁵-substituted or unsubstituted 2 to 10 membered heteroalkylene. In embodiments, L²⁰⁵ is R²⁰⁵-substituted or unsubstituted C₃-C₈ cycloalkylene. In embodiments, L²⁰⁵ is R²⁰⁵-substituted or unsubstituted 3 to 8 membered heterocycloalkylene. In embodiments, L²⁰⁵ is R²⁰⁵-substituted or unsubstituted C₆-C₁₀ arylene. In embodiments, L²⁰⁵ is R²⁰⁵-substituted or unsubstituted 5 to 10 membered heteroarylene.

In embodiments, L²⁰⁵ is a bond, —NH—, —NR²⁰⁵—, —S—, —O—, —C(O)—, —C(O)O—, —OC(O)—, —NHC(O)—, —C(O)NH—, —NHC(O)NH—, —NHC(NH)NH—, —C(S)—, —CH(OH)—, or —C(CH₂)—. In embodiments, L²⁰⁵ is a bond. In embodiments, L²⁰⁵ is —NH—. In embodiments, L²⁰⁵ is —NR²⁰⁵—. In embodiments, L²⁰⁵ is —S—. In embodiments, L²⁰⁵ is —O—. In embodiments, L²⁰⁵ is —C(O)—. In embodiments, L²⁰⁵ is —C(O)O—. In embodiments, L²⁰⁵ is —OC(O)—. In embodiments, L²⁰⁵ is —NHC(O)—. In embodiments, L²⁰⁵ is —C(O)NH—. In embodiments, L²⁰⁵ is —NHC(O)NH—. In embodiments, L²⁰⁵ is —NHC(NH)NH—. In embodiments, L²⁰⁵ is —C(S)—. In embodiments, L²⁰⁵ is —CH(OH)—. In embodiments, L²⁰⁵ is —C(CH₂)—.

In embodiments, L²⁰⁵ is —(CH₂CH₂O)_{f}—. In embodiments, L²⁰⁵ is —(CH₂O)_{f}—. In embodiments, L²⁰⁵ is —(CH₂)_{f}—. In embodiments, L²⁰⁵ is —(CH₂)_{f}—NH—. In embodiments, L²⁰⁵ is —C(O)NH(CH₂)_{f}—NH—. In embodiments, L²⁰⁵ is —(CH₂CH₂O)_{f}—(CH₂)_{g}—NH—. In embodiments, L²⁰⁵ is —(CH₂)_{g}—. In embodiments, L²⁰⁵ is —(CH₂)_{g}—NH—. In embodiments, L²⁰⁵ is —NHC(O)—(CH₂)_{f}—NH—. In embodiments, L²⁰⁵ is —NHC(O)—(CH₂)_{f}—NH—. In embodiments, L²⁰⁵ is —NHC(O)—(CH₂CH₂O)_{f}—(CH₂)_{g}—NH—. In embodiments, L²⁰⁵ is —NHC(O)—(CH₂)_{g}—. In embodiments, L²⁰⁵ is —NHC(O)—(CH₂)_{g}—NH—. In embodiments, L²⁰⁵ is —C(O)NH(CH₂)_{f}—NH—. In embodiments, L²⁰⁵ is —C(O)NH—(CH₂CH₂O)_{f}—(CH₂)_{g}—NH—. In embodiments, L²⁰⁵ is —C(O)NH—(CH₂)_{g}—. In embodiments, L²⁰⁵ is —C(O)NH—(CH₂)_{g}—NH—. The symbol f is an integer from 0 to 8. In embodiments, f is 3. In embodiments, f is 1. In embodiments, f is 2. In embodiments, f is 0. The symbol g is an integer from 0 to 8. In embodiments, g is 3. In embodiments, g is 1. In embodiments, g is 2. In embodiments, g is 0.

R²⁰⁵ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, R^{205A}-substituted or unsubstituted alkyl (e.g., C₁-C₂₀, C₁₀-C₂₀, C₁-C₈, C₁-C₆, or C₁-C₄), R^{205A}-substituted or unsubstituted heteroalkyl (e.g., 2 to 20, 8 to 20, 2 to 10, 2 to 8, 2 to 6, or 2 to 4 membered), R^{205A}-substituted or unsubstituted cycloalkyl (e.g., C₃-C₈, C₃-C₆, or C₅-C₆), R^{205A}-substituted or unsubstituted heterocycloalkyl (e.g., 3 to 8, 3 to 6, or 5 to 6 membered), R^{205A}-substituted or unsubstituted aryl (e.g., C₆-C₁₀, C₁₀, or phenyl), or R^{205A}-substituted or unsubstituted heteroaryl (e.g., 5 to 10, 5 to 9, or 5 to 6 membered).

In embodiments, R²⁰⁵ is independently —NH₂. In embodiments, R²⁰⁵ is independently —OH. In embodiments, R²⁰⁵ is independently halogen. In embodiments, R²⁰⁵ is independently —CN. In embodiments, R²⁰⁵ is independently oxo. In embodiments, R²⁰⁵ is independently —CF₃. In embodiments, R²⁰⁵ is independently —COOH. In embodiments, R²⁰⁵ is independently —CONH₂. In embodiments, R²⁰⁵ is independently —F. In embodiments, R²⁰⁵ is independently —Cl. In embodiments, R²⁰⁵ is independently —Br. In embodiments, R²⁰⁵ is independently —I.

R^{201A}, R^{202A}, R^{203A}, R^{204A}, and R^{205A} are each independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L²⁰⁰ is

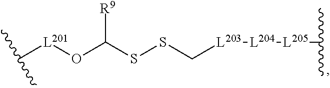

wherein L²⁰¹, L²⁰³, L²⁰⁴, L²⁰⁵, and R⁹ are as described herein. In embodiments, L²⁰⁰ is

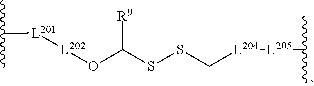

wherein L²⁰¹, L²⁰², L²⁰⁴, L²⁰⁵, and R⁹ are as described herein. In embodiments, L²⁰⁰ is

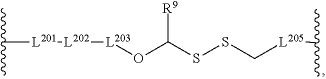

wherein L²⁰¹, L²⁰², L²⁰³, L²⁰⁵, and R⁹ are as described herein. In embodiments, L²⁰⁰ is

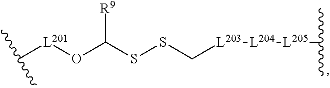

wherein L²⁰¹, L²⁰³, L²⁰⁴, L²⁰⁵, and R⁹ are as described herein. In embodiments, L²⁰⁰ is

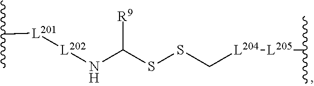

wherein L²⁰¹, L²⁰², L²⁰⁴, L²⁰⁵, and R⁹ are as described herein. In embodiments, L²⁰⁰ is

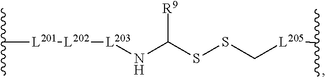

wherein L²⁰¹, L²⁰², L²⁰³, L²⁰⁵, and R⁹ are as described herein.

In embodiments, L²⁰⁰ is -L²⁰¹-O—CH(N₃)-L²⁰³-L²⁰⁴-L²⁰⁵-; and L²⁰¹, L²⁰³, L²⁰⁴, and L²⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L²⁰⁰ is -L²⁰¹-O—CH(N₃)-L²⁰³-L²⁰⁴-L²⁰⁵-; wherein L²⁰¹ is independently a substituted or unsubstituted C₁-C₄ alkylene or substituted or unsubstituted 8 to 20 membered heteroalkylene; L²⁰³ is independently a bond or substituted or unsubstituted 2 to 10 membered heteroalkylene; L²⁰⁴ is independently a bond, substituted or unsubstituted 4 to 18 membered heteroalkylene, or substituted or unsubstituted phenylene; and L²⁰⁵ is independently bond or substituted or unsubstituted 4 to 18 membered heteroalkylene. In embodiments, L²⁰⁰ is -L²⁰¹-O—CH(N₃)—CH₂—O-L²⁰⁴-L²⁰⁵-; wherein L²⁰¹ and L²⁰⁵ are independently a bond, —NH—, —O—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and L²⁰⁴ is unsubstituted phenylene.

In embodiments, L²⁰⁰ is

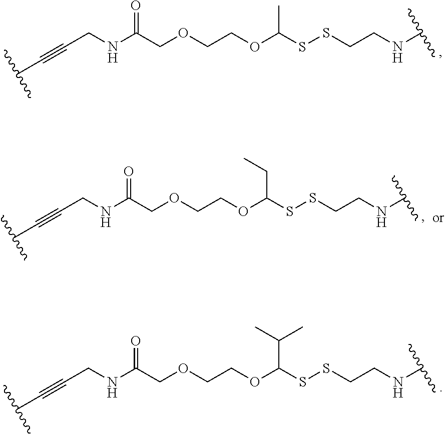 [SMILES: *C#CCNC(=O)COCCOC(C)SSCCN* ; *C#CCNC(=O)COCCOC(CC)SSCCN* ; *C#CCNC(=O)COCCOC(SSCCN*)C(C)C]

In embodiments, L²⁰⁰ is

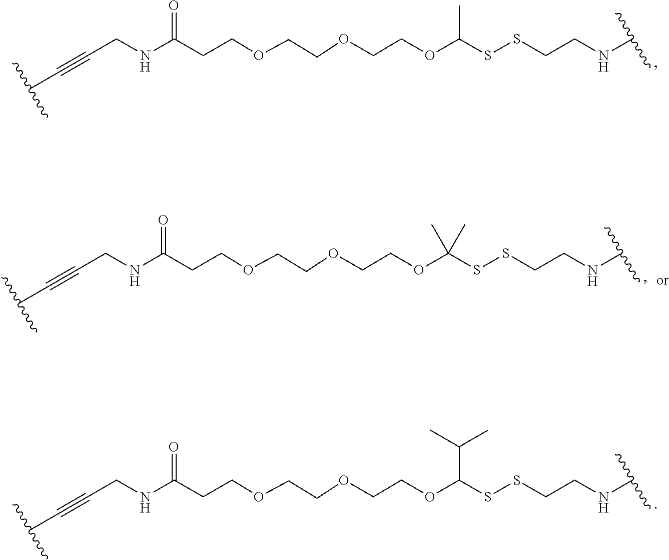 [SMILES: *C#CCNC(=O)CCOCCOCCOC(C)SSCCN* ; *C#CCNC(=O)CCOCCOCCOC(C)(C)SSCCN* ; *C#CCNC(=O)CCOCCOCCOC(SSCCN*)C(C)C]

In embodiments, L²⁰⁰ is

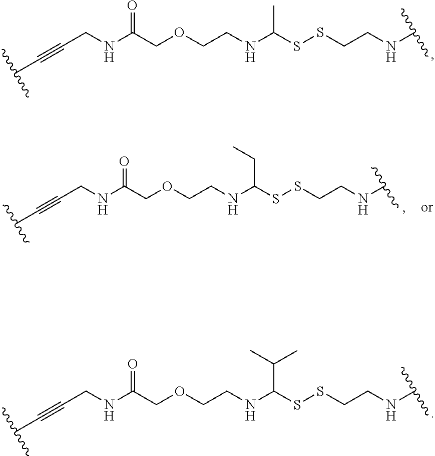 [SMILES: *C#CCNC(=O)COCCNC(C)SSCCN* ; *C#CCNC(=O)COCCNC(CC)SSCCN* ; *C#CCNC(=O)COCCNC(SSCCN*)C(C)C]

In embodiments, L²⁰⁰ is

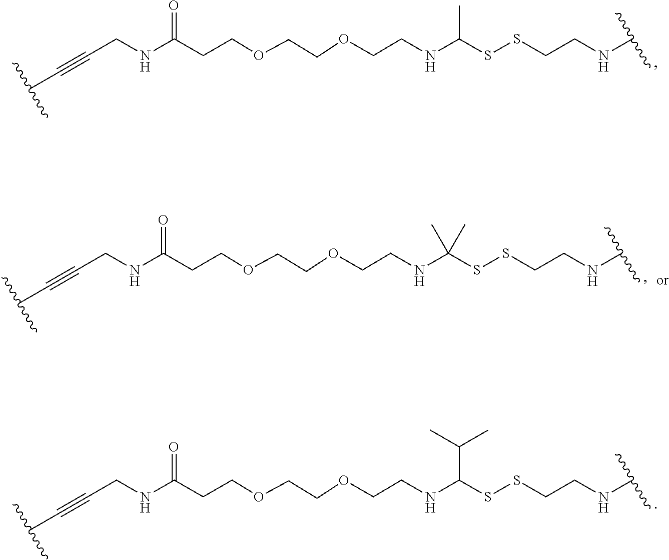 [SMILES: *C#CCNC(=O)CCOCCOCCNC(C)SSCCN* ; *C#CCNC(=O)CCOCCOCCNC(C)(C)SSCCN* ; *C#CCNC(=O)CCOCCOCCNC(SSCCN*)C(C)C]

In embodiments, L²⁰⁰ includes

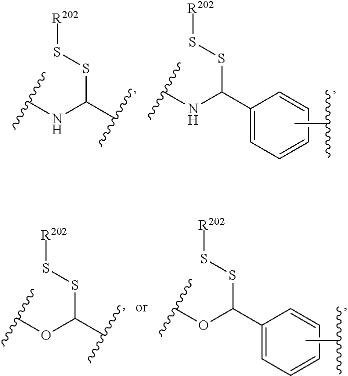

wherein, R²⁰² is unsubstituted C₁-C₄ alkyl. In embodiments, L²⁰⁰ is a cleavable linker including:

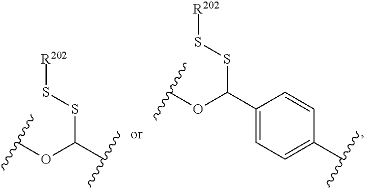

wherein R²⁰² is as described herein. In embodiments, L²⁰⁰ includes

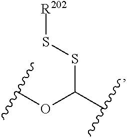

wherein R²⁰² is as described herein. In embodiments, L²⁰⁰ includes

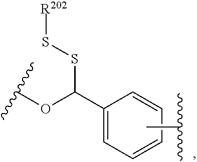

wherein R²⁰² is as described herein. In embodiments, at least one of L²⁰¹, L²⁰², L²⁰3, L²⁰⁴, and L²⁰⁵ independently includes

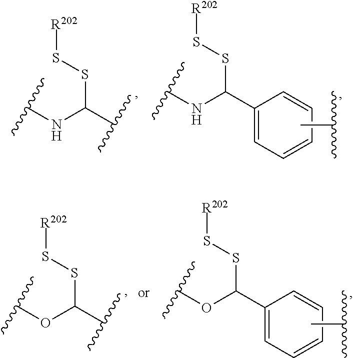

wherein R²⁰² is as described herein. In embodiments, R²⁰² is unsubstituted C₁-C₄ alkyl. In embodiments, R²⁰² is unsubstituted C₁ alkyl. In embodiments, R²⁰² is unsubstituted C₂ alkyl. In embodiments, R²⁰² is unsubstituted C₃ alkyl. In embodiments, R²⁰² is unsubstituted C₄ alkyl.

In embodiments, L²⁰⁰ is

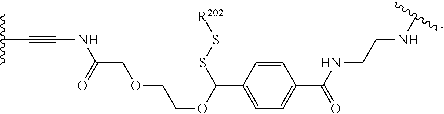

wherein R²⁰² is as described herein. In embodiments, L²⁰⁰ is

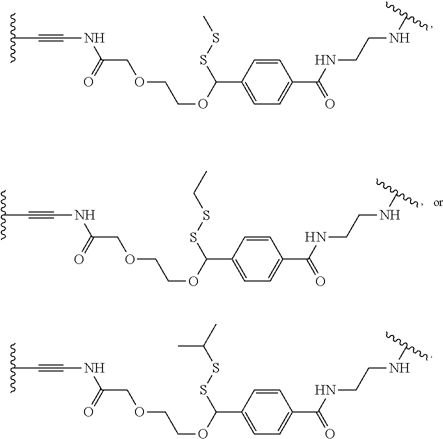 [SMILES: *C#CNC(=O)COCCOC(SSC)c1ccc(C(=O)NCCN*)cc1 ; *C#CNC(=O)COCCOC(SSCC)c1ccc(C(=O)NCCN*)cc1 ; *C#CNC(=O)COCCOC(SSC(C)C)c1ccc(C(=O)NCCN*)cc1]

In embodiments, L²⁰⁰ is

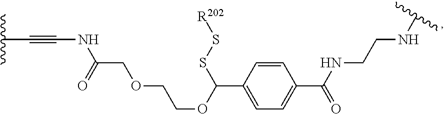

wherein R²⁰² is as described herein. In embodiments, L²⁰⁰ is

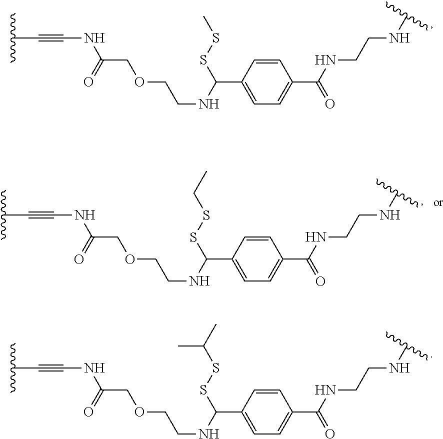 [SMILES: *C#CNC(=O)COCCNC(SSC)c1ccc(C(=O)NCCN*)cc1 ; *C#CNC(=O)COCCNC(SSCC)c1ccc(C(=O)NCCN*)cc1 ; *C#CNC(=O)COCCNC(SSC(C)C)c1ccc(C(=O)NCCN*)cc1]

In embodiments, L²⁰⁰ is

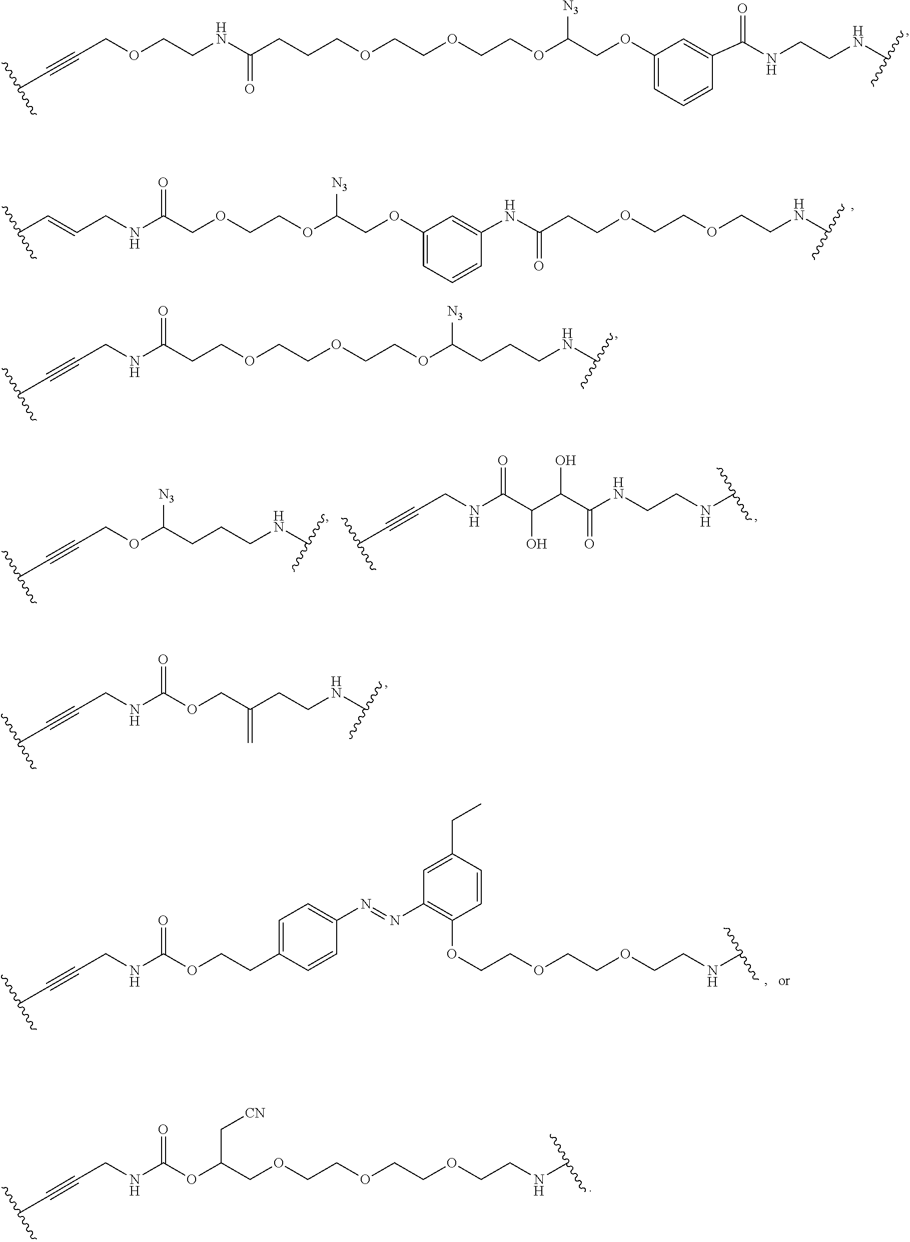

In embodiments, the retardant moiety is detectable (e.g., capable of being detected), wherein the maximum emission of the retardant moiety does not overlap with the maximum emission of the R⁴ moieties of each of the sequencing nucleotides (e.g., the maximum emission of the retardant moiety is less than 530 and greater than 680 nm). In embodiments, the retardant moiety is detectable, wherein the maximum emission of the retardant moiety is less than about 530 nm, less than about 520 nm, or less than about 500 nm. In embodiments, the retardant moiety is detectable, wherein the maximum emission of the retardant moiety is greater than about 650 nm, greater than about 700 nm, greater than about 750 nm, or greater than about 790 nm. In embodiments, the retardant moiety is detectable, wherein the maximum emission of the retardant moiety does not overlap with the maximum emission of the detectable label moiety. In embodiments, the maximum emission of the retardant moiety is at least 10, 15, 20, 25, 30, 35, 40, 45, or 50 nm below or above the maximum emission of the detectable label moiety. In embodiments, the maximum emission of the retardant moiety is at least 20 nm below or above the maximum emission of the detectable label moiety.

In embodiments, the maximum emission of the retardant moiety does not overlap with the maximum emission of the detectable labels used to identify the nucleotides used in a sequencing reaction. Typically, the emission spectrum of any fluorophore (e.g., a detectable label used in sequencing reactions and/or a retardant moiety described herein) is distributed over a broad wavelength range that varies between 30 and 200 nm. The bandwidth of emission is generally measured by the width of the spectral profile at 50 percent of the maximum quantum yield and is often referred to as the full-width at half maximum (FWHM). In embodiments, the FWHM of the detectable labels used in sequencing reactions (e.g., dA-dye1, dT-dye2, dC-dye3, and dT-dye4) does not significantly overlap with the FWHM of the retardant moiety. In embodiments, the emission profile of the detectable labels used in sequencing reactions (e.g., dA-dye1, dT-dye2, dC-dye3, and dT-dye4) overlaps with the emission profile of the retardant moiety, and the detection device includes a suitable restricted-wavelength bandpass emission filters such that the retardant moiety does not interfere with the detection of the sequencing nucleotides. In embodiments, the emission spectrum of the retardant moiety minimally overlaps with the emission spectrum of the detectable labels used to identify the nucleotides used in a sequencing reaction. In embodiments, the degree of overlap between the retardant moiety spectrum and the detectable labels used in sequencing reactions may be quantified using means known in the art, such as the Szymkiewicz-Simpson coefficient or Jaccard index. For example, in embodiments the retarding moiety is a fluorophore that is not detected or capable of being detected during detection of a sequencing nucleotide.

In embodiments, the retardant moiety is fluorescent (e.g., blue), however the emission maximum is outside the detectable channels used for sequencing (e.g., green, yellow, orange, red). For example, the retardant moiety may include a cyanine, rhodamine, 4,4-difluoro-4-bora-3a,4a-diaza-s-indacene (BODIPY), squaraine, phthalocyanine, or porphyrin derivatives provided the emission wavelength does not interfere with detection of the sequencing nucleotides. Chemical substitutions to the core can shift the emission wavelength, for example adding dicyanovinyls to squaraine moiety enhances NIR fluorescence properties. For example, the retardant moiety may be detectable, wherein the emission maximum is outside the range of detection for the sequencing nucleotides, which is typically about 530 nm to about 750 nm for four color sequencing or about 520 nm to about 660 nm for two color sequencing (see for example the compositions described in U.S. Pat. Nos. 9,222,132 and 9,453,258).

In embodiments, the retardant moiety is non-fluorescent. In embodiments, the retardant moiety is a quencher. The quencher may provide an additional benefit by quenching (i.e., absorbing) any remaining fluorescence before the next sequencing cycle. For example, following incorporation and detection of a labeled sequencing nucleotide, a chase nucleotide containing a quencher moiety is introduced and incorporated to any available primed templates (i.e., a primed template with a free 3′-OH). The chase nucleotide containing a quencher may absorb and decrease the fluorescent intensity of any long-lived fluorescent states such that when the next sequencing cycle is initiated the primed templates are all dark by reducing any background fluorescence.

In embodiments, the retardant moiety is a quenching moiety. In embodiments, the retardant moiety is non-fluorescent. In embodiments, the retardant moiety is a quencher. The quencher may provide an additional benefit by quenching (i.e., absorbing) any remaining fluorescence before the next sequencing cycle. For example, quenching moieties reduce signal cross-talk thereby simplifying nucleotide detection. Non-limiting examples of quenching moieties include monovalent species of Dabsyl (dimethylaminoazobenzenesulfonic acid), Black Hole Quenchers (BHQ) (e.g., (BHQ), BHQ-2, and BHQ-3), BMN Quenchers (e.g., BMN-Q460, BMN-Q535, BMN-Q590, BMN-Q620, BMN-Q650) Qxl, Tide Quenchers (e.g., TQ2, TQ3), Iowa black FQ, Iowa black RQ, Deep Dark Quencher (e.g., DDQ I, DDQ II), or IRDye QC-1. In embodiments, the retardant moiety is BMN-Q460, Dabcyl, DDQ-I, BMN-Q535, HHQ-1, TQ2, BMN-Q620, BMN-Q590, BHQ-2, TQ3, BMN-Q650, or BBQ-650. In embodiments, the retardant moiety is a quenching moiety capable of quenching fluorescence in the range of 400-530 nm, 480-580 nm, 550-650 nm, 480-720 nm, or 550-720 nm.

In embodiments, the retardant moiety is a dye that is not detected under conditions (i.e., the same wavelength) used to detect dyes used for sequencing nucleotides. In embodiments, the retardant moiety is does not absorb and/or emit light in the same wavelengths as the detectable moiety. In embodiments, the retardant moiety is does not absorb and/or emit light in the same wavelengths as the detectable moiety (i.e. R⁴), which is typically about 530 nm to about 750 nm for four color sequencing or about 520 nm to about 660 nm for two color sequencing. In embodiments, the retardant moiety does not comprise biotin, TCO (trans-cyclooctyne), DBCO (dibenzocyclooctyne), tetrazine, streptavidin or azido. In embodiments, the retardant moiety does not comprise phenylboronic acid (PDBA), quadricyclane, norbornene, cyclooctyne, alkyne, cyclooctene, salicylhydroxamic acid (SHA), ni bis(dithiolene), nitrile oxide. In embodiments, the retardant moiety is not capable of interacting (e.g., covalently or non-covalently) with a second, optionally different, chemical moiety (e.g., complementary anchor moiety binder). For example, the retardant moiety is not a bioconjugate reactive group capable of interacting (e.g., covalently) with a complementary bioconjugate reactive group (e.g., complementary anchor moiety reactive group). In embodiments, the retardant moiety is not a click chemistry reactant moiety. In embodiments, the retardant moiety is not capable of non-covalently interacting with a second chemical moiety (e.g., complementary affinity anchor moiety binder).

In embodiments, R⁸ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —SF₅, substituted or unsubstituted alkyl (e.g., C₁-C₂₀, C₁₀-C₂₀, C₁-C₈, C₁-C₆, or C₁-C₄), substituted or unsubstituted heteroalkyl (e.g., 2 to 20 membered, 8 to 20 membered, 5 to 16 membered, 2 to 10 membered, 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenylene), substituted or unsubstituted heteroaryl (e.g., 5 to 10 membered, 5 to 9 membered, or 5 to 6 membered), a polyphosphate moiety, or nucleic acid moiety.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, or —SF₅. In embodiments, R⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R⁸ is a polyphosphate moiety, or a nucleic acid moiety (e.g., a polyT moiety). In embodiments, R⁸ is R^{8A}-substituted or unsubstituted alkyl, R^{8A}-substituted or unsubstituted heteroalkyl, R^{8A}-substituted or unsubstituted cycloalkyl, R^{8A}-substituted or unsubstituted heterocycloalkyl, R^{8A}-substituted or unsubstituted aryl, R^{8A}-substituted or unsubstituted heteroaryl. R^{8A} is oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R⁸ is

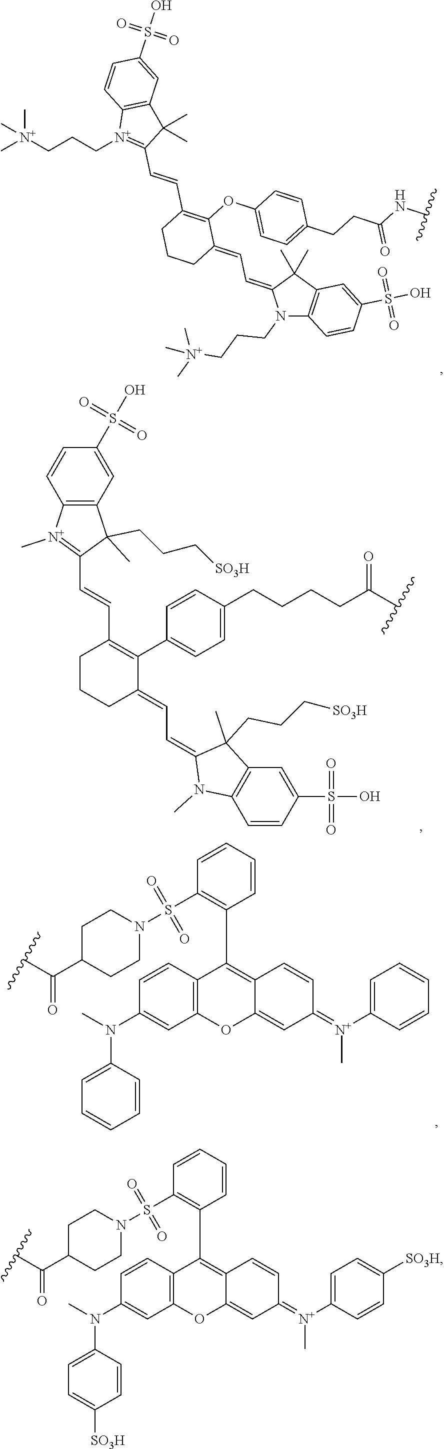 [SMILES: *NC(=O)CCc1ccc(OC2=C(/C=C/C3=[N+](CCC[N+](C)(C)C)c4ccc(S(=O)(=O)O)cc4C3(C)C)CCC/C2=C\C=C2\N(CCC[N+](C)(C)C)c3ccc(S(=O)(=O)O)cc3C2(C)C)cc1 ; *C(=O)CCCCc1ccc(C2=C(/C=C/C3=[N+](C)c4ccc(S(=O)(=O)O)cc4C3(C)CCCS(=O)(=O)O)CCC/C2=C\C=C2\N(C)c3ccc(S(=O)(=O)O)cc3C2(C)CCCS(=O)(=O)O)cc1 ; *C(=O)C1CCN(S(=O)(=O)c2ccccc2-c2c3cc/c(=[N+](/C)c4ccccc4)cc-3oc3cc(N(C)c4ccccc4)ccc23)CC1 ; *C(=O)C1CCN(S(=O)(=O)c2ccccc2-c2c3cc/c(=[N+](/C)c4ccc(S(=O)(=O)O)cc4)cc-3oc3cc(N(C)c4ccc(S(=O)(=O)O)cc4)ccc23)CC1]

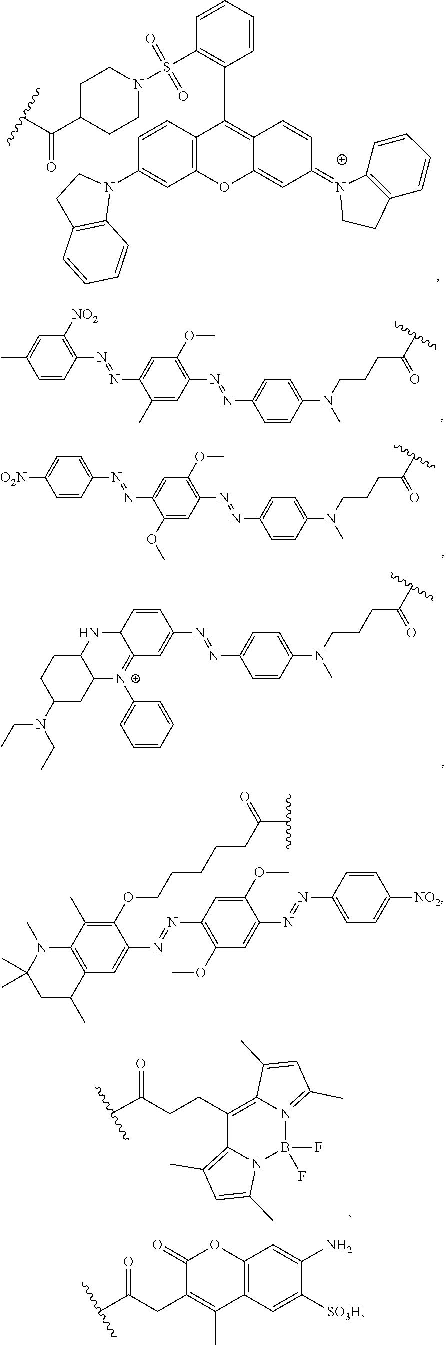

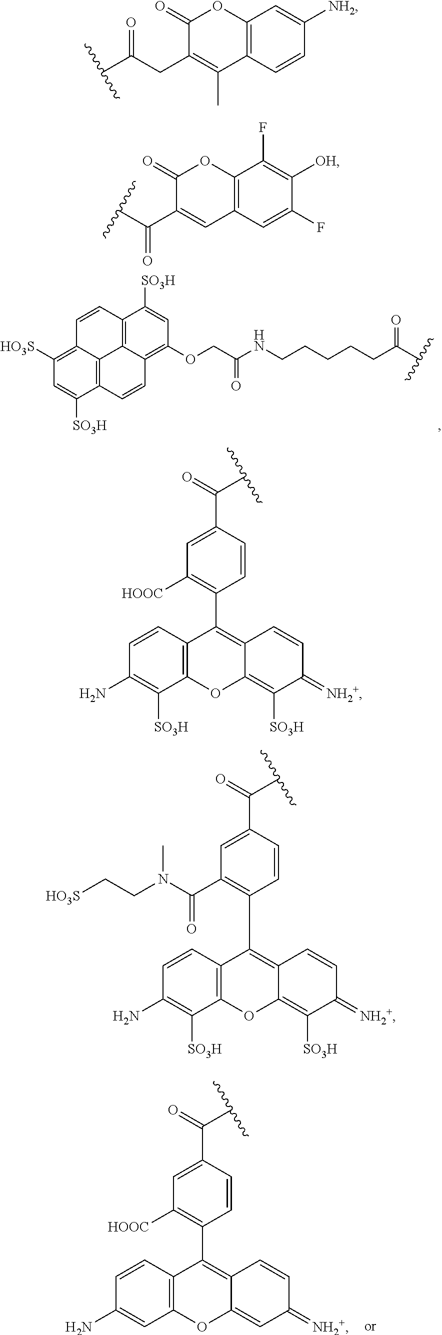

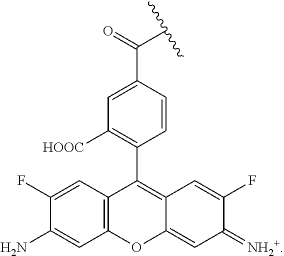

In embodiments, R⁸ is

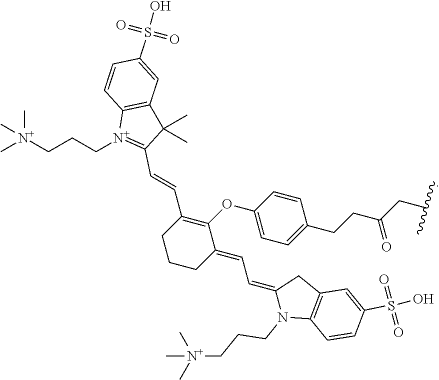 [SMILES: *CC(=O)CCc1ccc(OC2=C(/C=C/C3=[N+](CCC[N+](C)(C)C)c4ccc(S(=O)(=O)O)cc4C3(C)C)CCC/C2=C\C=C2/Cc3cc(S(=O)(=O)O)ccc3N2CCC[N+](C)(C)C)cc1]

In embodiments, R⁸ is

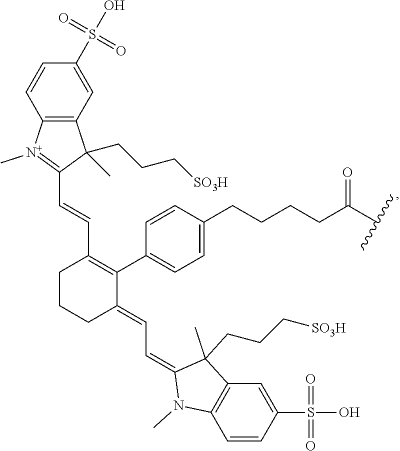 [SMILES: *C(=O)CCCCc1ccc(C2=C(/C=C/C3=[N+](C)c4ccc(S(=O)(=O)O)cc4C3(C)CCCS(=O)(=O)O)CCC/C2=C\C=C2\N(C)c3ccc(S(=O)(=O)O)cc3C2(C)CCCS(=O)(=O)O)cc1]

In embodiments, R⁸ is

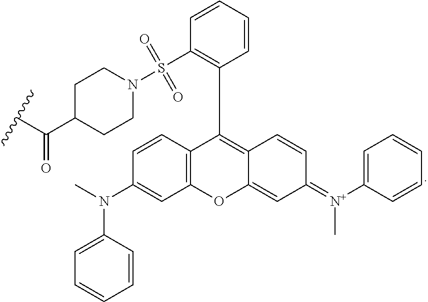 [SMILES: *C(=O)C1CCN(S(=O)(=O)c2ccccc2Cc2c3cc/c(=[N+](/C)c4ccccc4)cc-3oc3cc(N(C)c4ccccc4)ccc23)CC1]

In embodiments, R⁸ is

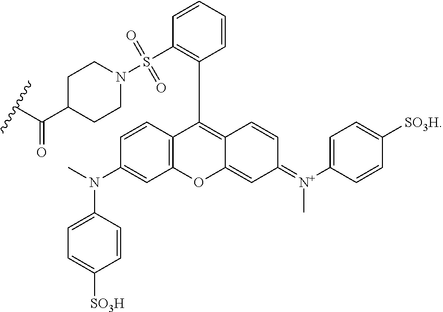 [SMILES: *C(=O)C1CCN(S(=O)(=O)c2ccccc2Cc2c3cc/c(=[N+](/C)c4ccc(S(=O)(=O)O)cc4)cc-3oc3cc(N(C)c4ccc(S(=O)(=O)O)cc4)ccc23)CC1]

In embodiments, R⁸ is

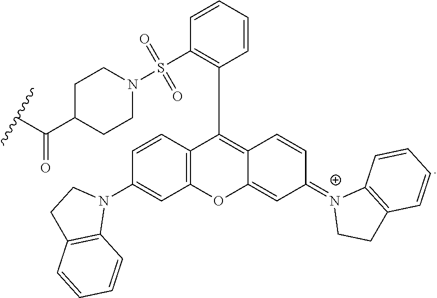 [SMILES: *C(=O)C1CCN(S(=O)(=O)c2ccccc2Cc2c3cc/c(=[N+]4/CCc5ccccc54)cc-3oc3cc(N4CCc5ccccc54)ccc23)CC1]

In embodiments, R⁸ is

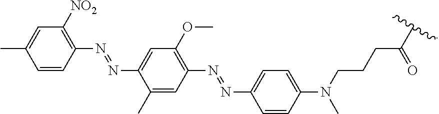 [SMILES: *C(=O)CCCN(C)c1ccc(/N=N/c2cc(C)c(/N=N/c3ccc(C)cc3[N+](=O)[O-])cc2OC)cc1]

In embodiments, R⁸ is

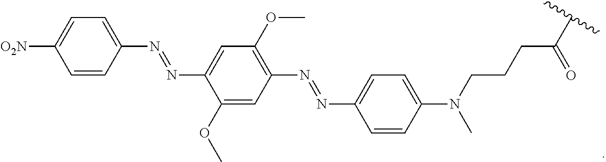 [SMILES: *C(=O)CCCN(C)c1ccc(/N=N/c2cc(OC)c(/N=N/c3ccc([N+](=O)[O-])cc3)cc2OC)cc1]

In embodiments, R⁸ is

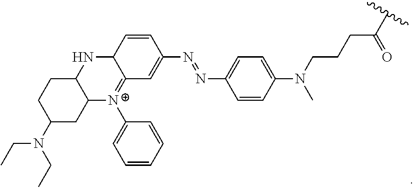 [SMILES: *C(=O)CCCN(C)c1ccc(/N=N/C2=CC3=[N+](c4ccccc4)C4CC(N(CC)CC)CCC4NC3C=C2)cc1]

In embodiments, R⁸ is

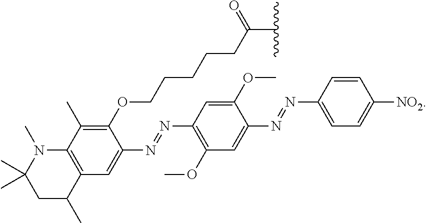 [SMILES: *C(=O)CCCCCOc1c(/N=N/c2cc(OC)c(/N=N/c3ccc([N+](=O)[O-])cc3)cc2OC)cc2c(c1C)N(C)C(C)(C)CC2C]

In embodiments, R⁸ is

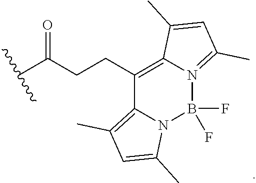

In embodiments, R⁸ is

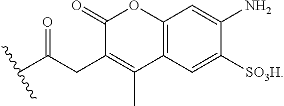 [SMILES: *C(=O)Cc1c(C)c2cc(S(=O)(=O)O)c(N)cc2oc1=O]

In embodiments, R⁸ is

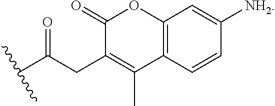 [SMILES: *C(=O)Cc1c(C)c2ccc(N)cc2oc1=O]

In embodiments, R⁸ is

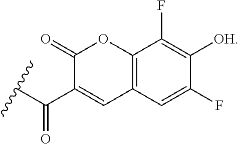 [SMILES: *C(=O)c1cc2cc(F)c(O)c(F)c2oc1=O]

In embodiments, R⁸ is

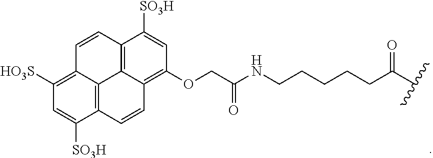 [SMILES: *C(=O)CCCCCNC(=O)COc1cc(S(=O)(=O)O)c2ccc3c(S(=O)(=O)O)cc(S(=O)(=O)O)c4ccc1c2c43]

In embodiments, R⁸ is

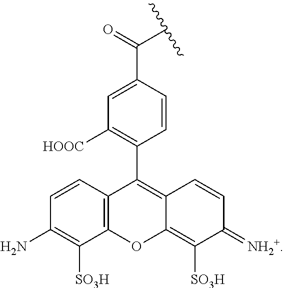

In embodiments, R⁸ is

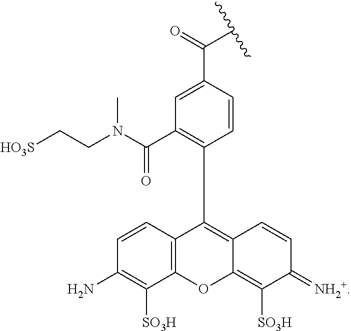

In embodiments, R⁸ is

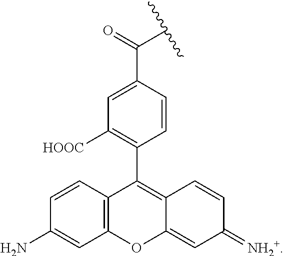

In embodiments, R⁸ is

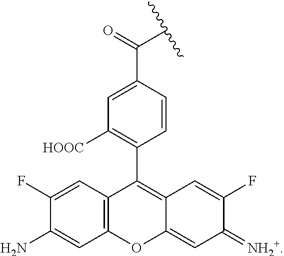

In embodiments, R⁸ is

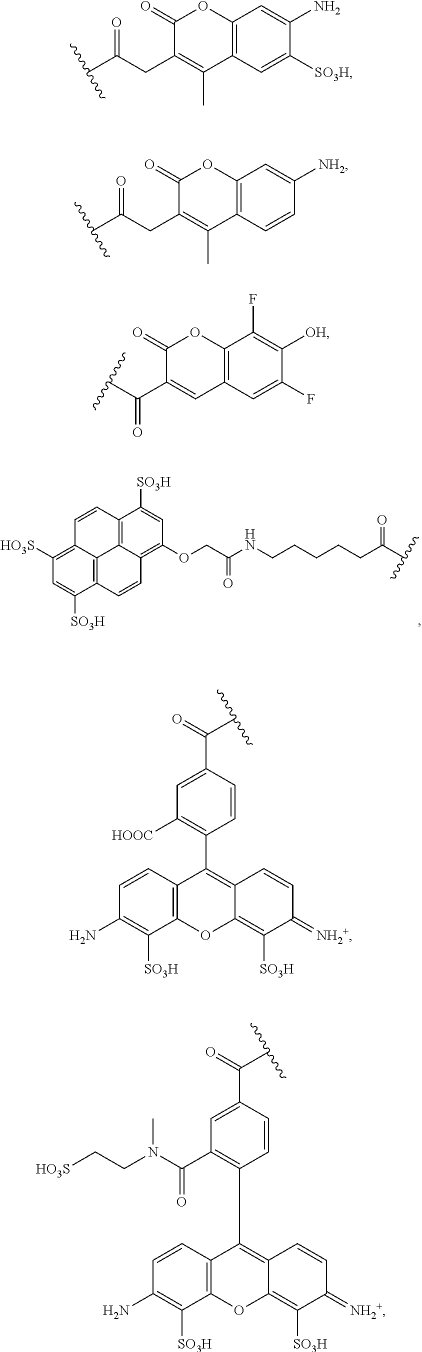

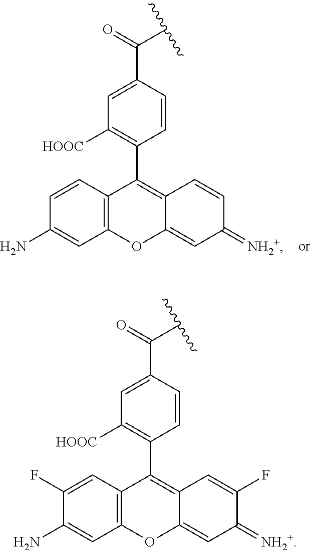

In embodiments, R⁸ is

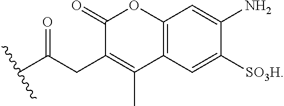 [SMILES: *C(=O)Cc1c(C)c2cc(S(=O)(=O)O)c(N)cc2oc1=O]

In embodiments, R⁸ is

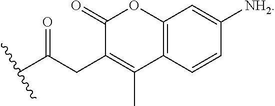 [SMILES: *C(=O)Cc1c(C)c2ccc(N)cc2oc1=O]

In embodiments, R⁸ is

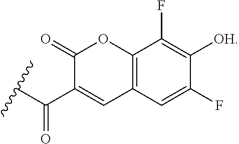 [SMILES: *C(=O)c1cc2cc(F)c(O)c(F)c2oc1=O]

In embodiments, R⁸ is

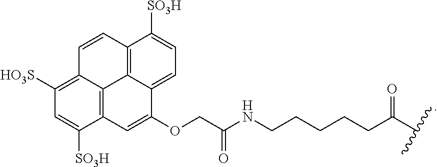 [SMILES: *C(=O)CCCCCNC(=O)COc1cc2c(S(=O)(=O)O)cc(S(=O)(=O)O)c3ccc4c(S(=O)(=O)O)ccc1c4c32]

In embodiments, R⁸ is

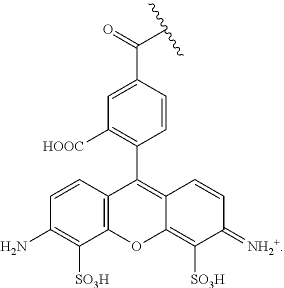

In embodiments, R⁸ is

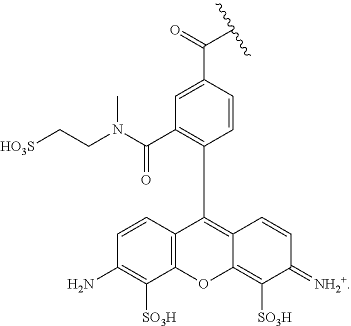

In embodiments, R⁸ is

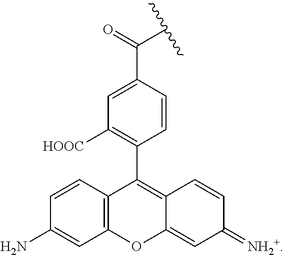

In embodiments, R⁸ is

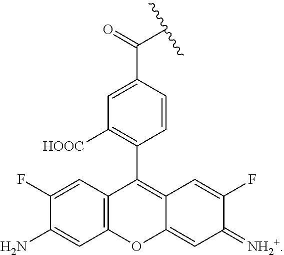

In embodiments, R⁸ is

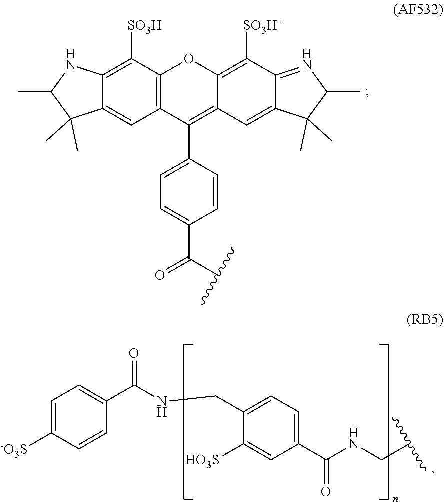

wherein n is 4;

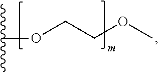

wherein m is 24 (PEG24);

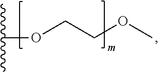

wherein m is 12 (PEG12); or

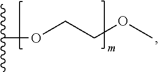

wherein m is 4 (PEG4). In embodiments, R⁸ is

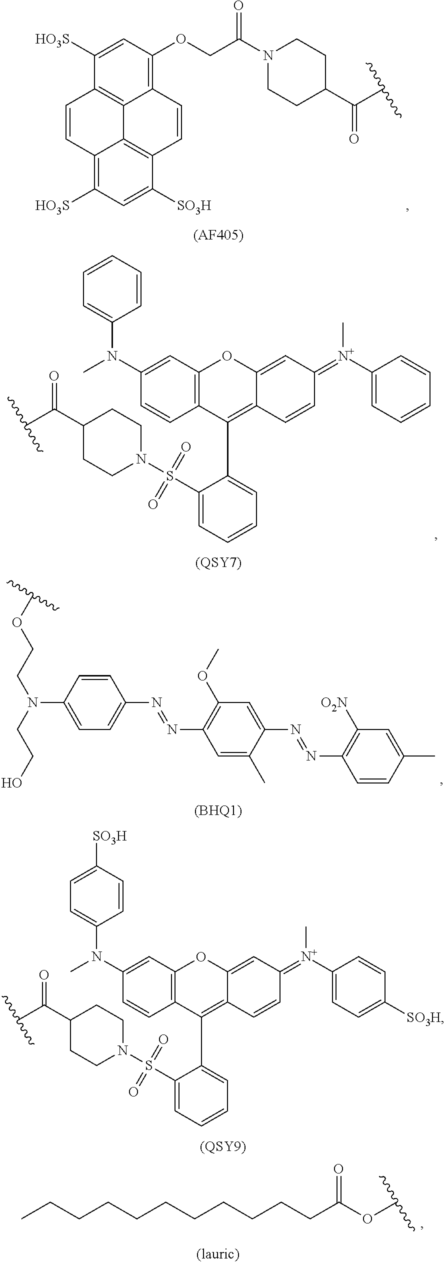

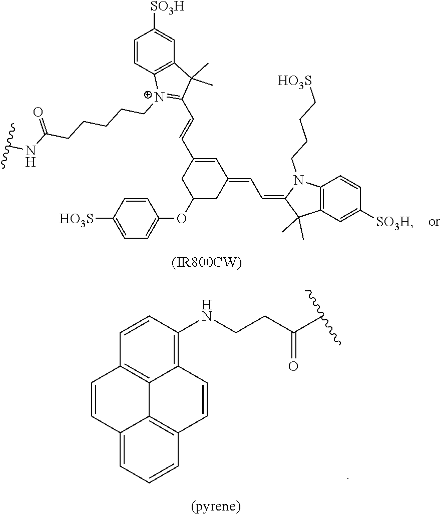 [SMILES: *NC(=O)CCCCC[N+]1=C(/C=C/C2=CC(=C/C=C3\N(CCCCS(=O)(=O)O)c4ccc(S(=O)(=O)O)cc4C3(C)C)/CC(Oc3ccc(S(=O)(=O)O)cc3)C2)C(C)(C)c2cc(S(=O)(=O)O)ccc21 ; *C(=O)CCNc1ccc2ccc3cccc4ccc1c2c34]

In embodiments, R⁸ is

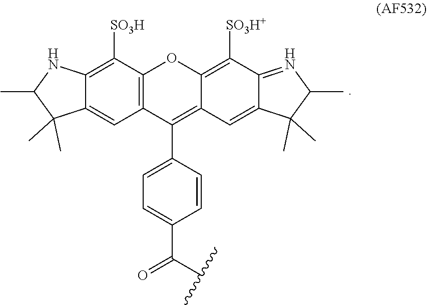

In embodiments, R⁸ is

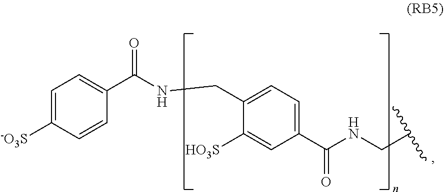

wherein n is 4. In embodiments, R⁸ is

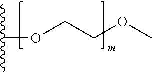

wherein m is 24 (PEG24). In embodiments, R⁸ is

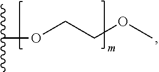

wherein m is 12 (PEG12). In embodiments, R⁸ is

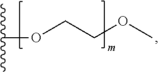

wherein m is 4 (PEG4).

In embodiments, R⁸ is

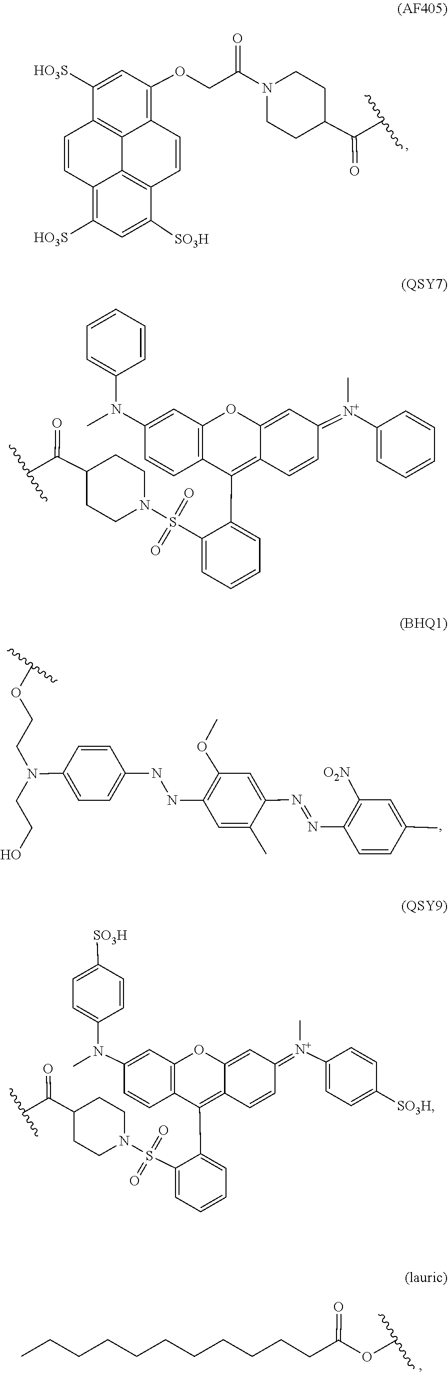

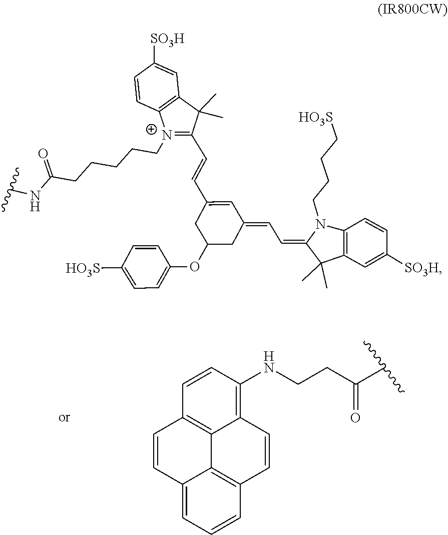 [SMILES: *NC(=O)CCCCC[N+]1=C(/C=C/C2=CC(=C/C=C3\N(CCCCS(=O)(=O)O)c4ccc(S(=O)(=O)O)cc4C3(C)C)/CC(Oc3ccc(S(=O)(=O)O)cc3)C2)C(C)(C)c2cc(S(=O)(=O)O)ccc21 ; *C(=O)CCNc1ccc2ccc3cccc4ccc1c2c34]

(pyrene). In embodiments, R⁸ is

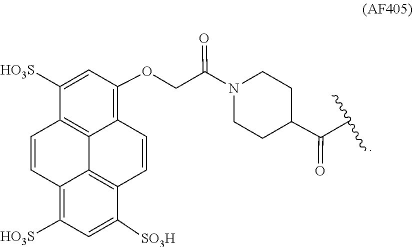 [SMILES: *C(=O)C1CCN(C(=O)COc2cc(S(=O)(=O)O)c3ccc4c(S(=O)(=O)O)cc(S(=O)(=O)O)c5ccc2c3c54)CC1]

In embodiments, R⁸ is

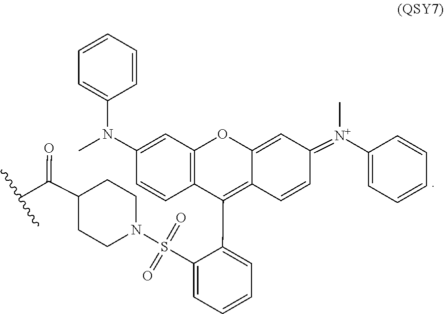 [SMILES: *C(=O)C1CCN(S(=O)(=O)c2ccccc2-c2c3cc/c(=[N+](/C)c4ccccc4)cc-3oc3cc(N(C)c4ccccc4)ccc23)CC1]

In embodiments, R⁸ is

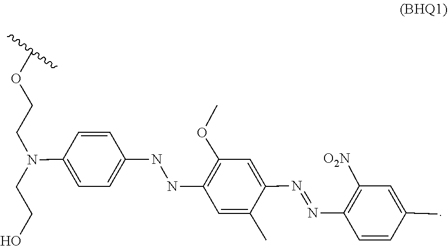 [SMILES: *OCCN(CCO)c1ccc(NNc2cc(C)c(/N=N/c3ccc(C)cc3[N+](=O)[O-])cc2OC)cc1]

In embodiments, R⁸ is

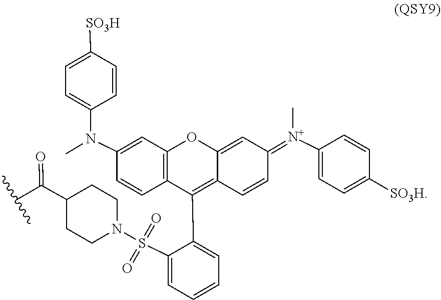 [SMILES: *C(=O)C1CCN(S(=O)(=O)c2ccccc2-c2c3cc/c(=[N+](/C)c4ccc(S(=O)(=O)O)cc4)cc-3oc3cc(N(C)c4ccc(S(=O)(=O)O)cc4)ccc23)CC1]

In embodiments, R⁸ is

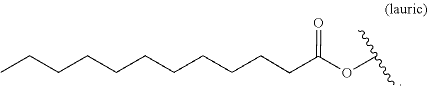 [SMILES: *OC(=O)CCCCCCCCCCC]

In embodiments, R⁸ is

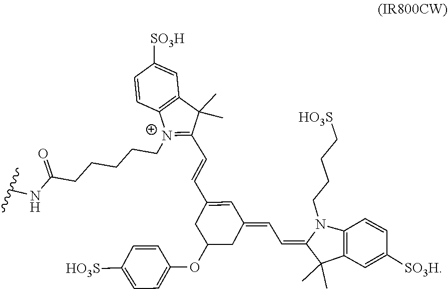 [SMILES: *NC(=O)CCCCC[N+]1=C(/C=C/C2=CC(=C/C=C3\N(CCCCS(=O)(=O)O)c4ccc(S(=O)(=O)O)cc4C3(C)C)/CC(Oc3ccc(S(=O)(=O)O)cc3)C2)C(C)(C)c2cc(S(=O)(=O)O)ccc21]

In embodiments, R⁸ is

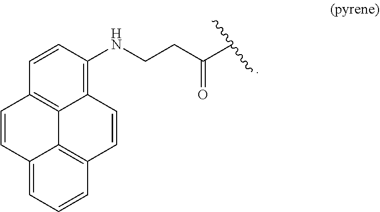 [SMILES: *C(=O)CCNc1ccc2ccc3cccc4ccc1c2c34]

In embodiments, R⁸ is a fused ring (e.g., a fused ring aryl, fused ring heteroaryl, fused ring cycloalkyl, or fused ring heterocycloalkyl).

In embodiments, R⁸ is unsubstituted C₁-C₁₂ or C₁-C₈ alkyl. In embodiments, R⁸ is unsubstituted C₁-C₁₂ alkyl. In embodiments, R⁸ is unsubstituted C₁-C₈ alkyl. In embodiments, R⁸ is unsubstituted C₁₂ alkyl. In embodiments, R⁸ is unsubstituted C₁₁ alkyl. In embodiments, R⁸ is unsubstituted C₁₀ alkyl. In embodiments, R⁸ is unsubstituted C₉ alkyl. In embodiments, R⁸ is unsubstituted C₈ alkyl. In embodiments, R⁸ is unsubstituted C₇ alkyl. In embodiments, R⁸ is unsubstituted C₆ alkyl. In embodiments, R⁸ includes PEG. In embodiments, R⁸ is

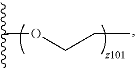

wherein z101 is independently an integer from 1 to 400. In embodiments, z101 is an integer from 1 to 300. In embodiments, z101 is an integer from 1 to 200. In embodiments, z101 is an integer from 100 to 300. In embodiments, z101 is an integer from 2 to 24. In embodiments, z101 is an integer from 2 to 18. In embodiments, z101 is 2, 4, 6, 8, 10, 12, 14, 16, 18, 20, 22, or 24. In embodiments, R⁸ is

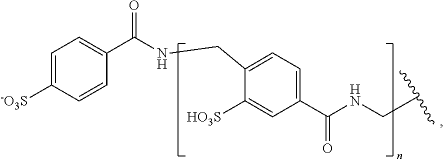

wherein n is an integer from 1 to 12.

In an aspect is provided a kit including a sequencing solution and a chase solution, wherein (a) the sequencing solution includes a plurality of sequencing nucleotides, (b) each nucleotide of the plurality of sequencing nucleotides include a detectable label moiety and a first reversible terminator moiety; (c) the chase solution includes a plurality of chase nucleotides, (d) each nucleotide of the plurality of chase nucleotides includes a retardant moiety and a second reversible terminator moiety, and (e) the retardant moieties differ in structure from the detectable label moieties. In embodiments, the solutions are independent, that is, they are not provided in a mixture. In embodiments, the kit includes instructions and/or components necessary to perform the methods described herein (e.g., nucleotides, buffers, salts, enzymes, polynucleotides, cleaving agents (e.g., reducing agents), and other aqueous solutions).

In embodiments, the kit described herein includes a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the polymerase in the kit is a bacterial DNA polymerase, eukaryotic DNA polymerase, archaeal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavus (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. In embodiments, the polymerase is 3PDX polymerase as disclosed in U.S. Pat. No. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, or Telomerase reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffer. In embodiments, the kit includes a buffered solution. For example, the sequencing solution and/or the chase solution may include a buffer such as ethanolamine (EA), tris(hydroxymethyl)aminomethane (Tris), glycine, a carbonate salt, a phosphate salt, a borate salt, 2-dimethyalaminomethanol (DMEA), 2-diethyalaminomethanol (DEEA), N,N,N′,N′-tetramethylethylenediamine (TEMED), and N,N,N′,N′-tetraethylethylenediamine (TEEDA), and combinations thereof. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, Bicine, Tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can comprise one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffer includes PEG (polyethylene glycol), PVP (polyvinylpyrrolidone), trehalose, ficoll, or dextran. In embodiments, the buffer includes additives such as Tween-20 or NP-40.

In embodiments, the kit includes nucleotides in a buffer. In embodiments, the kit includes a buffer. For example, the sequencing solution and/or the chase solution may include a buffer such as ethanolamine (EA), tris(hydroxymethyl)aminomethane (Tris), glycine, a carbonate salt, a phosphate salt, a borate salt, 2-dimethyalaminomethanol (DMEA), 2-diethyalaminomethanol (DEEA), N,N,N′,N′-tetramethylethylenediamine (TEMED), and N,N,N′,N′-tetraethylethylenediamine (TEEDA), and combinations thereof. For example, the buffer may Tris-HCl (pH 9.2 at 25° C.), ammonium sulfate, MgCl₂, 0.1% Tween® 20, and dNTPs.

In embodiments, the kit includes a solid support (e.g., a flow cell). Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008).

In embodiments, the kit includes a composition including: (a) labeled nucleotides including a free 3′-OH, (b) labeled nucleotides lacking a free 3′-OH (e.g., reversibly terminated nucleotides), and (c) one or more depleting reagents for decreasing the amount of the nucleotides including a free 3′-OH, wherein the one or more depleting reagents include: (i) one or more depletion polynucleotides and a depletion polymerase that is active to selectively incorporating the nucleotides including a free 3′-OH, wherein the depletion polynucleotide is free in solution; or (ii) one or more nucleotide cyclases active to selectively cyclize the nucleotides including a free 3′-OH. In embodiments, the composition is stored in a single container. In embodiments, each nucleotide type (e.g., modified dATP, dTTP, dCTP, and dGTP) of composition is stored in a different container with one or more depleting reagents. In embodiments, the composition is stored at about 2° C.-8° C., about 20° C.-30° C., or about 4° C.-37° C. In embodiments, the composition is stored at about 4° C. to about 30° C.

In embodiments, the kit includes a plurality of primers for amplifying and/or for sequencing nucleic acids isolated from the sample. The kit may provide at least 1, 2, 3, 4, 5, 6, 7, 8, 9, 10, 20, 50, 100, 200, 500, 1000, or more primers. The kit may provide between about 1-3, 1-10, 5-20, 1-1000, 10-500, 20-200, or 50-100 primers. In embodiments, the primers include 5, 10, 15, 20, 25, 30, 40, 50, 100, 150, 200 or more nucleotides.

In an aspect is provided a composition including i) a plurality of chase nucleotides, ii) a depletion polynucleotide, and iii) a polymerase including an amino acid sequence that is at least 80% identical to a continuous 500 amino acid sequence within SEQ ID NO: 1, at least one mutation at amino acid position 32 or an amino acid position functionally equivalent to amino acid position 32; a mutation at amino acid position 34 or an amino acid position functionally equivalent to amino acid position 34; or a mutation at amino acid position 584 or an amino acid position functionally equivalent to amino acid position 584.

In embodiments, the polymerase is exo-/exo-variant (i.e., does not include 3′-5′ or 5′-3′ exonuclease activity). Examples of mutations giving rise to an exo⁻/exo⁻ variants include mutations at positions in a parent polymerase corresponding to positions in SEQ ID NO: 1 identified as follows: 32 and 34. In embodiments, the polymerase includes a valine, threonine, glycine, or alanine at amino acid position 32. In embodiments, the polymerase includes a valine at amino acid position 32. In embodiments, the polymerase includes a threonine at amino acid position 32. In embodiments, the polymerase includes a glycine at amino acid position 32. In embodiments, the polymerase includes an alanine at amino acid position 32. In embodiments, the polymerase includes a serine at amino acid position 32. In embodiments, the polymerase includes a valine, threonine, glycine, or alanine at amino acid position 34. In embodiments, the polymerase includes a valine at amino acid position 34. In embodiments, the polymerase includes a threonine at amino acid position 34. In embodiments, the polymerase includes a glycine at amino acid position 34. In embodiments, the polymerase includes an alanine at amino acid position 34. In embodiments, the polymerase includes a serine at amino acid position 34.

In embodiments, the polymerase includes an amino acid substitution at position 584. The amino acid substitution at position 584 may be a serine, glycine, threonine, asparagine, or alanine substitution. The amino acid substitution at position 584 may be a serine substitution. In embodiments, the substitution at position 584 includes a polar amino acid (e.g., threonine, asparagine, or glutamine). In embodiments, the amino acid substitution at position 584 is a selenocysteine. In embodiments, the substitution at position 584 includes a serine at amino acid position 584. In embodiments, the substitution at position 584 includes a glycine at amino acid position 584. In embodiments, the substitution at position 584 includes a threonine at amino acid position 584. In embodiments, the substitution at position 584 includes an asparagine at amino acid position 584. In embodiments, the substitution at position 584 includes an alanine at amino acid position 584. In embodiments, the depletion polymerase includes the sequence described in SEQ ID NO: 1. In embodiments, the depletion polymerase includes the sequence described in SEQ ID NO: 2.

In embodiments, the depletion polymerase includes the sequence:

(SEQ ID NO: 1)
VISYDNYVTILDEETLKAWIAKLEKAPVFAFDTETDSLDNISANLVGLSF
AIEPGVAAYIPVAHDYLDAPDQISRERALELLKPLLEDEKALKVGQNLKY
DRGILANYGIELRGIAFDTMLESYILNSVAGRHDMDSLAERWLKHKTITF
EEIAGKGKNQLTFNQIALEEAGRYAAEDADVTLQLHLKMWPDLQKHKGPL
NVFENIEMPLVPVLSRIERNGVKIDPKVLHNHSEELTLRLAELEKKAHEI
AGEEFNLSSTKQLQTILFEKQGIKPLKKTPGGAPSTSEEVLEELALDYPL
PKVILEYRGLAKLKSTYTDKLPLMINPKTGRVHTSYHQAVTATGRLSSTD
PNLQNIPVRNEEGRRIRQAFIAPEDYVIVSADYSQIELRIMAHLSRDKGL
LTAFAEGKDIHRATAAEVFGLPLETVTSEQRRSAKAINFGLIYGMSAFGL
ARQLNIPRKEAQKYMDLYFERYPGVLEYMERTRAQAKEQGYVETLDGRRL
YLPDIKSSNGARRAAAERAAINAPMQGTAADIIKRAMIAVDAWLQAEQPR
VRMIMQVHDELVFEVHKDDVDAVAKQIHQLMENCTRLDVPLLVEVGSGEN
WDQAH

In embodiments, the depletion polymerase includes the sequence:

(SEQ ID NO: 2)
MVISYDNYVTILDEETLKAWIAKLEKAPVFAFATATDSLDNISANLVGLS
FAIEPGVAAYIPVAHDYLDAPDQISRERALELLKPLLEDEKALKVGQNLK
YDRGILANYGIELRGIAFDTMLESYILNSVAGRHDMDSLAERWLKHKTIT
FEEIAGKGKNQLTFNQIALEEAGRYAAEDADVTLQLHLKMWPDLQKHKGP
LNVFENIEMPLVPVLSRIERNGVKIDPKVLHNHSEELTLRLAELEKKAHE
IAGEEFNLSSTKQLQTILFEKQGIKPLKKTPGGAPSTSEEVLEELALDYP
LPKVILEYRGLAKLKSTYTDKLPLMINPKTGRVHTSYHQAVTATGRLSST
DPNLQNIPVRNEEGRRIRQAFIAPEDYVIVSADYSQIELRIMAHLSRDKG
LLTAFAEGKDIHRATAAEVFGLPLETVTSEQRRSAKAINFGLIYGMSAFG
LARQLNIPRKEAQKYMDLYFERYPGVLEYMERTRAQAKEQGYVETLDGRR
LYLPDIKSSNGARRAAAERAAINAPMQGTAADIIKRAMIAVDAWLQAEQP
RVRMIMQVHDELVFEVHKDDVDAVAKQIHQLMENSTRLDVPLLVEVGSGE
NWDQAH.

### III. Methods

The present disclosure provides methods for determining the identity of one or more nucleotide residues in an extension product. Such methods can be used, for example, to determine the sequence of target DNA, including partial and whole genomes, exomes, transcriptomes, and the like. Such methods comprise combining in a reaction mixture a plurality of identical primed template polynucleotides (e.g., DNA molecules), a polymerase, distinguishable sequencing nucleotides that include a reversible terminator moiety and a detectable label moiety covalently bound to the sequencing nucleotide via a cleavable linker, and distinguishable, chase nucleotides that include a reversible terminator moiety and a retarding moiety covalently bound to the chase nucleotide via a cleavable linker.

In an aspect is provided a method of sequencing a template polynucleotide, the method including: a) contacting a first primer hybridized to a first template polynucleotide with a first sequencing nucleotide including a first reversible terminator moiety and a first detectable label moiety covalently bound to the first sequencing nucleotide via a first cleavable linker, incorporating the first sequencing nucleotide into the first primer with a polymerase thereby forming a first extended primer polynucleotide, and detecting the first sequencing nucleotide; b) contacting a second primer hybridized to a second template polynucleotide with a first chase nucleotide including a first retarding moiety covalently bound to the first chase nucleotide via a first chase cleavable linker; and incorporating the first chase nucleotide into the second primer with a polymerase thereby forming a second extended primer polynucleotide; c) removing the first reversible terminator moiety, the first detectable label moiety, and the first retarding moiety; and d) contacting the first extended primer polynucleotide with a second sequencing nucleotide including a second reversible terminator moiety and a second detectable label moiety covalently bound to the second nucleotide via a second cleavable linker, incorporating the second sequencing nucleotide into the first extended primer polynucleotide with a polymerase, thereby extending the first extended primer polynucleotide, and detecting the second sequencing nucleotide. In embodiments, the first template polynucleotide is sequenced by detection of the first sequencing nucleotide and second sequencing nucleotide. In embodiments, the first template polynucleotide is sequenced by detection of the first sequencing nucleotide and second sequencing nucleotide and repeating this process iteratively. In embodiments, the first template polynucleotide is immobilized to a solid support. In embodiments, the second template polynucleotide is immobilized to the same solid support. In embodiments, the first template polynucleotide is within a plurality (e.g., a cluster) of immobilized template polynucleotides. In embodiments, the second template polynucleotide is within the same plurality (e.g., a cluster) of immobilized template polynucleotides. In embodiments, the first sequencing nucleotide has a detectable label moiety that is not the same as the first retarding moiety on the first chase nucleotide. In embodiments, step b) is repeated one or more times (i.e., consecutively contacting a primer hybridized to a template polynucleotide with a chase nucleotide). In embodiments, step b) is repeated 1, 2, 3, 4, or 5 times before step c).

In an aspect is provided a method of sequencing a template polynucleotide, the method including: a) contacting a primer hybridized to a first template polynucleotide with a first sequencing nucleotide including a first reversible terminator moiety and the first sequencing nucleotide is coupled to a first detectable label moiety, binding (e.g., hydrogen bonding) the first sequencing nucleotide to a complementary nucleotide of the template polynucleotide, and detecting the first sequencing nucleotide; b) contacting a primer hybridized to a second template polynucleotide with a first chase nucleotide including a first retarding moiety coupled to the first chase nucleotide; and incorporating the first chase nucleotide into the second primer with a polymerase thereby forming an extended primer polynucleotide; c) removing the first reversible terminator moiety, the first detectable label moiety, and the first retarding moiety. In embodiments, the method further includes contacting the extended primer polynucleotide with a second sequencing nucleotide including a second reversible terminator moiety and the second sequencing nucleotide is coupled to a second detectable label moiety, binding (e.g., hydrogen bonding) the second sequencing nucleotide to a complementary nucleotide of the template polynucleotide, and detecting the second sequencing nucleotide.

In another aspect is provided a method of sequencing a template polynucleotide, the method including: contacting a double stranded nucleic acid molecule comprising a primer oligonucleotide hybridized to the template polynucleotide with a first plurality of nucleotide analogues and binding a nucleotide analogue with a polymerase to a complementary nucleotide of the double-stranded nucleic acid molecule thereby forming a first polymerase-complex, wherein each nucleotide analogue is associated with a distinguishable detectable moiety; detecting the polymerase-complex and removing the nucleotide analogue; contacting the first polymerase complex with a second plurality of nucleotide analogues and binding a nucleotide analogue with a polymerase to a complementary nucleotide of said double-stranded nucleic acid molecule thereby forming a second polymerase-complex, wherein each nucleotide analogue is not associated with a distinguishable detectable moiety. In embodiments, the nucleotide analogue is associated with a retarding moiety (e.g., covalently linked to a retarding moiety).

In an aspect is provided a method of sequencing a template polynucleotide, including executing a sequencing cycle including (i) extending a first complementary polynucleotide that is hybridized to the template nucleic acid by incorporating a first sequencing nucleotide using a polymerase; and (ii) detecting a label that identifies the first nucleotide; executing a chase cycle including extending a second complementary polynucleotide in one or more dark cycles, wherein each dark cycle includes extending the second complementary polynucleotide by one or more chase nucleotides using the polymerase, without performing a detection event to identify chase nucleotides incorporated during the dark cycle; and executing a sequencing cycle including (i) extending the first or the second complementary polynucleotide by incorporating a second sequencing nucleotide using a polymerase; and (ii) detecting a label that identifies the second nucleotide, thereby sequencing a template nucleic acid.

In an aspect is a method of sequencing a plurality of polynucleotides immobilized on a solid support, wherein each polynucleotide is hybridized to a sequencing primer, the method including: a) contacting the solid support with a plurality of sequencing nucleotides comprising a detectable label (e.g., sequencing nucleotides as described herein), b) contacting the solid support with a plurality of chase nucleotides comprising a retarding moiety (e.g., chase nucleotides as described herein), c) detecting the detectable label before, during, or after step b), thereby identifying the sequencing nucleotide; and d) repeating steps a), b), and c) to sequence a plurality of polynucleotides. In embodiments, step d) includes repeating for 5, 10, 15, 20, 25, 30, 35, 40, 45, 50 or more cycles, wherein each cycle includes steps a), b), and c). In embodiments, step d) includes repeating for 50, 75, 100, 150, 200, 250, 300 or more cycles, wherein each cycle includes steps a), b), and c). In embodiments, the method generates one or more sequencing reads.

In embodiments, each sequencing nucleotide can be distinguished from one another by the dye molecule associated with the nucleobase (e.g., dye 1 is associated with adenine, dye 2 with cytosine, etc.), under conditions to allow incorporation of one sequencing nucleotides into at least some of the plurality of identical primed template polynucleotide molecules to form a (or a population of) distinguishable, blocked extension product(s). In embodiments, a distinguishable, sequencing nucleotide is incorporated into about 99%, 98%, 97%, 96%, 95%, 90%, 85%, 80%, 75%, 70%, 65%, 60%, 55%, 50%, 45%, 40%, 35%, 30%, 25%, or 20% of the plurality of identical primed template DNA molecules. Additionally, a chase nucleotide can also be incorporated into at least some of the plurality of identical primed template polynucleotide molecules to form a (or a population of) distinguishable, blocked extension product(s). In embodiments, a distinguishable, chase nucleotide is incorporated into about 99%, 98%, 97%, 96%, 95%, 90%, 85%, 80%, 75%, 70%, 65%, 60%, 55%, 50%, 45%, 40%, 35%, 30%, 25%, or 20% of the plurality of identical primed template polynucleotide molecules.

In embodiments, the first sequencing nucleotide and chase (e.g., the first chase) nucleotide include the same nucleobase (i.e., adenine, guanine, cytosine or thymine/uracil). In embodiments, first sequencing nucleotide and chase (e.g., the first chase) nucleotide include the same reversible terminator moiety. In embodiments, first sequencing nucleotide and chase (e.g., the first chase) nucleotide include the same cleavable linker. In embodiments, first sequencing nucleotide and chase (e.g., the first chase) nucleotide include the same nucleobase, the same reversible terminator moiety and the same cleavable linker, and the retarding moiety (e.g., the first retarding moiety) differ in structure from the first detectable label moiety (i.e., the first sequencing nucleotide and chase (e.g., the first chase) nucleotide only differ by the detectable label moiety and retarding moiety). In embodiments, the first sequencing nucleotide and chase (e.g., the first chase) nucleotide include the same reversible terminator moiety (e.g., the sequencing nucleotide and the chase nucleotide each include a reversible terminator moiety having the same structure).

In embodiments, the first sequencing nucleotide and second sequencing nucleotide include the same reversible terminator moiety. In embodiments, first sequencing nucleotide and second sequencing nucleotide include the same cleavable linker. In embodiments, first sequencing nucleotide and the second sequencing nucleotide include a first and second detectable label moiety, which are the same. In embodiments, the first sequencing nucleotide and the second sequencing nucleotide include the same nucleobase (i.e., adenine, guanine, cytosine or thymine/uracil). In embodiments, the first sequencing nucleotide and second sequencing nucleotide include the same nucleobase, the same reversible terminator moiety, the same cleavable linker, and the same detectable label moiety (i.e., the first and second sequencing nucleotides are the same). In embodiments, the first sequencing nucleotide and second sequencing nucleotide include a different reversible terminator moiety. In embodiments, the first sequencing nucleotide and second sequencing nucleotide include a different cleavable linker. In embodiments, first sequencing nucleotide and the second sequencing nucleotide include a first and second detectable label moiety, which are different from one another. In embodiments, the first sequencing nucleotide and the second sequencing nucleotide include a different nucleobase (i.e., adenine, guanine, cytosine or thymine/uracil). In embodiments, the first sequencing nucleotide and second sequencing nucleotide include a different nucleobase, different reversible terminator moiety, different cleavable linker, and different detectable label moiety.

In embodiments, the first template polynucleotide and second template polynucleotide comprise the same sequence of nucleotides. In embodiments, the first template polynucleotide and second template polynucleotide include the same number of nucleotides so that the first sequencing nucleotide and chase nucleotide incorporate at equivalent positions on the first template polynucleotide and second template polynucleotide, respectively. In embodiments, the first template polynucleotide and second template polynucleotide have the same sequence of nucleotides (i.e., they are copies of each other). In embodiments, the first template polynucleotide and second template polynucleotide have substantially the same sequence of nucleotides (i.e., greater than 99% identical). In embodiments, the first template polynucleotide and second template polynucleotide are within the same plurality (e.g., a cluster) of immobilized template polynucleotides. In embodiments, the plurality of immobilized template polynucleotides have substantially the same sequence of nucleotides. In embodiments, a plurality of template polynucleotides includes multiple copies of the same template polynucleotide sequence, or a complement thereof. When immobilized at a discrete location (i.e., an amplification site), this may be referred to as a cluster of polynucleotides templates. In embodiments, each polynucleotide template within the plurality or within the cluster has the same sequence, or a complementary sequence thereof.

In embodiments, the template polynucleotide is in solution or immobilized on a solid substrate, wherein the solid substrate optionally is gold, quartz, silica, plastic (e.g., polypropylene), glass, diamond, silver, or metal and optionally is configured as a bead, chip, well, wafer, filter, or slide. When the solid substrate is glass, template polynucleotide immobilization methods include the use of hydrogels or direct covalent linkage, for example, using silanes, e.g., amino-silanes, epoxy-silanes, and aldehyde-silanes. Additionally, when the template polynucleotides optionally are attached/bound to the solid substrate by covalent site-specific coupling chemistry compatible with DNA, other suitable chemistries include (i) alkyne-labeled, (ii) bound to the solid substrate via polyethylene glycol (PEG) molecules and the solid substrate is azide-functionalized, or (iii) immobilized on the solid substrate via an azido linkage, or an alkynyl linkage. Other representative embodiments of non-covalent attachment include those based on biotin-streptavidin interactions. In embodiments, the solid substrate is a porous medium. In embodiments, the solid support includes a polymer layer, wherein the template polynucleotides are immobilized to the polymer layer.

In embodiments, the solid support includes a plurality of wells (e.g., a billion or more wells). In embodiments, the wells (e.g., each well) is separated from each other by about 0.2 μm to about 2.0 μm. In embodiments, the wells (e.g., each well) is separated from each other by about 0.3 μm to about 2.0 μm. In embodiments, the wells (e.g., each well) is separated from each other by about 0.4 μm to about 2.0 μm. In embodiments, the wells (e.g., each well) is separated from each other by about 0.5 μm to about 2.0 μm. In embodiments, the wells (e.g., each well) is separated from each other by about 1.0 μm to about 2.0 μm. In embodiments, the wells (e.g., each well) is separated from each other by about 1.0 μm to about 1.5 μm. In embodiments, the wells of the solid support are all the same size. In embodiments, the solid support includes wells that are from about 0.1 μm to about 3 μm in diameter. In embodiments, the solid support includes wells that are from about 0.2 μm to about 3 μm in diameter. In embodiments, the solid support includes wells that are from about 0.3 μm to about 3 μm in diameter. In embodiments, the solid support includes wells that are from about 0.4 μm to about 3 μm in diameter. In embodiments, the solid support includes wells that are from about 0.5 μm to about 3 μm in diameter. In embodiments, the solid support includes wells that are from about 0.6 μm to about 3 μm in diameter. In embodiments, the solid support includes wells that are from about 0.7 μm to about 3 μm in diameter. In embodiments, the solid support includes wells that are from about 0.8 μm to about 3 μm in diameter. In embodiments, the solid support includes wells that are from about 0.9 μm to about 3 μm in diameter. In embodiments, the solid support includes wells that are from about 1.0 μm to about 3 μm in diameter. In embodiments, the solid support includes wells that are from about 0.1 μm to about 2 μm in diameter. In embodiments, the solid support includes wells that are from about 0.2 μm to about 2 μm in diameter. In embodiments, the solid support includes wells that are from about 0.3 μm to about 2 μm in diameter. In embodiments, the solid support includes wells that are from about 0.4 μm to about 2 μm in diameter. In embodiments, the solid support includes wells that are from about 0.5 m to about 2 μm in diameter. In embodiments, the solid support includes wells that are from about 0.6 m to about 2 μm in diameter. In embodiments, the solid support includes wells that are from about 0.7 μm to about 2 μm in diameter. In embodiments, the solid support includes wells that are from about 0.8 μm to about 2 μm in diameter. In embodiments, the solid support includes wells that are from about 0.9 μm to about 2 μm in diameter. In embodiments, the solid support includes wells that are from about 1.0 μm to about 2 μm in diameter. In embodiments, the solid support includes wells that are from about 1.0 μm to about 1.5 μm in diameter.

In embodiments, the solid support includes a polymer, photoresist or hydrogel layer. In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

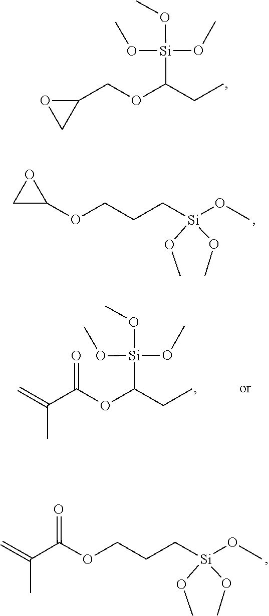

or a copolymer thereof.

In embodiments, the solid support includes a resist (e.g., a photoresist or nanoimprint resist including a crosslinked polymer matrix attached to the solid support). For example, the solid support surface, but not the surface of the wells, is coated in an organically modified ceramic polymer (ORMOCER®, registered trademark of Fraunhofer-Gesellschaft zur Förderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, or US 2010/0160478, each of which is incorporated herein by reference.

In embodiments, the solid support includes a resist (e.g., a photoresist or nanoimprint resist including a crosslinked polymer matrix attached to the solid support). In embodiments, the solid support includes a photoresist, alternatively referred to herein as a resist). In embodiments, the photoresist is a silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, or a organically modified ceramic polymer resist. In embodiments, the photoresist is a silsesquioxane resist. In embodiments, the photoresist is an epoxy-based polymer resist. In embodiments, the photoresist is a poly(vinylpyrrolidone-vinyl acrylic acid) copolymer resist. In embodiments, the photoresist is an Off-stoichiometry thiol-enes (OSTE) resist. In embodiments, the photoresist is an amorphous fluoropolymer resist. In embodiments, the photoresist is a crystalline fluoropolymer resist. In embodiments, the photoresist is a polysiloxane resist. In embodiments, the photoresist is an organically modified ceramic polymer resist. In embodiments, the photoresist includes polymerized alkoxysilyl methacrylate polymers and metal oxides (e.g., SiO₂, ZrO, MgO, Al₂O₃, TiO₂ or Ta₂O₅). In embodiments, the photoresist includes polymerized alkoxysilyl acrylate polymers and metal oxides (e.g., SiO₂, ZrO, MgO, Al₂O₃, TiO₂ or Ta₂O₅). In embodiments, the photoresist includes metal atoms, such as Si, Zr, Mg, Al, Ti or Ta atoms. In embodiments, the solid support is a glass slide about 75 mm by about 25 mm.

In embodiments, the wells are separated from each other by interstitial regions including a polymer layer as described herein (e.g., an amphiphilic copolymer). In embodiments, the solid support further includes a photoresist, wherein the photoresist does not contact the bottom of the well. In embodiments, the polymer layer is substantially free of overlapping amplification clusters. In embodiments, the solid support does not include a polymer (e.g., the solid support is a patterned glass slide). In embodiments, the wells do not include a polymer (e.g., an amphiphilic polymer as described herein). In embodiments, the solid support further includes a photoresist, wherein the photoresist is in contact the bottom of the well and the interstitial space. In embodiments, the wells include a polymer (e.g., an amphiphilic polymer and/or resist as described herein).

In embodiments, the template polynucleotide is immobilized to a solid support at a discrete site. In embodiments, each discrete site includes a plurality of oligonucleotide moieties covalently attached to said site via a bioconjugate linker. In embodiments, the solid support further includes oligonucleotide moieties capable of annealing to an adapter of a library nucleic acid molecule. The term “library” merely refers to a collection or plurality of template nucleic acid molecules which share common sequences at their 5′ ends (e.g., the first end) and common sequences at their 3′ ends (e.g., the second end). The term “adapter” as used herein refers to any linear oligonucleotide that can be ligated to a nucleic acid molecule, thereby generating nucleic acid products that can be sequenced on a sequencing platform (e.g., an Illumina or Singular Genomics' G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences or S1 and S2 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

In embodiments, the template polynucleotide includes spacer nucleotides. Including spacer nucleotides in the linker puts the target polynucleotide in an environment having a greater resemblance to free solution. This can be beneficial, for example, in enzyme-mediated reactions such as sequencing-by-synthesis. It is believed that such reactions suffer less steric hindrance issues that can occur when the polynucleotide is directly attached to the particle or is attached through a very short linker (e.g., a linker comprising about 1 to 3 carbon atoms). Spacer nucleotides form part of the oligonucleotide moiety but do not participate in any reaction carried out on or with the oligonucleotide (e.g., a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacers, although other nucleotides and combinations thereof can be used. In embodiments, the linker includes 10, 11, 12, 13, 14, or 15 T spacer nucleotides. In embodiments, the linker includes 12 T spacer nucleotides. Spacer nucleotides are typically included at the 5′ ends of oligonucleotide which are attached to the particle. Attachment can be achieved via a phosphorothioate present at the 5′ end of the oligonucleotide, an azide moiety, a dibenzocyclooctyne (DBCO) moiety, or any other bioconjugate reactive moiety (e.g., a bioconjugate moiety as described herein).

In embodiments, the polymerase is DNA polymerase, which includes a 9° N polymerase or variant thereof. In other embodiments, the DNA polymerase is E. coli DNA polymerase I, bacteriophage T4 DNA polymerase, SEQUENASE™ (genetically engineered T7 DNA polymerase having little to no 3′ to 5′ exonuclease activity; ThermoFisher Scientific), and Taq DNA polymerase, or a variant of each thereof.

In embodiments, the sequencing nucleotides in the reaction mixture include two, three, or four species of sequencing nucleotides, each of which includes a reversible terminator moiety and a detectable label moiety covalently bound to the sequencing nucleotide via a cleavable linker. In embodiments, the sequencing nucleotides all have the same reversible terminator moiety. In embodiments, the sequencing nucleotides all have the same detectable label moiety. In embodiments, the sequencing nucleotides all have the same cleavable linker. In embodiments, the sequencing nucleotides all have the same reversible terminator moiety, the same detectable label moiety, and the same cleavable linker. A label can also be removed or modified by cleaving the label while leaving the linker intact, so long as the detectable signal from the label (e.g., a dye) is reduced sufficiently to allow identification of a subsequently added label molecule to an extended nucleic acid chain. In embodiments, for each polymerase extension cycle, only one nucleotide will be incorporated. In embodiments using fluorescent labels, a fluorescent image is taken to determine which base has been incorporated based on the color codes. In embodiments, the label molecules can be removed, and the reversible terminator can be subsequently or simultaneously removed (as can occur if both cleavage reactions are enzymatic reactions and can be carried out in the same buffer). Once the label and blocking groups are removed, the next SBS cycle can be initiated.

In embodiments, the chase nucleotides in the reaction mixture include two, three, or four species of nucleotides, each of which includes a reversible terminator moiety and a retarding moiety covalently bound to the nucleotide via a cleavable linker. In embodiments, the chase nucleotide analogues are nucleotides with a 3′-reversible terminator moiety that may be unblocked for extension in a subsequent SBS cycle having a retardant moiety. In embodiments, the chase nucleotides all have the same retarding moiety. In embodiments, the chase nucleotides all have the same detectable label moiety. In embodiments, the chase nucleotides all have the same cleavable linker. In embodiments, the chase nucleotides all have the same reversible terminator moiety, the same retarding moiety, and the same cleavable linker. In embodiments, the retarding moiety is not detected under the same conditions used to detect the sequencing nucleotides. Incorporation of a chase nucleotide into a growing DNA strand that is complementary to the template DNA molecule is under conditions to ensure the efficient production of extension products in a given SBS cycle. As will be appreciated, extension of all primed DNA template molecules, and their extension products, is critical to ensure accurate DNA sequencing. Incorporation of a chase nucleotide into a primed template DNA molecule that was not extended by a sequencing nucleotide allows for formation of a population of unlabeled, blocked extension product(s).

In embodiments, a template polynucleotide can include any nucleic acid of interest. Template polynucleotides can include DNA, RNA, peptide nucleic acid, morpholino nucleic acid, locked nucleic acid, glycol nucleic acid, threose nucleic acid, mixtures thereof, and hybrids thereof. In embodiments, the template polynucleotide is obtained from one or more source organisms. As used herein the term “organism” is not necessarily limited to a particular species of organism but can be used to refer to the living or self-replicating particle at any level of classification, which comprises the template polynucleotide. For example, the term “organism” can be used to refer collectively to all of the species within the genus Salmonella or all of the bacteria within the kingdom Eubacteria. A template polynucleotide can comprise any nucleotide sequence. In some embodiments, the template polynucleotide can include a selected sequence or a portion of a larger sequence. In embodiments, sequencing a portion of a target nucleic acid or a fragment thereof can be used to identify the source of the target nucleic acid.

In embodiments, the primer is hybridized to the template polynucleotide. In embodiments, the primer is about 10 to 100 nucleotides in length. In embodiments, the primer is about 15 to about 75 nucleotides in length. In embodiments, the primer is about 25 to about 75 nucleotides in length. In embodiments, the primer is about 15 to about 50 nucleotides in length. In embodiments, the primer is about 10 to about 20 nucleotides in length. In embodiments, the primer is about 10, 11, 12, 13, 14, 15, 16, 17, 18, 19, or about 20 nucleotides in length. In embodiments, the primer is about 20, 21, 22, 23, 24, 25, 26, 27, 28, 29, or about 30 nucleotides in length. In embodiments, the primer is about 30, 31, 32, 33, 34, 35, 36, 37, 38, 39, or about 40 nucleotides in length. In embodiments, the primer is about 40, 41, 42, 43, 44, 45, 46, 47, 48, 49, or about 50 nucleotides in length. In embodiments, the primer is greater than 30 nucleotides in length. In embodiments, the primer is greater than 40 nucleotides in length. In embodiments, the primer is greater than 50 nucleotides in length. In embodiments, the primer is no less than 20 nucleotides. In embodiments, the primer is about 15 to about 35 nucleotides in length.

In embodiments, step d) extends the same template polynucleotide of step a) so that two sequencing nucleotides are included in the extension strand (i.e. the extended polynucleotide from the first primer). In embodiments, a third primer hybridized to a template polynucleotide is contacted with a second chase nucleotide having a second retarding moiety covalently bound to the nucleotide via a second chase cleavable linker. In embodiments, the third primer is the same as the second primer of step b) so that there are two chase nucleotides included in the same extension strand. In embodiments, the third primer is on a different template polynucleotide than the template polynucleotide of step b) so that two separate extension strands each have a chase nucleotide. In embodiments, each of the template polynucleotide described in steps a) to d) are different templates from one another which are found in the same cluster of polynucleotides as found in sequencing by synthesis (SBS) process. In embodiments, step e) (i.e., contacting of a third primer hybridized to a third template polynucleotide with a second chase nucleotide that is incorporated into the primer with a polymerase) can occur at the same time as step d) (i.e., when a second sequencing nucleotide is contacted with the first extended primer polynucleotide). In embodiments, step e) (i.e., contacting of a third primer hybridized to a third template polynucleotide with a second chase nucleotide that is incorporated into the primer with a polymerase) can occur following step d) (i.e., after a second sequencing nucleotide is incorporated into the first extended primer polynucleotide). In embodiments, step b) is repeated after step d).

In embodiments, the methods further comprise removal of any unbound sequencing nucleotides or chase nucleotides (e.g., a fluidic exchange that washes and removes any unbound nucleotides). Removal of unbound nucleotides may occur at any step of the methods described herein (e.g., after contacting with a sequencing solution but prior to contacting with a chase solution, or during detection. In embodiments, contact of the chase nucleotide with a second primer is initiated before the sequencing reaction is complete (i.e., 95%-100% of the primed template polynucleotides have incorporated a sequencing nucleotide) but after a sufficient percentage of the primed template polynucleotides have been extended by incorporating sequencing nucleotides so that the identity of the added sequencing nucleotide can be determined. In embodiments, addition of chase nucleotides is initiated after the sequencing reaction is about 25% to less than 95% complete, about 40% to about 80% complete, about 45% to about 75% complete, or about 50% to about 70% complete. In embodiments, addition of chase nucleotides is initiated after the sequencing reaction is about 50% complete. Completion of the sequencing reaction may include any value or subrange within the recited ranges, including endpoints.

As described herein, a cycle may refer to a sequencing cycle (i.e., a cycle that includes detecting a characteristic signature indicating that a sequencing nucleotide was incorporated into the primer), or a cycle may refer to an extension cycle (e.g., a dark cycle, wherein the cycle does not include detecting a characteristic signature but a chase nucleotide was incorporated into the primer).

In embodiments, the methods described herein result in a cycle (e.g., cycle including extension, chase, image, cleave, and/or wash/fluid movement steps), wherein each repetition of steps (a), (b) and (c) is a cycle. In embodiments, each cycle between about 1 minute and about 40 minutes long. In embodiments, the cycle is between about 1 minute and about 30 minutes long. In embodiments, the cycle is between about 1 minute and about 20 minutes long. In embodiments, the cycle is between about 1 minute and about 15 minutes long. In embodiments, the cycle is between about 1 minute and about 10 minutes long. In embodiments, the cycle is between about 1 minute and about 5 minutes long. In embodiments, the cycle is between about 1 minute and about 3 minutes long. In embodiments, the cycle is between about 1 minute and about 2 minutes long. The length of the cycle may include any value or subrange within the recited ranges, including endpoints.

In embodiments, the methods described herein result in a sequencing cycle that is at least about 10%, at least about 20%, at least about 30%, at least about 40%, at least about 50%, or at least about 60% faster than a conventional SBS sequencing cycle (e.g., a sequencing cycle that does not include simultaneous imaging during step (a) or step (b)). In embodiments, the methods described herein result in a combined extension, chase, and image steps within a cycle that is at least about 10%, at least about 20%, at least about 30%, at least about 40%, at least about 50%, or at least about 60% faster than a conventional SBS sequencing cycle. In embodiments, said methods described herein result in a total sequencing reaction (i.e., having “n” iterations) that is at least about 10%, at least about 20%, at least about 30%, at least about 40%, at least about 50%, or at least about 60% faster than a conventional SBS sequencing cycle (having “n” iterations).

In embodiments, a cycle is the repetition of steps (a), (b) and (c), wherein each cycle is performed two or more (e.g., at least 2, 5, 10, 15, 20, 25, or 30) times performing a series of cycles, wherein each cycle is a first ordered cycle or a second ordered cycle, In a first ordered cycle, the first primer contacts the sequencing solution first and the second primer contacts the chase nucleotide second, wherein in a second ordered cycle, the second primer contacts the chase nucleotide first and the first primer contacts the sequencing solution second and wherein the series of cycles is performed according to a non-cyclic sequence.

In embodiments, each cycle (e.g., the repetition of steps (a), (b) and (c)) is performed for 1-200 times. In embodiments, each cycle is performed at least 20 times, 30 times, at least 40 times, at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, or at least 200 times. In embodiments, each cycle is performed 2, 3, 4, 5, 6, 7, 8, 9, 10, 11, 12, 13, 14, 15, 16, 17, 18, 19, 20 or more times thereby performing a series of cycles. In embodiments, the series of cycles includes at least 2 cycles. In embodiments, the series of cycles includes at least 5 cycles. In embodiments, the series of cycles includes at least 8 cycles. In embodiments, the series of cycles includes at least 10 cycles. In embodiments, the series of cycles includes at least 15 cycles. In embodiments, the series of cycles includes at least 20 cycles. In embodiments, the series of cycles includes at least 25 cycles. In embodiments, the series of cycles includes at least 30 cycles. In embodiments, the series of cycles includes at least 40 cycles, or at least 50 cycles. In embodiments, the series of cycles includes at least 75 cycles, at least 100 cycles, at least 150 cycles, or at least 200 cycles. In embodiments, the series of cycles includes greater than 2 cycles. In embodiments, the series of cycles includes greater than 5 cycles. In embodiments, the series of cycles includes greater than 8 cycles. In embodiments, the series of cycles includes greater than 10 cycles. In embodiments, the series of cycles includes greater than 15 cycles. In embodiments, the series of cycles includes greater than 20 cycles. In embodiments, the series of cycles includes greater than 25 cycles. In embodiments, the series of cycles includes greater than 30 cycles. In embodiments, the series of cycles includes greater than 40 cycles, or greater than 50 cycles. In embodiments, the series of cycles includes greater than 75 cycles, greater than 100 cycles, greater than 150 cycles, or greater than 200 cycles.

In embodiments, the nucleotide types of the first extension solution and the nucleotide types of the second extension solution differ across one or more cycles. In embodiments, the nucleotide types of the first extension solution and the nucleotide types of the second extension solution are the same across one or more cycles. A “nucleotide type”, as used herein, refers to a particular nucleobase of a nucleotide triphosphate. For example, a nucleotide type may be a purine nucleotide (i.e., adenine and guanine) or pyrimidine nucleotides (i.e., cytosine and thymine). In embodiments, a first nucleotide type is an adenine nucleotide, or analog thereof. In embodiments, a second nucleotide type is a guanine nucleotide, or analog thereof. In embodiments, a third nucleotide type is a cytosine nucleotide, or analog thereof. In embodiments, a fourth nucleotide type is a thymine nucleotide, or analog thereof.

In embodiments, the concentration of chase nucleotides used in any of the methods described herein is between 0.5× to 10× the concentration of sequencing nucleotides. In embodiments, the concentration of chase nucleotides used in any of the methods described herein is between 1× to 10× the concentration of sequencing nucleotides. In embodiments, the concentration of chase nucleotides used in any of the methods described herein is between 2× to 5× the concentration of sequencing nucleotides. In embodiments, the concentration of chase nucleotides used in any of the methods described herein is 3× the concentration of sequencing nucleotides. In embodiments, the concentration of chase nucleotides to sequencing nucleotides is about 1:1, 2:1, 3:1, 4:1 or 5:1. In embodiments, the concentration of chase nucleotides to sequencing nucleotides is 1:1. In embodiments, the concentration of chase nucleotides to sequencing nucleotides is 2:1. In embodiments, the concentration of chase nucleotides to sequencing nucleotides is 3:1. In embodiments, the concentration of chase nucleotides to sequencing nucleotides is 4:1. In embodiments, the concentration of chase nucleotides to sequencing nucleotides is 5:1. In embodiments, the concentration of chase nucleotides to sequencing nucleotides is about 1:1. In embodiments, the concentration of chase nucleotides to sequencing nucleotides is about 2:1. In embodiments, the concentration of chase nucleotides to sequencing nucleotides is about 3:1. In embodiments, the concentration of chase nucleotides to sequencing nucleotides is about 4:1. In embodiments, the concentration of chase nucleotides to sequencing nucleotides is about 5:1.

In embodiments, detection of the sequencing nucleotides includes detection of the detectable label moiety (e.g., first detectable label moiety, second detectable label moiety). In embodiments, the detectable label moiety is directly detectable or is secondary label that can be indirectly detected, for example, via direct or indirect interaction with a primary label. Labels includes dyes, chromophores, combinatorial fluorescence energy transfer labels, electrophores, fluorophores, mass labels, and radiolabels. For example, detectable labels include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), modified oligonucleotides (e.g., moieties described in PCT/US2015/022063, which is incorporated herein by reference). In embodiments, the detectable label moiety (e.g., first detectable label moiety, second detectable label moiety) is a fluorophore.

In embodiments, detection of the sequencing nucleotide includes directing an excitation beam at the fluorophore to generate a fluorescent emission that is detected by a sensor array. To determine the emission spectrum of a particular fluorophore, the wavelength of maximum absorption (i.e., excitation maximum) is determined and the fluorophore is excited at this wavelength. In embodiments, the excitation beam excites the fluorophore to the maximum emission. Following excitation, the fluorophore emits a fluorescent signal that can be monitored at the wavelength of maximum intensity, known as the emission maximum. In embodiments, the fluorophore is excited at the excitation wavelength and its presence detected by monitoring of an emission beam at an emission wavelength. In embodiments, the chase nucleotide has a retardant moiety which is a detectable label. In embodiments, the detectable label of the retardant moiety emits a signal so that the maximum emission does not overlap with the maximum emission of the detectable label moiety of the sequencing nucleotide. In embodiments, maximum emission of the detectable label of the retardant moiety is at least 20 nm below or above maximum emission of the detectable label moiety of the sequencing nucleotide.

In embodiments, sequencing includes sequencing-by-synthesis, sequencing-by-binding, sequencing by ligation, or pyrosequencing. In embodiments, generating a first sequencing read or a second sequencing read includes a sequencing by synthesis process. In embodiments, generating a first sequencing read or a second sequencing read includes a sequencing-by-binding. As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide. In embodiments, sequencing includes generating a sequencing read. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and 6,274,320, each of which is incorporated herein by reference in its entirety). In pyrosequencing, released Ppi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label moiety that indicates the identity of a nucleotide in the target polynucleotide, detecting the detectable label moiety, and repeating the extending and detecting steps. In embodiments, said methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments the template polynucleotide is an RNA transcript. RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the template polynucleotide is pre-mRNA. In embodiments, the template polynucleotide is heterogeneous nuclear RNA (hnRNA). In embodiments, the template polynucleotide is a single stranded RNA nucleic acid sequence. In embodiments, the template polynucleotide is an RNA nucleic acid sequence or a DNA nucleic acid sequence (e.g., cDNA). In embodiments, the template polynucleotide is a cDNA target nucleic acid sequence. In embodiments, the template polynucleotide is genomic DNA (gDNA), mitochondrial DNA, chloroplast DNA, episomal DNA, viral DNA, or complementary DNA (cDNA). In embodiments, the template polynucleotide is coding RNA such as messenger RNA (mRNA), and non-coding RNA (ncRNA) such as transfer RNA (tRNA), microRNA (miRNA), small nuclear RNA (snRNA), or ribosomal RNA (rRNA).

In embodiments, the template polynucleotides are RNA nucleic acid sequences or DNA nucleic acid sequences. In embodiments, the template polynucleotides are RNA nucleic acid sequences or DNA nucleic acid sequences from the same cell. In embodiments, the template polynucleotides are RNA nucleic acid sequences. In embodiments, the RNA nucleic acid sequence is stabilized using known techniques in the art. For example, RNA degradation by RNase should be minimized using commercially available solutions (e.g., RNA Later®, RNA Protect®, or DNA/RNA Shield®). In embodiments, the sample polynucleotides are messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the template polynucleotide is pre-mRNA. In embodiments, the template polynucleotide is heterogeneous nuclear RNA (hnRNA). In embodiments, the template polynucleotide is mRNA, tRNA (transfer RNA), rRNA (ribosomal RNA), or noncoding RNA (such as lncRNA (long noncoding RNA)). In embodiments, the template polynucleotides are on different regions of the same RNA nucleic acid sequence. In embodiments, the template polynucleotide is cDNA target nucleic acid sequences and before step i), the RNA nucleic acid sequences are reverse transcribed to generate the cDNA target nucleic acid sequences. In embodiments, the template polynucleotide is not reverse transcribed to cDNA. When mRNA is reverse transcribed an oligo(dT) primer can be added to better hybridize to the poly A tail of the mRNA. The oligo(dT) primer may include between about 12 and about 25 dT residues. The oligo(dT) primer may be an oligo(dT) primer of between about 18 to about 25 nt in length.

In embodiments of a method herein, the template polynucleotide is about 50 to about 1500 nucleotides in length. In some embodiments of a method herein, the template polynucleotide is about 50 to about 500 nucleotides in length. In some embodiments, the template polynucleotide is greater than 100 nucleotides in length. In embodiments, the template polynucleotide is about 500 nucleotides in length. In embodiments, the template polynucleotide is about 5 to about 250 nucleotides in length. In embodiments, the template polynucleotide is about 5 to about 200 nucleotides in length. In embodiments, the template polynucleotide is about 5 to about 150 nucleotides in length. In embodiments, the template polynucleotide is about 5 to about 100 nucleotides in length. In embodiments, the template polynucleotide is about 5 to about 60 nucleotides in length. In embodiments, the template polynucleotide is about 5 to about 50 nucleotides in length. In embodiments, the template polynucleotide is about 5 to about 40 nucleotides in length. In embodiments, the template polynucleotide is about 10 to about 250 nucleotides in length. In embodiments, the template polynucleotide is about 10 to about 200 nucleotides in length. In embodiments, the template polynucleotide is about 10 to about 150 nucleotides in length. In embodiments, the template polynucleotide is about 10 to about 100 nucleotides in length. In embodiments, the template polynucleotide is about 10 to about 60 nucleotides in length. In embodiments, the template polynucleotide is about 10 to about 50 nucleotides in length. In embodiments, the template polynucleotide is about 10 to about 45 nucleotides in length. In embodiments, the template polynucleotide is about 10 to about 40 nucleotides in length. In embodiments, the template polynucleotide is about 15 to about 100 nucleotides in length. In embodiments, the template polynucleotide is about 15 to about 90 nucleotides in length. In embodiments, the template polynucleotide is about 15 to about 80 nucleotides in length. In embodiments, the template polynucleotide is about 15 to about 70 nucleotides in length. In embodiments, the template polynucleotide is about 15 to about 60 nucleotides in length. In embodiments, the template polynucleotide is about 15 to about 50 nucleotides in length. In embodiments, the template polynucleotide is about 15 to about 40 nucleotides in length. In embodiments, the template polynucleotide is about 15 to about 30 nucleotides in length. In embodiments, the template polynucleotide is about 20 to about 35 nucleotides in length. In embodiments, the template polynucleotide is about 20 to about 30 nucleotides in length. In embodiments, the template polynucleotide is about 25 to about 30 nucleotides in length. In embodiments, the template polynucleotide is about 25 to about 35 nucleotides in length. In embodiments, the template polynucleotide is about 30 to about 50 nucleotides in length. In embodiments, the template polynucleotide is about 30 to about 75 nucleotides in length. In embodiments, the template polynucleotide is about 50 to about 150 nucleotides in length. In some embodiments, the oligonucleotide moiety is about 75 to about 200 nucleotides in length.

In embodiments of a method herein, each sequencing cycle includes a probability of an incorrect base call that is less than 1 in 100. In embodiments, each sequencing cycle includes a probability of an incorrect base call that is less than 1 in 1,000. In embodiments, each sequencing cycle includes a probability of an incorrect base call that is less than 1 in 10,000.

In embodiments of a method herein, greater than 85% of the templates are in phase following each sequencing cycle. In embodiments, greater than 90% of the templates are in phase following each sequencing cycle. In embodiments, greater than 91% of the templates are in phase following each sequencing cycle. In embodiments, greater than 92% of the templates are in phase following each sequencing cycle. In embodiments, greater than 93% of the templates are in phase following each sequencing cycle. In embodiments, greater than 94% of the templates are in phase following each sequencing cycle. In embodiments, greater than 95% of the templates are in phase following each sequencing cycle. In embodiments, greater than 96% of the templates are in phase following each sequencing cycle. In embodiments, greater than 97% of the templates are in phase following each sequencing cycle. In embodiments, greater than 98% of the templates are in phase following each sequencing cycle. In embodiments, greater than 99% of the templates are in phase following each sequencing cycle. In embodiments, greater than 99.9% of the templates are in phase following each sequencing cycle. In embodiments, greater than 80% of the templates are in phase after 50 sequencing cycles. In embodiments, greater than 60% of templates are in phase after 100 sequencing cycles. The percentage of templates in phase represents the average fraction of in-phase templates among clusters analyzed in a sequencing run.

In embodiments of a method herein, each sequencing cycle includes a probability of an incorrect base call that is less than 1 in 100 for about 200 to 1,000 nucleotide incorporations. In some embodiments, each sequencing cycle includes a probability of an incorrect base call that is less than 1 in 1,000 for about 200 to 1,000 nucleotide incorporations. In some embodiments, each sequencing cycle includes a probability of an incorrect base call that is less than 1 in 10,000 for about 200 to 1,000 nucleotide incorporations. In other embodiments, each sequencing cycle includes a probability of an incorrect base call that is less than 1 in 100 for about 300 to 1,000 nucleotide incorporations. In embodiments, each sequencing cycle includes a probability of an incorrect base call that is less than 1 in 1,000 for about 300 to 1,000 nucleotide incorporations. In embodiments, each sequencing cycle includes a probability of an incorrect base call that is less than 1 in 10,000 for about 300 to 1,000 nucleotide incorporations. In embodiments, each sequencing cycle includes a probability of an incorrect base call that is less than 1 in 100 for about 500 to 1,000 nucleotide incorporations. In embodiments, each sequencing cycle includes a probability of an incorrect base call that is less than 1 in 1,000 for about 500 to 1,000 nucleotide incorporations. In embodiments, each sequencing cycle includes a probability of an incorrect base call that is less than 1 in 10,000 for about 500 to 1,000 nucleotide incorporations. In embodiments, each sequencing cycle includes a probability of an incorrect base call that is less than 1 in 100 for about 750 to 1,000 nucleotide incorporations. In embodiments, each sequencing cycle includes a probability of an incorrect base call that is less than 1 in 1,000 for about 750 to 1,000 nucleotide incorporations. In embodiments, each sequencing cycle includes a probability of an incorrect base call that is less than 1 in 10,000 for about 750 to 1,000 nucleotide incorporations. In embodiments, each sequencing cycle includes a probability of an incorrect base call that is less than 1 in 100 for about 900 to 1,000 nucleotide incorporations. In embodiments, each sequencing cycle includes a probability of an incorrect base call that is less than 1 in 1,000 for about 900 to 1,000 nucleotide incorporations. In embodiments, each sequencing cycle includes a probability of an incorrect base call that is less than 1 in 10,000 for about 900 to 1,000 nucleotide incorporations.

In an aspect is provided a method of detecting an incorporated sequencing nucleotide, the method including: i) contacting a solid support including a plurality of template polynucleotides with a plurality of chase nucleotides, wherein each chase nucleotide includes a retarding moiety covalently bound to the chase nucleotide via a cleavable linker, and wherein a first fraction of the plurality of template polynucleotides are hybridized to an unblocked primer; and a second fraction of the plurality of template polynucleotides are hybridized to a blocked primer, wherein the blocked primer includes the incorporated sequencing nucleotide at a 3′ end of the blocked primer; ii) incorporating one of the chase nucleotides into the unblocked primer with a polymerase; and iii) detecting the incorporated sequencing nucleotide.

In embodiments, the blocked primer includes a 3′ blocking moiety. In embodiments, the blocking moiety is thermolabile, acid-labile, redox-labile, or photolabile. In further embodiments, the blocking moiety has a modified nucleotide at the 3′ end of the blocked primer. In embodiments, the modified nucleotide includes a 3′ reversible terminator and a detectable label moiety attached via a cleavable linker. In embodiments, the template polynucleotide strand further includes a second primer region that is not blocked. In embodiments, the second primer region has an open (i.e., free 3′-OH) position in which a nucleotide can be added. In embodiments, template polynucleotide strands having the unblocked primer region is contacted with a mixture of chase nucleotides that include a retardant moiety covalently bound to the nucleotide via a cleavable linker, and this unblocked primer incorporates one of the chase nucleotides, as described herein. Following incorporation of the chase nucleotide, the modified nucleotide at the 3′ end of the blocked primer is detected.

In embodiments, the template polynucleotide strands are attached to a solid substrate. The template polynucleotide strands may be attached by any conventional technique for attaching polynucleotides sequences to solid substrates. For example, the surface of the solid substrate may be coated with linker molecules that in turn attach to an end of the universal template strands. As a further example, the surface of the solid substrate array may be functionalized through silanization or by coating with agarose. This creates a solid substrate that is coated with a plurality of anchor sequences. In embodiments, the solid substrate may be a microelectrode array. The solid substrate that is coated with template polynucleotide strands may be reused multiple times.

In embodiments, the solid support includes a plurality of template polynucleotides, wherein each polynucleotide is attached to the solid support at a 5′ end of the polynucleotide. In embodiments, the solid support is selected from a flow cell, bead, chip, capillary, plate, membrane, wafer, comb, pin, nanoparticle, multi-well container, or unpatterned solid support. In embodiments, the solid support is contained within a flow cell. In embodiments, the solid support is a flow cell. In embodiments, the solid support is a bead. In embodiments, the solid support is a nanoparticle. In embodiments, the solid support is substantially planar. In embodiments, the solid support is a multiwell container. In embodiments, the solid support is an unpatterned solid support.

In an aspect is provided a method of extending a primer, the method including: contacting a primer hybridized to a template polynucleotide with a first plurality of nucleotides (e.g., a sequencing solution), followed by contacting the primer with a second plurality of nucleotides (e.g., a chase solution); and in the presence of a polymerase, incorporating a nucleotide from the first plurality (e.g., the sequencing solution) or incorporating a nucleotide from the second plurality (e.g., the chase solution) to extend the primer. In an aspect is provided a method of extending a primer, the method including contacting a primer hybridized to a template polynucleotide with a sequencing solution, followed by contacting the primer with a chase solution; and in the presence of a polymerase, incorporating a nucleotide from the sequencing solution or incorporating a nucleotide from the chase solution to extend the primer. In embodiments, the (a) the sequencing solution includes a plurality of sequencing nucleotides, (b) each nucleotide of the plurality of sequencing nucleotides includes a detectable label moiety (e.g., associated with a nucleobase) and a first reversible terminator moiety; (c) the chase solution includes a plurality of chase nucleotides, (d) each nucleotide of the plurality of chase nucleotides including a retardant moiety and a second reversible terminator moiety, and (e) the retardant moieties differ in structure from the detectable label moieties. In embodiments, the chase solution and sequencing solution are independent solutions (i.e., they are not mixtures containing both sequencing and chase nucleotides). In embodiments, prior to introducing a new solution (e.g., prior to contacting the primer with a chase solution) the solution currently contacting the primer is removed from the reaction vessel (e.g., subject to a fluidic exchange and washed).

In embodiments, the method further includes detecting the detectable label moiety i) prior to contacting the primer with the chase solution, or ii) after contacting the primer with the chase solution. In embodiments, the method includes detecting the detectable label moiety during contacting of the primer with the chase solution.

In embodiments, the method further includes removing (a) the first or second reversible terminator moiety, and (b) the detectable label moiety or the retardant moiety. In embodiments, removing includes contacting the nucleotide with a cleaving agent (e.g., a reducing agent).

In embodiments, the method includes repeating contacting the extended primer with the sequencing solution, followed by contacting the extended primer with the chase solution.

In an aspect is provided a method of sequencing a plurality of template polynucleotides, the method including: (a) contacting a plurality of primers hybridized to template polynucleotides with a chase solution in the presence of a polymerase; wherein a fraction of the plurality of primers include a 3′ terminal nucleotide including a first detectable label moiety and a first reversible terminator moiety; wherein the chase solution includes a plurality of chase nucleotides, each nucleotide in the plurality of chase nucleotides including a retardant moiety and a second reversible terminator moiety; (b) detecting the first detectable label moiety of the 3′ terminal nucleotide; (c) removing the first detectable label moiety, the retardant moiety, and the first and second reversible terminator moieties from nucleotides of the plurality of primers; (d) contacting the plurality of primers hybridized to template polynucleotides with a sequencing solution, wherein the sequencing solution includes a plurality of sequencing nucleotides, each nucleotide of the plurality of sequencing nucleotides including a second detectable label moiety and a third reversible terminator moiety; and wherein a fraction of the plurality of primers incorporate a nucleotide of the plurality of sequencing nucleotides; and (e) repeating steps (a)-(d) thereby sequencing the template polynucleotides.

In yet another aspect is provided a method of sequencing a plurality of template polynucleotides, the method including: i) contacting a substrate including a plurality of immobilized template polynucleotides with a sequencing solution including a plurality of sequencing nucleotides, each nucleotide of the plurality of sequencing nucleotides including a detectable label moiety and a first reversible terminator moiety, wherein each immobilized template polynucleotide includes one or more primers hybridized thereto; and in the presence of a polymerase, extending the one or more primers with a nucleotide to generate extended primers; ii) contacting the substrate with a chase solution including a plurality of chase nucleotides, each nucleotide of the plurality of chase nucleotides including a retardant moiety and a second reversible terminator moiety; iii) detecting the detectable label moiety so as to identify one or more nucleotides incorporated into the extended primers; iv) removing the first and second reversible terminator moieties, the detectable label moiety, and the retardant moiety; and v) repeating steps i) to iv) to sequence the plurality of immobilized template polynucleotides. In embodiments, the method further includes detecting the retardant moiety prior to step iv).

In an aspect is provided a method of detecting templates in a cluster, the method including: (a) contacting a cluster including a plurality of templates with a plurality of chase nucleotides in the presence of a polymerase, each nucleotide of the plurality of chase nucleotides including a retardant moiety and a reversible terminator moiety; wherein a fraction of the plurality of templates in the cluster include reversible-terminated, labeled nucleotides incorporated at the 3′ ends of primers hybridized to the fraction of the plurality of templates; and (b) detecting one or more of the retardant moieties incorporated by primer extension, thereby detecting templates. In embodiments, the method further includes detecting the labeled nucleotides. In embodiments, the method includes removing the reversible terminator moiety, a label of the labeled nucleotides, and the retardant moiety.

In embodiments, following incorporation of one of the plurality of chase nucleotides by primer extension, the incorporation rate of a subsequent nucleotide is decreased. In embodiments, following incorporation of one of the plurality of chase nucleotides by primer extension, the incorporation rate of a subsequent nucleotide is decreased by a factor of about 3 to about 10. In embodiments, following incorporation of one of the plurality of chase nucleotides by primer extension, the incorporation rate of a subsequent nucleotide is decreased by a factor of about 2. In embodiments, following incorporation of one of the plurality of chase nucleotides by primer extension, the incorporation rate of a subsequent nucleotide is decreased by a factor of about 3. In embodiments, following incorporation of one of the plurality of chase nucleotides by primer extension, the incorporation rate of a subsequent nucleotide is decreased by a factor of about 4. In embodiments, following incorporation of one of the plurality of chase nucleotides by primer extension, the incorporation rate of a subsequent nucleotide is decreased by a factor of about 5. In embodiments, following incorporation of one of the plurality of chase nucleotides by primer extension, the incorporation rate of a subsequent nucleotide is decreased by a factor of about 6. In embodiments, following incorporation of one of the plurality of chase nucleotides by primer extension, the incorporation rate of a subsequent nucleotide is decreased by a factor of about 7. In embodiments, following incorporation of one of the plurality of chase nucleotides by primer extension, the incorporation rate of a subsequent nucleotide is decreased by a factor of about 8. In embodiments, following incorporation of one of the plurality of chase nucleotides by primer extension, the incorporation rate of a subsequent nucleotide is decreased by a factor of about 9. In embodiments, following incorporation of one of the plurality of chase nucleotides by primer extension, the incorporation rate of a subsequent nucleotide is decreased by a factor of about 10. In embodiments, following incorporation of one of the plurality of chase nucleotides by primer extension, the incorporation rate of a subsequent nucleotide is decreased by a factor of about 11. In embodiments, following incorporation of one of the plurality of chase nucleotides by primer extension, the incorporation rate of a subsequent nucleotide is decreased by a factor of about 12. In embodiments, following incorporation of one of the plurality of chase nucleotides by primer extension, the incorporation rate of a subsequent nucleotide is decreased by a factor of about 13. In embodiments, following incorporation of one of the plurality of chase nucleotides by primer extension, the incorporation rate of a subsequent nucleotide is decreased by a factor of about 14. In embodiments, following incorporation of one of the plurality of chase nucleotides by primer extension, the incorporation rate of a subsequent nucleotide is decreased by a factor of about 15. In embodiments, following incorporation of one of the plurality of chase nucleotides by primer extension, the incorporation rate of a subsequent nucleotide is decreased by a factor of about 16. In embodiments, following incorporation of one of the plurality of chase nucleotides by primer extension, the incorporation rate of a subsequent nucleotide is decreased by a factor of about 17. In embodiments, following incorporation of one of the plurality of chase nucleotides by primer extension, the incorporation rate of a subsequent nucleotide is decreased by a factor of about 18. In embodiments, following incorporation of one of the plurality of chase nucleotides by primer extension, the incorporation rate of a subsequent nucleotide is decreased by a factor of about 19. In embodiments, following incorporation of one of the plurality of chase nucleotides by primer extension, the incorporation rate of a subsequent nucleotide is decreased by a factor of about 20.

In embodiments, each nucleotide of the plurality of sequencing nucleotides has the formula:

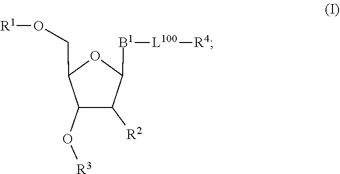

wherein, B¹ is a nucleobase; R¹ is a triphosphate or thiotriphosphate; R² is hydrogen or —OH; R³ is independently a reversible terminator; R⁴ is independently a detectable label moiety; and L¹⁰⁰ is a cleavable linker. In embodiments, each nucleotide of the plurality of chase nucleotides has the formula:

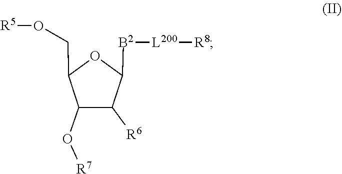

(II); wherein, B² is a nucleobase; R⁵ is a triphosphate or thiotriphosphate; R⁶ is hydrogen or —OH; R⁷ is independently a reversible terminator or hydrogen; R⁸ is independently a retardant moiety; and L²⁰⁰ is a cleavable linker.

In embodiments, the plurality of chase nucleotides all include the same R⁸ moiety. In embodiments, the plurality of chase nucleotides all include the same R⁷ moiety. In embodiments, the plurality of chase nucleotides all include the same L²⁰⁰ moiety.

In embodiments, the first sequencing nucleotide has the formula:

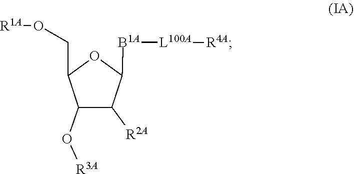

wherein, B^{1A} is a nucleobase; R^{1A} is a triphosphate or thiotriphosphate; R^{2A} is hydrogen or —OH; R^{3A} is the first reversible terminator moiety; R^{4A} is the first detectable label moiety; and L^{100A} is the first cleavable linker. In embodiments, B^{1A} is any value of B¹ as described herein. In embodiments, R^{1A} is any value of R¹ as described herein. In embodiments, R^{2A} is any value of R² as described herein. In embodiments, R^{3A} is any value of R³ as described herein. In embodiments, R^{4A} is any value of R⁴ as described herein. In embodiments, L^{100A} is any value of L¹⁰⁰ as described herein.

In embodiments, the second sequencing nucleotide has the formula:

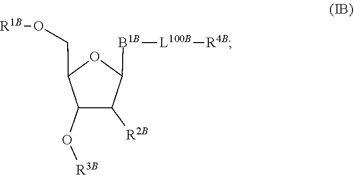

wherein, B^{1B} is a nucleobase; R^{1B} is a triphosphate or thiotriphosphate; R^{2B} is hydrogen or —OH; R^{3B} is the second reversible terminator moiety; R^{4B} is the second detectable label moiety; and L^{100B} is the second cleavable linker. In embodiments, B^{1B} is any value of B¹ as described herein. In embodiments, R^{1B} is any value of R¹ as described herein. In embodiments, R^{2B} is any value of R² as described herein. In embodiments, R^{3B} is any value of R³ as described herein. In embodiments, R^{4B} is any value of R⁴ as described herein. In embodiments, L¹⁰⁰B is any value of L¹⁰⁰ as described herein.

In embodiments, the first chase nucleotide has the formula:

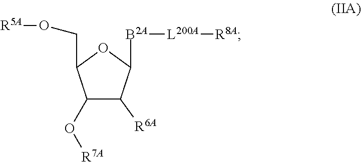

wherein, B^{2A} is a nucleobase; R^{5A} is a triphosphate or thiotriphosphate; R^{6A} is hydrogen or —OH; R^{7A} is the first chase reversible terminator moiety; R^{8A} is the first retarding moiety; and L^{200A} is the first chase cleavable linker. In embodiments, B^{2A} is any value of B² as described herein. In embodiments, R^{5A} is any value of R⁵ as described herein. In embodiments, R^{6A} is any value of R⁶ as described herein. In embodiments, R^{7A} is any value of R⁷ as described herein. In embodiments, R^{8A} is any value of R⁸ as described herein. In embodiments, L^{200A} is any value of L²⁰⁰ as described herein.

In embodiments, the second chase nucleotide has the formula:

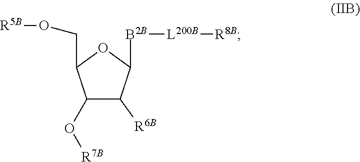

wherein, B^{2B} is a nucleobase; R^{5B} is a triphosphate or thiotriphosphate; R^{6B} is hydrogen or —OH; R^{7B} is the second chase reversible terminator moiety; R^{8B} is the second retarding moiety; and L^{200B} is the second chase cleavable linker. In embodiments, B^{2B} is any value of B² as described herein. In embodiments, R^{5B} is any value of R⁵ as described herein. In embodiments, R^{6B} is any value of R⁶ as described herein. In embodiments, R^{7B} is any value of R⁷ as described herein. In embodiments, R^{8B} is any value of R⁸ as described herein. In embodiments, L^{200B} is any value of L²⁰⁰ as described herein.

In embodiments, the retardant moiety is detectable, wherein the maximum emission of the retardant moiety is less than about 530 nm, less than about 520 nm, or less than about 500 nm.

In embodiments, the retardant moiety is detectable, wherein the maximum emission of the retardant moiety is greater than about 650 nm, greater than about 700 nm, greater than about 750 nm, or greater than about 790 nm.

In embodiments, the retardant moiety is detectable, wherein the maximum emission of the retardant moiety does not overlap with the maximum emission of the detectable label moiety. In embodiments, the maximum emission of the retardant moiety is at least 20, 25, 30, 35, 40, 45, or 50 nm below or above the maximum emission of the detectable label moiety. In embodiments, the maximum emission of the retardant moiety is at least 20, 25, 30, 35, 40, 45, or 50 nm below or above the maximum emission of the detectable label moiety.

In embodiments, the retardant moiety is non-fluorescent. In embodiments, the retardant moiety is a quencher (e.g., a quenching moiety).

In embodiments, the retardant moiety is not detected under conditions used to detect the sequencing nucleotides.

In embodiments, B¹ and B² are each independently a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof. In embodiments, B¹ and B² are each independently

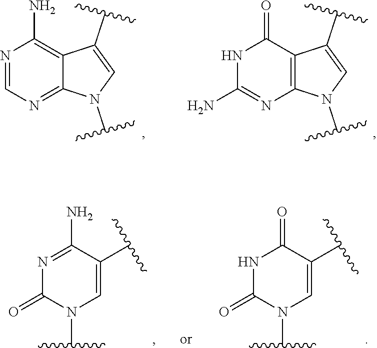

In embodiments, B¹ and B² are each independently

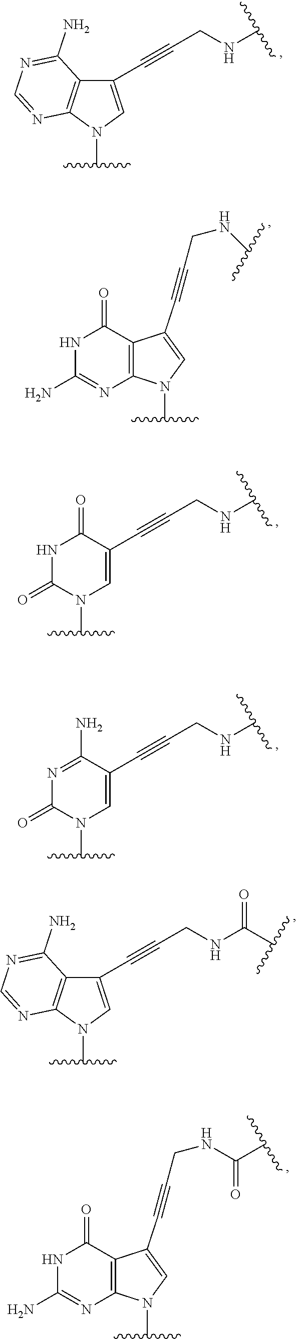 [SMILES: *NCC#Cc1cn(*)c2ncnc(N)c12 ; *NCC#Cc1cn(*)c2nc(N)[nH]c(=O)c12 ; *NCC#Cc1cn(*)c(=O)[nH]c1=O ; *NCC#Cc1cn(*)c(=O)nc1N ; *C(=O)NCC#Cc1cn(*)c2ncnc(N)c12 ; *C(=O)NCC#Cc1cn(*)c2nc(N)[nH]c(=O)c12]

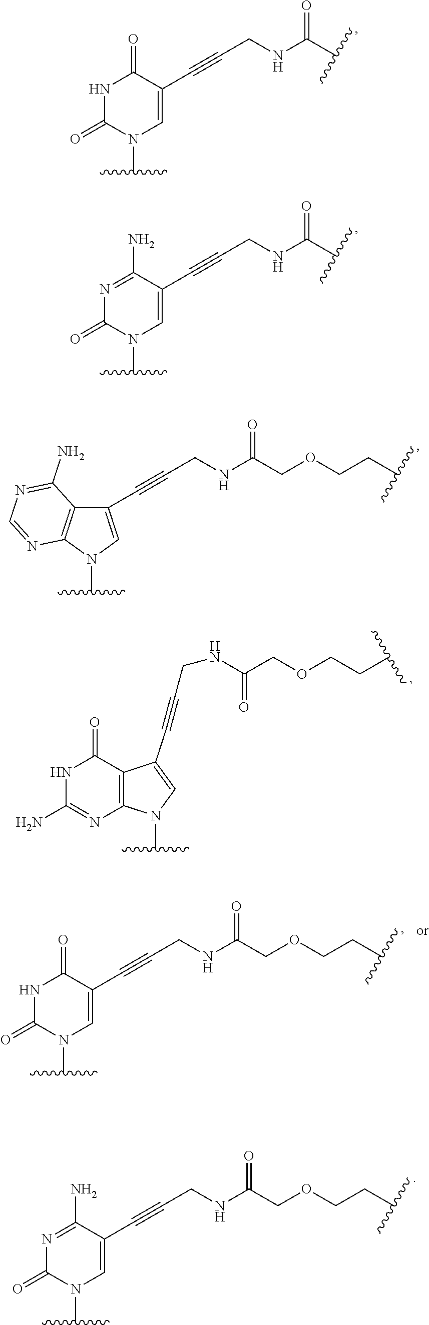 [SMILES: *C(=O)NCC#Cc1cn(*)c(=O)[nH]c1=O ; *C(=O)NCC#Cc1cn(*)c(=O)nc1N ; *CCOCC(=O)NCC#Cc1cn(*)c2ncnc(N)c12 ; *CCOCC(=O)NCC#Cc1cn(*)c2nc(N)[nH]c(=O)c12 ; *CCOCC(=O)NCC#Cc1cn(*)c(=O)[nH]c1=O ; *CCOCC(=O)NCC#Cc1cn(*)c(=O)nc1N]

In embodiments, L¹⁰⁰ and L²⁰⁰ are each independently a cleavable linker including:

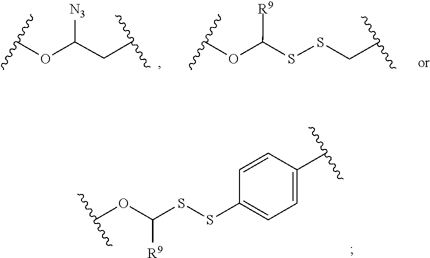

wherein, R⁹ is as described herein, including embodiments.

In embodiments, L¹⁰⁰ and L²⁰⁰ are each independently a cleavable linker including:

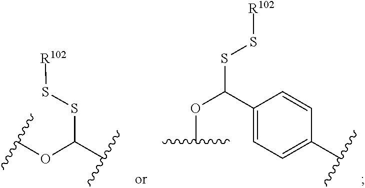

wherein, R¹⁰² is unsubstituted C₁-C₄ alkyl.

### EXAMPLES

### Example 1. Nucleotides Containing Retardant Moieties

In a typical SBS process, many millions to billions of DNA fragments are sequenced in a massively parallel manner. For a given genome, this is accomplished by preparing a sequencing library through random fragmentation of a DNA or cDNA sample followed by 5′ and 3′ adapter ligation. Amplification techniques (e.g., PCR) are then used to amplify the number of DNA molecules in the library, followed by purification. The library is then denatured and loaded into a flow cell where fragments are captured on a lawn of surface-bound oligonucleotides complementary to a portion of the library adapters. Each captured fragment is then amplified through solid-phase amplification techniques (e.g., isothermal bridge amplification) into a distinct, clonal cluster containing thousands of template DNA molecules of identical nucleotide sequence, with the flow cell containing millions to billions of such clusters.

At each step of nucleotide base addition in an SBS cycle, DNA polymerase catalyzes the incorporation of fluorescently labeled, reversibly blocked deoxyribonucleotide triphosphate (dNTP) terminators into growing DNA strands. Nucleotides (e.g., dA, dC, dG, dT, and/or dU) are modified by attaching a unique cleavable fluorophore to the specific location of the nucleobase and capping the 3′-OH group of the nucleotide sugar with a small reversible moiety (also referred to herein as a reversible terminator) so that they are still recognized by DNA polymerase as substrates. The reversible terminator temporarily halts the polymerase reaction after nucleotide incorporation while the fluorophore signal is detected. After incorporation and signal detection, the fluorophore and the reversible terminator are cleaved to resume the polymerase reaction in the next cycle. The emission wavelength and intensity for each cluster are used to identify the particular base added in a given cycle.

The accuracy of a sequencing read depends in part on the cluster of polynucleotides illuminating in unison, that is, where all of the identical templates incorporate the same nucleotide type (e.g., green-labeled dA nucleotides). The intensity of the cluster is directly proportional to the quantity of labeled nucleotides incorporated, so when all of the templates incorporate the same nucleotide type and emit the same fluorescent signal, the sequencing device and corresponding basecalling algorithm is able to confidently assign the identity of the incorporated nucleotide. Maintaining this synchrony is important to allow for accurate and long sequencing reads (i.e., a greater number of consecutive sequencing cycles). For example, at the start of a sequencing reaction, after initial hybridization of the sequencing primer, 100% of the strands within the cluster are synchronized. As the strands are extended, individual strands may fall behind or extend faster than the majority of the strands due to incorporation errors or enzyme stalling. This loss of synchronization is amplified as the number of sequencing rounds increases and eventually, the background noise from the unsynchronized strands becomes too great to accurately call the correct base. Some strands may extend faster when the reversible terminator of the nucleotide to be incorporated is removed prematurely, or the sequencing solution of reversibly terminated nucleotides contains impurities (e.g., natural nucleotides or modified nucleotides bearing a 3′ hydroxyl group), resulting in the clusters of monoclonal amplicons being out-of-phase. Alternatively, some strands may fall behind due to inefficient nucleotide incorporation. As used herein, the term “out-of-phase” or “dephasing” refers to phenomena in sequencing by synthesis that is caused by incomplete removal of the 3′ reversible terminators and fluorophores, and/or failure to complete nucleotide incorporation of a portion of DNA strands within clusters for a given sequencing cycle.

Methods to avoid dephasing include adding a plurality of nucleotides that include a 3′ blocking moiety to fill in any primed templates that were not extended during a given labeled-nucleotide extension (i.e., a sequencing) cycle. While these nucleotides are not detectable, they are typically capable of maintaining phasing within the cluster following each sequencing cycle. However, these nucleotides are susceptible to the same degradation and impurities as sequencing nucleotides. Occasionally during manufacturing and/or storage, the solution of reversibly terminated nucleotides contains impurities (e.g., natural nucleotides or modified nucleotides bearing a 3′ hydroxyl group) or the reversible terminator of the nucleotide is removed prematurely. Without a reversible terminator present on the nucleotide, an additional nucleotide is capable of being incorporated and detected during a sequencing cycle, resulting in dephasing from surrounding amplicons in the cluster. Described herein are nucleotides that include a retardant moiety, such that if the reversible terminator is prematurely removed from the nucleotide, incorporation of the next nucleotide is slowed or halted completely due to the presence of a retardant moiety. Addition of non-detectable nucleotides should help increase overall rate of incorporation while decreasing the rate of misincorporation of sequencing nucleotides in any given sequencing cycle. Further, the non-detectable nucleotides should retain storage stability and cleave at a rate that does not slow the speed of a sequencing cycle.

Initial experiments to assess whether a retardant moiety on a nucleotide without a reversible terminator (RT) slows down the incorporation of a subsequent nucleotide addition into a growing DNA strand were conducted. A reversible terminated nucleotide, and nucleotide containing a retardant moiety (but not a reversible terminator) is incorporated into a primer hybridized to a template at 55° C. The reversible terminator is cleaved, and a solution of labeled nucleotides is added to the primed templates. Measuring the label at different time points allows one to calculate a halftime of incorporation. The average halftime of the next nucleotide to be incorporated was orders of magnitude larger (e.g., the average halftime for all four nucleotides was measured to be about 13.5 minutes, or about 810 seconds) for the nucleotide containing a retardant moiety and no 3′ reversible terminator. In contrast, a nucleotide without a retardant moiety has an incorporation halftime of about 15-30 seconds under the same experimental conditions. The retardant slows incorporation of the next nucleotide to be incorporated, even in the absence of a reversible terminator on the nucleotide.

Additional experimentation was performed to determine the effect having a retarding moiety had upon each type of nucleobase incorporation during sequencing. Incorporation half times were measured for chase nucleotide with different nucleobases (adenine (A), guanine (G), cytosine (C), and thymine (T)), each having a different retarding moiety (VT1, VT2, VT3, VT4, or VT5) connected to the nucleobase via a cleavable linker. The incorporation score is a direct reflection of the incorporation half time; +++ refers to 4 to 14 seconds, ++ refers to 15 to 25 seconds, and + refers to greater than 25 seconds. The next base incorporation score reflects the incorporation half time for the next base, C refers to 20 to 100 seconds, B refers to 101 to 200 seconds, and A refers to greater than 200 seconds. The structure of the retarding moiety affects the rate of incorporation for different nucleobases, for example VT1 varies between ++ for incorporating G and A and has a score of +++ when incorporating C and T. For this assay, the chase nucleotides did not have a 3′-reversible terminator moiety (i.e., the nucleotides used in this assay have a retarding moiety attached with a cleavable linker and possess a 3′-OH). The kinetics of the next base to be incorporated, following successful incorporation of the chase nucleotide demonstrates the retardant effect of the retarding moiety. Incorporation of a nucleotide having a retarding moiety resulted in a significant increase in the incorporation halftime of the next nucleotide. This effect is readily observed in Table 2, wherein the next base incorporation halftime, which is typically about 5-15 seconds under these experimental conditions, is 10 to 20 times slower.

The structures of the VT compounds are as follows: VT1 is

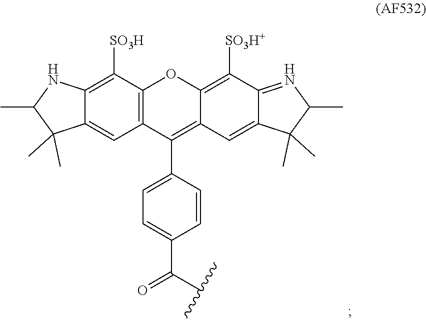

### VT2 is

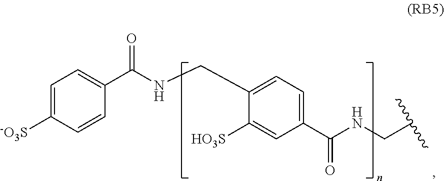

wherein n is 4; VT3 is

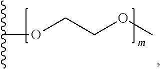

wherein m is 24 (PEG24); VT4 is

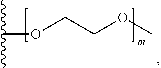

wherein m is 12 (PEG12); and VT5 is

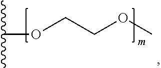

wherein m is 4 (PEG4), wherein the

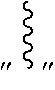

represents the attachment point to the cleavable linker L²⁰⁰. Additional retarding moieties tested include

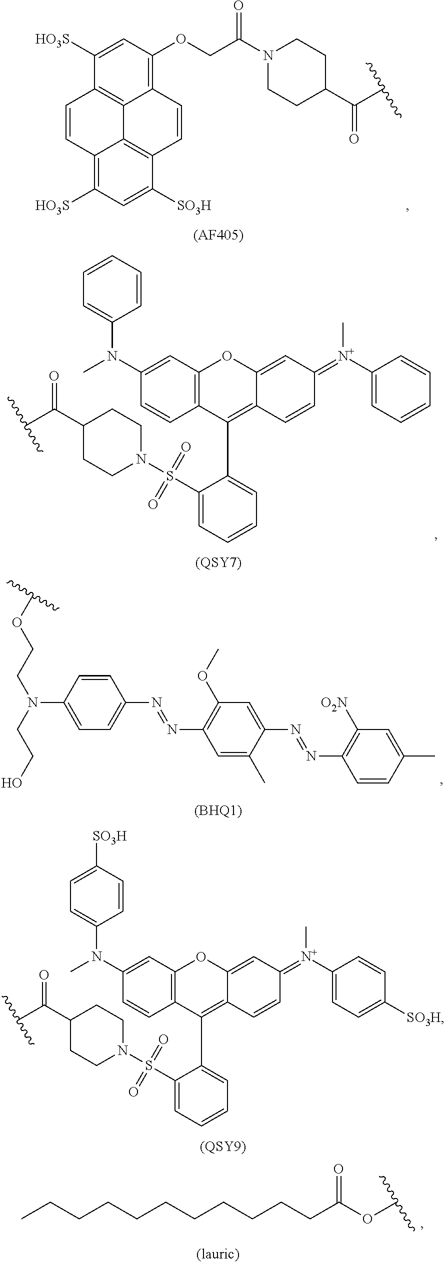

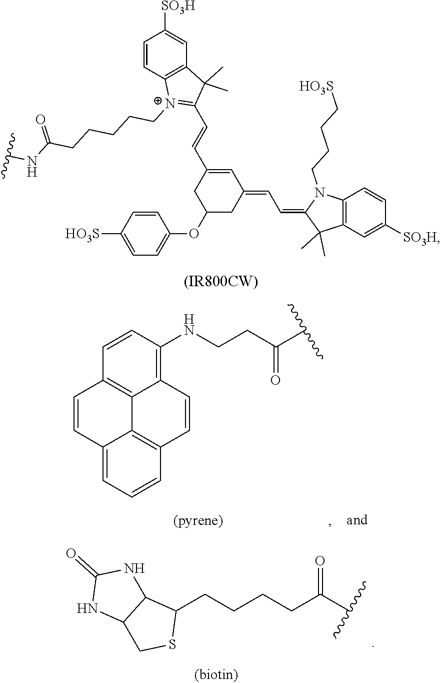 [SMILES: *NC(=O)CCCCC[N+]1=C(/C=C/C2=CC(=C/C=C3\N(CCCCS(=O)(=O)O)c4ccc(S(=O)(=O)O)cc4C3(C)C)/CC(Oc3ccc(S(=O)(=O)O)cc3)C2)C(C)(C)c2cc(S(=O)(=O)O)ccc21 ; *C(=O)CCNc1ccc2ccc3cccc4ccc1c2c34 ; *C(=O)CCCCC1SCC2NC(=O)NC21]

The biotin moiety was further reacted with a labeled streptavidin to further confirm incorporation. Despite being relatively smaller than typical fluorescent dyes (e.g., a rhodamine dye

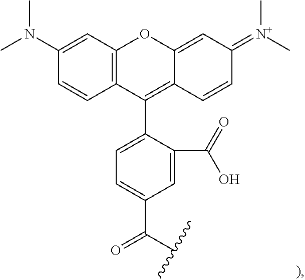

biotin had an incorporation score of +(i.e., incorporation halftime of 28 seconds relative to an incorporation halftime of 7 seconds for a rhodamine dye under the same experimental conditions). Additionally, following cleavage of the linker, the linker remnant containing the biotin and biotin-streptavidin complex was found to nonspecifically bind to additional components within the reaction vessel, resulting in a significant background signal that persisted for greater than 10 minutes. These non-specific interactions present downstream complications in sequencing reactions as signals may be difficult to detect from the surrounding background when the signal to noise ratio is too low. Reducing the formation of reactive groups and non-specific binding events becomes more important as in situ sequencing approaches (i.e., sequencing one or more nucleic acid molecule within a cell) are considered. Within a cell, many different types of proteins (e.g., antibodies, receptors, organelles, hormones and enzymes) often contain bioconjugate reactive moieties capable of covalently or non-covalently binding with cleaved linkers. Therefore, a retarding moiety with minimal reactivity is preferred. Without wishing to be bound by any theory, the tetrahydrothiophene portion of the biotin may react non-preferably with thiol moieties remaining following cleavage of disulfide bonds (e.g., cleaving disulfide containing cleavable linkers), or with the disulfide linkers themselves, which results in fouling and premature cleavage of the linker and/or a disulfide containing reversible terminator moiety of another labeled modified nucleotide. This premature cleavage of sequencing nucleotides (e.g., removing the reversible terminator and/or the dye) results in asynchronous shifts in sequencing runs that are detrimental to sequencing accuracy. Further complications may include out-of-phase clusters of monoclonal amplicons, reduced sequencing accuracy and limited sequencing read lengths.

In order to obtain long read lengths, there needs to be an effective solution to the synchrony problems in ensemble-based SBS. One such phase loss effect relates to an “incomplete extension” (IE) event or error (also referred to herein as a “lag error”). An IE event may occur during a sequencing reaction, when one or more nucleotide species fails to incorporate into one or more nascent extension strand(s) during a given extension round of the sequencing cycle. This may result in that particular extension strand being out of position relative to rest of the population of extension strands (e.g., certain template extension strands lack a nucleotide and fall behind the main template population). IE events may arise, for example, due of a lack of nucleotide availability to a portion of the template/polymerase complexes of a population. Alternatively, or in addition, IE events may be caused by a defective or absent polymerase, or an incorporated nucleotide that does not have a free 3′ OH available (e.g., retains a reversible terminator) for nucleotide polymerization. Another such phase loss effect relates to a “carry forward” (CF) event or error (also referred to herein as a “lead error”). A CF event may occur as a result of an improper additional extension of a nascent strand by incorporation of one or more nucleotide species into a sequencing strand position that is ahead and thus out of phase with the sequencing strand position of the rest of the population. CF events may arise, for example, because of the misincorporation of a nucleotide species, or in certain instances, due to contamination from free nucleotides remaining from a previous cycle (e.g., which may result from an insufficient or incomplete washing of the reaction chamber). For example, a small fraction of a “dT” nucleotide cycle may be present or carry forward to a “dC” nucleotide cycle. The presence of both nucleotides may lead to an undesirable extension of a fraction of the growing strands where the “dT” nucleotide is incorporated in addition to the “dC” nucleotide such that multiple different nucleotide incorporations events take place where only a single type of nucleotide incorporation would normally be expected. Alternatively, some strands may extend faster when the reversible terminator of the nucleotide to be incorporated is not present. Errors or phasing issues related to IE and CF events (alternatively referred to as phasing and/or prephasing errors) may be exacerbated over time because of the accumulation of such events, causing degradation of sequence signal or sequence quality over time and an overall reduction in the practical read length of the system (e.g., the number of nucleotides that can be sequenced for a given template). The present disclosure provides improvement of sequencing performance (e.g., efficiency and/or accuracy of sequencing) by utilizing the methods and compositions as described herein.

TABLE 2
Kinetic effects of chase nucleotides on different nucleobases. The
incorporation score is a direct reflection of the incorporation half time; +++ refers to 4 to 14
seconds, ++ refers to 15 to 25 seconds, and + refers to greater than 25 seconds. The next base
incorporation score reflects the incorporation half time for the next base, C refers to 20 to 100
seconds, B refers to 101 to 200 seconds, and A refers to greater than 200 seconds.

| Nucleobase | Retarding Moiety | Incorporation score | Next base incorporation score |
|---|---|---|---|
| G | Control | +++ | |
| | VT5 | +++ | B |
| | VT4 | +++ | |
| | VT3 | ++ | B |
| | VT2 | +++ | A |
| | VT1 | ++ | A |
| A | Control | +++ | |
| | VT5 | +++ | C |
| | VT4 | +++ | C |
| | VT3 | + | C |
| | VT2 | ++ | |
| | VT1 | ++ | A |
| T | Control | +++ | |
| | VT5 | ++ | B |
| | VT4 | ++ | B |
| | VT3 | + | A |
| | VT2 | +++ | B |
| | VT1 | +++ | A |
| C | Control | +++ | |
| | VT5 | +++ | B |
| | VT4 | +++ | A |
| | VT3 | ++ | A |
| | VT2 | +++ | |
| | VT1 | +++ | A |

In embodiments, the chase nucleotides as described herein are similar in structure to labeled sequencing nucleotides (e.g., nucleotides containing a reversible-terminator and a cleavable linker-linked dye, such as those depicted in Formula I), except that these chase nucleotides include a retardant moiety rather than a detectable label at the corresponding position (see for example Formula II which includes R⁸ as a retardant moiety). The inclusion of the retardant moiety creates a redundancy by doubly-terminating the nucleotide, thereby slowing down the incorporation of subsequent nucleotides and reducing the lead percent and phasing errors during sequencing runs. That is, in embodiments, the chase nucleotide includes a first terminator (e.g., a 3Y-reversible terminator) and a second terminator (e.g., a nucleobase-linked dye). A doubly-terminated nucleotide is useful if during storage the reversible terminator or cleavable linker prematurely degrades, another terminator is present. For example, if the nucleotides experience 1% degradation of the reversible terminator or the cleavable linker during storage, the solution would have about 1% loss of the 3′ terminator, about 1% loss of the linker, and about 0.01% loss of both on the same molecule.

In embodiments, the retarding moiety of the chase nucleotides is not a bioconjugate reactive moiety. In embodiments, the retarding moiety of the chase nucleotides is not an anchor moiety capable of interacting (e.g., covalently or non-covalently) with a second, optionally different, chemical moiety (e.g., a complementary anchor moiety binder). The anchor moiety is a bioconjugate reactive group capable of interacting (e.g., covalently) with a complementary bioconjugate reactive group (e.g., complementary anchor moiety reactive group). In embodiments, an anchor moiety is a click chemistry reactant moiety. In embodiments, the anchor moiety (an “affinity anchor moiety”) is capable of non-covalently interacting with a second chemical moiety (e.g., complementary affinity anchor moiety binder). Non-limiting examples of an anchor moiety include biotin, azide, trans-cyclooctene (TCO) and phenyl boric acid (PBA). In embodiments, an affinity anchor moiety (e.g., biotin moiety) interacts non-covalently with a complementary affinity anchor moiety binder (e.g., streptavidin moiety). In embodiments, an anchor moiety (e.g., azide moiety, trans-cyclooctene (TCO) moiety, phenyl boric acid (PBA) moiety) covalently binds a complementary anchor moiety binder (e.g., dibenzocyclooctyne (DBCO) moiety, tetrazine (TZ) moiety, salicylhydroxamic acid (SHA) moiety). In embodiments, the retarding moiety is not an anchor moiety. In embodiments, the retarding moiety is not capable of forming a bioconjugate linker.

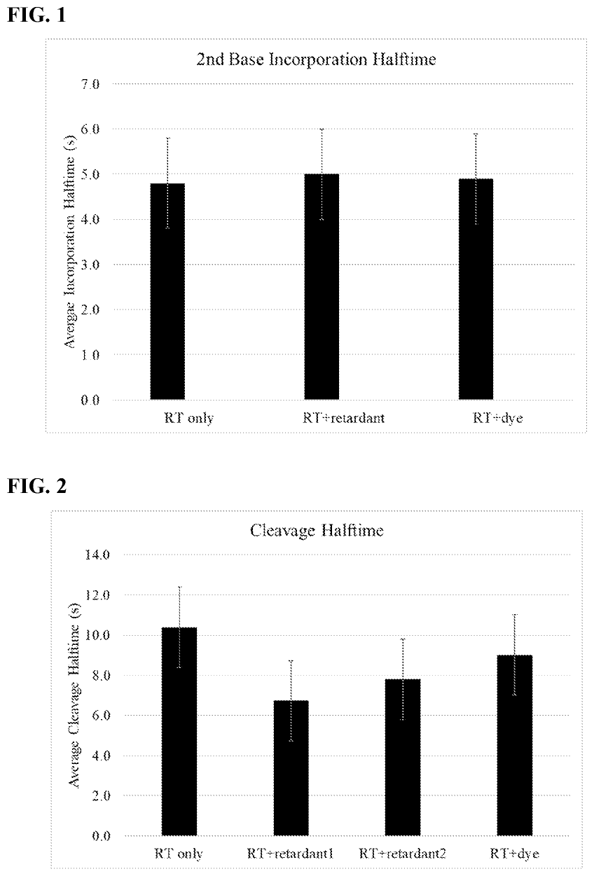

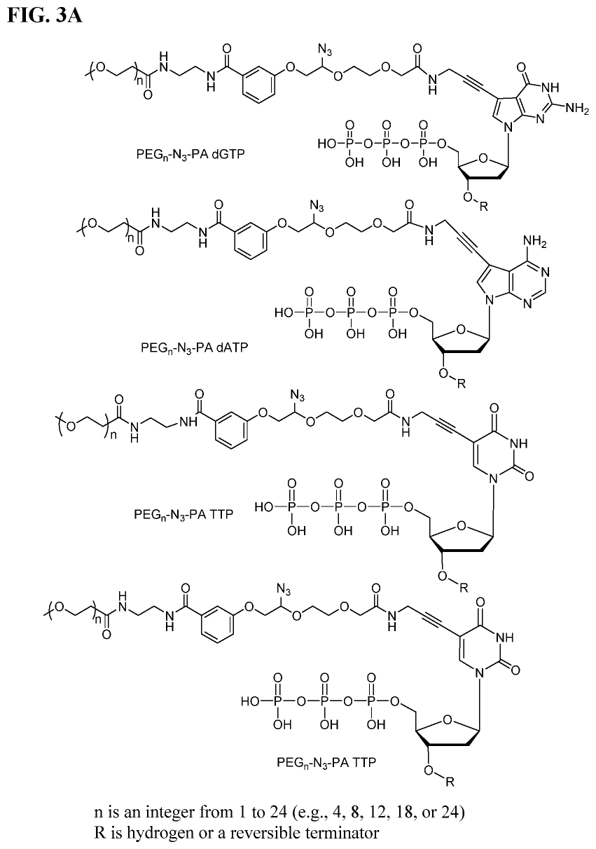

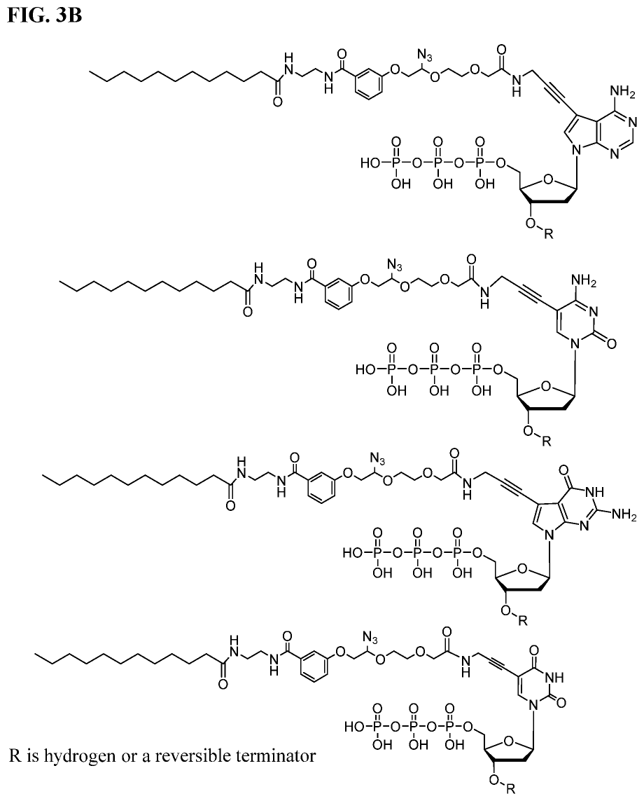

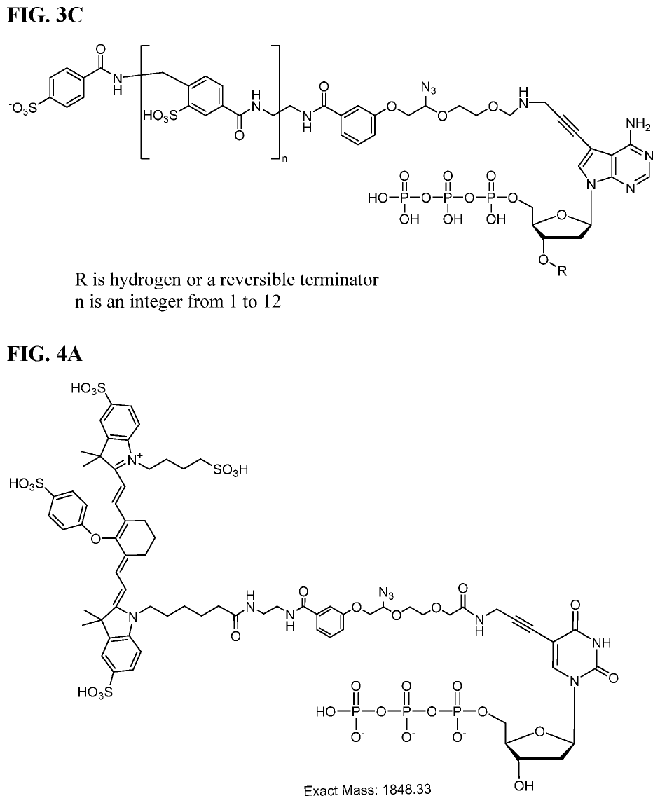

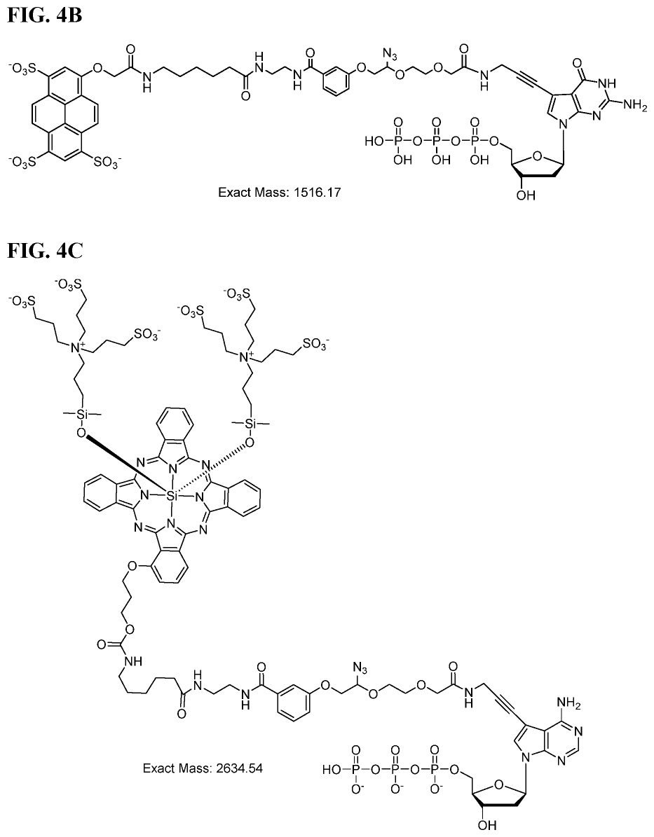

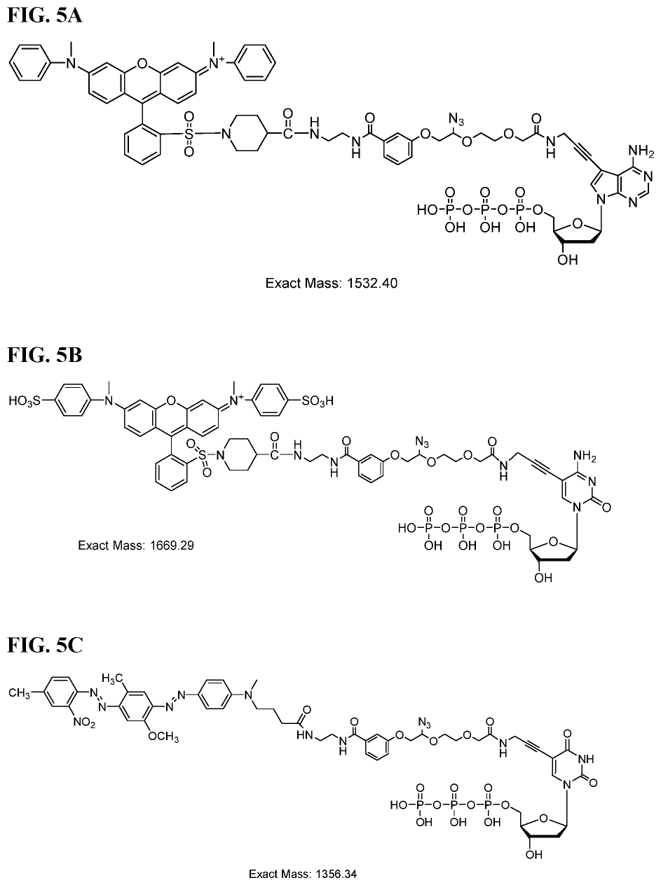

Additional experiments were performed to assess whether i) doubly-terminated nucleotides (i.e., a retardant moiety on a nucleotide containing a 3′ reversible terminator (RT)) performs comparably to a nucleotide bearing a ii) 3′ reversible terminator or iii) a nucleotide containing both a 3′ reversible terminator and a dye. The same cleavable linker is used to link the retardant moiety in i) as is used to link the dye in iii). The 2^{nd} base incorporation halftimes are reported in FIG. 1. Briefly, a 3′-reversible terminated nucleotide, nucleotide containing 3′-reversible terminator and a dye, and nucleotide containing a 3′-reversible terminator and a retardant moiety are incorporated into a primed template at 65° C. The cleavable linker and the reversible terminator are cleaved and a solution of labeled nucleotides is added to the primed templates. The average halftime is quantified and suggests the retardant moiety (e.g., RT+retardant) does not impact subsequent base incorporation, even in the absence of a reversible terminator (RT) compared to a nucleotide containing only a reversible terminator (e.g., RT-only) or a reversibly terminated chase nucleotide containing a detectable moiety (e.g., RT+dye).

An important property of a reversible terminator on a nucleotide is that it can be rapidly cleaved under conditions that do not adversely affect the DNA (i.e., mild conditions) so the next nucleotide may be incorporated. FIG. 2 reports the cleavage halftime rates for different 3′-reversible terminated (RT) nucleotides. To calculate the cleavage half time, each nucleotide was incorporated into a growing DNA strand immobilized on a solid support. Excess nucleotides were washed away. Next, a cleavage solution containing THPP as a reducing agent was introduced for controlled periods of time. The cleavage reaction was carried out at 55° C., in a buffered solution at elevated pH. The results indicate that chase nucleotides containing a retardant moiety (e.g., RT+retardant1 or RT+retardant2) are cleaved at approximately the same rate as the nucleotides containing only a reversible terminator (e.g., RT only) or a reversibly terminated chase nucleotide containing a detectable moiety (e.g., RT+dye). The same cleavable linker is used to link the retardant moiety as is used to link the dye. The cleavage halftime may be further optimized by modifying the reaction conditions (e.g., elevating temperature to 65° C., increasing the concentration of the reducing agent, or a combination thereof). Retardant moiety 1 has the formula:

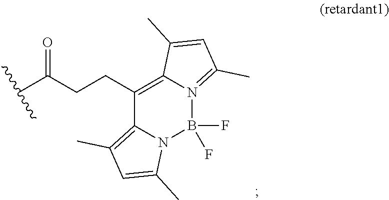

Retardant moiety 2 has the formula

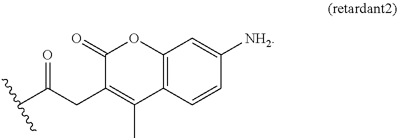 [SMILES: *C(=O)Cc1c(C)c2ccc(N)cc2oc1=O]

In embodiments, the retardant moieties are detectable which can serve as an additional quality control check to determine how many sequencing nucleotides in a cluster were not incorporated.

In embodiments, the retardant moiety is fluorescent (e.g., blue), however the emission maximum is outside the detectable channels used for sequencing (e.g., green, yellow, orange, red). For example, the retardant moiety may include a cyanine, rhodamine, 4,4-difluoro-4-bora-3a,4a-diaza-s-indacene (BODIPY), squaraine, phthalocyanine, or porphyrin derivatives provided the emission wavelength does not interfere with detection of the sequencing nucleotides. Chemical substitutions to the core can shift the emission wavelength, for example adding dicyanovinyls to squaraine moiety enhances NIR fluorescence properties. For example, the retardant moiety may be detectable, wherein the emission maximum is outside the range of detection for the sequencing nucleotides, which is typically about 530 nm to about 750 nm for four color sequencing or about 520 nm to about 660 nm for two color sequencing.

In embodiments, the retardant moiety is non-fluorescent. In embodiments, the retardant moiety is a quencher. The quencher may provide an additional benefit by quenching (i.e., absorbing) any remaining fluorescence before the next sequencing cycle. For example, following incorporation and detection of a labeled sequencing nucleotide, a chase nucleotide containing a quencher moiety is introduced and incorporated to any available primed templates (i.e., a primed template with a free 3′-OH). The chase nucleotide containing a quencher may absorb and decrease the fluorescent intensity of any long-lived fluorescent states such that when the next sequencing cycle is initiated the primed templates are all dark by reducing any background fluorescence.

Experimental data show that using the chase nucleotides as described herein provides comparable sequencing accuracy and percent perfect reads in sequencing runs compared to using a nucleotide mixture comprised of nucleotides having a 3′-reversible terminator with no retarding moiety linked to the nucleotide. Further chase nucleotides have comparable lag (% terminators that fall back or fail to advance during a cycle of sequencing), lead (% terminators that leap ahead or over-incorporate during a cycle of sequencing) compared to chase terminators having a 3′-reversible terminator and no retarding moiety linked to the nucleotide. In a sequencing run of Salmonella samples, sequencing with sequencing nucleotides following by incubation with chase nucleotides as described herein have shown up to >99.85% sequencing accuracy.

Provided herein is a modified nucleotide for use in sequencing which comprises both a reversible terminator and a retardant moiety attached to the base, wherein the retardant moiety acts as a secondary terminator. This modified nucleotide may be useful in reducing lead dephasing. Accordingly, the discovery of chase terminators which decrease the incidence of phasing errors provides a great advantage in SBS applications over existing chase nucleotides. For example, the chase nucleotides described herein result in lower out-of-phase values and permit longer sequencing read lengths.

### EMBODIMENTS

The present disclosure provides the following additional illustrative embodiments.

Embodiment P-1. A method of extending a primer, said method comprising: contacting a primer hybridized to a template polynucleotide with a sequencing solution, followed by contacting the primer with a chase solution; and in the presence of a polymerase, incorporating a nucleotide from the sequencing solution or incorporating a nucleotide from the chase solution to extend the primer; wherein (a) the sequencing solution comprises a plurality of sequencing nucleotides, (b) each nucleotide of the plurality of sequencing nucleotides comprises a detectable label moiety and a first reversible terminator moiety; (c) the chase solution comprises a plurality of chase nucleotides, (d) each nucleotide of the plurality of chase nucleotides comprising a retardant moiety and a second reversible terminator moiety, and (e) the retardant moieties differ in structure from the detectable label moieties.

Embodiment P-2. The method of Embodiment P-1, wherein each nucleotide of the plurality of sequencing nucleotides has the formula:

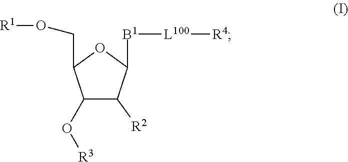

wherein B¹ is a nucleobase; R¹ is a triphosphate or thiotriphosphate; R² is hydrogen or —OH; R³ is independently a reversible terminator; R⁴ is independently a detectable label moiety; and L¹⁰⁰ is a cleavable linker;
and wherein each nucleotide of the plurality of chase nucleotides has the formula:

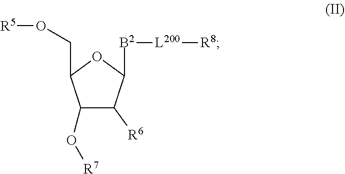

wherein, B² is a nucleobase; R⁵ is a triphosphate or thiotriphosphate; R⁶ is hydrogen or —OH; R⁷ is independently a reversible terminator; R⁸ is independently a retardant moiety; and L²⁰⁰ is a cleavable linker.

Embodiment P-3. The method of Embodiment P-1, wherein the retardant moiety is detectable, wherein the maximum emission of the retardant moiety is less than about 530 nm, less than about 520 nm, or less than about 500 nm.

Embodiment P-4. The method of Embodiment P-1, wherein the retardant moiety is detectable, wherein the maximum emission of the retardant moiety is greater than about 650 nm, greater than about 700 nm, greater than about 750 nm, or greater than about 790 nm.

Embodiment P-5. The method of Embodiment P-1, wherein the retardant moiety is detectable, and wherein the maximum emission of the retardant moiety does not overlap with the maximum emission of the detectable label moiety.

Embodiment P-6. The method of Embodiment P-5, wherein the maximum emission of the retardant moiety is at least 20 nm below or above the maximum emission of the detectable label moiety.

Embodiment P-7. The method of Embodiment P-1, wherein the retardant moiety is non-fluorescent.

Embodiment P-8. The method of Embodiment P-7, wherein the retardant moiety is a quencher.

Embodiment P-9. The method of any one of Embodiments P-2 to P-8, wherein B¹ and B² are each independently a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

Embodiment P-10. The method of any one of Embodiments P-2 to P-8, wherein B¹ and B² are each independently

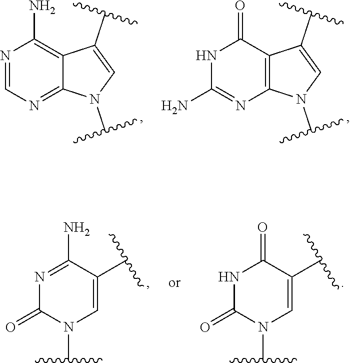 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

Embodiment P-11. The method of any one of Embodiments P-2 to P-8, wherein B¹ and B² are each independently

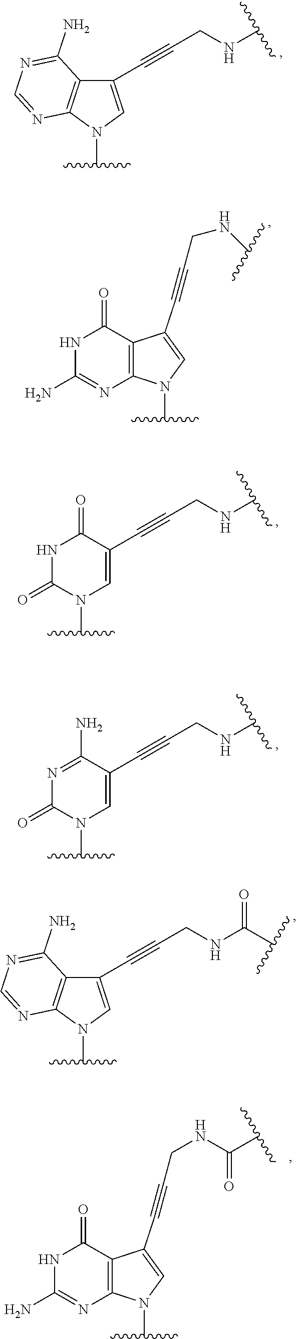 [SMILES: *NCC#Cc1cn(*)c2ncnc(N)c12 ; *NCC#Cc1cn(*)c2nc(N)[nH]c(=O)c12 ; *NCC#Cc1cn(*)c(=O)[nH]c1=O ; *NCC#Cc1cn(*)c(=O)nc1N ; *C(=O)NCC#Cc1cn(*)c2ncnc(N)c12 ; *C(=O)NCC#Cc1cn(*)c2nc(N)[nH]c(=O)c12]

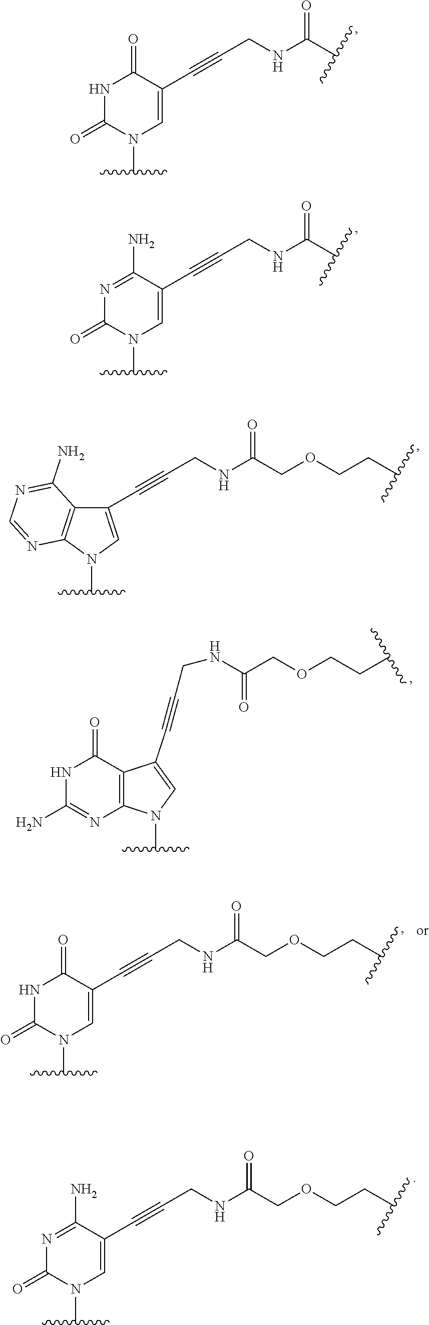 [SMILES: *C(=O)NCC#Cc1cn(*)c(=O)[nH]c1=O ; *C(=O)NCC#Cc1cn(*)c(=O)nc1N ; *CCOCC(=O)NCC#Cc1cn(*)c2ncnc(N)c12 ; *CCOCC(=O)NCC#Cc1cn(*)c2nc(N)[nH]c(=O)c12 ; *CCOCC(=O)NCC#Cc1cn(*)c(=O)[nH]c1=O ; *CCOCC(=O)NCC#Cc1cn(*)c(=O)nc1N]

Embodiment P-12. The method of any one of Embodiments P-2 to P-11, wherein L¹⁰⁰ and L²⁰⁰ are each independently a cleavable linker comprising:

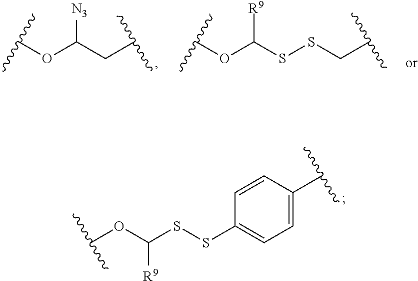

wherein, R⁹ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P-13. The method of any one of Embodiments P-2 to P-11, wherein L¹⁰⁰ and L²⁰⁰ are each independently a cleavable linker comprising:

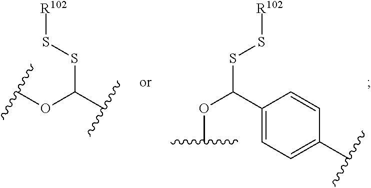

wherein R¹⁰² is unsubstituted C₁-C₄ alkyl.

Embodiment P-14. The method of any one of Embodiments P-1 to P-13, further comprising detecting the detectable label moiety i) prior to contacting the primer with the chase solution, or ii) after contacting the primer with the chase solution.

Embodiment P-15. The method of any one of Embodiments P-1 to P-13, further comprising detecting the detectable label moiety during contacting of the primer with the chase solution.

Embodiment P-16. The method of any one of Embodiments P-1 to P-15, further comprising removing (a) the first or second reversible terminator moiety, and (b) the detectable label moiety or the retardant moiety.

Embodiment P-17. The method of any one of Embodiments P-1 to P-16, further comprising repeating contacting the extended primer with the sequencing solution, followed by contacting the extended primer with the chase solution.

Embodiment P-18. A method of sequencing a plurality of template polynucleotides, said method comprising: (a) contacting a plurality of primers hybridized to template polynucleotides with a chase solution in the presence of a polymerase; wherein a fraction of the plurality of primers comprise a 3′ terminal nucleotide comprising a first detectable label moiety and a first reversible terminator moiety; wherein the chase solution comprises a plurality of chase nucleotides, each nucleotide in the plurality of chase nucleotides comprising a retardant moiety and a second reversible terminator moiety; (b) detecting the first detectable label moiety of the 3′ terminal nucleotide; (c) removing the first detectable label moiety, the retardant moiety, and the first and second reversible terminator moieties from nucleotides of the plurality of primers; (d) contacting the plurality of primers hybridized to template polynucleotides with a sequencing solution, wherein the sequencing solution comprises a plurality of sequencing nucleotides, each nucleotide of the plurality of sequencing nucleotides comprising a second detectable label moiety and a third reversible terminator moiety; and wherein a fraction of the plurality of primers incorporate a nucleotide of the plurality of sequencing nucleotides; and (e) repeating steps (a)-(d) thereby sequencing the template polynucleotides.

Embodiment P-19. The method of Embodiment P-18, wherein following incorporation of one of the plurality of chase nucleotides by primer extension, the incorporation rate of a subsequent nucleotide is decreased.

Embodiment P-20. The method of Embodiment P-18, wherein following incorporation of one of the plurality of chase nucleotides by primer extension, the incorporation rate of a subsequent nucleotide is decreased by a factor of about 3 to about 10.

Embodiment P-21. A method of sequencing a plurality of template polynucleotides, said method comprising: i) contacting a substrate comprising a plurality of immobilized template polynucleotides with a sequencing solution comprising a plurality of sequencing nucleotides, each nucleotide of the plurality of sequencing nucleotides comprising a detectable label moiety and a first reversible terminator moiety, wherein each immobilized template polynucleotide includes one or more primers hybridized thereto; and in the presence of a polymerase, extending the one or more primers with a nucleotide to generate extended primers; ii) contacting the substrate with a chase solution comprising a plurality of chase nucleotides, each nucleotide of the plurality of chase nucleotides comprising a retardant moiety and a second reversible terminator moiety; iii) detecting the detectable label moiety so as to identify one or more nucleotides incorporated into the extended primers; iv) removing the first and second reversible terminator moieties, the detectable label moiety, and the retardant moiety; and v) repeating steps i) to iv) to sequence the plurality of immobilized template polynucleotides.

Embodiment P-22. The method of Embodiment P-21, further comprising detecting the retardant moiety prior to step iv).

Embodiment P-23. A method of detecting templates in a cluster, said method comprising: (a) contacting a cluster comprising a plurality of templates with a plurality of chase nucleotides in the presence of a polymerase, each nucleotide of the plurality of chase nucleotides comprising a retardant moiety and a reversible terminator moiety; wherein a fraction of the plurality of templates in the cluster comprise reversibly-terminated, labeled nucleotides incorporated at the 3′ ends of primers hybridized to the fraction of the plurality of templates; and (b) detecting one or more of the retardant moieties incorporated by primer extension, thereby detecting templates.

Embodiment P-24. The method of Embodiment P-23, further comprising detecting the labeled nucleotides.

Embodiment P-25. The method of Embodiments P-23 or P-24, further comprising removing the reversible terminator moiety, a label of the labeled nucleotides, and the retardant moiety.

Embodiment P-26. The method of any one of Embodiments P-18 to P-22, wherein each nucleotide of the plurality of sequencing nucleotides has the formula:

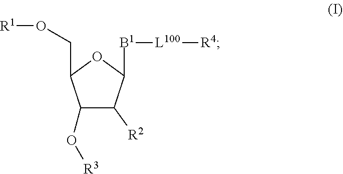

and each nucleotide of the plurality of chase nucleotides has the formula:

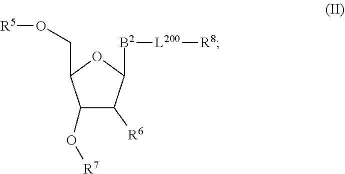

wherein B¹ and B² are each independently a nucleobase; R¹ and R⁵ are each independently a triphosphate or thiotriphosphate; R² and R⁶ are each independently hydrogen or —OH; R³ and R⁷ are each independently a reversible terminator; R⁴ is independently a detectable moiety; R⁸ is independently a retardant moiety; and L¹⁰⁰ and L²⁰⁰ are each independently a cleavable linker.

Embodiment P-27. The method of Embodiment P-26, wherein the plurality of chase nucleotides all comprise the same R⁸ moiety.

Embodiment P-28. A kit comprising a sequencing solution and a chase solution, wherein (a) the sequencing solution comprises a plurality of sequencing nucleotides, (b) each nucleotide of the plurality of sequencing nucleotides comprise a detectable label moiety and a first reversible terminator moiety; (c) the chase solution comprises a plurality of chase nucleotides, (d) each nucleotide of the plurality of chase nucleotides comprises a retardant moiety and a second reversible terminator moiety, and (e) the retardant moieties differ in structure from the detectable label moieties.

Embodiment P-29. The kit of Embodiment P-28, wherein each nucleotide of the plurality of sequencing nucleotides has the formula:

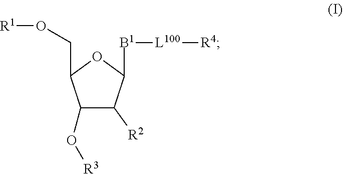

and each nucleotide of the plurality of chase nucleotides, has the formula:

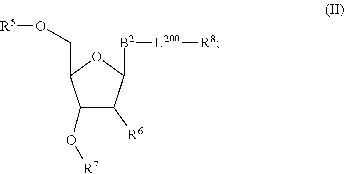

wherein, B¹ and B² are each independently a nucleobase; R¹ and R⁵ are each independently a triphosphate or thiotriphosphate; R² and R⁶ are each independently hydrogen or —OH; R³ and R⁷ are each independently a reversible terminator; R⁴ is independently a detectable moiety; R⁸ is independently a retardant moiety; and L¹⁰⁰ and L²⁰⁰ are each independently a cleavable linker.

Embodiment P-30. The kit of Embodiment P-28, wherein the retardant moiety is detectable, wherein the maximum emission of the retardant moiety is less than about 530 nm, less than about 520 nm, or less than about 500 nm.

Embodiment P-31. The kit of Embodiment P-28, wherein the retardant moiety is detectable, and wherein the maximum emission of the retardant moiety is greater than about 650 nm, greater than about 700 nm, greater than about 750 nm, or greater than about 790 nm.

Embodiment P-32. The kit of Embodiment P-28, wherein the retardant moiety is detectable, and wherein the maximum emission of the retardant moiety does not overlap with the maximum emission of the detectable label moiety.

Embodiment P-33. The kit of Embodiment P-32, wherein the maximum emission of the retardant moiety is at least 20 nm below or above the maximum emission of the detectable label moiety.

Embodiment P-34. The kit of Embodiment P-29, wherein R⁸ is

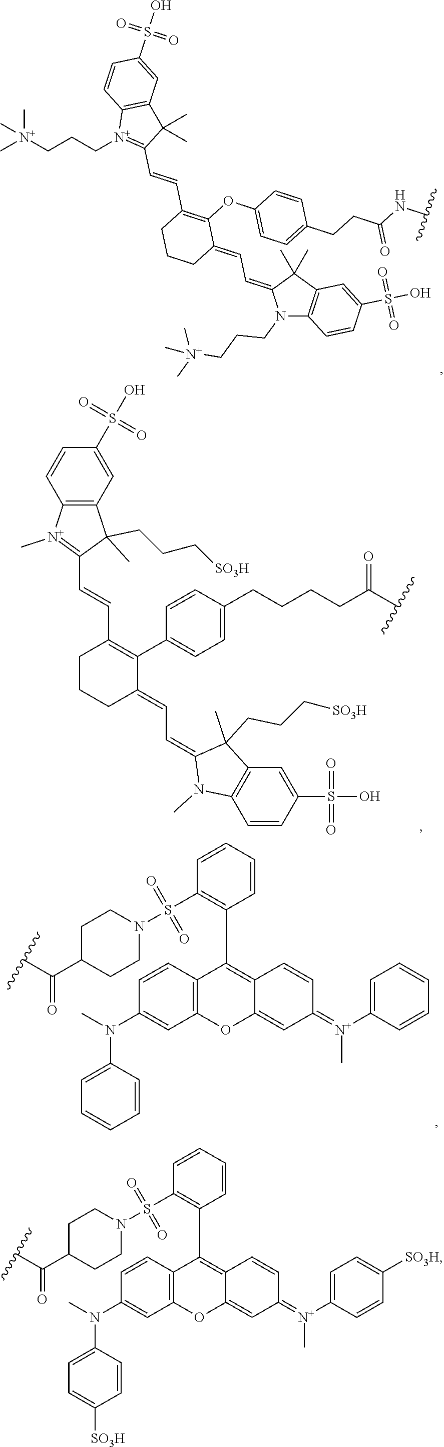 [SMILES: *NC(=O)CCc1ccc(OC2=C(/C=C/C3=[N+](CCC[N+](C)(C)C)c4ccc(S(=O)(=O)O)cc4C3(C)C)CCC/C2=C\C=C2\N(CCC[N+](C)(C)C)c3ccc(S(=O)(=O)O)cc3C2(C)C)cc1 ; *C(=O)CCCCc1ccc(C2=C(/C=C/C3=[N+](C)c4ccc(S(=O)(=O)O)cc4C3(C)CCCS(=O)(=O)O)CCC/C2=C\C=C2\N(C)c3ccc(S(=O)(=O)O)cc3C2(C)CCCS(=O)(=O)O)cc1 ; *C(=O)C1CCN(S(=O)(=O)c2ccccc2-c2c3cc/c(=[N+](/C)c4ccccc4)cc-3oc3cc(N(C)c4ccccc4)ccc23)CC1 ; *C(=O)C1CCN(S(=O)(=O)c2ccccc2-c2c3cc/c(=[N+](/C)c4ccc(S(=O)(=O)O)cc4)cc-3oc3cc(N(C)c4ccc(S(=O)(=O)O)cc4)ccc23)CC1]

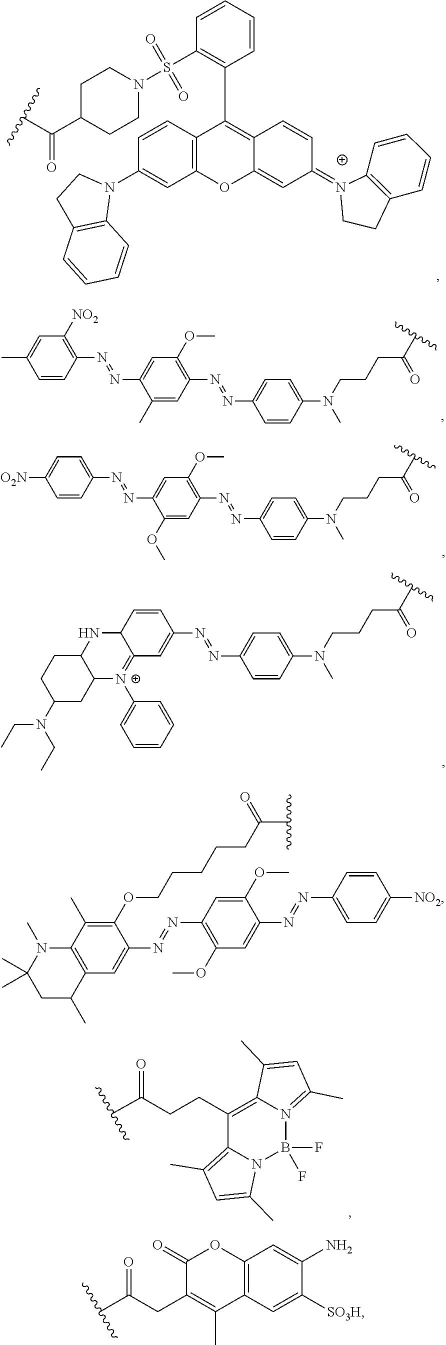

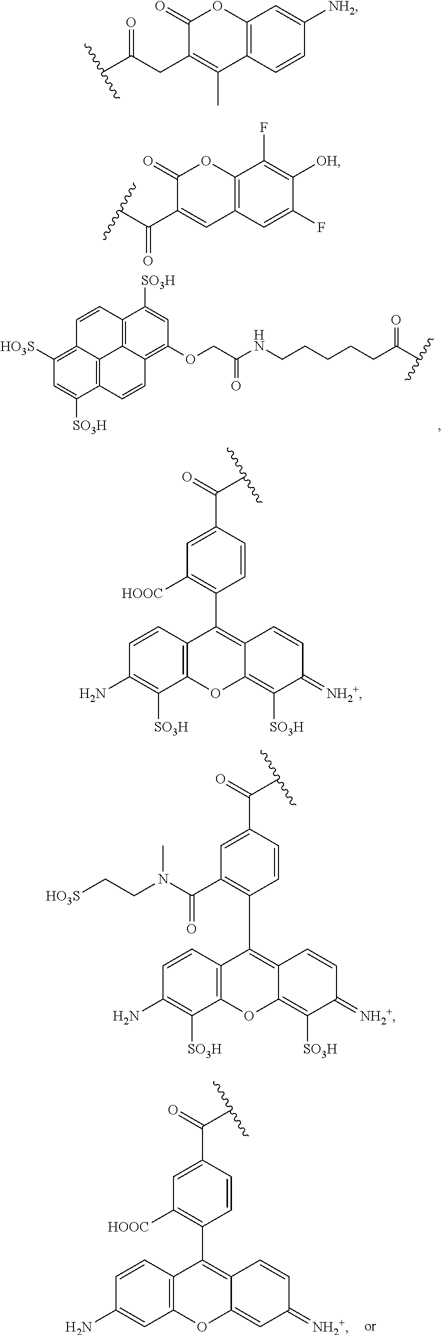

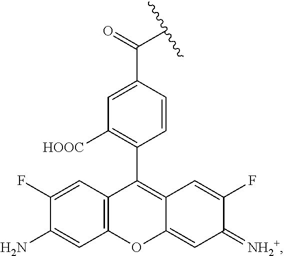

### Additional Embodiments

Embodiment 1. A method of sequencing a template polynucleotide, said method comprising: a) contacting a first primer hybridized to a first template polynucleotide with a first sequencing nucleotide comprising a first reversible terminator moiety and a first detectable label moiety covalently bound to the first sequencing nucleotide via a first cleavable linker, incorporating the first sequencing nucleotide into the first primer with a polymerase, thereby forming a first extended primer polynucleotide, and detecting the first sequencing nucleotide; b) contacting a second primer hybridized to a second template polynucleotide with a first chase nucleotide comprising a first retarding moiety covalently bound to the first chase nucleotide via a first chase cleavable linker; and incorporating the first chase nucleotide into the second primer with a polymerase, thereby forming a second extended primer polynucleotide; c) removing the first reversible terminator moiety, the first detectable label moiety, and the first retarding moiety; and d) contacting the first extended primer polynucleotide with a second sequencing nucleotide comprising a second reversible terminator moiety and a second detectable label moiety covalently bound to the second nucleotide via a second cleavable linker, incorporating the second sequencing nucleotide into the first extended primer polynucleotide with a polymerase, thereby extending the first extended primer polynucleotide, and detecting the second sequencing nucleotide.

Embodiment 2. The method of Embodiment 1, further comprising: e) contacting a third primer hybridized to a third template polynucleotide with a second chase nucleotide comprising a second retarding moiety covalently bound to the second chase nucleotide via a second chase cleavable linker; and incorporating the second chase nucleotide into the third primer with a polymerase.

Embodiment 3. The method of Embodiment 1 or Embodiment 2, wherein the first sequencing nucleotide and the first chase nucleotide comprise the same nucleobase.

Embodiment 4. The method of any one of Embodiments 1 to 3, wherein the first template polynucleotide and second template polynucleotide comprise the same sequence.

Embodiment 5. The method of any one of Embodiments 1 to 4, further comprising removing any unbound first sequencing nucleotide, second sequencing nucleotide, first chase nucleotide, or second chase nucleotide.

Embodiment 6. The method of any one of Embodiments 1 to 5, wherein the first chase nucleotide further comprises a first chase reversible terminator moiety.

Embodiment 7. The method of any one of Embodiments 2 to 6, wherein the second chase nucleotide further comprises a second chase reversible terminator moiety.

Embodiment 8. The method of any one of Embodiments 1 to 7, wherein the first sequencing nucleotide has the formula:

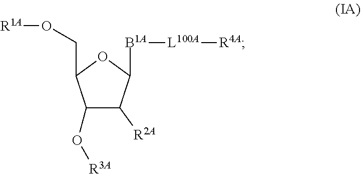

wherein, B^{1A} is a nucleobase; R^{1A} is a triphosphate or thiotriphosphate; R^{2A} is hydrogen or —OH; R^{3A} is the first reversible terminator moiety; R^{4A} is the first detectable label moiety; and L^{100A} is the first cleavable linker.

Embodiment 9. The method of any one of Embodiments 1 to 8, wherein the second sequencing nucleotide has the formula:

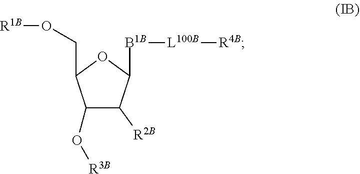

wherein, B^{1B} is a nucleobase; R^{1B} is a triphosphate or thiotriphosphate; R^{2B} is hydrogen or —OH; R^{3B} is the second reversible terminator moiety; R^{4B} is the second detectable label moiety; and L^{100B} is the second cleavable linker.

Embodiment 10. The method of any one of Embodiments 1 to 9, wherein the first chase nucleotide has the formula:

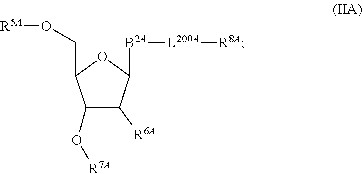

wherein, B^{2A} is a nucleobase; R^{2A} is a triphosphate or thiotriphosphate; R^{6A} is hydrogen or —OH; R^{7A} is the first chase reversible terminator moiety; R^{8A} is the first retarding moiety; and L^{200A} is the first chase cleavable linker.

Embodiment 11. The method of any one of Embodiments 2 to 10, wherein the second chase nucleotide has the formula:

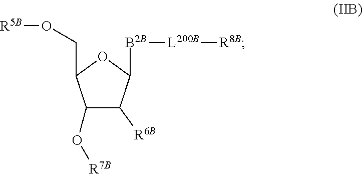

wherein, B^{2B} is a nucleobase; R^{5B} is a triphosphate or thiotriphosphate; R^{6B} is hydrogen or —OH; R^{7B} is the second chase reversible terminator moiety; R^{8B} is the second retarding moiety; and L^{200B} is the second chase cleavable linker.

Embodiment 12. The method of any one of Embodiments 1 to 11, wherein the first detectable label moiety or the second detectable label moiety is a fluorophore.

Embodiment 13. The method of any one of Embodiments 1 to 12, wherein detecting the first sequencing nucleotide or the second sequencing nucleotide comprises directing an excitation beam at the fluorophore and generating a fluorescent emission that is detected by a sensor array.

Embodiment 14. The method of any one of Embodiments 1 to 12, wherein detecting the first sequencing nucleotide or the second sequencing nucleotide comprises exciting the fluorophore with an excitation beam at an excitation wavelength and detecting an emission beam at an emission wavelength.

Embodiment 15. The method of Embodiment 14, wherein the first retarding moiety is capable of being detected at a wavelength less than the excitation wavelength.

Embodiment 16. The method of any one of Embodiments 1 to 15, wherein the first retarding moiety is a first chase detectable label moiety, and wherein the maximum emission of the first retarding moiety does not overlap with the maximum emission of the first detectable label moiety or the second detectable label moiety.

Embodiment 17. The method of Embodiment 16, wherein the maximum emission of the first retarding moiety is at least 20 nm below or above the maximum emission of the first detectable label moiety or second detectable label moiety.

Embodiment 18. The method of any one of Embodiments 1 to 17, wherein the first retarding moiety is non-fluorescent.

Embodiment 19. The method of any one of Embodiments 1 to 18, wherein the first retarding moiety is not detected.

Embodiment 20. The method of any one of Embodiments 2 to 19, wherein the second retarding moiety is a second chase detectable label moiety, and wherein the maximum emission of the second retarding moiety does not overlap with the maximum emission of the first detectable label moiety or the second detectable label moiety.

Embodiment 21. The method of Embodiment 20, wherein the maximum emission of the second retarding moiety is at least 20 nm below or above the maximum emission of the first detectable label moiety or the second detectable label moiety.

Embodiment 22. The method of any one of Embodiments 2 to 21, wherein the second retarding moiety is non-fluorescent.

Embodiment 23. The method of any one of Embodiments 2 to 22, wherein the second retarding moiety is not detected.

Embodiment 24. The method of any one of Embodiments 8 to 23, wherein BIA and B^{1B} are independently a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

Embodiment 25. The method of any one of Embodiments 8 to 23, wherein B^{1A} and B^{1B} are independently

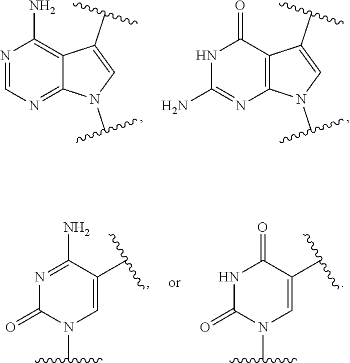 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

Embodiment 26. The method of any one of Embodiments 8 to 23, wherein B^{1A} and B^{1B} are independently

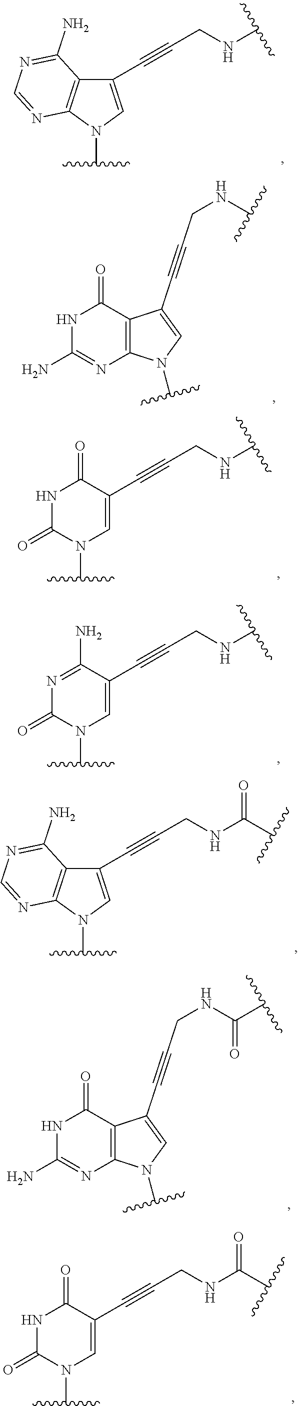

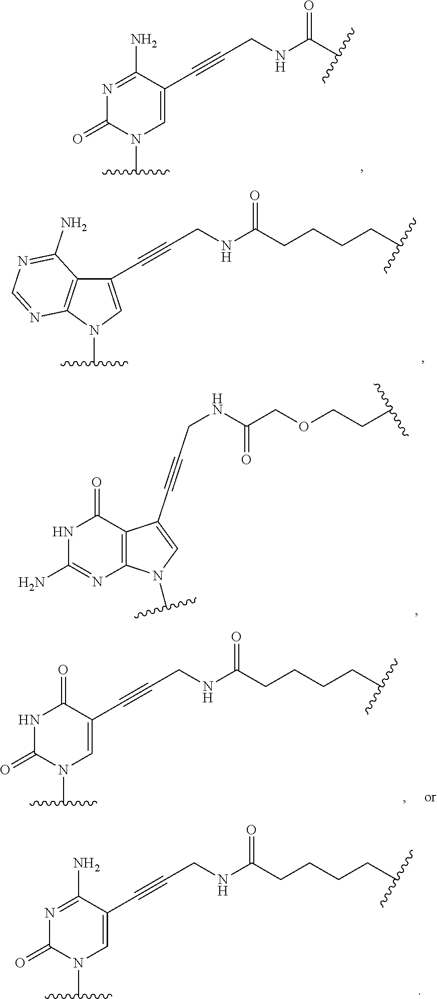 [SMILES: *C(=O)NCC#Cc1cn(*)c(=O)nc1N ; *CCCCC(=O)NCC#Cc1cn(*)c2ncnc(N)c12 ; *CCOCC(=O)NCC#Cc1cn(*)c2nc(N)[nH]c(=O)c12 ; *CCCCC(=O)NCC#Cc1cn(*)c(=O)[nH]c1=O ; *CCCCC(=O)NCC#Cc1cn(*)c(=O)nc1N]

Embodiment 27. The method of any one of Embodiments 10 to 26, wherein B^{2A} and B^{2B} are independently a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

Embodiment 28. The method of any one of Embodiments 10 to 26, wherein B^{2A} and B²B are independently

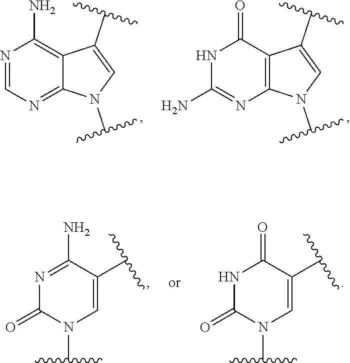 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

Embodiment 29. The method of any one of Embodiments 10 to 26, wherein B^{2A} and B^{2B} are independently

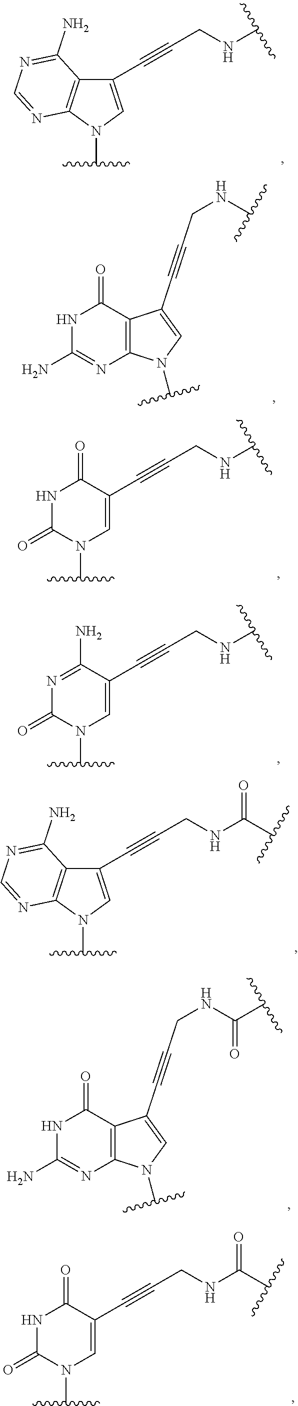

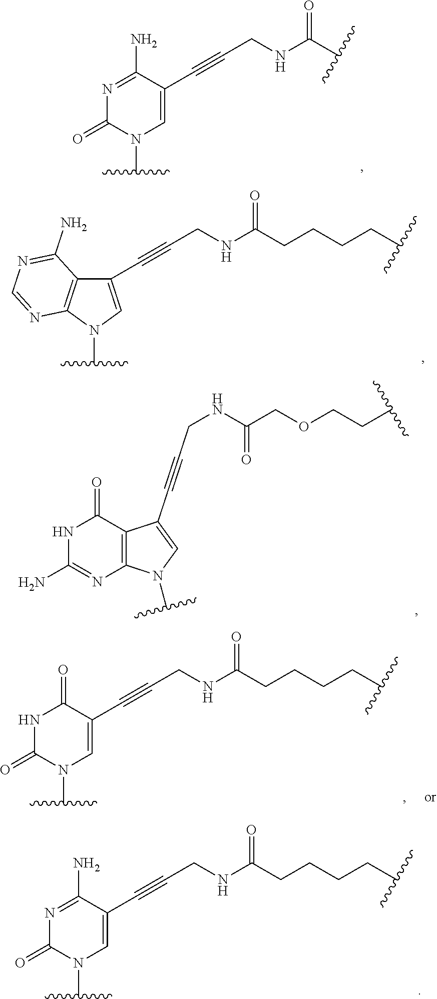 [SMILES: *C(=O)NCC#Cc1cn(*)c(=O)nc1N ; *CCCCC(=O)NCC#Cc1cn(*)c2ncnc(N)c12 ; *CCOCC(=O)NCC#Cc1cn(*)c2nc(N)[nH]c(=O)c12 ; *CCCCC(=O)NCC#Cc1cn(*)c(=O)[nH]c1=O ; *CCCCC(=O)NCC#Cc1cn(*)c(=O)nc1N]

Embodiment 30. The method of any one of Embodiments 8 to 29, wherein L^{100A} and L^{100B} independently comprise:

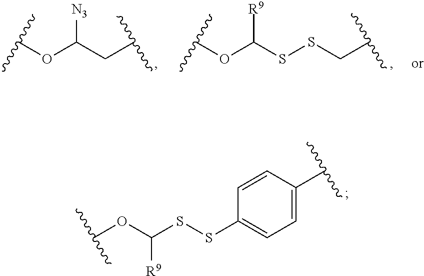

wherein R⁹ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 31. The method of any one of Embodiments 8 to 30, wherein L^{100A} and L^{100B} independently comprise:

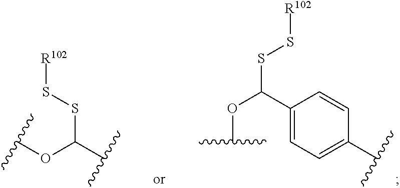

wherein R¹⁰² is independently unsubstituted C₁-C₄ alkyl.

Embodiment 32. The method of any one of Embodiments 10 to 31, wherein L^{200A} and L^{200B} independently comprise:

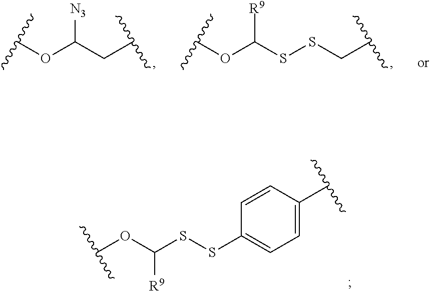

wherein R⁹ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 33. The method of any one of Embodiments 10 to 31, wherein L^{200A} and L^{200B} independently comprise:

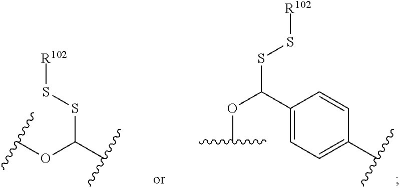

wherein R¹⁰² is independently unsubstituted C₁-C₄ alkyl.

Embodiment 34. The method of any one of Embodiments 1 to 33, comprising detecting the first sequencing nucleotide before step b) or after step b).

Embodiment 35. The method of any one of Embodiments 1 to 34, further comprising detecting the first sequencing nucleotide during step b).

Embodiment 36. The method of any one of Embodiments 1 to 35, further comprising repeating a cycle of step a), step b), and step c) for 1 to 200 cycles.

Embodiment 37. The method of any one of Embodiments 1 to 36, wherein the first retarding moiety is

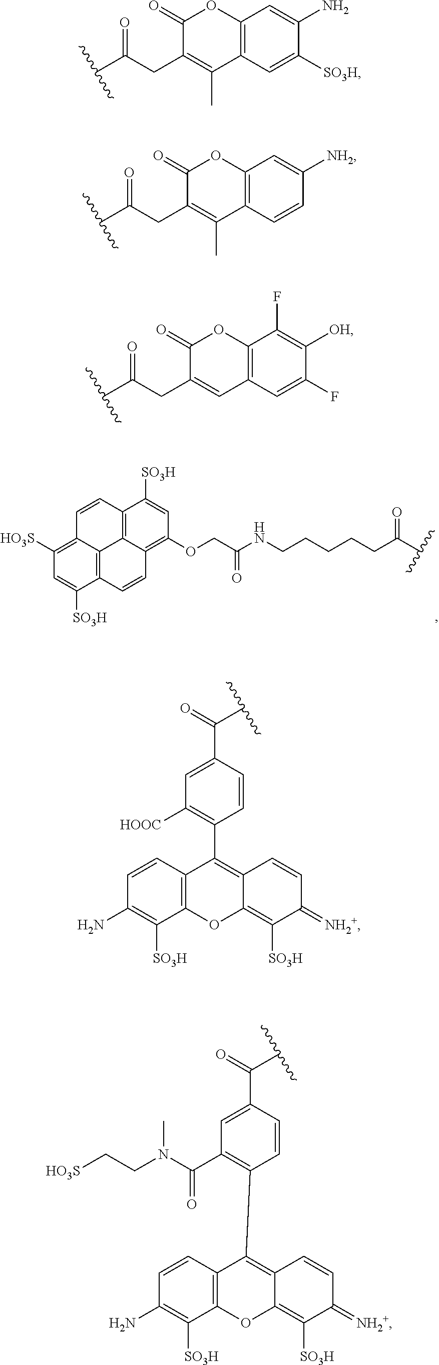

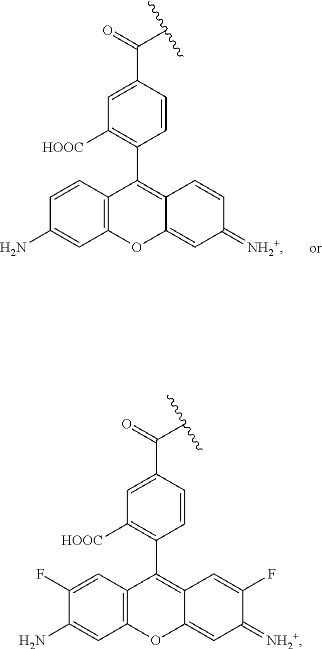

Embodiment 38. The method of any one of Embodiments 2 to 37, wherein the second retarding moiety is

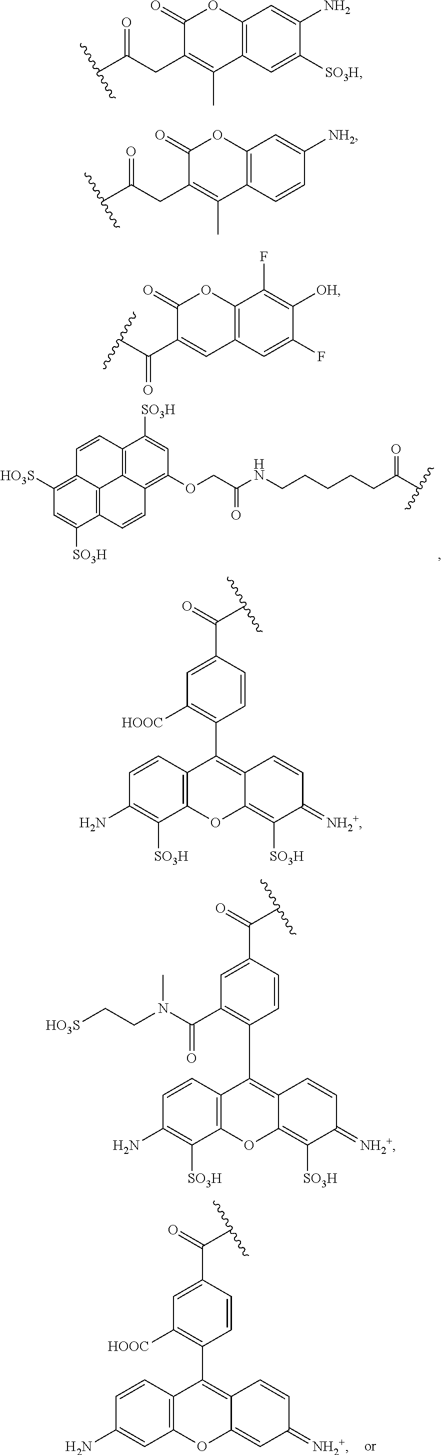

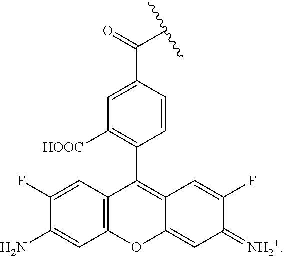

Embodiment 39. A method of detecting an incorporated sequencing nucleotide, said method comprising: i) contacting a solid support comprising a plurality of template polynucleotides with a plurality of chase nucleotides, wherein each chase nucleotide comprises a retarding moiety covalently bound to the chase nucleotide via a cleavable linker, and wherein a first fraction of the plurality of template polynucleotides are hybridized to an unblocked primer; and a second fraction of the plurality of template polynucleotides are hybridized to a blocked primer, wherein the blocked primer comprises the incorporated sequencing nucleotide at a 3′ end of the blocked primer; ii) incorporating one of said chase nucleotides into said unblocked primer with a polymerase; and iii) detecting the incorporated sequencing nucleotide.

Embodiment 40. A kit comprising a sequencing solution and a chase solution, wherein (a) the sequencing solution comprises a plurality of sequencing nucleotides, wherein each sequencing nucleotide of the plurality of sequencing nucleotides comprises a detectable label moiety and a reversible terminator; and (b) the chase solution comprises a plurality of chase nucleotides, wherein each chase nucleotide of the plurality of chase nucleotides comprises a retarding moiety and a reversible terminator.

Embodiment 41. The kit of Embodiment 40, wherein the sequencing solution comprises: (i) a plurality of adenine nucleotides, or analogs thereof; (ii) a plurality of thymine nucleotides, or analogs thereof, or a plurality of uracil nucleotides, or analogs thereof; (iii) a plurality of cytosine nucleotides, or analogs thereof, and (iv) a plurality of guanine nucleotides, or analogs thereof.

Embodiment 42. The kit of Embodiment 40 or Embodiment 41, wherein (i) each nucleotide of the plurality of adenine nucleotides, or analogs thereof comprises a first detectable label; (ii) each nucleotide of a plurality of thymine nucleotides, or analogs thereof, or a plurality of uracil nucleotides, or analogs thereof, comprises a second detectable label moiety; (iii) each nucleotide of a plurality of cytosine nucleotides, or analogs thereof, of the plurality comprises a third detectable label moiety; and (iv) each nucleotide of a plurality of guanine nucleotides, or analogs thereof, comprises a fourth detectable label moiety, and the detectable label moieties are different.

Embodiment 43. The kit of any one of Embodiments 40 to 42, wherein the chase solution comprises: (i) a plurality of adenine nucleotides, or analogs thereof; (ii) a plurality of thymine nucleotides, or analogs thereof, or a plurality of uracil nucleotides, or analogs thereof; (iii) a plurality of cytosine nucleotides, or analogs thereof, and (iv) a plurality of guanine nucleotides, or analogs thereof.

Embodiment 44. The kit of any one of Embodiments 40 to 43, wherein each of the chase nucleotides comprises the same retarding moiety.

Embodiment 45. The kit of any one of Embodiments 40 to 44, wherein one or more of the chase nucleotides and/or one or more of the sequencing nucleotides comprises a nucleotide with a free 3′-OH.

Embodiment 46. The kit of any one of Embodiments 40 to 45, further comprising one or more depletion polynucleotides and i) a depletion polymerase that is active to selectively incorporate the nucleotides comprising a free 3′-OH; or (ii) one or more nucleotide cyclases active to selectively cyclize the nucleotides comprising a free 3′-OH.

1. A method of sequencing a template polynucleotide, said method comprising:
a) contacting a first primer hybridized to a first template polynucleotide with a first sequencing nucleotide comprising a first reversible terminator moiety and a first detectable label moiety covalently bound to the first sequencing nucleotide via a first cleavable linker, incorporating the first sequencing nucleotide into the first primer with a polymerase, thereby forming a first extended primer polynucleotide, and detecting the first sequencing nucleotide;
b) contacting a second primer hybridized to a second template polynucleotide with a first chase nucleotide comprising a first retarding moiety covalently bound to the first chase nucleotide via a first chase cleavable linker; and incorporating the chase nucleotide into the second primer with a polymerase, thereby forming a second extended primer polynucleotide;
c) removing the first reversible terminator moiety, the first detectable label moiety, and the first retarding moiety; and
d) contacting the first extended primer polynucleotide with a second sequencing nucleotide comprising a second reversible terminator moiety and a second detectable label moiety covalently bound to the second nucleotide via a second cleavable linker, incorporating the second sequencing nucleotide into the first extended primer polynucleotide with a polymerase, thereby extending the first extended primer polynucleotide, and detecting the second sequencing nucleotide.

2. The method of claim 1, further comprising:
e) contacting a third primer hybridized to a third template polynucleotide with a second chase nucleotide comprising a second retarding moiety covalently bound to the second chase nucleotide via a second chase cleavable linker; and incorporating the second chase nucleotide into the third primer with a polymerase.

3. The method of claim 1, wherein the first sequencing nucleotide and the first chase nucleotide comprise the same nucleobase.

4. The method of claim 1, wherein the first template polynucleotide and second template polynucleotide comprise the same sequence.

5. The method of claim 1, further comprising removing any unbound first sequencing nucleotide, second sequencing nucleotide, first chase nucleotide, or second chase nucleotide.

6. The method of claim 1, wherein the first chase nucleotide further comprises a first chase reversible terminator moiety.

7. The method of claim 2, wherein the second chase nucleotide further comprises a second chase reversible terminator moiety.

8. The method of claim 1, wherein the first sequencing nucleotide has the formula: 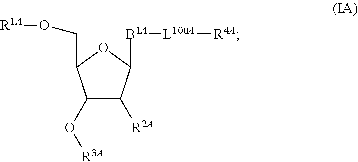
wherein,
B^{1A} is a nucleobase;
R^{1A} is a triphosphate or thiotriphosphate;
R^{2A} is hydrogen or —OH;
R^{3A} is the first reversible terminator moiety;
R^{4A} is the first detectable label moiety; and
L^{100A} is the first cleavable linker; and
wherein the second sequencing nucleotide has the formula: 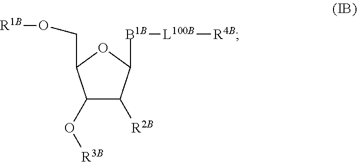
wherein,
B^{1B} is a nucleobase;
R^{1B} is a triphosphate or thiotriphosphate;
R^{2B} is hydrogen or —OH;
R^{3B} is the second reversible terminator moiety;
R^{4B} is the second detectable label moiety; and
L^{100B} is the second cleavable linker.

9. (canceled)

10. The method of claim 1, wherein the first chase nucleotide has the formula: 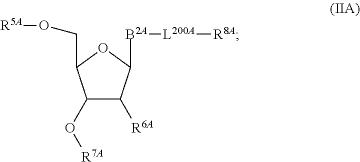
wherein,
B^{2A} is a nucleobase;
R^{5A} is a triphosphate or thiotriphosphate;
R^{6A} is hydrogen or —OH;
R^{7A} is the first chase reversible terminator moiety;
R^{8A} is the first retarding moiety; and
L^{200A} is the first chase cleavable linker.

11. (canceled)

12. The method of claim 1, wherein the first detectable label moiety or the second detectable label moiety is a fluorophore.

13. The method of claim 1, wherein detecting the first sequencing nucleotide or the second sequencing nucleotide comprises directing an excitation beam at the fluorophore and generating a fluorescent emission that is detected by a sensor array.

14. The method of claim 12, wherein detecting the first sequencing nucleotide or the second sequencing nucleotide comprises exciting the fluorophore with an excitation beam at an excitation wavelength and detecting an emission beam at an emission wavelength.

15. The method of claim 14, wherein the first retarding moiety is capable of being detected at a wavelength less than the excitation wavelength.

16. The method of claim 1, wherein the first retarding moiety is a first chase detectable label moiety, and wherein the maximum emission of the first retarding moiety does not overlap with the maximum emission of the first detectable label moiety or the second detectable label moiety.

17. The method of claim 16, wherein the maximum emission of the first retarding moiety is at least 20 nm below or above the maximum emission of the first detectable label moiety or the second detectable label moiety.

18. The method of claim 1, wherein the first retarding moiety is non-fluorescent.

19. The method of claim 1, wherein the first retarding moiety is not detected.

20. The method of claim 2, wherein the second retarding moiety is a second chase detectable label moiety, and wherein the maximum emission of the second retarding moiety does not overlap with the maximum emission of the first detectable label moiety or the second detectable label moiety.

21. The method of claim 20, wherein the maximum emission of the second retarding moiety is at least 20 nm below or above the maximum emission of the first detectable label moiety or the second detectable label moiety.

22. The method of claim 2, wherein the second retarding moiety is non-fluorescent.

23. The method of claim 2, wherein the second retarding moiety is not detected.

24. The method of claim 8, wherein B^{1A} and B^{1B} are independently a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

25. The method of claim 8, wherein B^{1A} and B^{1B} are independently

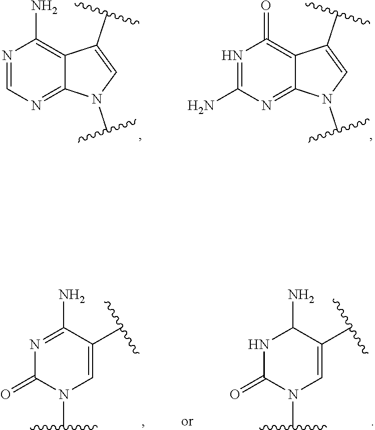

26. The method of claim 8, wherein B^{1A} and B^{1B} are independently

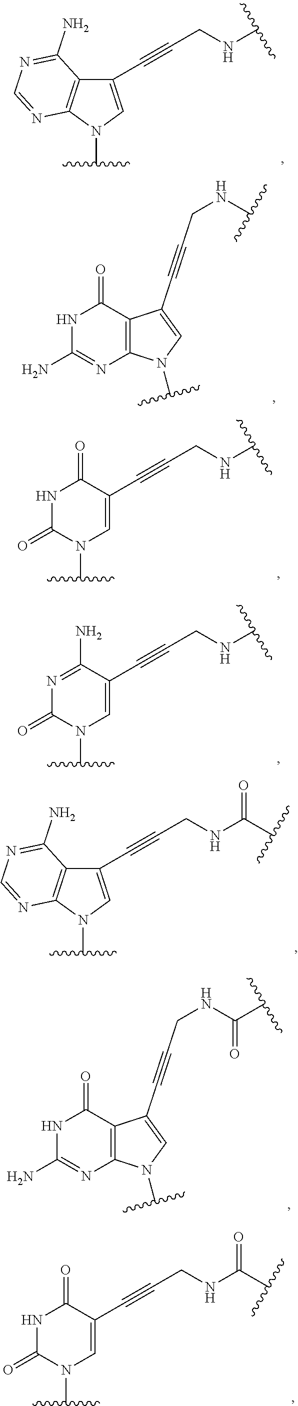

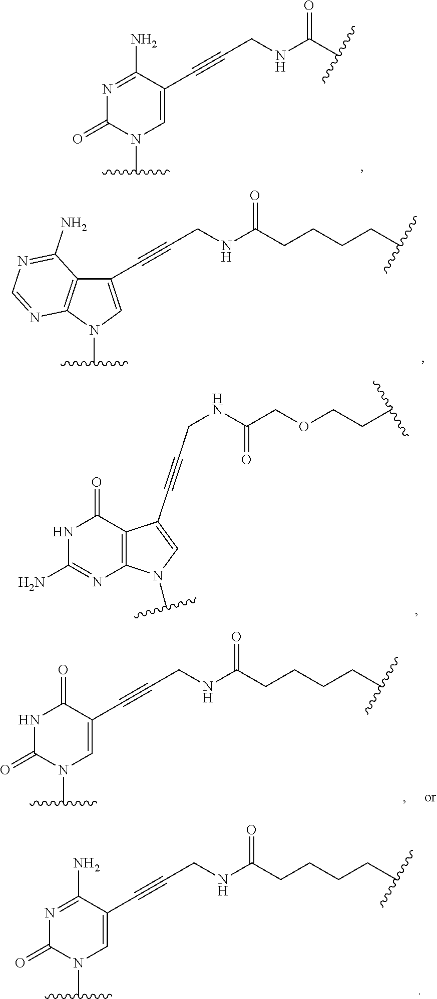 [SMILES: *C(=O)NCC#Cc1cn(*)c(=O)nc1N ; *CCCCC(=O)NCC#Cc1cn(*)c2ncnc(N)c12 ; *CCOCC(=O)NCC#Cc1cn(*)c2nc(N)[nH]c(=O)c12 ; *CCCCC(=O)NCC#Cc1cn(*)c(=O)[nH]c1=O ; *CCCCC(=O)NCC#Cc1cn(*)c(=O)nc1N]

27. The method of claim 10, wherein B^{2A} is a divalent cytosine or a derivative thereof, a divalent guanine or a derivative thereof, a divalent adenine or a derivative thereof, a divalent thymine or a derivative thereof, a divalent uracil or a derivative thereof, a divalent hypoxanthine or a derivative thereof, a divalent xanthine or a derivative thereof, a divalent 7-methylguanine or a derivative thereof, a divalent 5,6-dihydrouracil or a derivative thereof, a divalent 5-methylcytosine or a derivative thereof, or a divalent 5-hydroxymethylcytosine or a derivative thereof.

28. The method of claim 10, wherein B^{2A} is

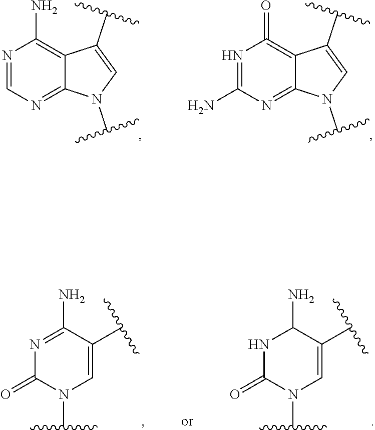

29. The method of claim 10, wherein B^{2A} is

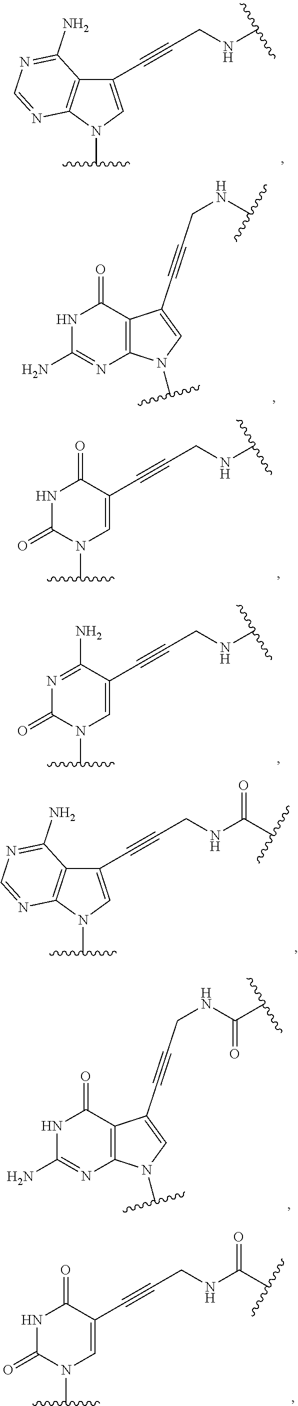

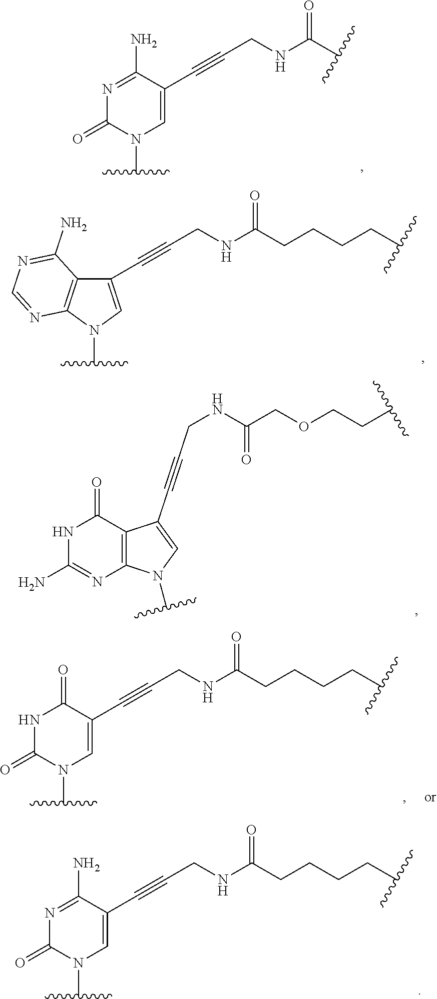 [SMILES: *C(=O)NCC#Cc1cn(*)c(=O)nc1N ; *CCCCC(=O)NCC#Cc1cn(*)c2ncnc(N)c12 ; *CCOCC(=O)NCC#Cc1cn(*)c2nc(N)[nH]c(=O)c12 ; *CCCCC(=O)NCC#Cc1cn(*)c(=O)[nH]c1=O ; *CCCCC(=O)NCC#Cc1cn(*)c(=O)nc1N]

30. The method of claim 8, wherein L^{100A} and L^{100B} independently comprise: 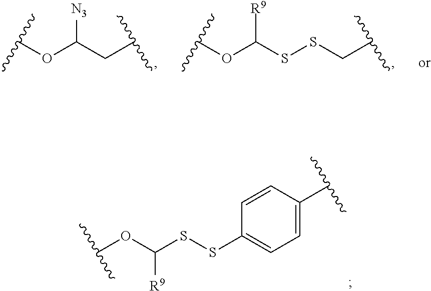
wherein R⁹ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

31. The method of claim 8, wherein L^{100A} and L^{100B} independently comprise: 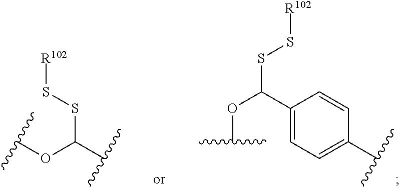
wherein R¹⁰² is independently unsubstituted C₁-C₄ alkyl.

32. The method of claim 10, wherein L^{200A} comprises: 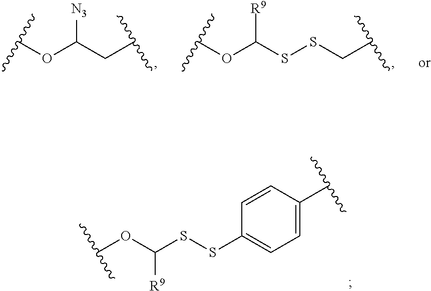
wherein R⁹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

33. The method of claim 10, wherein L^{200A} comprises: 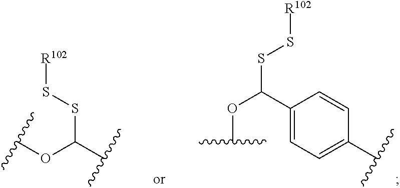
wherein R¹⁰² is unsubstituted C₁-C₄ alkyl.

34. The method of claim 1, comprising detecting the first sequencing nucleotide before step b) or after step b).

35. The method of claim 1, further comprising detecting the first sequencing nucleotide during step b).

36. The method of claim 1, further comprising repeating a cycle of step a), step b), and step c) for 1 to 200 cycles.

37. The method of claim 1, wherein the first retarding moiety is

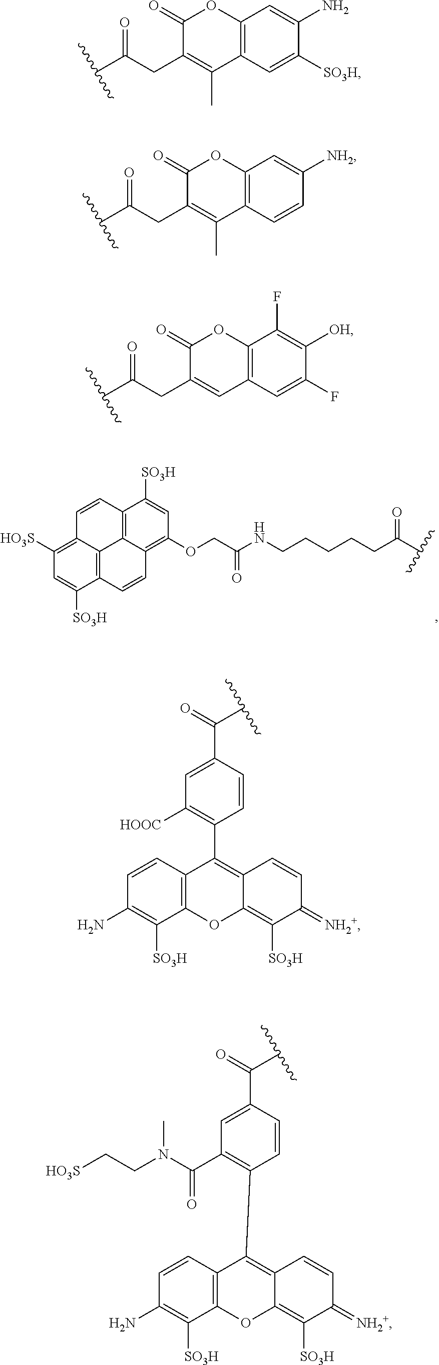

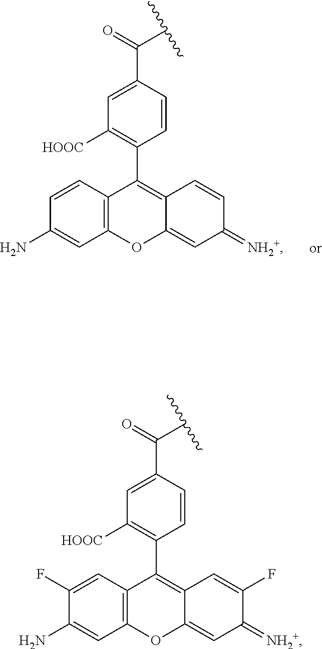

38. The method of claim 2, wherein the second retarding moiety is

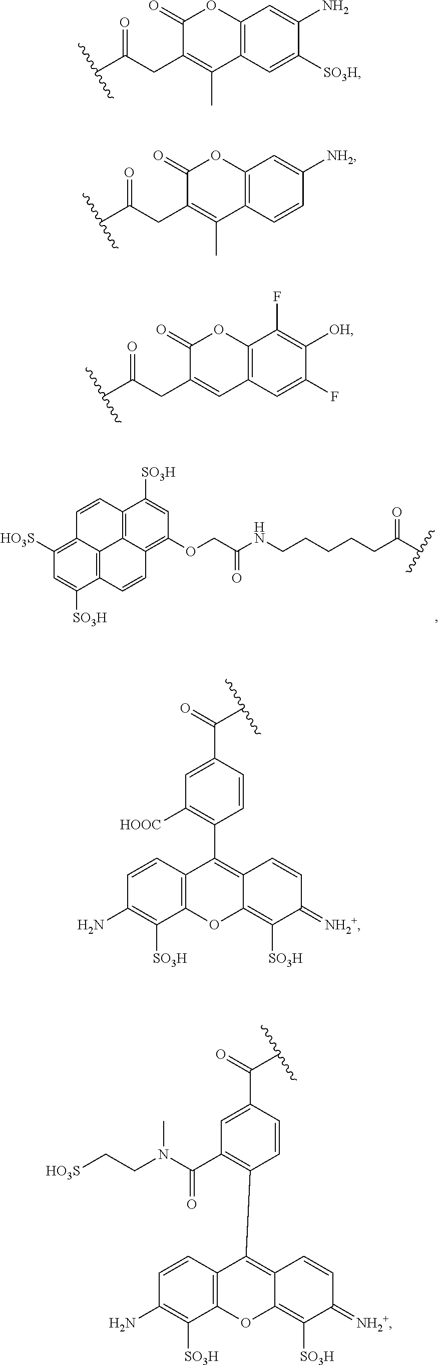

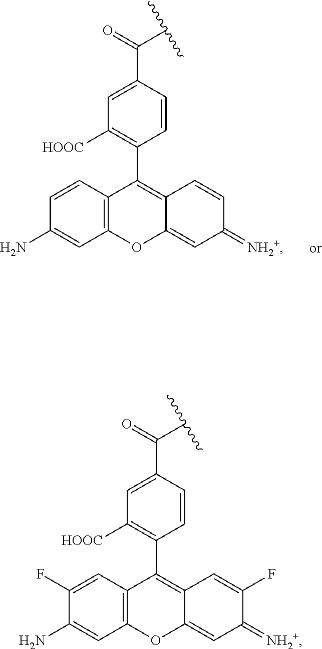

39. A method of detecting an incorporated sequencing nucleotide, said method comprising:
i) contacting a solid support comprising a plurality of template polynucleotides with a plurality of chase nucleotides, wherein each chase nucleotide comprises a retarding moiety covalently bound to the chase nucleotide via a cleavable linker, and wherein a first fraction of the plurality of template polynucleotides are hybridized to an unblocked primer; and a second fraction of the plurality of template polynucleotides are hybridized to a blocked primer, wherein the blocked primer comprises the incorporated sequencing nucleotide at a 3′ end of the blocked primer;
ii) incorporating one of said chase nucleotides into said unblocked primer with a polymerase; and
iii) detecting the incorporated sequencing nucleotide.

40. A kit comprising a sequencing solution and a chase solution, wherein
(a) the sequencing solution comprises a plurality of sequencing nucleotides, wherein each sequencing nucleotide of the plurality of sequencing nucleotides comprises a detectable label moiety and a reversible terminator; and
(b) the chase solution comprises a plurality of chase nucleotides, wherein each chase nucleotide of the plurality of chase nucleotides comprises a retarding moiety and a reversible terminator.

41. The kit of claim 40, wherein the sequencing solution comprises:
(i) a plurality of adenine nucleotides, or analogs thereof;
(ii) a plurality of thymine nucleotides, or analogs thereof, or a plurality of uracil nucleotides, or analogs thereof;
(iii) a plurality of cytosine nucleotides, or analogs thereof; and
(iv) a plurality of guanine nucleotides, or analogs thereof.

42. The kit of claim 41, wherein
(i) each nucleotide of the plurality of adenine nucleotides, or analogs thereof comprises a first detectable label;
(ii) each nucleotide of a plurality of thymine nucleotides, or analogs thereof, or a plurality of uracil nucleotides, or analogs thereof, comprises a second detectable label moiety;
(iii) each nucleotide of a plurality of cytosine nucleotides, or analogs thereof, of the plurality comprises a third detectable label moiety; and
(iv) each nucleotide of a plurality of guanine nucleotides, or analogs thereof, comprises a fourth detectable label moiety, and the detectable label moieties are different.

43. The kit of claim 40, wherein the chase solution comprises:
(i) a plurality of adenine nucleotides, or analogs thereof;
(ii) a plurality of thymine nucleotides, or analogs thereof, or a plurality of uracil nucleotides, or analogs thereof,
(iii) a plurality of cytosine nucleotides, or analogs thereof; and
(iv) a plurality of guanine nucleotides, or analogs thereof.

44. The kit of claim 40, wherein each of the chase nucleotides comprises the same retarding moiety.

45. The kit of claim 40, wherein one or more of the chase nucleotides and/or one or more of the sequencing nucleotides comprises a nucleotide with a free 3′-OH.

46. The kit of claim 40, further comprising one or more depletion polynucleotides and i) a depletion polymerase that is active to selectively incorporate the nucleotides comprising a free 3′-OH; or (ii) one or more nucleotide cyclases active to selectively cyclize the nucleotides comprising a free 3′-OH.
